(19) United States
(12) Patent Application Publication
(10) Pub. No.: US 2005/0186677 A1
(43) Pub. Date: Aug. 25, 2005

(54) Novel mutated mammalian cells and animals

(76) Inventors: Glenn Friedrich, Houston, TX (US); Brian Zambrowicz, The Woodlands, TX (US); Arthur T. Sands, The Woodlands, TX (US)

Correspondence Address: Lance K. Ishimoto;Lexicon Genetics Incorporated, 8800 Technology Forest Place, The Woodlands, TX, 77381, US

(21) Appl. No.: 10/964,549

(22) Filed: Oct. 13, 2004

Related U.S. Application Data
(63) Continuation of application No. 09/750,456, filed on Dec. 28, 2000, now abandoned.

(51) Int. Cl. C12N 5/08; C12N 15/86; C12N 5/06

(52) U.S. Cl. 435/456; 435/366; 435/354

(57) ABSTRACT

Novel mutated mammalian cells are provided that have been characterized by identifying the sequence of the genes that have been mutated. Preferably, novel mutated cells are murine ES cells that stably incorporate retroviral gene trap constructs in the specifically identified genes. The novel mutated cells and animals are useful in functional genomic analysis, and in the discovery and development of new therapeutic and diagnostics agents and methods.

The present application claims priority to U.S. application Ser. No. 09/728,445 which was filed Nov. 30, 2000 which claimed priority to U.S. Provisional Application No. 60/168,358 which was filed Dec. 1, 1999. The present application incorporates U.S. Pat. No. 6,080,576 and U.S. application Ser. Nos. 08/726,867, 08/728,963, 08/907,598, 08/942,806, 60/109,302, and 09/276,533 and their respective disclosures herein by reference in their entirety.

### 1.0. FIELD OF THE INVENTION

The present invention is in the field of molecular genetics. The application discloses novel mutated cells that are generated by process involving the insertion of at least a portion of a genetically engineered viral vector into the chromosome. The specifically disclosed recombinant vector allows for the rapid identification of the gene that has been mutated by using nucleotide or amino acid sequence information to identify the gene that has been mutated by the vector. When mutated embryonic stem cell clones are produced, such cells can be used to produce mutant animals capable of germline transmission of the described mutated genes.

### 2.0. BACKGROUND OF THE INVENTION

Most mammalian genes are divided into exons and introns. Exons are the portions of the gene that are spliced into mRNA and encode the protein product of a gene. In genomic DNA, these coding exons are often divided by noncoding intron sequences. Although RNA polymerase transcribes both intron and exon sequences, the intron sequences must be removed from the transcript so that the resulting mRNA can be translated into protein. Accordingly, all mammalian, and most eukaryotic, cells have the machinery to splice exons to produce mRNA. Gene trap vectors have been designed to insert into the introns of genes in a manner that allows the cellular splicing machinery to splice vector encoded exons to cellular mRNAs. Commonly, gene trap vectors contain selectable marker sequences that are preceded by strong splice acceptor sequences and are not preceded by a promoter. Thus, when such vectors integrate into a gene, the cellular splicing machinery splices exons from the trapped gene onto the 5′ end of the selectable marker sequence. Typically, such selectable marker genes can only be expressed if the vector encoding the gene has integrated into an intron. The resulting gene trap events are subsequently identified by selecting for cells that can survive selective culture.

Gene trapping has generally proven to be an efficient method of mutating large numbers of genes. The insertion of the gene trap vector creates a mutation in the trapped gene, and also provides a molecular tag for ease of identifying the gene that has been trapped. When ROSAβgeo was used to trap genes it was demonstrated that at least 50% of the resulting mutations resulted in a phenotype when examined in mice. This indicates that the gene trap insertion vectors are useful mutagens. Although a powerful tool for mutating genes, the potential of the method has historically been limited by the difficulty in identifying the trapped genes. Methods that have been used to identify trap events rely on the fusion transcripts resulting from the splicing of exon sequences from the trapped gene to sequences encoded by the gene trap vector. Common gene identification protocols used to obtain sequences from these fusion transcripts include 5′ RACE, cDNA cloning, and cloning of genomic DNA surrounding the site of vector integration. However, these methods have proven labor intensive, not readily amenable to automation, and generally impractical for high-throughput.

More recently, vectors have been developed that rely on a new strategy of gene trapping that uses a vector that contains a selectable marker gene preceded by a promoter and followed by a splice donor sequence instead of a polyadenylation sequence. These vectors do not provide selection unless they integrate into a gene and subsequently trap downstream exons which provide a polyadenylation sequence. Integration of such vectors into the chromosome results in the splicing of the selectable marker gene to 3′ exons of the trapped gene. These vectors provide a number of advantages. They can be used to trap genes regardless of whether the genes are normally expressed in the cell type in which the vector has integrated. In addition, cells harboring such vectors can be screened using automated (e.g., 96 -well plate format) gene identification assays such as 3′ RACE (see generally, Frohman, 1994, PCR Methods and Applications, 4:S40-S58). Using these vectors it is possible to produce large numbers of mutations and rapidly identify the mutated, or trapped, gene by DNA sequence analysis.

### 3.0. SUMMARY OF THE INVENTION

The subject invention provides numerous isolated mammalian mutant cell clones that are each characterized by the insertion of a mutagenic genetically engineered polynucleotide sequence into a gene identifiable as corresponding to one or more of the OMNIBANK gene trapped sequences (GTSs) disclosed in Sequence Listing.

The subject invention further contemplates a mutated cell, and particularly a mutated ES cell, and the animals derived from such ES cell that stably maintain a genetically engineered mutation in a gene identifiable as corresponding to one of the disclosed GTSs.

### 4.0. DESCRIPTION OF THE SEQUENCE LISTING AND FIGURES

The Sequence Listing is a compilation of nucleotide sequences obtained by sequencing clonal lines of gene trapped murine ES cells.

FIGS. 1A-1C present a diagrammatic representation of representative gene trap vectors used to generate the described sequences.

FIG. 2 provides an index to the Sequence Listing and the corresponding database accession numbers for the genes that have been mutated according to the present invention.

### 5.0. DETAILED DESCRIPTION OF THE INVENTION

The current invention relates to novel mutated mammalian cells that are each characterized by the insertion of a recombinant (i.e., genetically engineered) mutagenic polynucleotide sequence into a gene identifiable as corresponding to one of the GTSs of SEQ ID NOS: 1-891.

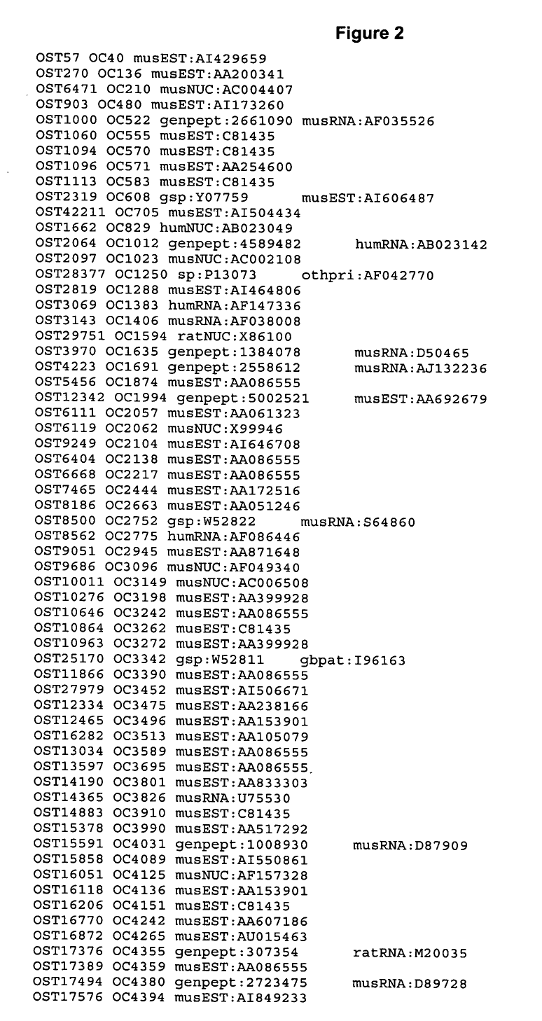

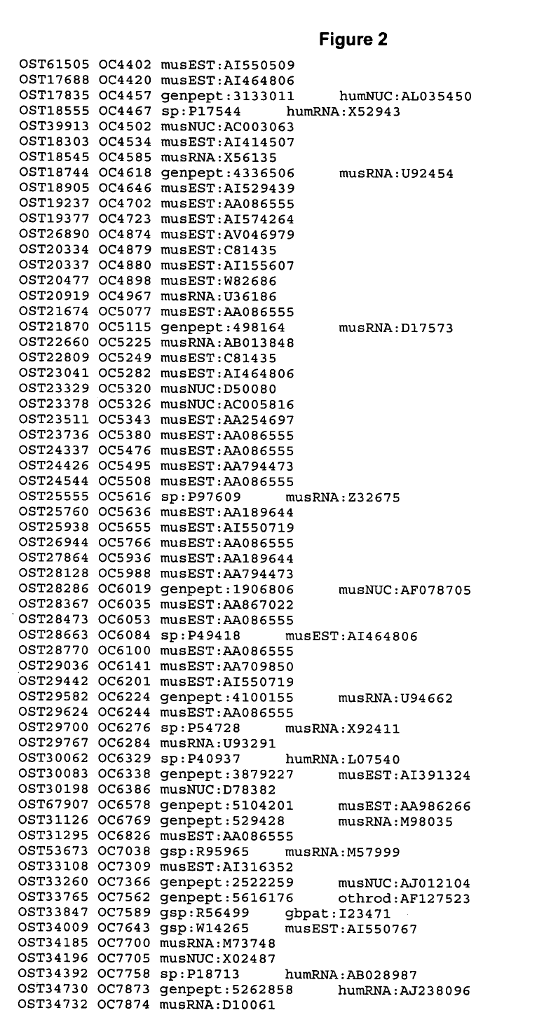

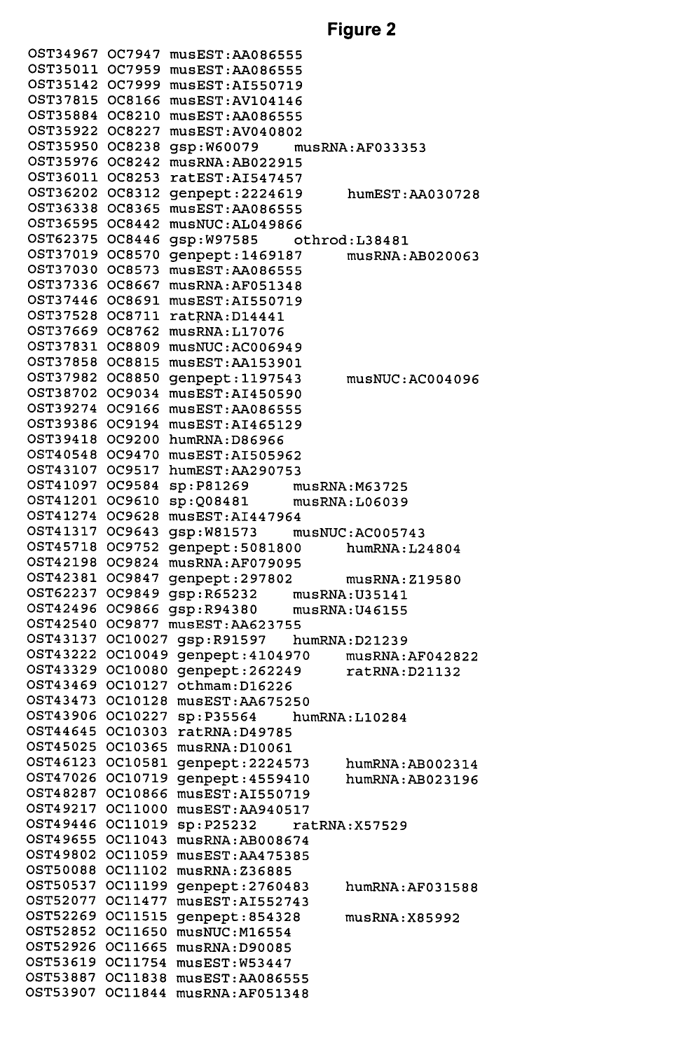

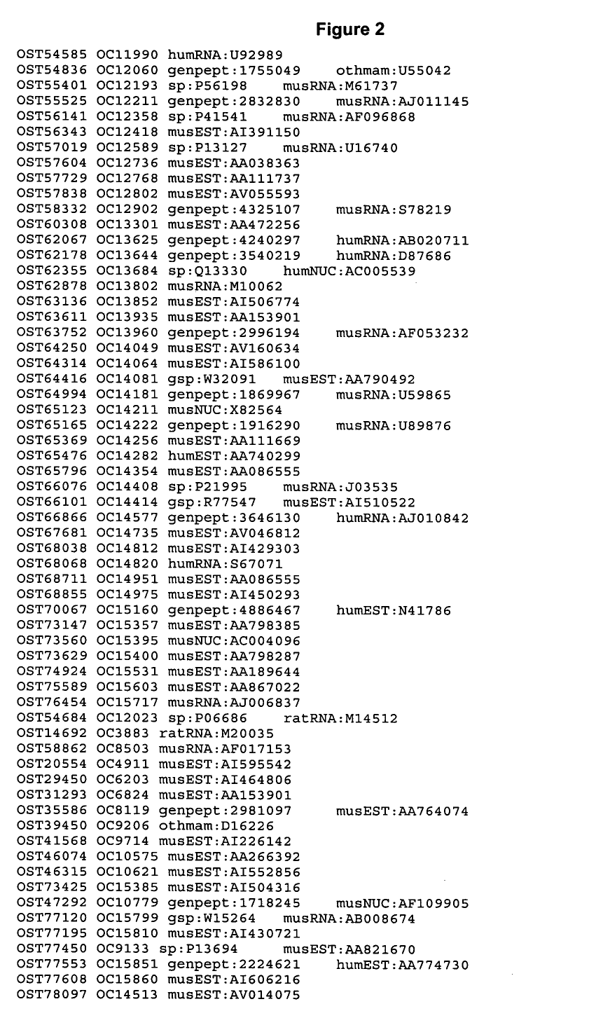

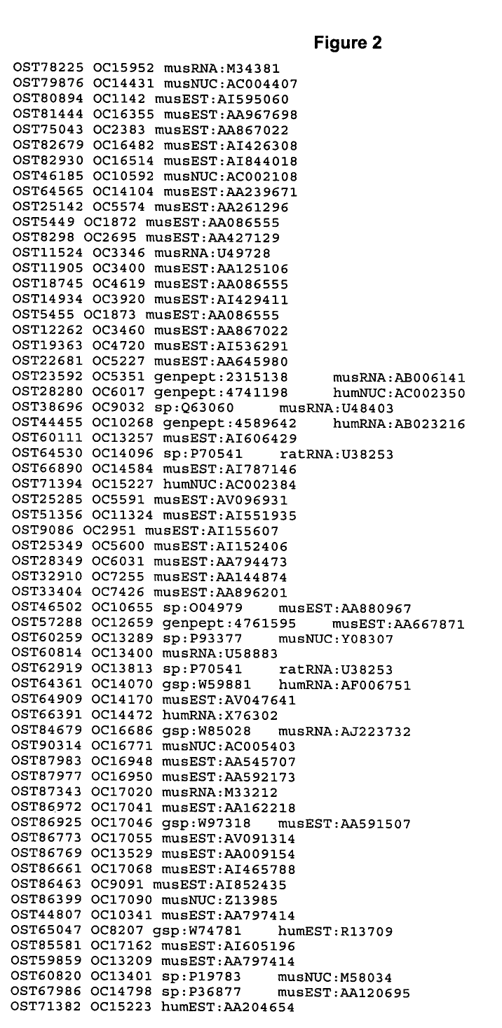

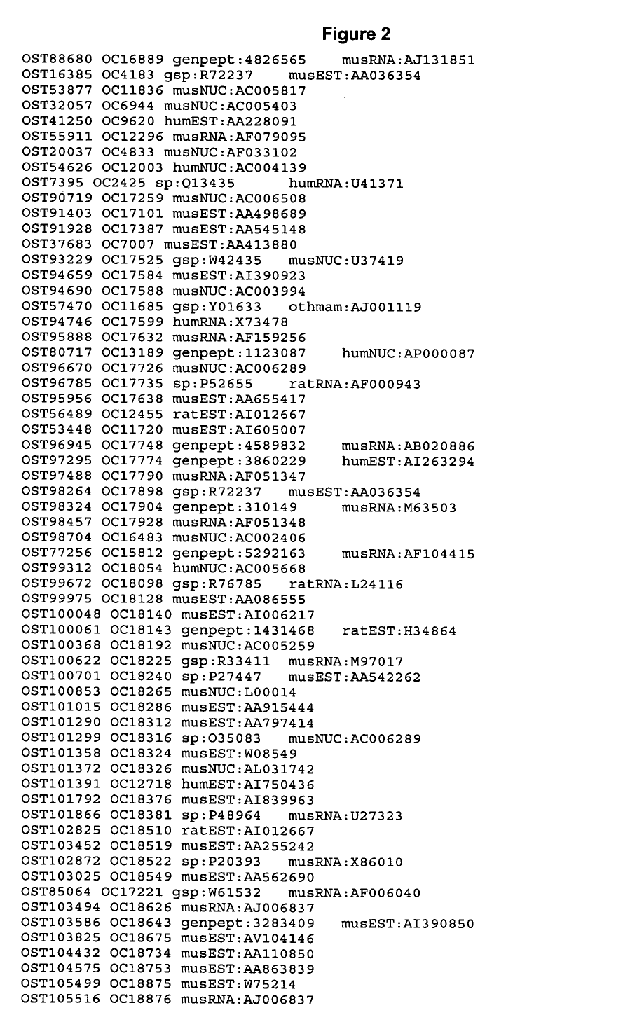

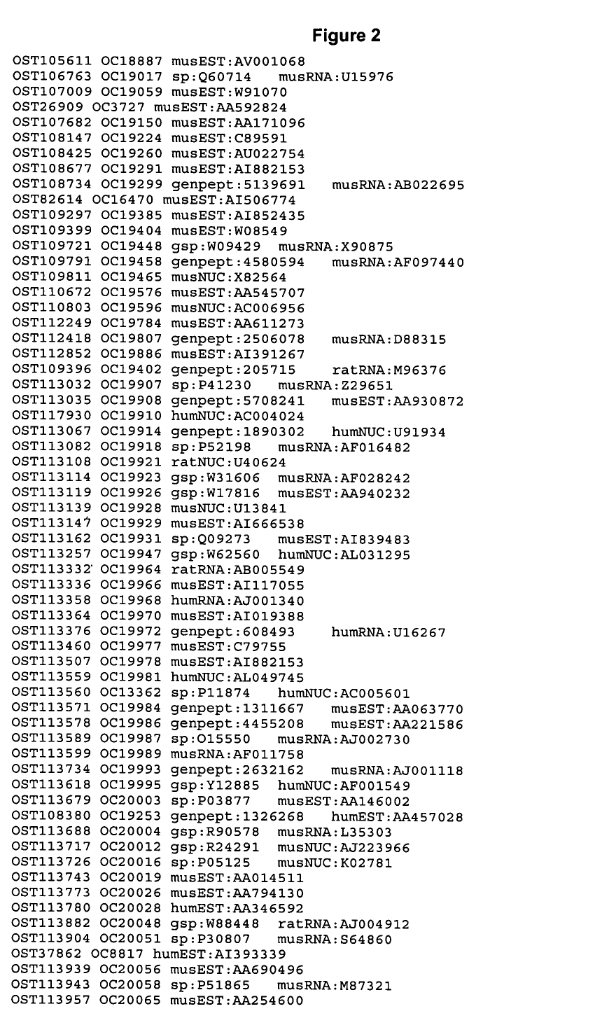

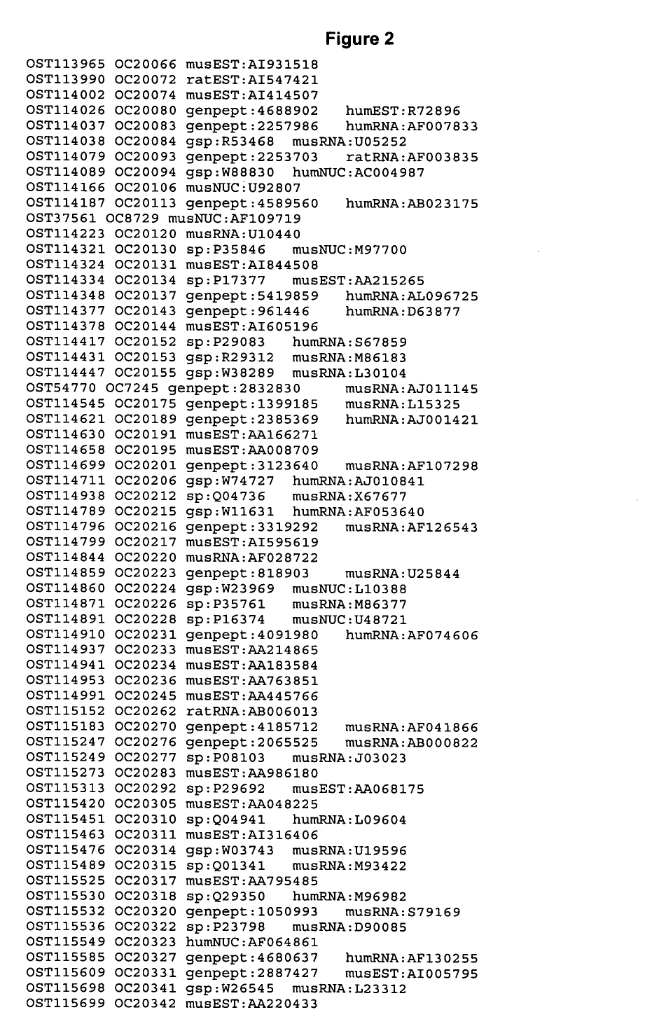

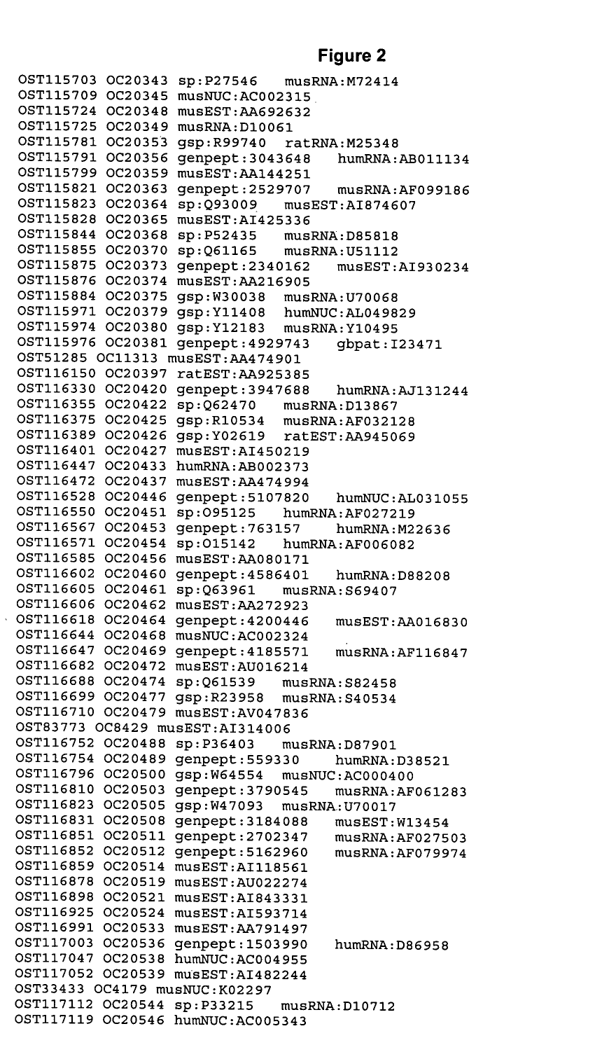

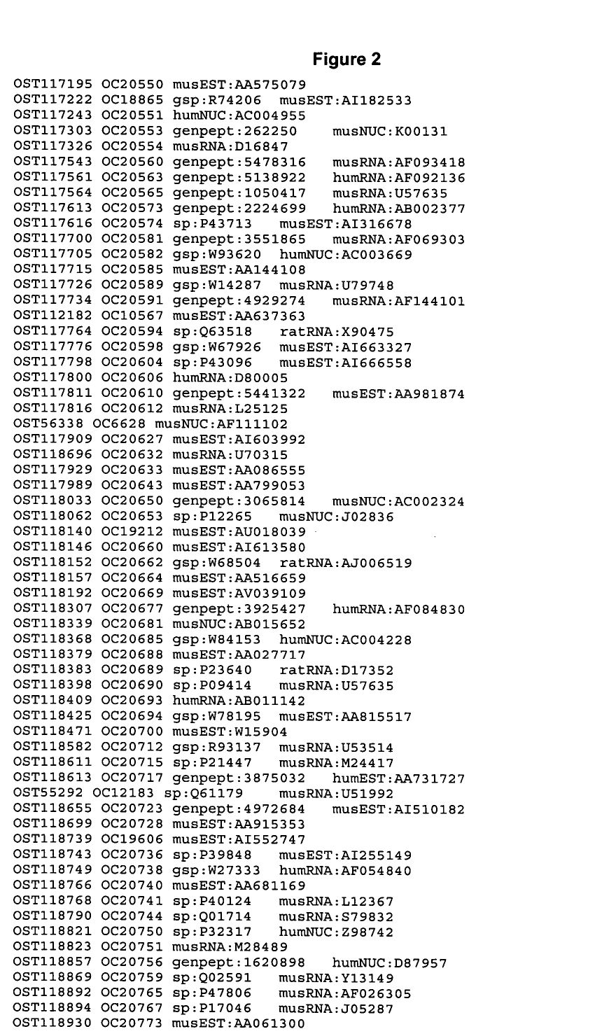

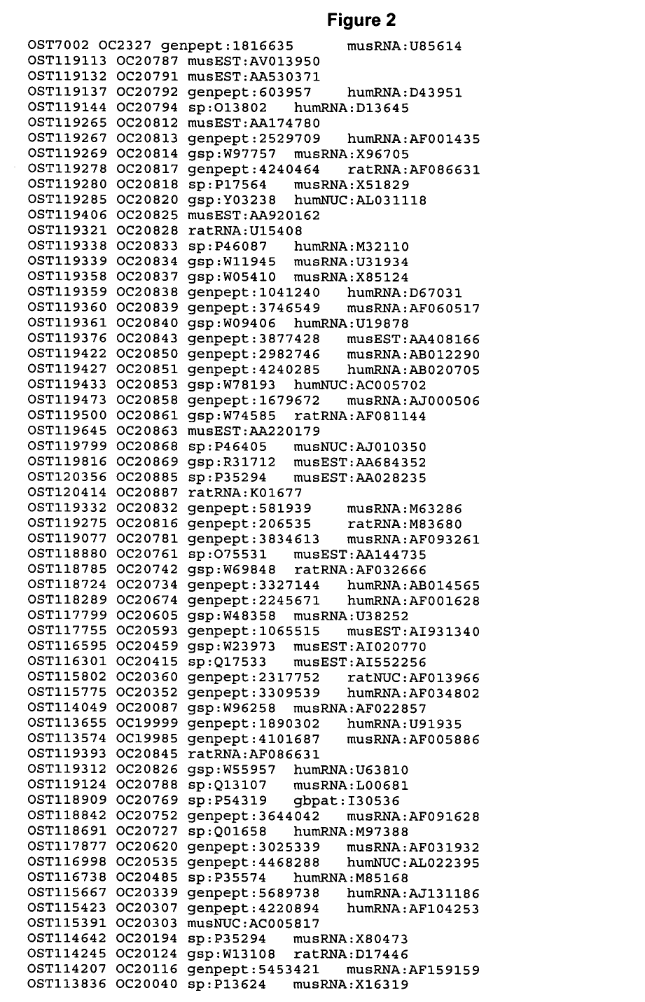

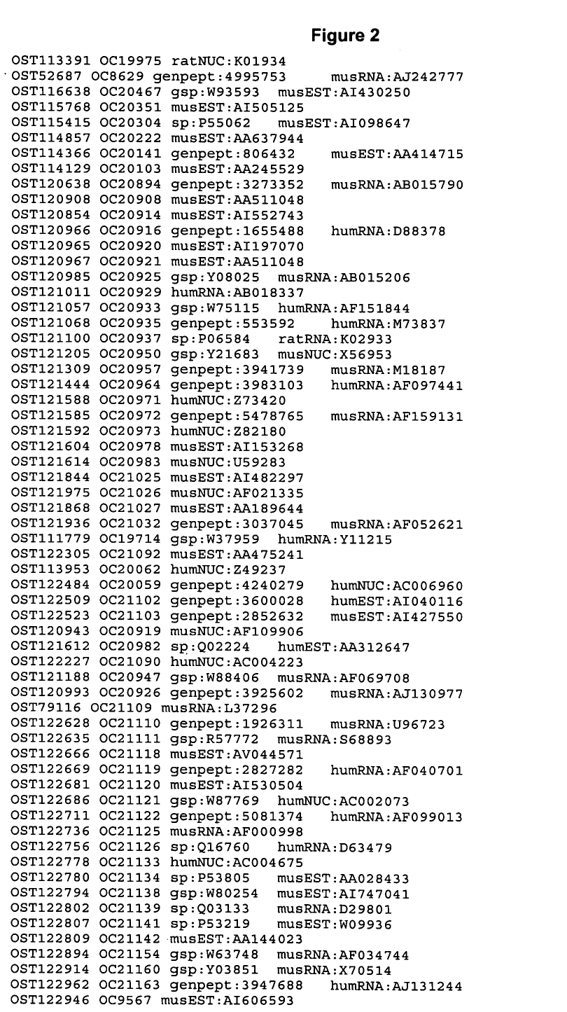

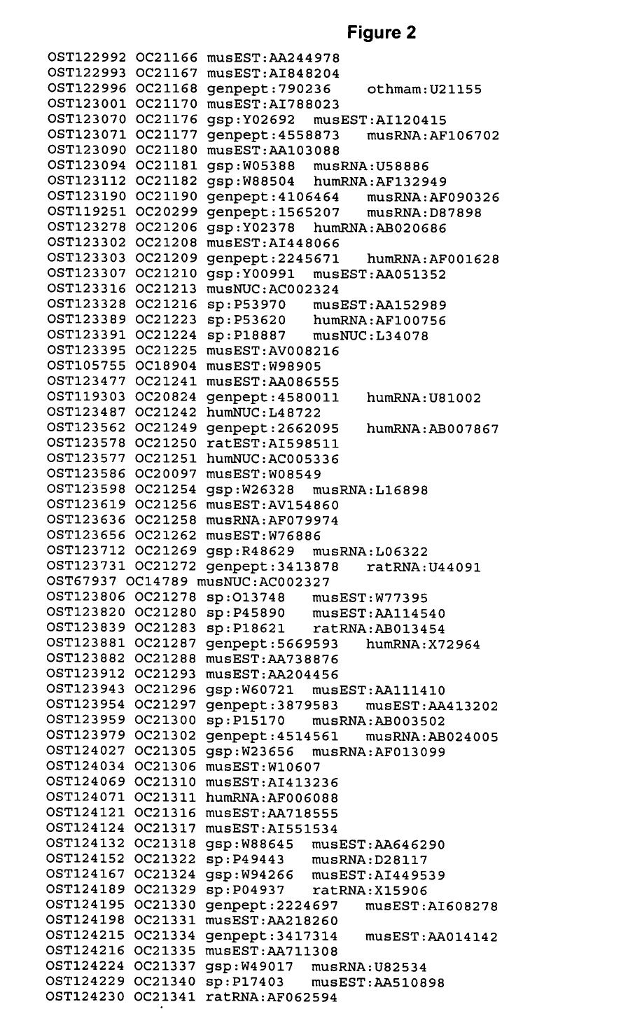

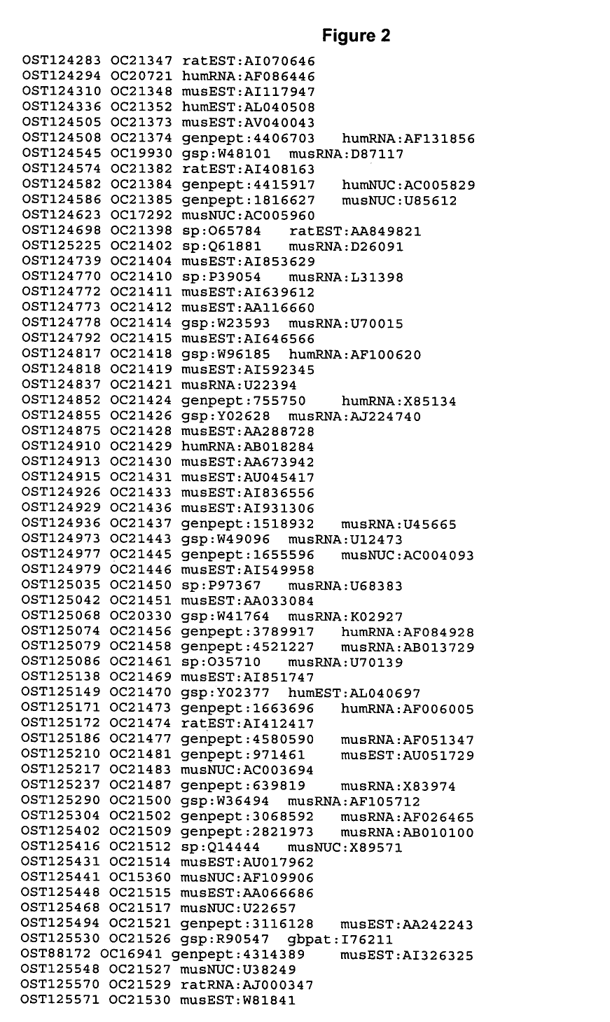

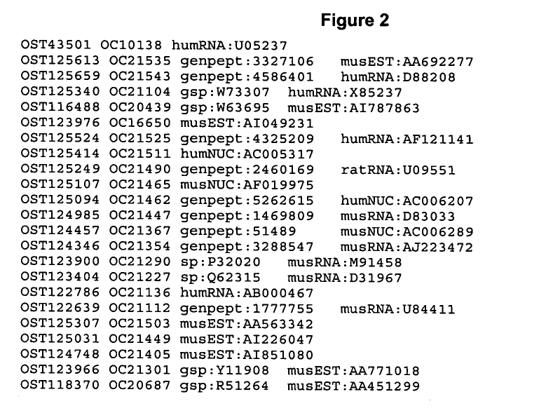

For the purposes of the present invention, the term “identifiable” is to be construed as indicating that a mammalian cell, and preferably, a murine ES cell, has been mutated by the insertion of a polynucleotide sequence of recombinantly manipulated origin at a genetic locus that normally comprises polynucleotide sequence, and/or post-spliced exonic sequence, that is at least partially described in one of the GTSs of Sequence Listing. One method of determining whether one of the described mutated mammalian cells has a mutation in a gene of interest is by comparing the polynucleotide sequence (or a corresponding amino acid sequence) of the GTS identifying the mutated locus to the full length sequence of the gene. Alternatively, such searches can be conducted by comparing the described GTS sequence to a well known database (such as, but not limited to GENBANK) using established computer algorithms including, but not limited to, BLASTX, FASTA, BLASTN, BLASTP, TBLASTN, and TBLASTX using the default parameters used, for example, at the National Center for Biotechnology Information web site (www.ncbi.nlm.nih.gov). The GTSs reported in the Sequence Listing have been compared to such a database (GENBANK), and the accession numbers of the genes that have been mutated are presented in FIG. 2. Accordingly, an additional aspect of the subject invention includes mutated mammalian, preferably murine, cells, or isolated cell lines, that have at least one engineered mutation in a gene identified by GENBANK or GENESEQ (for example) accession number in FIG. 2.

As used herein, the terms “mutated” or “mutation” mean that the genetic locus has been altered by a process involving the integration or incorporation of a genetically engineered polynucleotide sequence into the genome of the cell with the result that the subsequent levels of activity of the product normally encoded by the locus is altered (i.e., reduced, increased, or substantially ablated). In those instances where the mutation substantially completely disrupts the expression or activity of the product normally encoded by the locus (i.e., a null mutation), a cell that is heterozygous for the mutated allele will typically produce about one half of the product of a nonmutated cell (via a gene dosage effect), and about twice the amount of product produced by a cell that is homozygous for the mutant allele.

The term “recombinantly manipulated” shall mean that such compositions comprising such molecules or polynucleotides have been genetically engineered using molecular biology methodologies in vitro or ex vivo (see generally, Sambrook et al., 1989, Molecular Cloning, A Laboratory Manual, Cold Springs Harbor Press, N.Y.; and Ausubel et al., 1989, Current Protocols in Molecular Biology, Green Publishing Associates and Wiley Interscience, N.Y.).

Where, the specifically exemplified mammalian cells, i.e., embryonic stem cells (Lex-1 cells from murine strain A129), are mutated by a process involving the insertion of at least a portion of a genetically engineered vector sequence into the gene of interest, the mutated embryonic stem cells can be microinjected into blastocysts which are subsequently introduced into pseudopregnant female hosts and carried to term using established methods such as those described in, for example, “Mouse Mutagenesis”, 1998, Zambrowicz et al., eds., Lexicon Press, The Woodlands, Tex., and periodic updates thereof, herein incorporated by reference. The resulting chimeric animals are subsequently bred to produce offspring capable of germline transmission of an allele containing the engineered mutation in the gene of interest.

An alternative method of producing mutated cells and animals in the specifically exemplified genes involves the process of gene targeting by homologous recombination using methods such as those exemplified in U.S. application Ser. No. 09/171,642, which is herein incorporated by reference in its entirety. Mutations produced using such methods include, but are not limited to knockout mutations, “knockin” mutations (where a human gene, for example, is used to replace its murine orthologs), can be conditional, can include point mutations, and mutations that activate gene expression. Some of the mutations described above (conditional mutations, point mutations, etc.) can be produced via processes that involve the substantial removal of vector encoded sequences (often recombines mediated) subsequent to the incorporation of the recombinantly manipulated sequences into the genome.

### 5.1. MUTATED MAMMALIAN CELLS OF THE PRESENT INVENTION

The presently described mutated cells have genetically engineered mutations in genes identifiable as corresponding to, or normally comprising, at least a portion of a sequence reported in the Sequence Listing as SEQ ID NOS: 1-891. Additional embodiments of the present invention are cells comprising engineered mutations in homologs, paralogs, orthologs, etc., of the mutated genes disclosed in the Sequence Listing. Such homologs, paralogs, and orthologs include genes having sequences that hybridize to one or more of the disclosed GTSs of SEQ ID NOS: 1-891 under stringent, or preferably highly stringent, conditions. Hybridization conditions also provide an alternative means of identifying the mutated genes corresponding to the GTSs reported in the sequence listing. Typically, such genes will be identifiable because a disclosed GTS, or portion thereof, shall hybridize to the gene under stringent conditions.

By way of example and not limitation, high stringency hybridization conditions can be defined as follows: Prehybridization of filters containing DNA to be screened is carried out for 8 h to overnight at 65° C. in a buffer containing 6×SSC, 50 mM Tris-HCl (pH 7.5), 1 mM EDTA, 0.02% PVP, 0.02% Ficoll, 0.02% BSA, and 500 μMg/ml denatured salmon sperm DNA. Filters are hybridized for 48 h at 65° C. in prehybridization mixture containing 100 μg/ml denatured salmon sperm DNA and 5-20×10⁶ cpm of ³²P-labeled probe (alternatively, as in all hybridizations described herein, approximately 42, 44, 46, 48, 50, 52, 54, 56, 58, 62, 64, 66, 68, 70, or about 72 degrees or more can be used). The filters are then washed in approximately 1× wash mix (10× wash mix contains 3M NaCl, 0.6M Tris base, and 0.02M EDTA, alternatively, as with all washes described herein, 2×, 3×, 4×, 5×, 6× wash mix, or more, can be used) twice for 5 minutes each at room temperature, then in 1× wash mix containing 1% SDS at 60° C. (alternatively, as in all washes described herein, approximately 42, 44, 46, 48, 50, 52, 54, 56, 58, 62, 64, 66, 68, 70, or about 72 degrees or more can be used) for about 30 min, and finally in 0.3× wash mix (alternatively, as in all final washes described herein, approximately, 0.2×, 0.4×, 0.6×, 0.8×, 1×, or any concentration between about 2× and about 6× can be used in conjunction with a suitable wash temperature) containing 0.1% SDS at 60° C. (alternatively, approximately 42, 44, 46, 48, 50, 52, 54, 56, 58, 62, 64, 66, 68, 70, or about 72 degrees or more can be used) for about 30 min. The filters are then air dried and exposed to x-ray film for autoradiography. In an alternative protocol, washing of filters is done for 37° C. for 1 h in a solution containing 2×SSC, 0.01% PVP, 0.01% Ficoll, and 0.01% BSA. This is followed by a wash in 0.1×SSC at 50° C. for 45 min before autoradiography. Another example of hybridization under highly stringent conditions is hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (Ausubel F. M. et al., eds., 1989, Current Protocols in Molecular Biology, Vol. I, Green Publishing Associates, Inc., and John Wiley & sons, Inc., New York, at p. 2.10.3). Alternatively, moderately stringent conditions can be used (e.g., .washing in 0.2×SSC/0.1% SDS at 420°C. (Ausubel et al., 1989, supra). Moderately stringent conditions can be additionally defined, for example, as follows: Filters containing DNA are pretreated for 6 h at 55° C. in a solution containing 6×SSC, 5× Denhart's solution, 0.5% SDS and 100 μg/ml denatured salmon sperm DNA. Hybridizations are carried out in the same solution and 5-20×10 cpm ³²P-labeled probe is used. Filters are incubated in hybridization mixture for 18-20 h at 55° C. (alternatively, as in all hybridizations described herein, approximately 42, 44, 46, 48, 50, 52, 54, 56, 58, 62, 64, 66, 68, 70, or about 72 degrees or more can be used in combination with a suitable concentration of salt). The filters are then washed in approximately 1× wash mix (10× wash mix contains 3M NaCl, 0.6M Tris base, and 0.02M EDTA, alternatively, as with all washes described herein, 2×, 3×, 4×, 5×, 6× wash mix, or more, can be used) twice for 5 minutes each at room temperature, then in 1× wash mix containing 1% SDS at 60° C. (alternatively, as in all washes described herein, approximately, 42, 44, 46, 48, 50, 52, 54, 56, 58, 62, 64, 66, 68, 70, or about 72 degrees or more can be used) for about 30 min, and finally in 0.3× wash mix (alternatively, as in all final washes described herein approximately 0.2×, 0.4×, 0.6×, 0.8×, 1×, or any concentration between about 2× and about 6× can be used in conjunction with a suitable wash temperature) containing 0.1% SDS at 60° C. (alternatively, approximately 42, 44, 45, 48, 50, 52, 54, 56, 58, 62, 64, 66, 68, 70, or about 72 degrees or more can be used) for about 30 min. The filters are then air dried and exposed to x-ray film for autoradiography.

In an alternative protocol, washing of filters is done twice for 30 minutes at 60° C. in a solution containing 1×SSC and 0.1% SDS. Filters are blotted dry and exposed for autoradiography.

Other conditions of moderate stringency which may be used are well-known in the art. For example, washing of filters can be done at 37° C. for 1 h in a solution containing 2×SSC, 0.1% SDS. Another example of hybridization under moderately stringent conditions is washing in 0.2×SSC/0.1% SDS at 42° C. (Ausubel et al., 1989, supra). Such less stringent conditions may also be, for example, low stringency hybridization conditions. By way of example and not limitation, procedures using such conditions of low stringency are as follows (see also Shilo and Weinberg, 1981, Proc. Natl. Acad. Sci. USA 78:6789-6792): Filters containing DNA are pretreated for 6 h at 40° C. in a solution containing 35% formamide, 5×SSC, 50 OmM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10⁶ cpm ³²P-labeled probe is used. Filters are incubated in hybridization mixture for 18-20 h at 40° C. (alternatively, as in all hybridizations described herein, approximately 42, 44, 46, 48, 50, 52, 54, 56, 58, 62, 64, 66, 68, 70, or about 72 degrees or more can be used). The filters are then washed in approximately 1× wash mix (10× wash mix contains 3 M NaCl, 0.6M Tris base, and 0.02M EDTA, alternatively, as with all washes described herein, 2×, 3×, 4×, 5×, 6× wash mix, or more, can be used) twice for five minutes each at room temperature, then in 1× wash mix containing 1% SDS at 60° C. (alternatively, as in all washes described herein, approximately 42, 44, 46, 48, 50, 52, 54, 56, 58, 62, 64, 66, 68, 70, or about 72 degrees or more can be used) for about 30 min, and finally in 0.3× wash mix (alternatively, as in all final washes described herein, approximately, 0.2×, 0.4×, 0.6×, 0.8×, 1×, or any concentration between about 2× and about 6× can be used in conjunction with a suitable wash temperature) containing 0.1% SDS at 60° C. (alternatively, approximately 42, 44, 46, 48, 50, 52, 54, 56, 58, 62, 64, 66, 68, 70, or about 72 degrees or more can be used) for about 30 min. The filters are then air dried and exposed to x-ray film for autoradiography. In yet another alternative protocol, washing of filters is done for 1.5 h at 55° C. in a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are then blotted dry and exposed for autoradiography. If necessary, filters are washed for a third time at 65-68° C. and reexposed to film. Other conditions of low stringency which may be used are well known in the art (e.g., as employed for cross-species hybridizations). Preferably, GTS variants identified or isolated using the above methods will also encode a functionally equivalent gene product (i.e., protein, polypeptide, or domain thereof, encoding or otherwise associated with a function or structure at least partially encoded by the complementary GTS).

Low stringency conditions are well known to those of skill in the art, and will vary predictably depending on the specific organisms from which the library and the labeled sequences are derived. For guidance regarding such conditions see, for example, Sambrook et al., 1989, Molecular Cloning, A Laboratory Manual, Cold Springs Harbor Press, N.Y.; and Ausubel et al., 1989, Current Protocols in Molecular Biology, Green Publishing Associates and Wiley Interscience, N.Y.

The identification of homologs, heterologs, or paralogs of SEQ ID NOS: 1-891 in other, preferably related, species can be useful for developing additional animal model systems that are closely related to humans for purposes of drug discovery. Genes at other genetic loci within the genome that encode proteins which have extensive homology to one or more domains of the gene products encoded by SEQ ID NOS: 1-891 can also be identified via similar techniques. In the case of cDNA libraries, such screening techniques can identify clones derived from alternatively spliced transcripts in the same or different species.

Techniques useful to disrupt a gene in a cell and especially an ES cell that may already have a disrupted gene are disclosed in copending U.S. patent application Ser. Nos. 08/726,867; 08/728,963; 08/907,598; and 08/942,806, all of which are hereby incorporated herein by reference in their entirety, are within the scope of the current invention to disrupt a gene that encodes a polynucleotide of the current invention.

### 5.2. USES OF THE DESCRIBED MUTATED GENES AND ANIMALS

The described mutated cells and animals are used to investigate and define the cellular and biological functions of the mutated genes. Producing a scientific model that accurately accounts for the large number of genes, proteins, and macromolecules within a single cell has thus far proved beyond the capabilities of existing computer technology. It should thus not be surprising that the far more complex task of modeling the various intricacies, cross and direct redundancies, and interrelated functions of the various metabolic and catabolic processes that occur within a single cell has also proven largely intractable to algorithmic methods of modeling and prediction. Even if one assumes that computer modeling of inherently chaotic/heuristic processes will rapidly mature in the near future, such methods, at best, can only provide predictions that subsequently require practical validation. Several decades of empirical data have proven that mutant phenotypes provide a valuable source of such validation. The mutated diploid mammalian cells of the present invention will initially exist as mutated diploid cells that are heterozygous (except where genes on the X or Y chromosomes are mutated) for the mutations identified in the sequence listing. As such, via a “gene dosage” effect, the mutated cells can typically be characterized by the fact that they produce about one half of the mutated transcript/activity relative to cells having two nonmutated or wild type copies of the corresponding gene.

When mutant animals are produced from the mutated cells, heterozygous animals capable of germline transmission of the mutated allele can be bred to produce embryos or offspring that are homozygous for the mutant allele. Such animals or embryos are a rich source of tissues and cells that do not express physiologically relevant amounts of the mutated genes or activities encoded thereby. Accordingly, an additional embodiment of the present invention are mutant cells and animals that have homozygous mutations in genes identifiable as corresponding to the GENBANK, or other database accession, numbers provided in FIG. 2, or are identifiable as a homologs, paralog, or orthologs of a sequence provided in the Sequence Listing.

In addition to providing important information regarding the functional role of a given gene in its nonmutated state (i.e., you learn about the function of the gene by discerning the effects of reducing or ablating the activity normally encoded by the gene), the described mutated cells and animals can be used as disease models, or in assays for compounds or genes (via gene delivery or transgenic methods) that compensate for the mutant phenotype and that can be used to treat diseases and disorders related to the observed phenotype. Alternatively, such products and genes can also be used to enhance desirable, if not normal, symptoms related to the observed phenotypes.

The gene replacement/delivery therapies described above should be capable of delivering gene sequences to the cell types within patients which express the peptide or protein having the desired activity.

The examples below are provided to illustrate the subject invention. These examples are provided by way of illustration and are not included for the purpose of limiting the invention in any way whatsoever.

### 6.0. EXAMPLES

### 6.1. Generation of a Library of Mutated Mouse ES Cells Defined by GTS Sequences

The retroviral vector VICTR 3, described in detail in U.S. application Ser. No. 08/728,963, filed Oct. 11, 1996, was used to generate a library of gene trapped ES cell clones that represent a portion of the described GTSs. A plasmid containing the VICTR 3 cassette was constructed by conventional cloning techniques and designed to employ the features described above. Namely, the cassette contained a PGK promoter directing transcription of an exon that encodes the puro marker and ends in a canonical splice donor sequence. At the end of the puromycin exon, sequences were added as described that allow for the annealing of two nested PCR and sequencing primers. The vector backbone was based on pBluescript KS+ from Stratagene Corporation.

The plasmid construct was linearized by digestion with Sca I which cuts at a unique site in the plasmid backbone. The plasmid was then transfected into the mouse ES cell line AB2.2 by electroporation using a BioRad Genepulser apparatus. After the cells were allowed to recover, gene trap clones were selected by adding puromycin to the medium at a final concentration of 3 μg/ml. Positive clones were allowed to grow under selection for approximately 10 days before being removed and cultured separately for storage and to determine the sequence of the disrupted gene.

Total RNA was isolated from an aliquot of cells from each of 18 gene trap clones chosen for study. Five micrograms of this RNA was used in a first strand cDNA synthesis reaction using the “RS” primer. This primer has unique sequences (for subsequent PCR) on its 5′ end and nine random nucleotides or nine T (thymidine) residues on it's 3′ end. Reaction products from the first strand synthesis were added directly to a PCR with outer primers specific for the engineered sequences of puromycin and the “IRS” primer. After amplification, an aliquot of reaction products were subject to a second round of amplification using primers internal, or nested, relative to the first set of PCR primers. This second amplification provided more reaction product for sequencing and also provided increased specificity for the specifically gene trapped DNA.

The products of the nested PCR were visualized by agarose gel electrophoresis, and seventeen of the eighteen clones provided at least one band that was visible on the gel with ethidium bromide staining. Most gave only a single band which is an advantage in that a single band is generally easier to sequence. The PCR products were sequenced directly after excess PCR primers and nucleotides were removed by filtration in a spin column (Centricon-100, Amicon). DNA was added directly to dye terminator sequencing reactions (purchased from ABI) using the standard M13 forward primer a region for which was built into the end of the puro exon in all of the PCR fragments.

Subsequent studies have used both VICTR 3 and VICTR 20. Like VICTR 3, VICTR 20 is exemplary of a family of vectors that incorporate two main functional units: a sequence acquisition component having a strong promoter element (phosphoglycerate kinase 1) active in ES cells that is fused to the puromycin resistance gene (or other exon sequence) that is followed by a synthetic consensus splice donor (SD) sequence and lacks an operatively positioned polyadenylation sequence downstream from the SD sequence (PGKpuroSD); and 2) a mutagenic component that incorporates a splice acceptor sequence fused to a selectable and/or calorimetric marker gene and followed by a polyadenylation sequence (for example, SAβgeopA, SAneopA, SAIRESneopA, or SAIRESβgeopA).

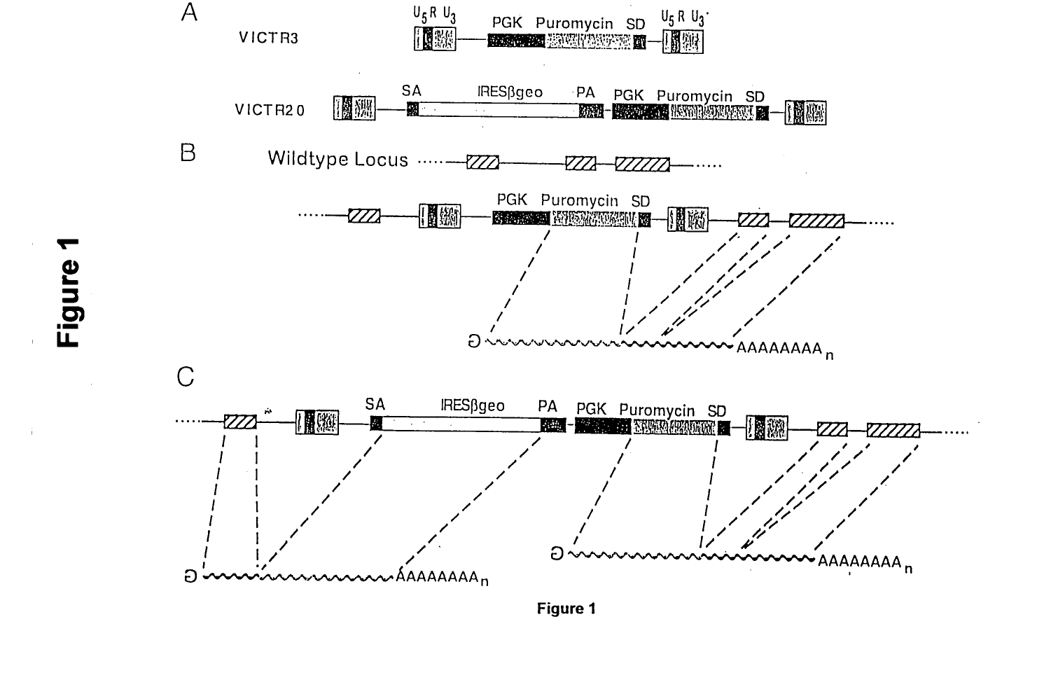

Also like VICTR 3, stop codons have been engineered into all three reading frames in the region between the 3′ end of the selectable marker and the splice donor site. A diagrammatic description of structure and functions of VICTRs 3 and 20 is provided in FIG. 1.

When VICTRs 3, 20, and various variations thereof such as the vectors and methods described in U.S. application Ser. Nos. 09/276,533, and 60/095,989 (the disclosures of which are herein incorporated by reference), were used in the commercial scale application of the presently disclosed invention, many mutagenized ES cell clones were rapidly engineered and obtained. Sequence analysis obtained from these clones has identified a wide variety of sequences. Each of the sequences presented in SEQ ID NOS: 1-891 identify novel mutations in the coding regions of mammalian genes that identifiable as corresponding to the sequences presented in the Sequence Listing. Alternatively, the described mutated cells are described by the database (GENBANK, GENSEQ, etc.) accession numbers for the corresponding genes that have been mutated (see FIG. 2). The described mutated cells, and preferably ES cells, provide a valuable resource for defining, evaluating, or validating the biological function or disease/pharmaceutical relevance of each of these genes.

The cloned 3′ RACE products resulting after the target ES cells were infected with one of the described gene trap vectors were purified using conventional column chromatography, (e.g., S300 and G-50 columns), and the products were recovered by centrifugation. Purified PCR products were quantified by fluorescence using PicoGreen (Molecular Probes, Inc., Eugene Oreg.) as per the manufacturer's instructions.

Dye terminator cycle sequencing reactions with AmpliTaq® FS DNA polymerase (Perkin Elmer Applied Biosystems, Foster City, Calif.) were carried out using approximately 7 pmoles of sequencing primer, and approximately 30-120 ng of 3′ template. Unincorporated dye terminators were removed from the completed sequencing reactions using G-50 columns as described above. The reactions were dried under vacuum, resuspended in loading buffer, and electrophoresed through a 6% Long Ranger acrylamide gel (FMC BioProducts, Rockland, Me.) on an ABI Prism® 377 with XL upgrade as per the manufacturer's instructions. The sequences of the resulting amplicons, or GTSs, are described in SEQ ID NOS: 1-891. All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the above-described modes for carrying out the invention which are obvious to those skilled in the field of molecular biology or related fields are intended to be within the scope of the following claims.

1. A genetically engineered mammalian cell that has been mutated by a process comprising the insertion of a recombinantly manipulated polynucleotide sequence into a gene in said genetically engineered mammalian cell wherein said gene is identifiable as corresponding to at least one of SEQ ID NOS: 1-891.

2. The genetically engineered mammalian cell of claim 1, wherein said cell is murine.

3. A cell according to claim 2, wherein said cell is an embryonic stem cell.

4. The genetically engineered mammalian cell of claim 1, wherein said polynucleotide sequence is present on a viral vector.

5. A cell according to claim 4, wherein said viral vector is a retroviral vector.

6. A cell according to claim 4, wherein said viral vector additionally comprises regions of targeting DNA that facilitate gene targeting by homologous recombination.

7. An isolated murine embryonic stem cell line comprising an engineered retroviral gene trap vector in at least one gene comprising a polynucleotide sequence first disclosed in one of SEQ ID NOS: 1-891.


SEQUENCE LISTING (891 sequences)

SEQ ID NO: 1 - DNA, 449 bp, Mus musculus
  misc_feature (1)...(449): n = A,T,C or G
aggccttgaagagatccgcaaatgtggantgaaaaacttccgnaacatccaggntgatgaggctaatttattgacttggcaagggcttattgttcctgacaaccctccatatgataagggagccttcagaattgaaatcaactttccagcagagtatccattcaaaccacccaagatcacatttaaaacaaagatctaccaccctaacatcgatgagaaggggcaggtctgtctgccagtcattagtgctgaaaactggaagccagccaccaagactgaccaagtaatccagtccctcatagcactggtgaatgacccccagcctgagcacccactccgggctgacctagctgaagaatactctaaggaccgtaaaaaattctgtaagaatgctgaagagtttacaaagaaatatggggaaaaagccgacctgggggcttaaaatctgc

SEQ ID NO: 2 - DNA, 397 bp, Mus musculus
  misc_feature (1)...(397): n = A,T,C or G
atttgccagctgaaggcatttacattcggagttccgcggactcttcanaggctgtaaaaatgccggagtcacnaaagcctgcaaaaagctgttgcttgccttgctgctgctgctgctactgccgtccctgcccgtgctgcaagctgctactcctgctgctgctgctgctgcttgttgctgattgttgcggttgctggttgctgtttactgtttgctgccggctggattgctggattgctgattgccagtcgggnaaatcctggnnataaaaatccancttgccccanaaaacangacnaccctnnaggaagtattctaatgatttcaatncgccctttccccctctnacctttctctcttaccttatgctgggggattggaagggtggcaaaaaaaa

SEQ ID NO: 3 - DNA, 285 bp, Mus musculus
  misc_feature (1)...(285): n = A,T,C or G
gatggagaaactgaggttccgcatngaancaggctgattcagacctcgctgccactgagtatgagaccacctggggtgaaacttagatggctctattgttctgtcacctgccgccactcctctccaagacactgttacctgcccttgagatattccggagacagcatcccgcagatcacctacaggctggcgacaggcaccaagaatggattggtgggggatgggcctcccctctttatacgcacagagtcttagtaaattttgggtccttgaacagaaaaaaaa

SEQ ID NO: 4 - DNA, 287 bp, Mus musculus
  misc_feature (1)...(287): n = A,T,C or G
caggagtaacaagtgtggaggtcnaagganaactcttggaatctcttctatcctatcacatcngtctcaggcatcnaacatgggccaactcctcaaacttggtancctggnacttnaatanccatcttantgctcaaaaaaggtttctcctatgaagaccgtgtntcctggctgtcctagaatttgctgtgcnnaccaacctggcctnnaactcataaacntttacctgcctctgcctctaaagtgttgggattnaaagcatttcccactatgtaacaaacaaaaaa

SEQ ID NO: 5 - DNA, 417 bp, Mus musculus
  misc_feature (1)...(417): n = A,T,C or G
cgcccatcctaaattcctagcttgctcggggtaggagnttcctgttcattcgttctgatanncccactgntgaaaactgaggngccacactcgtatcnctntcnacnggacctaacattattcggganggaactctggtccncncccccatcnttcaatnnnnntgtcgtggngattggnttngaaccntgntcagnataaagctgaaaaaaaccccattgnctnncatttncaccangaccgggaagngtntnatatcnaggatccgaataaggctcttcaaggcttctttgaccgagaaggagaanagttagaatatgaatttgatgagcnaggacacatcacttggctctgcagggtgaggttacctgtagatgattcnnctgggaaacagctggtggctgaggccattcactc

SEQ ID NO: 6 - DNA, 173 bp, Mus musculus
gtctagaatgtatgtgccatgctgggaacaggcctggatcgtctgcaagagcagccatcggtcttagctgctaactcctctctcagtccattttgaatacgattctgaggtgacgcttccctgtcaccacccttaacatgctctcatcgtaccctctggtcaaataaaaaaaa

SEQ ID NO: 7 - DNA, 167 bp, Mus musculus
  misc_feature (1)...(167): n = A,T,C or G
tctagatgtatgtgccatgctgggaaccangcctggatcgtctgcaagagcagncatcggtcttagntgctaactcctctctcagtccatttgaatacnattctgaggtgacgcttccctgtcgncacccttaacatgctctcatcgtaccctctgaaaaaaaaaaa

SEQ ID NO: 8 - DNA, 191 bp, Mus musculus
  misc_feature (1)...(191): n = A,T,C or G
attctccatccacaaattatgcagtcaagtttcctcnaatttggggtcaatacacacagagtgcaatggatgaacctcgctctggagaaatcacctgcataatcatggtatttccccttccaggatttatacanaagcagtcttccaaattctgaaacctgatgcccatcacatactttgggctgagaaaa

SEQ ID NO: 9 - DNA, 157 bp, Mus musculus
  misc_feature (1)...(157): n = A,T,C or G
ggtctagattatgtgcatgctgggaacaagcctggatcgtctgcaagagcagncatcggtcttagctgctaactcctctctcagtccatntgaaatacgattctgaggtgacgcttccctgtcnncacccttaacatgctctcatcgtaccctctga

SEQ ID NO: 10 - DNA, 273 bp, Mus musculus
  misc_feature (1)...(273): n = A,T,C or G
ggaatacaaagggccagggtccggctgcgtctcctggatcttgagctgacatctaggttcctgggtgggaccacaaatncaactatactggaggctgatgcanttctcttaggactccaagagatcagggactcaaaatcaagagaggagcaacccagcaagtaatggactgcgcttgctgtatttgaacccattttgagcccagaaaactataaataaataaataattgaataaatttatnataaatgaaaagtaatcctgttaaaaaaaaa

SEQ ID NO: 11 - DNA, 370 bp, Mus musculus
  misc_feature (1)...(370): n = A,T,C or G
actgaggtgacctttgaaagttaacaggggtgccatgggctgagtaagataaacggaactgctgttcaaccccgcccagtctggggatgagtcattgttttcaaatttatgaacaaagcacacaaattaagaataatacaagcaagaaacagcagagagacacaaaaggacatagaatccctgatcctcactgtgatctggtaacactagttctagggctggagagagagatgctcagggcaagtgctcttacagagaacatgagttcaattcccagaacccacatggtacctcacaactgcctatttcatacatgtggggcagaacaccgatacacatacttttttttttaanaattgaaaaaaaaaaa

SEQ ID NO: 12 - DNA, 448 bp, Mus musculus
  misc_feature (1)...(448): n = A,T,C or G
ctcctttagtgagacngccctaggcagaccacntaccnttacccttctgaaatctcggtactggaggatctnggccnggatcctgatacccccattgcccccttagcnacccatcctgacnaggaggaggaggaggaggaggaagaggaggagttgcttctgcngcctgggctccagggcgggctgcgcaccaaggccctantcgtggatgantcctgccggcggtgaccatcttccantttgggcgcatacctccttccttccttccttcccgtacctgaagcttctggagtctggatgattcanagccgacanacctggcngggatcctgcctggcagggaagggggttgancntnttctgacttgcttttcccatcccatttctggggcnnanccnttatcctaaatttacccctaactcatggtgtttcccgtgggtgacctaa

SEQ ID NO: 13 - DNA, 394 bp, Mus musculus
  misc_feature (1)...(394): n = A,T,C or G
tcctgatactgcttgntnggggtagagctncctgctcntacnttctgccnnaccnnatgctggaacctgagnatcnacataattgcaccttctcttctggaccagacattattcggcgggaatcgggtccccaccccctgacgtcatacctggtgtctcanccnnngnatttgaactgtgatcatctgagccaaaatannccaaagtatanaagtcaacaaaggcaanatgctgnanatnaaatggaaaaccantcattganaacatcatcncctctntnctccgaggacncttcggtgcggatctgggatatcccnnanggtgggctgaaacggaacntnaccgagggactcctggagctcnatgganacatgcgccatgtggggctggtgga

SEQ ID NO: 14 - DNA, 461 bp, Mus musculus
  misc_feature (1)...(461): n = A,T,C or G
cgcaccggattgggcagcantaacnacactgcaanaggancctnctgcacacgtttattggganagtntgattgnagaggtgannagaccccgagcccaaaactggtgctgcttatataggcctangagaggantgtctcacacctggattggttatgcactacgcctcatttgcatgatcctcatctgattggctactctctctctgtacctcacaaagcctcattntcatacctcatttgcatgtctcacacctgattggttatantctccaaaccccctttatcctgcccggggcnngcantgtctttgcaaaaaactttactgcatatgttcacattggttgtttgtccaaacttatgcgtggtggccancagtantcagtgccactctgcaacggcacntgtggcttcccacatctccccctttttgttttaataaaatgaggctgggactaggcc

SEQ ID NO: 15 - DNA, 301 bp, Mus musculus
aagtgttccctaagaaggttgtcaccctgacggaagaacggaaagcccaacagctccagcgcctcctggacatgaagagcaaccccatacagggcctggctgcccactgggattatgaaaagaaggagtggaaaaagtgaccaacgtcccacgtctgccgccagcccctgcaaactgcttctgcagcccaggagacaatcatcccctcccctcccctcccctccctcctcctcctgagtcctgtctccactcccttctccaataaaagcagcctgctttgttctgcttgcaaaaaaaaaaa

SEQ ID NO: 16 - DNA, 285 bp, Mus musculus
  misc_feature (1)...(285): n = A,T,C or G
aggttcctaaaatcanactctgtcatcggnaagtgnagactgtaacaaactgggaccctcacatgccnataagcantcaaaaatatttnctgggagctggananatgggctcagcagttaaaaacactgactgctcttccggangtcctgtgttcaaatcccagcaaccacagggtaactcacaaccatctgtaatganatctgatgccctcttcgggtgtgtctnaaaacagctacagtgtccttacatataataaataaataaatctttttttttaaagaata

SEQ ID NO: 17 - DNA, 100 bp, Mus musculus
  misc_feature (1)...(100): n = A,T,C or G
ttgccccaaattggctgtagccacacanacatgagaatgtcagatctcattccggatgaagccctgagaagggcaatcgagagccacaacaagaaaaaaa

SEQ ID NO: 18 - DNA, 269 bp, Mus musculus
  misc_feature (1)...(269): n = A,T,C or G
catggaatggacgggacaaggaaggcaaacattcacctggaancaaaanctgtgctgctttntggcttatttacagtanntttncncaaccggcaagcttgtgcantnnnagcaagaactcctcctgcctgcctccggaataagctgttgaattcttgtttcttccagcgcggtgtctgcctgcacgctgccatgcgtcctgccatgatnataatggactgaccctctgaaactgtaagccagccccaantaaattttgtccttgataa

SEQ ID NO: 19 - DNA, 312 bp, Mus musculus
aaggttgtcctcggaggccaaaggcgtcagatacgacacctggaagttgaatttacagaattgccagctgcccaacatggattcttggaaccaaacctttagaattcaacaaacaaacaacccagggggctggagagatggctcagcactcaagagcactgactgctgctcttccagaggtcctgagttcaattcccagcaaccacatggtggttcacaaccatctgtaatgagttccaatgccctcttctggtgtgtctgaagacagcgacataaaatgaataaatctttttaaaaacccaaaaaaaaaaa

SEQ ID NO: 20 - DNA, 177 bp, Mus musculus
  misc_feature (1)...(177): n = A,T,C or G
aagaaacgttttgtaagtgaagganatggaggccgtcttaaacctgaaagctactgagcacagcaactcttacagtcttaggtgttaccaanaacanatctcaactgtaaaagttgtcagtacaggtttcctttncctttngtttttgttattgttttttttttttttttttttttt

SEQ ID NO: 21 - DNA, 273 bp, Mus musculus
  misc_feature (1)...(273): n = A,T,C or G
tcatggagaataacgggaacaaccnaaanctccgggtttgcgttgccacctgcaaccganctgactantccntacnggccccggatcatgtncggcatcaaaacnaagcccgcgtnctttgagttggangtggtggtgctcggctcccacctgattgangactatggctcctgctccctganctgcttgagttccagtcctgacttcctttggtgatgaacagcagcatggaantgtaagccaaataaacccttncctccccaaaaaaaaaaa

SEQ ID NO: 22 - DNA, 204 bp, Mus musculus
  misc_feature (1)...(204): n = A,T,C or G
gggttccaagactgccctttgggattcaaacgggagccgctttggactgactggctgccttcctcctcctccccctcctcccccttctgcttcagatttatttattatatgtaagtacactgtagcagttgtaggacacactagacgagggcaccagatctcattgtgggtggttgngnaccncccaccatgggggggctggga

SEQ ID NO: 23 - DNA, 427 bp, Mus musculus
  misc_feature (1)...(427): n = A,T,C or G
gcttggccaagattcaccagggcctgctgtcctcacctgtggatccaccagccagccatggtatagaggtattcccagcaagtcaaagatgctgcattccggcgtatactcaaagtcggagacgcccgtgaatcgcacattgacgtccagacctgtggccagtttcgggagcaccgtcattgcatcgtccacattctggtgggagaaaagacaagatggctggtctagccttccctccctgccacccaccatcccatgtttcatgtctggctttcttacaaaccgtttgtttcatccacctcttacctcaaacccccttttgtgagacagagccctggctgtattgaaactcactctgtanaccaagcttgcccttgaactcacagacatctacctctctctgcttcccaagtgctgtaattaaagg

SEQ ID NO: 24 - DNA, 265 bp, Mus musculus
  misc_feature (1)...(265): n = A,T,C or G
gggttccaagactgccctttgggattcaaacgggagccgctttggactgactggctgccttcctcctcctccccctcctnccccttctgcttcagatttatttattatatgtaagtacactgtancagttgtaggacacactagacgagggcaccagatctcattgtgggtggnngatgannncatcancatacaggctacacaggaccancaacggaatgaggagtaggcccggatcatggntgagtntgagatcacagagcag

SEQ ID NO: 25 - DNA, 335 bp, Mus musculus
gatggtgcctgaacccaactgattaagcaggcaggcagaaacgtgtcaccatcagctaccattgtgccctcatgcataaatgaagtcctgatggaaaacagacaaacaaacaaacaaacaaacatctctacacactaagccgaaaacaaatctataccaaactgctaaaataaatggaaaatacttaatttgagcctggctgttttgaaaattggaaatccttcttcaaaatgaaaatcaccgcaaaacaatctaaagtcatctacaggccaactagggtgtttgttgcttgccaaacttccaataaattctgcttgaaaccttaaaaaaaaaaa

SEQ ID NO: 26 - DNA, 587 bp, Mus musculus
  misc_feature (1)...(587): n = A,T,C or G
gccggcctccactcctccatgtccngngcctgcntctccnnccacttgtntcccntcaccnccatnaccatgtanancggggtgancaatgtgggcnttcaccaccaccatcaccattctccccatcgtctccaccaccacaagcaccaccaccgccaccaccatgctcatggagcccgccgctaaggccaatagatagcatctacctggctctgccctccatccagcancaacatgagtggatcctgatgctgccacggaaaaggatgtgcctctgggataaaagggggtactgggaccctggtcctgcttgggtttcatttgcatatgagcccttctccctatgtgcaacaganaccacctgggtgccctctacacaatgtgngggcttgancttcgagcaaggctggctgantttcctaccttgtgactgcaactcctggtgacctggaaaataacaagggttctggcanaaacaaaacccnggtgaaaatgaaaattcttctgattggggaaggcctgtgatnaacttccaggggcnaantgtggttagggcattcactgcttcttgggttttaaaaataaaa

SEQ ID NO: 27 - DNA, 180 bp, Mus musculus
agggttccaagactgccctttgggattcaaacgggagccgctttggactgactggctgccttcctcctcctccccctcctcccccttctgcttcagatttatttattatatgtaagtacactgtagcagttgtaggacacactagacgagggcaccagatctcattgtgggtggttgcga

SEQ ID NO: 28 - DNA, 232 bp, Mus musculus
  misc_feature (1)...(232): n = A,T,C or G
gggttccaagactgccctttgggattcaaacgggagccgctttggactgactggctgccttcctcctcctccccctcctcccccttctgcttcagatttatttattatatgtaagtacactgtagcagttgtaggacacactagacgagggcaccagatctcattgtgggtggttgngaaccacccaccatgtggtngctgggatntgaactcaggatcttcagaagagcag

SEQ ID NO: 29 - DNA, 213 bp, Mus musculus
ggaattttactacttcacaaatggagaatgatgctacaaagttaccatggtgaagctgacaaataccactcaaatgatgatgacatagaagtaccttcctcctgctgctctgatcccataattggttactagatgatgttttctgacatggatttcgattttgggtacctgttactttggcactattcttttttttttttttttttttttttt

SEQ ID NO: 30 - DNA, 735 bp, Mus musculus
  misc_feature (1)...(735): n = A,T,C or G
gaaacttatctccctccccatatcctgaaactgctgnctcggngtgtgacctcctgctcgtncgttctgncncncccactgctggaacnnnanganccgcncacatgcagctnnctactggaccataaattattctgngggaatctggtccccnccccctnccttcttnnctggtgtcgcnccgntgtgatntgacccttgatcggacaatctcctccagggntggccanggatcnatcccttanagtaactcaagagttccncatgctgtgttatctggactcccactaantgcagcactgtgccttcggaacnacaccatcnagaatgtatcccttcntcnctccagctngacnactaccaagatctccaccaccctttcttccttctgggacatcgtgtnccaacccantggtccatatccaggccctgctgtactantcccnggccccacnaggtcatntntntcaacaaaatgtgcccgntccaaaanctaccnncgccnaatancgcacncacagatccanttggtcctttaagtacacngggatccatgtcttctggaccttgggcaccangaatgggaaggcagcatcctaatgtgccatatctatttccanaatcatctcccactcctcctcttccantgttaggancacacctgtttgcattggttcnnntgccnccgggggccgggaaccncancgcatatcctnatcctgaagcgtcttccca

SEQ ID NO: 31 - DNA, 677 bp, Mus musculus
  misc_feature (1)...(677): n = A,T,C or G
aatgcagacataaagcctttacttggagaacagatcatactccaagggaacatcatcagcatcaccaccagcagcaccatcagcagcaacancagcagcagcagcaacagccacccccaccaatacctgcaaatggccancaggccagcagccagaatnaangcttgactattgacctgaagaattttaggaaaccaggagagaagacctttacacagcgttnccgtctctttgtgggcaatcttccccctgatntcactgaggaggaaatgagganactatttgagaaatatggaaaagcaggcgaanttttcattcataaggataaaggctttggctttattcgcttggaaacacgaaccctagcggaaattgccaaagtgganctggacaacatgcccctccgtgggaagcaactgcgaatgcgctttgcctgtcacagtgcatcccttanagtccgcaaccttcctcagtacgtgtccaacgaactgctggaanaaccttttctgtgttcggccangtggaaaaggctgtaatcattgtggatgaccgaagaaggnccccgggaaaagcattgtttattctcaaggaaaccagctgctccgaaanctctggananatcagtgaaggntccttcntgctgantacatttcctcngcctgtgaatgtggacctat

SEQ ID NO: 32 - DNA, 305 bp, Mus musculus
  misc_feature (1)...(305): n = A,T,C or G
atagacacatagagaaggagacaagaagaagaagagtctaagagtgtcncgagttccagtacctctagcagtgacagttcggccagtgagtcttcatcagagagtgagacatccgcctcatcctcttcagaagacagtgactcagatgaaagcctctctagctcctcctcctcctcctcttcctcggcctgttcgtcttcctcttcctcctcttcctcctcctcctcctcggactcggactccagctcgtccagcagcagcagcagcagcagttctgagagcagctctgatgatgagccacagaa

SEQ ID NO: 33 - DNA, 226 bp, Mus musculus
  misc_feature (1)...(226): n = A,T,C or G
tgtcctggcactctccgactccagcagcagtagcagctccagcgactcanaagcagaggggaaggagtatgccgctggctgcaagaacatgaacgctcctcagacaaggccaagaaacccaaggtgaagaaggagaagatggagaagaaggagaagaaggagaagaaggagaaggagaagaagccccccattgacttattaaacttgcagccctttccgctcccca

SEQ ID NO: 34 - DNA, 459 bp, Mus musculus
  misc_feature (1)...(459): n = A,T,C or G
aggtccaacagaagatatggcccagattaaaggtatcctcaagatctggattaaaggcttgtgtcaatccagatccaaagcctgtgacatcacacctcaagattcagatcaaagcctatgtcttccaggctcaagatccanancacaagtgtgcccaccatttctggattgcagttcattccagatatagtcaagttgacaactgagaagaaccatcacaggttataaacacctatcatttgctctcattttccccatatgttctctgctcatgtgagatcctgacttgaagtgtttcccccaagcttgttttccctgatctctaaaaatacagccagaaagaagctctttgttctctatagccagcaaaaagcctttttgtttctataccttacaaccagagatgcaataattgtaaaaagacctagccaggcacttgcctggctccctgtgccaagg

SEQ ID NO: 35 - DNA, 234 bp, Mus musculus
  misc_feature (1)...(234): n = A,T,C or G
ctgattacagattctgctatgttctgtgccctangaaaccgtgncantagaagtgagtagatctgttttgtatagttacccacaataagcttggacctgaaccaaccacccagcagcactgcctgcctctgtgtataagctnttatggngnnngcctttatanagatgtccctgggatggccccaacataagagagacatctgcactaanaacaacaacaacaacaacaacaac

SEQ ID NO: 36 - DNA, 237 bp, Mus musculus
  misc_feature (1)...(237): n = A,T,C or G
tgctgggcaccatgtgtggtctagggcacagaanagncagaagaagacatcanggtcccctggagcggatgttatcagctgccctgcaagtgctgggaactcaacctaagccttctccaaaaaccaggagtactgtgaagcacctggccgtctctccagtcaccaacaacaacgctgcttgttagcaaatattttgatagcagcaacaagaaattgcctaatataaggtctcaccca

SEQ ID NO: 37 - DNA, 170 bp, Mus musculus
caagactgccctttggtattcaaacgggagccgctttggactgactggctgccttcctcctcctccccctcctcccccttctgcttcagatttatttattatatgtaagtacactgtagcagttgtaggacacactagacgagggcaccagatctcattgtgggtggttg

SEQ ID NO: 38 - DNA, 158 bp, Mus musculus
ggtctagatgtatgtgccatgctgggaaccaggcctggatcgtctgcaagagcagccatcggtcttagctgctaactcctctctcagtccatttgaatacgattctgaggtgacgcttccctgtcaccacccttaacatgctctcatcgtaccctctg

SEQ ID NO: 39 - DNA, 261 bp, Mus musculus
  misc_feature (1)...(261): n = A,T,C or G
agtctcgaaaggtaagtctggncaccatgtgtgtagnnggcacggaaccntnagaataanacatnatgnngcctggagcggatgttatcagctgccctgcaagtgctgggaactcaacctaagccttctccaaaaaccaggagtactgtgaagcacctggccgtctctccagtcaccaacaacaacgctgcttgttagcaaatattttgatagcagcaacaagaaattgcctaatataaggtctcacccataaaaaaaaaa

SEQ ID NO: 40 - DNA, 259 bp, Mus musculus
aagttggaaattgcaatactcctaaaccaaatttctttgattttgaaggaaagcaaaaatgggaggcatggaaagccctcggtgactcaagccctagccaagcaatgcaggaatatatcgcggcagttaaaaaactagatccaggatggaatcctcaggttcctgccctccttgagttcctgtcctgacttcctttgatgctgaactgtgatgaggaagcataagccgaataaaccctttgctctccgaaaaaaaaaaa

SEQ ID NO: 41 - DNA, 177 bp, Mus musculus
  misc_feature (1)...(177): n = A,T,C or G
gggttccaagactgccctttgggattcaaacgggagccgctttggactgactggctgccttcctcctcctccccctcctcccccttctgcttcagatttattnattatatgtaagtacactgtagcagttgtaggacacactagacgagggcaccagatctcattgtgggtggttgt

SEQ ID NO: 42 - DNA, 256 bp, Mus musculus
agggtctcttctctctactcagcaccagctgatctggaagccactctatagagcagactgactttgaaatcacagagccacatcagtctctgcctccccagtgctgaaattaaaggcgcacaccacccgctctggtgacatttttctttaaaaaatgaacaaaaaagggttgcgtttgccagtttttaaaaaagaattgccttttcgatatttgttttgaataataaaatcttacattttgagtgaaaaaaaaaaa

SEQ ID NO: 43 - DNA, 138 bp, Mus musculus
  misc_feature (1)...(138): n = A,T,C or G
ggaggaattttctcagcaaacatcatttaaaaggaaattaatggcgaaaatggggcattgggaaaccatgggggaggggggagactgananaatganaatgaataatggcaaaataaataaataaataaataactaaa

SEQ ID NO: 44 - DNA, 305 bp, Mus musculus
gggagaaactcactcgccaaacaacgtctcgtggtatcctggctgactctggccgcttcagcagagcctggctgttcctgctggattgtgctactgctacagattcatgtttggtgtttgctattgaactgggctgcttatatcctgacacggaagagtggaatcgcccaaagcaagtacttctaatcaggtccacaactccctttgcctttaaccttgctttttccctacctctggttggtgggctaaaagggagggttaagaaccccaaataaagtcagttttgaaaaacctaaaaaaaaaaa

SEQ ID NO: 45 - DNA, 238 bp, Mus musculus
gggaacattgtggcaggaagacggaagtagtgctggagaggagactggagagaaacactgggcctgtccctgtgatgcacttctgccagcaaggccacgcctcccagtccctgaagtgtcactccctgaagatgaagcattcaaatatgagtcgatgagagccattcttgttcacgtcgccatagttatggaggttggacccagcactcaagactgttataaagaatttttgctttgg

SEQ ID NO: 46 - DNA, 363 bp, Mus musculus
  misc_feature (1)...(363): n = A,T,C or G
gggttccaagactgccctttgggattcaaacgggagccgctttggactgactggctgccttcctcctcctccccctcctcccccttctgcttcagatttatttattatatgtaagtacactgtagcagttgtaggacacactagacgagggcaccagatctcattgtgggtggttgngaaccacccaccatgtggttgctgggattngaactcangatctnnataagagnagccagggctntaaaccgntgagccatctctccagcctnctgcattccttcttaangcatgaatgancccnacctangaagacatgctgccntacctccttttcnagccattttccctttcaccatatactca

SEQ ID NO: 47 - DNA, 272 bp, Mus musculus
  misc_feature (1)...(272): n = A,T,C or G
gggttccaagactgccctttgggattcaaacgggagccgctttggactgactggctgccttcctcctcctccccctnctcccccttctgcttcagatttatttattatatgtaagtacactgnagcagttgnaggacacactagacgagggcaccagatctcattgtgggtggntgancncnanngaancagnggnnggtgggngacaanngnanggnngttagaacagaagacnggtgnatanaccnagntactattacgtccatgagact

SEQ ID NO: 48 - DNA, 401 bp, Mus musculus
  misc_feature (1)...(401): n = A,T,C or G
gggtccagaaaaggagatggatgccatacaggccaaggaagaagtgaaaaaggcccgtgaagaagatgaggaggatctgctgatggggaagtttcacaggcacaaccatttcagcagattccacaggcagggcgaactatagtcatcctcactctgcatgctgagtggcccagctgcaactctaataatacacatttgaatgatcacatangnangganantngnccagnttctgaaaaacaatcccctgtttgtgttagggtcccccgtttcttctatagacccctatgaccccagggtctgcaataagaagtttctgttgactgcatgtggtggcacactcctttaatctcaggacttgagaggcagaggcagacntatctgggatttccacatcagcc

SEQ ID NO: 49 - DNA, 372 bp, Mus musculus
  misc_feature (1)...(372): n = A,T,C or G
gagagagagagagagagagagagagagagagagagagatgagatanaggntggaggcctcgnggagcagcctgtgtgcacctgactggnccagnaggaacacagtgtncagagaagctcctgtccccctgtcccctctgggtgccaaataatgggagatgtgcactgtctgatcacatattcccgtctnctgccctntgcccagttaaggttaaggntgatgaataancccttggattattctgagganctgaccannccacagaagaaagcannccctgtancttccctggtcatttcccaagaatcttcctgccctgttgagacttgccccaagtctagggcagtcttcaagcaagcacctcttgcttgg

SEQ ID NO: 50 - DNA, 161 bp, Mus musculus
  misc_feature (1)...(161): n = A,T,C or G
tgaggcctatatgtatgagccatgctgggaaccaggcctggatcgtctgcaagagcagccatcggtcttagctgctaactcctntctcagtccatttgaatacgattctgaggtgacgcttccctgtcaccacccttaacatgctctcatcgtaccctctg

SEQ ID NO: 51 - DNA, 335 bp, Mus musculus
ggtgatttctctgtgccaaagcctccagaggacaatataaaagacgtgtgtacaacgtcacccaggaaaacaaagaagaagaatatagcttgccagtggggagttagtcttgccttttcttttttcctttgaatcagagtctcactggatagctctggctactctgggactctctatgtagaccaggctggcctgagtgctgggactaaagggtgtgtgccactacccccagctaaattggtgtgttctggagagattcttttttatgtttggcccattttaattctttcaggcatcttactactgctgttttaaagtttttttcttttaaacca

SEQ ID NO: 52 - DNA, 473 bp, Mus musculus
  misc_feature (1)...(473): n = A,T,C or G
ctttcagggacccgctgggaagacgaagagagaccagatcatactccagatcattaaggattttaaacccccatgttcctcgtgcggcgctcaccaccaccccccggctctggccatggccacactgagggtccatcccgaagcccaagccaaggtggatgtgttccgtgaagacctgtgtagcaagacagagaacctgcttgggagctattttcccaagaaaatctcagagttggatgcgttcttaaaggagccagctctcaatgaagccaacctgagcaatctgaaggctccattggacatcccagtacccgatccagtcaaagagaaagagaaggaggagcggaagaagcaacaggagaaggaagagaaggaagagaagaagaaaggggacgaagacnacaaaggtccccctgtggnccanttgaactgcatgagaaaaatgngggcctnctgcaacggctaaaacctgg

SEQ ID NO: 53 - DNA, 323 bp, Mus musculus
  misc_feature (1)...(323): n = A,T,C or G
agggggcaaggccatgtgggacaccccanacactgtcctcttanagcgccgggcgggccattaggcaggcgctgctcacaagccgaggcgtgtctctggcagctcctccgactccagcagcagtagcagctccagcgactcagaggcagaggggaaggagtatgccgctggctgcaagaagcatgaacgctcctcagacaaggccaagaaacccaaggtgaagaaggagaagatggagaagaaggagaagaaggagaagaaggagaaggagaagaaggccccccattgacttattaaacttgcagccctttccgctccccaaa

SEQ ID NO: 54 - DNA, 352 bp, Mus musculus
ggaccctgtgtgctgtgcaatcaagacccaaactagaagaagggtctgttttactgctcttggaaggccacgtctaaaattaaaccacgttcaggggaaactgtgcacccaaatatttctcagtcgcaccccagcctcacaaacctattctagggctggaaaagctggctcagccgttataagccctggctgcactaccagaggacccagcctcaatttccagcacccacatggcagctcataactgtgtagatccagttctagaggatccaacaccctcacacatacatgcaagcaaaacaccaatgcacagaaaatgaaaagaaataaatcgttaagaacaaaaacaaaa

SEQ ID NO: 55 - DNA, 305 bp, Mus musculus; identical to SEQ ID NO: 44

SEQ ID NO: 56 - DNA, 158 bp, Mus musculus; identical to SEQ ID NO: 38

SEQ ID NO: 57 - DNA, 274 bp, Mus musculus
  misc_feature (1)...(274): n = A,T,C or G
tactgagcctcacggtttcttntggctggaactgcagttcatgcntggngattgccgagtggattgnagtgcatctgctgatgatttgagtctgtgctgtgctaacggacttcactacaacncccgattggaatcacccccaaagaactgnttgtataacgaggtccacaactctgtttcctatctttccccatgacctttgctgggtggtgggctaaaagggaggttgaagcgtttaagaacccttattaaagtaggtttggaaaaaaaaaaa

SEQ ID NO: 58 - DNA, 221 bp, Mus musculus
  misc_feature (1)...(221): n = A,T,C or G
gtgcaaaaagaagaagaagaagaagaagaanaagangaagangaagaagganaaggacanngacaaggagaacggagaangagcaaggaaaaggagaacggtacagnatnngnggagagaatattnctaaaatatttntgnaatatttgatatgtaatcattacattnagtcaaaaaattcanatttctagaaaatataaagtttttttttgtcgattcct

SEQ ID NO: 59 - DNA, 253 bp, Mus musculus
  misc_feature (1)...(253): n = A,T,C or G
actccaggagaagaaggaagtngtggaggaggcagagaatggaagagatgcacctgccaatgggaacgctcaaaatgaggaaaatggggagcaggaggctgacaatgaggtagatgaagaagaggaagaaggtggggaggaagaggaggaggaggaagaaggtgacggtgaggaggaggatggagatgaanatgangangctgangctcctacgggcaatcgggtngctnaggatgatgatgatgaccatgtg

SEQ ID NO: 60 - DNA, 226 bp, Mus musculus
  misc_feature (1)...(226): n = A,T,C or G
agggttccaagactgccctttgggattcaaacgggagccgctttggactgactggctgccttcctcctcctccccctcctcccccttctgcttcagatttatttattatatgtaagtacactgtagcagttgtaggacacactagacgagggcaccagatctcattgtgggtggttgcaaacgnnngacacactngacgagggcaccagatctcattgtgggtgga

SEQ ID NO: 61 - DNA, 323 bp, Mus musculus
gcatcccttcgtcagcagagtcaccagcaataaggctcttcgggagctggtggccgaggcaaaggcggaggtaatggaggagattgaggatggcagggaggacggagaagaggaggatgctgtggatgctgttccgggctggagagatggctcaacggttaagagcactgactgctcttctcaaaggtcccgagttcaaatcccagcaaccacatggtggctcacaaccatctgcaatgagatctgacgccctcttctggtgtgtctgaagacagctacagtgtacttatatataataataaataaatctttaaaaaaaaaaa

SEQ ID NO: 62 - DNA, 201 bp, Mus musculus
  misc_feature (1)...(201): n = A,T,C or G
cacattcacgaccacacctaaacctacagaatcagaaacgccactagtggggtcggtcctgaaacctggttaacaagctcccaaggtgaatctgatggccgctttccctaagcagtagagaccctttttcgtgtatggtgctgaaaatttaancgtcctctgtccccaaatcagtgaaaaatcnggatttaaaaaaaaaaa

SEQ ID NO: 63 - DNA, 372 bp, Mus musculus
gagactgacccgctcctcaagatttattatatgtacgtacactgtcgctgtcttcagacaccccagaagagggcatcagatctcattaaggatggttgtgagccaccatgtggttgctgggatttgaactcaggacctctggaacagcagtcagtgctcttaaccgctgagccatttctccaacccttaagagtgtattttatttccttttgatttcctgcaagtatcatggtttcagttttctttctggctgtaagactccattgtatgtccgaaccacattttatctgttttacgggccagttggctgcttctgttcttttgcctattgcaatcgctacagcaataaacatggatgtgtaaaaaaaaaaa

SEQ ID NO: 64 - DNA, 284 bp, Mus musculus
  misc_feature (1)...(284): n = A,T,C or G
ggttccctaaaatcagactctgtcatcggcaagtggagactgtaacangactgggaccctcacatgccgataagcagtcaagaatattttctgggagctggagagatgggctcagcagttaagaacactgactgctcttccggaggtcctgtgttcaaatcccagcaaccacagggtaactcacaaccatctgtaatgagatctgatgccctcttcgggtgtgtctgaagacagctacagtgtccttacatataataaataaataaatctttttttttaaagaa

SEQ ID NO: 65 - DNA, 324 bp, Mus musculus
  misc_feature (1)...(324): n = A,T,C or G
gtttgaatttgagtgccgtgctcgaggtgccgacatcttancctacccacctgtggttgcnggcggnaatcgctccaacactttgcactatgtgaagaataatcaactcatcaaggtaatcaccagaattcccacccctgcgttctgctcctggagaggcctggtccccagtagagagctccacacctctcccttagcaaatggagtgccacacgccttcaatcccagcactcgggatttctaagttcgaggccagcctggtctacagagtgagttccaggacagccagggctatacagagaaaccctgtctcaaaaaaaaaaa

SEQ ID NO: 66 - DNA, 431 bp, Mus musculus
  misc_feature (1)...(431): n = A,T,C or G
ggaggacacccacagtttccgcttttgccggcatcgagaggctgcagctctgtttcgctctaaaaaccgctgccgccgctcatctggatcttcatccactgtacgccgtcgccgtcccccagtgctaggggtgggctgagctggagagacctgttgcccagtaaagaagaatacttgtgatggtttgtatatgctcttccctgggagaggcacttttagaaggtgtggccctgttggagtaggtgtgtcactgtgggtgtgggctttaagaccctcatctagctgcctggaagccagtctcccactagcagccttcagatgaagatgtacaactctcagctcctnctgcactaanncctgccaagatgctgccctgctcccaccttgatgacaatggactgaacctctgaacctgtaagcggcccaattaa

SEQ ID NO: 67 - DNA, 427 bp, Mus musculus
gtcgtgtcactgctgttgctatcctgactggactgaactggactgctggtgtatctgtgaggtgtttgcgaatggattgagctgccactgcctgctgacctgtgaactcaactgctgatttccagacaacacagacaggagttgttgcaaagagcctttatagacaggcccgcgcccccttccccgctcctgtatcatttcttttccattacctctggtgggtggtgggttacaagagaggttaaagcatttaggaaccatcattaaaaacagggtttgacatggaggcgcatgcctttaatcccagcactcaggaggcagaggcaggcggatttctgagttctgagtttgaggccagcctggtctacaaagtgagctccaggacagccagagctataaagagaaaccctgtcttgaaaaaaaaaaa

SEQ ID NO: 68 - DNA, 303 bp, Mus musculus
  misc_feature (1)...(303): n = A,T,C or G
gcacatgagtgttctgcctgcatggatgactgtacatgcatggctcatgcagtgatgaaggagaccagaagaggtccttgggaatagagttacaaacaattttgaaccaccatgtgggtgctgagaattgaacctatgtcctgtggaagatcagccagtgctcttaaccactgagaaatctctccatcacctattcttgtggtttggtttttctttttctttctttctttcttttttttttttcnananngggtttttttgggtanccctggctgncctggaactaactttgtaaaccaggtt

SEQ ID NO: 69 - DNA, 214 bp, Mus musculus
  misc_feature (1)...(214): n = A,T,C or G
gaaggagaagaaggaagttgtggaggaggcnnagaatggaagagatgcncctgccaatgggaacgctcaaaatgaggaaaatggggagcaggaggctgacaatgaggtatatgaagaanaggaagaaggtggggaggaagaggaggaggaggaataatgtgactgtnaagatgatgatntannacnataattnntaccatttcgtattgctaat

SEQ ID NO: 70 - DNA, 547 bp, Mus musculus
agaagagaagcagacacaagatgaaaccctgccaaaaaatggaaggaaacctagaaaaggaggatgagccaaagcctgaggaagagccaaagccggaggaaaagcccgaggaggggcaggaacccgaggaggaggagaaatccgaagaaacttttcgggagaggctgatccggtctctgcaggattttcaggaggatatacacaacaggcacctgagcagtgatgatctgtttagagatgtggaggagctagatgagatcaggaaagtgagaaacaaacttatagtgacgcgttggaaggctaaccgaagccatccttacccctatttaatgtagcttggcttgctttcccttttatctgatgttaccattgcgatctagctttcttttcattagcattttcctgataagacctttgcccatcttcctactttgaatctgttgctgttagtggctttagctgtatctcttgttgtctgtatcttactgttgattatatttgaaccaatctataagtctctggctttaagctttactgatttgtaaaa

SEQ ID NO: 71 - DNA, 258 bp, Mus musculus
  misc_feature (1)...(258): n = A,T,C or G
ctactgaacccgagagaatgcngccgggaatcangggccccttgcccttcattcatnactagcgttgcaccnttnaaagttgagccttgatcagaaaaaaaagaccctnaaaaaaactgnctgccccttgaaaacttacctacctaaagtacagggataatagcttctttgcccactgtgactctcagcccttaagactgtttggctctgcactaatttttttttattagtctgtaataaaatacctacattttatta

SEQ ID NO: 72 - DNA, 174 bp, Mus musculus
agggttccaagactgccctttgggattcaaacgggagccgctttggactgactggctgccttcctcctcctccccctcctcccccttctgcttcagatttatttattatatgtaagtacactgtagcagttgtaggacacactagacgagggcaccagatctcattgtgggtgg

SEQ ID NO: 73 - DNA, 364 bp, Mus musculus
aagaagcctgctcattctgaatttgaacatcggttctgcattactcctgaaagatgtcctgcagtcagtgtccgagcacattcctgccacagcagccttaaatgaagttggggtttacaaactggctcagcaagacgttgagattctgcttaatttgaggacaaattggcctaacactggaaaataatgcatcttcgagaaaagtgtttaacaaagacaagagttgcctttaatcctagcacttgggaggcagaggcagatggatttctgagatcgaggccagcctggtctacaaagtgagttccaggacagccagggccacacagagaaaccctgtcttggaaaacaaacacaaacaaaaaaa

SEQ ID NO: 74 - DNA, 274 bp, Mus musculus
aggtatttggcccagcgacatcagagacaagaagagacacagatgagagacaagcacatgaaagatgcttaccatccttcgccatggcaaaaagtgaaattaaaaagtcgaggggacattactgtgcatctgatggagacatgaggacccgtagggcttgctggccagccagtctagcccagtcagtaagctccaggttccattgtgcctgtggtcttcaagagctctgcacacccacagacataaaaataaagttcaggtgaaataaaaaaaa

SEQ ID NO: 75 - DNA, 159 bp, Mus musculus
  misc_feature (1)...(159): n = A,T,C or G
ggtctagatgtatgtgccatgctgggaaccaggcctggatcgtctgcaagagcagccatcggtcttagctgctaactcctctctcagtccatttgaatacgattctgaggtgacngcttccctgtcaccacccttaacatgctctcatcgtaccctctg

SEQ ID NO: 76 - DNA, 282 bp, Mus musculus
  misc_feature (1)...(282): n = A,T,C or G
atacgacacctggaagttgaatntacagaattgccagctgcccaacatggatncttggaaccaaacctttagaattcaacaaacaaacaacccagggggctggagagatggctcagcactcaagagcactgactgctgctcttccagaggtcctgagttcaattcccagcaaccacatggtggttcacaaccatctgtaatgagttccaatgccctcttctggtgtgtctgaagacagcgacataaaatgaataaatctttttaaaaacccaaaaaaaaaaa

SEQ ID NO: 77 - DNA, 523 bp, Mus musculus
  misc_feature (1)...(523): n = A,T,C or G
catatgcccacatcangcattcctcggnctcacctgcgctctgacctggggcagcagttagaggaggntccttcttnatcttcctcctctgagactnagcctctgcctgcaggaacatgtatcccagagccagtgggcctnttacaaactactgaagcccctgggcccaaaggtatcaagggcatcaagggtactgctcctgagcacggccagcagacctggcagtcaccctgcaatccctatagcagtgggcaacgtccatcgggactgacttatgctggcctgccacctgtagggcgtggtgatgacattgcccaccactgctgctgctgcccttgctgctcctgctgccactgtcctcgcttctgccgttgtcacagctgttgtgttatctcctagctgactattgaacctccagggctgtgcagcccaggttcctgctcaatgccnaagtgttgctggacatcangagcanccgttgcatgagcatcaccatttctgcctgacaggggacctgtcacca

SEQ ID NO: 78 - DNA, 326 bp, Mus musculus
  misc_feature (1)...(326): n = A,T,C or G
ggcgaacccccccaacccccagcctgnaacaggctgatccagaccttgaccttgctgccactaaggagattacctagggtggagccttcctgcctagatgactctattgttcagccactgctgctcttctncttcaagacactgctacctgctgagaggaccctgagctattctagagacttcatcctatcctgcaggtcatcacctgcagctggttccaggctccaagaatggattaacgggaatgggccgcccttctcccccccaccccctttataagcacagttttctattaaacattcgagctttgatcaggaaaaaaaaaa

SEQ ID NO: 79 - DNA, 336 bp, Mus musculus
  misc_feature (1)...(336): n = A,T,C or G
ttgaagcgttccaagactgccctttgggattcaaacgggagccgctttggactgactggctgccttcctcctcctccccctcctcccccttctgcttcagatttatttattatatgtaagtacactgtagcagttgtaggacacactagacgagggcaccagatctcattgtgggtggttgtgaaccacccaccatgtggttgctgggatttgaactcaggatctacanaagagcagtnagggctntaaaccgntgagccatntctccagcctcctgcattccttcttaaggcntgaantgatcccanncataggaagactgacttgcccttcctt

SEQ ID NO: 80 - DNA, 370 bp, Mus musculus
  misc_feature (1)...(370): n = A,T,C or G
ggtgctgtctgctccgatctcccttatgcttcctggtttgagtccttctgccagtttgctcagtatcgttgctccaaccatgtctactatgccaagaggnnacnntgctcncagncnnncnccatnctatncccnaacactntnaaggangtggaggcttctncagaantggctgacacttccatgaccanctggcattgnganccatgccacagagtgagacctcncanantgtgnaccnnagntcccagccctancttggcagcttgcaagtctgtaactccagtcctgggagatccaacgcccccttctggcctccacaagcactgcatgaatccaggcaaaacacctatacacataaaataaaaaa

SEQ ID NO: 81 - DNA, 260 bp, Mus musculus
taacagcctcctctatgatcatggcccggctgcatgctgagccacggagaaggagaagaagaagataaaggaggatgagaagggttggggcggtgcccgacctctgaagaggagaggagacaggcgaccatgggagcaagtcttcagcaccacatgatggggactctggagttgccgctcaggctgtctggggtcctggctgactggccaccgtctttctcatcaataaaaacatcgttgtgcattcttaaaaaaaaaaa

SEQ ID NO: 82 - DNA, 158 bp, Mus musculus; identical to SEQ ID NO: 38

SEQ ID NO: 83 - DNA, 270 bp, Mus musculus
  misc_feature (1)...(270): n = A,T,C or G
agactctgtcatcggcaagtggagactgtaacangactgggaccctcacatgccgataagcagtcaagaatattttctgggagctggagagatgggctcagcagttaagaacactgactgctcttccggaggtcctgtgttcaaatcccagcaaccacagggtaactcacaaccatctgtaatgagatctgatgccctcttcgggtgtgtctgaagacagctacagtgtccttacatataataaataaataaatctttttttttaaagaa

SEQ ID NO: 84 - DNA, 338 bp, Mus musculus
  misc_feature (1)...(338): n = A,T,C or G
gttgctcagagaagcacagtcttcaagaccccatacccgaggaggaggaggaggaggaggaggagcccatcggagtggctgtggaggangagcgtttncaccagnagggagccccaanagcatccnataaaagctgtgccattatgtgaacttgtcaagacatggtcgatcttcctcaagctagaancatggagtcctgtatacagtgcagagccttctcagaagcacatgatatttttgtatttcctttatgtgaatttttaagctgcgaatctgatggccttaatttcctttttgacactgaaagttttgtnaaagaaatcctatccatacattat

SEQ ID NO: 85 - DNA, 265 bp, Mus musculus
  misc_feature (1)...(265): n = A,T,C or G
agcctctcttaacattttccntggcaaatctntcagtgcctggctntatgtggtctcccntcccccagcctgaaacctgcttgctnaggggtggagcttcccgctcantcgttctgccacgcccactgntgnaacctgccgntttgctgtttgnagctacacacgtggtcaccgtgctactgnattccganattatttggcggnaattgggcccntccccnttccttnataacggagtgccaaaatagtaaaaattgaaccttga

SEQ ID NO: 86 - DNA, 240 bp, Mus musculus
  misc_feature (1)...(240): n = A,T,C or G
gcccgaaaaacaaaaccagaggaaggggtgagagaaaaanntgcaaatggggaactgaaaaattattgaagggctggtgaggtgactcagtggaaaaaggagcttgctgtcaagtctgaagaccagagttcaattcccaggacccacctggtggaagttgncttcaaactgccccatgaatattcaaacacaagcatgtatattcatacaaaataaataaatataatttaaaaaaaaaaa

SEQ ID NO: 87 - DNA, 220 bp, Mus musculus
  misc_feature (1)...(220): n = A,T,C or G
gggttccaagactgccctttgggattcaaacgggagccgctttggactgactggctgccttcctcctcctccccctcctcccccttctgcttcagatttatttattatatgtaagtacactgtagcagttgtaggacacactagacgagggcaccagatctcttgtgggtggttgngaaccacccaccntgtggttgctgggatttgaactcaggatctt

SEQ ID NO: 88 - DNA, 198 bp, Mus musculus
cggttccaagactgccctttgggattcaaacgggagccgctttggactgactggctgccttcctcctcctccccctcctcccccttctgcttcagatttatttattatatgtaagtacactgtagcagttgtaggacacactagacgagggcaccagatctcattgtgggtggttgtgaaccacccaccatgtggaag

SEQ ID NO: 89 - DNA, 320 bp, Mus musculus
agttgaaggcctccagttaagagattacctcaaatctgaaataaggctttggacttgtgaacagtattggaattgttgaagaatatggggacttttgaagttgtcctggacatatgtttgcattataagatgacaatgaaggctgccaggaaaactcagaaagtattgtattaccttttgacttgagttcaatccccaaagccaaagtgtggaaggagagagctgattcctgcaacttcttctgacttctatagcactcacatatatcgggcacatgcatgagtatgcacacacacacacacacacacacacagatcgaa

SEQ ID NO: 90 - DNA, 173 bp, Mus musculus
tccaagactgccctttgggattcaaacgggagccgctttggactgactggctgccttcctcctcctccccctcctcccccttctgcttcagatttatttattatatgtaagtacactgtagcagttgtaggacacactagacgagggcaccagatctcattgtgggtggttgt

SEQ ID NO: 91 - DNA, 335 bp, Mus musculus
  misc_feature (1)...(335): n = A,T,C or G
acaggctacacacagccggtggctacatgtggnaacatctncagnggntgttgaagagtccgtggtggcatctnntgcngcaatgnggaatgcctgggctgtcctctgncatggctggcccntggcctgttgaccacagaananancagggtcanagggctcaagcncacagnccccttggggagatcgcacatagcagggttctcaacctgtgggttacaacccctttgagggtcaaacgaccctttcacaagagttacgtattagctatcctggatgtcagatatttacattacaatgtataacagcagcaaaattacagttatgaagtagca

SEQ ID NO: 92 - DNA, 235 bp, Mus musculus
  misc_feature (1)...(235): n = A,T,C or G
caaagccacccaccctntgacttctatttactttacaatgatgttagccccccncnnntccaaaantnatgacannggggggtttngnctctggncaaacatgacctccagagtttggcccttgcataaaggagaaaattaattccttcagttgttctctgacctccacatgcgtcctgtaacatgggtgtatgcatacacataaacacacatncacacgttgttaaataaataa

SEQ ID NO: 93 - DNA, 173 bp, Mus musculus
  misc_feature (1)...(173): n = A,T,C or G
gggttccaagactgccctttgggattcaaacgggagccgntttggactgactggctgccttcctcctcctccccctcctnccccttctgcttcagatnnattnattatatgnaagtacactgtagcagttgtaggacacactagacgagggcaccagatctcattgtgggtgg

SEQ ID NO: 94 - DNA, 205 bp, Mus musculus
gggttccaagactgccctttgggattcaaacgggagccgctttggactgactggctgccttcctcctcctccccctcctcccccttctgcttcagatttatttattatatgtaagtacactgtagcagttgtaggacacactagacgagggcaccagatctcattgtgggtggttgtgaaccacgcaccatgtggttgctgagat

SEQ ID NO: 95 - DNA, 315 bp, Mus musculus
  misc_feature (1)...(315): n = A,T,C or G
cactgttttgtaactattttgtaaggncacagtccaccagtanctttagggnacagggtnattttatncacancagcagnaaatgnaaaccaaaacaactccacaataaaattcttgngtttcttggggccaagaagtatgnacacgnataagnataaagnacactggccaagncatgtacctccagnagtttggcccttgcataaaggagaaaattaattccttcagttgttctctgacctccacatgcgtcctgtaacatgggtgtatgcatacacataaacacacatccacacgttgttaaataaataaaaa

SEQ ID NO: 96 - DNA, 340 bp, Mus musculus
ggcactccagttaagagattacctcaaatctgaaataaggctttggacttgtgaacacgtattggaattgttgaagaatatggggacttttgaagttgtcctggacatatgtttgcattataagatgacaatgaaggctgccaggaaaactcagaaagtattgtattaccttttgacttgagttcaatccccaaagccaaagtgtggaaggagagagctgattcctgcaacttcttctgacttctatagcactcacatatatcgggcacatgcatgagtatgcacacacacacacacacacacacacagatcgaaaatagttgtaataaaaaaaattaaa

SEQ ID NO: 97 - DNA, 507 bp, Mus musculus
  misc_feature (1)...(507): n = A,T,C or G
gatctggtggcttgggtgacagccagtttcctgcacatcccccatgctgaggacatccccaacacagtgactgtgggaaacagagtgggcttcttgctccgaccctacaatttcttcaatgaagatccctctatcttctcccctggcagcgtgtactttgagagggaccaggatgccgggctctgcagcatcaaccctgtggcctgcacccaacagctggcagactgtgtccccaacttaccctccttttcctatgaaggcttataggcctgaggctatggtgaggccccagggtaggagggatggaatgtggagtggagaaatggctcagcgattaagagcacttgactgntcttncagangnccctgnngttnanttccttgcaaccacatggtggntacaaccatctgtaatgggatctgatgctctcttctggtgtgtctgaagacagagacagtgtacccatatacataaaataaatcttttaaaaaattttaaaaaaagaa

SEQ ID NO: 98 - DNA, 128 bp, Mus musculus
tcagcaaacatcatttaaaaggaaattaatggcgaaaatggggcattgggaaaccatgggggaggggggagactgagagaatgagaatgaataatggcaaaataaataaataaataaataaataaaaa

SEQ ID NO: 99 - DNA, 179 bp, Mus musculus
tcagggctccaagactgccctttgggattcaaacgggagccgctttggactgactggctgccttcctcctcctccccctcctcccccttctgcttcagatttatttattatatgtaagtacactgtagcagttgtaggacacactagacgagggcaccagatctcattgtgggtggttg

SEQ ID NO: 100 - DNA, 282 bp, Mus musculus
aatcagactctgtcatcggcaagtggagactgtaacacgactgggaccctcacatgccgataaagcagtcaagaatattttctgggagctggagagatgggctcagcagttaagaacactgactgctcttccggaggtcctgtgttcaaatcccagcaaccacagggtaactcacaaccatctgtaatgagatctgatgccctcttcgggtgtgtctgaagacagctacagtgtccttacatataataaataaataaatctttttttttaaagaataaaaaa

SEQ ID NO: 101 - DNA, 216 bp, Mus musculus
  misc_feature (1)...(216): n = A,T,C or G
gagggttccaagactgccctttgggattcaaacgggagccgctttggactgactggctgccttcctcctcctccccctcctcccccttctgcttcagatttatttattatatgtaagtacactgtagcagttgtaggacacactagacgagggcaccagatctcattgtgggtggttgtgaaccncccaccatgnggntgccgggatttgcactca

SEQ ID NO: 102 - DNA, 343 bp, Mus musculus
aagggtctccctgcctagaacctgatatttaacactgagagatccacctggctctgcgccgctaaacacgccacctcgtgctagccaactttgaacttatgattctcttgtacactggtaacttggaagttacctctcgggggctggagagatggctcagtggttaagagactgactgctcttccagaggtcctgagttcaattccaagcacatggtggctcacaaccatctgtaatgggatctgatgccctcttctggtttgtctgaagacagatacagtgtactcgtcacatatataaaataaataaataaaatctttttttaaaaatttaaaaaaaaaaa

SEQ ID NO: 103 - DNA, 165 bp, Mus musculus
  misc_feature (1)...(165): n = A,T,C or G
gcctccagactgccctttgggattcaaacgggagccgctttggactgactggctgccttcctcctcctccncctnctgnccccttgtgcttcagattnatttattatatggnngtacactgtagcagttgtaggacacactagacgagggcaccagatctcattg

SEQ ID NO: 104 - DNA, 361 bp, Mus musculus
  misc_feature (1)...(361): n = A,T,C or G
gacgcatcctggactccacaatgaacgggacagttggacctaagtgggaagctgattatcaaagctnancttggggaanatatncnacnaattcccattcataatgaagacattacttacgatgaattagtgctaatgatgcagcgagtattcanaggaaagcttctgagtantgatgaagntacaataaagtataaggatgaaggcatggactgaagaccctcaccctagctgcctggaaatcagtattatgttagcagccttcagatgaagatgtagaactntcanntccttctggatcatgcctgcctggatgctgcatgttcctgccttgatgatantagaatgaacctctgaaccc

SEQ ID NO: 105 - DNA, 165 bp, Mus musculus
ggttccaagactgccctttgggattcaaacgggagccgctttggactgactggctgccttcctcctcctccccctcctcccccttctgcttcagatttatttattatatgtaagtacactgtagcagttgtaggacacactagacgagggcaccagatctcattg

SEQ ID NO: 106 - DNA, 349 bp, Mus musculus
gtaaaggcattgaaagagaagattgaatctgaaaaggggaaagatgcctttccggtagcaggtcagaagttaatttatgcaggcaaaatcctcagtgatgatactgctctcaaagaatataaaattgatgagaaaaactttgtggtggttatggtgacaaaacccaaagcagtgacaacagcagtgccagctacaacccagccatcaagtactcccagccccactacagtcagttcttccccagcagtggctgcggcccaggctccagctcccacccctgctctggctcccacttccctctgcctccctagtgctaggattaaaggcatgcactacaaccaaaaaaaaa

SEQ ID NO: 107 - DNA, 424 bp, Mus musculus
  misc_feature (1)...(424): n = A,T,C or G
gaggaggacctgccttctgctgatgagctggaggactggctggaggacgtgctggagggcgagatcaacacagaggatgacgacgactgatgacgacgatgacgatgatgacgatgatgatgacgactagatgactagtggccgtggcagccgcgtaccagccccacttgctccttacttgttgtacctttctgccatctgtgcccttccccgggctccttgggacactaggtcattctctactacagggccaactgtggtctgcatacagggtgcttgaggtcctggtctccctctccgaggagaaaaggagctgctttttcttagatcccagcccagttctctcatctgactttgctttnttncccccagacctttatctggatctcttnccgggcantnccccatactttaacgggttttc

SEQ ID NO: 108 - DNA, 352 bp, Mus musculus
ggttgagaagtaccggccacagaccctggccgatctcatttctcaccaggacattctgagtaccaacccttggtgagcacaacttcgtggcagtgggagcactctgctccaaagtggacaggaagtagagagctaaaggggtgggggagcaagcagtatgaccctcaaaggcacgagtttggtgacctgactcctccaatgaggctgtgcctctaaaccagtggctctcaaccttcctaaagatgtggcccttttaatacagttccccatgttgtggtgaccaccccccccaccataaaattatttcattgctacttaataaatgggaattgggtcctgttaaaaaaaaaaa

SEQ ID NO: 109 - DNA, 386 bp, Mus musculus
gtatttactcaagagaatgcaacctgtgcctacatagaccttcatacatgaacaatcatagcagcttcattcataacagctccaaatggggagcagctcaagtgcccatcaaggtatcctgacctcctgaccgggtccgtgaccttgggaagctgaacaccaacccctcttcacccagaggacgacgccagcggcaggtcatgggagcgcggccctcgcgacggcagatgactgaagctcggcgcctggctctaaccaagctgccccctgagctgctggtgcaggtgctgagccacgtgccgcctcgcgcgttagtgacccgctgccggccagtgtgccgggcctggcgcgacctggtagatggcccaagtatatggctgctgcaa

SEQ ID NO: 110 - DNA, 100 bp, Mus musculus
gaacaacgaccacgggaaggttgaaggataatcttctaataccagcactttgaatgagaatttgtttctctgccacaaactgatttttttttttaaaaaa

SEQ ID NO: 111 - DNA, 506 bp, Mus musculus
  misc_feature (1)...(506): n = A,T,C or G
actgagagtaaagacatttccacgtggacacctgatcatgtgcctaccctgactgaggctccccaggcatctntattgngggcagccgtgctacacgctcaactntgagcccccaacagtttgcaggctcccagccccggggtctgggcctnggccccaccatgtcgagcctcggcggtggctcccaggacgccggtggcagtagcagcagcagtaacaccaatagcagcagtggcagtggccaaaaggcaggaggaacagacaaaagtaccgcggtggccgccaccacggcgccgacctccgtggcagacgatgccccaccccctgagcgtcggaacaagagcggtatcatcagtgaacccctcaacaaaaacctggggggcttcccgaccactcttttnantactnttcntttggnaagcaagtggtggcggcggaaacatgatgggggtggagttttgntgacaaggcaacggcagcccgcagcctccctattggccaatggt

SEQ ID NO: 112 - DNA, 100 bp, Mus musculus
aatcgagatccgagatgaagactgaactttctcctcggcccggggcatgcagggcagggagttgacccaagaagagaagcttcagcttcgaaaaaaaaaa

SEQ ID NO: 113 - DNA, 189 bp, Mus musculus
cggttccaagactgccctttgggattcaaacgggagccgctttggactgactggctgccttcctcctcctccccctcctcccccttctgcttcagatttatttattatatgtaagtacactgtagcagttgtaggacacactagacgagggcaccagatctcattgtgggtggttgtgaaccacccgac

SEQ ID NO: 114 - DNA, 360 bp, Mus musculus
  misc_feature (1)...(360): n = A,T,C or G
taccctcagaggccagaagagggtgtcagagcccttgtaactggagtttgacggtcgtgagctgcgcatcttcaccatggcanacgatgatccctacggaactgggcaaatgtttcatttgaacactgctttgactcactcaatatttaatgcagaattatattcaccagaaataccactgtcaacagatggcccataccttcanatnttatancaaccaaaacagaggggatttnnattccgntatgtgtgtgaaggcccatcacacggagggcttcgggagcctctagtgagaagaacaagaaatcctaccccaggtcanaatttgccantntntgngggcctgcaaaggttatccgc

SEQ ID NO: 115 - DNA, 375 bp, Mus musculus
  misc_feature (1)...(375): n = A,T,C or G
gaaccctaaggactctgcaatatgaataattctntagagaacaccatctcatttgaagagtncatccgagtaaaggcgaggtctgtcccgcaacacaggatgaaggaatttctggactcactggcttctaagggaccagaagctcttcaggagttccagcagactgccaccaccaccatggtgtaccagcagggtgggaactgcatttatncagacagcactgaagtggctgggtctttgcttgaacttgcttgtccagtcacaaccagngttcaaccacaaccatctgtancaagatctgatgccctcttctggagtgtctgaagacagctacagtgtacttacatataataaataaatctttaaaaaaaaaaa

SEQ ID NO: 116 - DNA, 100 bp, Mus musculus
  misc_feature (1)...(100): n = A,T,C or G
gagccaggaagtaatcaagaaatcaaggagtttgcatccggntacaacgtcaaggttgacatgtacagnangatctgtgtaaatggggaccatcccacca

SEQ ID NO: 117 - DNA, 412 bp, Mus musculus
  misc_feature (1)...(412): n = A,T,C or G
gtttgtcaggatggtggataagaatatttacatcattcaaggagaaattaacattgttgttggtgccatcaaacgaaatgcccgatggagcacccatataccactggatgaagaacgggatccccttctgcacagtttcagtaacctgaaagaggtcttaaacagtgtgacagacccantggatgtcaaagagaaagaaatggccttataaaattcatttgtgtggagaacttgaagntacttcggaacttgataacttttagaaccatactctgtttccctaaagaacctcctaatttttttaagcctgatagtggtggtgcatgcctttaatcccagcactcaggagtcagagacaggtggatctttgatttgaggctacctggtctncagannaaggtccaaggccagt

SEQ ID NO: 118 - DNA, 350 bp, Mus musculus
  misc_feature (1)...(350): n = A,T,C or G
gacagaggtttacagnacagatggctgcgtntcagtcccctgctgtaaaaggttctatacctgcaacatcaacagttcagaatgttcttattaatccattgttaattgggtccaaaaacattcttattactactaatatggtatcacaaaatacagccgagtcagcaaatgcactgaaatgaaaacgtgaagacgacgatgatgatgaagatgaagatgaagatgatgatgatgatgatgatgactatgataatatgtagtctaaccacgatgcatgtaacctgtgttcttggacttggatccattgtactacaaattaaaaagcatgttagatgttttaaaaaaaaaaa

SEQ ID NO: 119 - DNA, 165 bp, Mus musculus
ttgagggatggctactgtcacctgaccactaacggacttcgagctggcgacatcaaaagaagcctctatttcaatattccaggcctgagaagcgttatctacagagagaaaggagttccaggacagccagggctacaagaagaaaccctgcctcaaaaaaaaaaa

SEQ ID NO: 120 - DNA, 327 bp, Mus musculus
  misc_feature (1)...(327): n = A,T,C or G
cccctaccacttccttcataactggaagtngcaccantaaacanntgagccttgatcatcacaaaaaacacgagantacanccactgtcaaagtggtgactagccactctgtggacaagaaaacaagtcaccccaatagaggtgggtccaagaggcgggactaanaaaacaaagcctgccnggccgggtgtggtggcgcacgcctttagtcctggcctttgggaggcagaggcaggcggatttctgagttcgaggccagcctggtctacagagtgagttccaggacagccagagctacatagagaaaccctgtctcaaaaaaaaaaa

SEQ ID NO: 121 - DNA, 799 bp, Mus musculus
  misc_feature (1)...(799): n = A,T,C or G
aaacaccgcctgagagcancttaangaggcctacnantcangacacttgatgataccacaccagntccaggtgggagatgcaggtctcgtctgacgacatcnttctgagaatcngaanccttgntaaatnggcncttgnatcgngctactnaccacccacactgctgtcaaagaanaaggcatntattntgcctttnacgntctagcgagcntctgannaatnaaccncnttgattgaccactctcatttntactctgatagaccctctaataatnttgctcttactgctgacgtgtggcccactantncttaataatctaatgacctttgttagagagaggattggtgctgnccagntgntagtattaanacaacnatnccagtctctgcataaccttgaaaaagaggttgcagtttagttctaagattagaactagtaactagaagaagtggggaatgaaagatcctggcagaatgtaaaaggacacagaagccctgaaattggcaagatagacgtcagtgttagcagaactagcttcactgatttagaaacntagaggtgcacaatgctttgggccactccttgaacctgngngtcttggcaatgttctgaccaaggtgtgtgcccattggntgganccttnattgaagactctttcccttggaccccntncccattaccccattttctttggggaattngnncnttggnttaanaatcntgggaaaaatcccccttaaatnttccccccttnttncnttttncccccccttggannggccctttttaaaaaacct

SEQ ID NO: 122 - DNA, 440 bp, Mus musculus
  misc_feature (1)...(440): n = A,T,C or G
gggtcccgacacccctttgaaaaccccctttttgtaaaagccaggcttggtagtgggcacctgtcatcccagcactcaggaggcagggtcaggaggattacaggttcaaagccagtctggtctatattagaaaaagaaaactgtccttttgctaaagaaaaattaaatgaatttttagggctgaaatctgtgggtaagatggagtctggagctttcctcttggaacagttaaccgaagaccctcatgggcaaggacatccgcttgactccccactcaccatcaggccacacttttaaagaaaaattgttctctaaccaggattgtaacaaatgtgtaaatagtatttctgagttgaaagtcgatggtgcaaatatgtatataacgtactgnatttttacaatgatcgtcgcatggctctccacccctttttggactcaag

SEQ ID NO: 123 - DNA, 435 bp, Mus musculus
  misc_feature (1)...(435): n = A,T,C or G
cttcccttgtcgctgctgaagacagcgcagaatcaccccatggtaagcactctgggccacctcccttcatgtgcccacagctcagtcttttgcagntcatgattgaaggcctggcaagatggctcaatgggtaaaggtgcttgctgccaagccgaaggacctgagttcaatccccaggacccatgtgatgatggaggggtncatgacttccctgaagntgttctctgccctgtntgcacacagacagaatggcaaatgcaatacaaaggaaaacgttcacaaagaaatctatagccgggcatggtggtgcacgcctttaatccccgcacttgggaggcagaggcaggcgaatttctgagttcgaggccancctggtctacaaagtgagtgccangacagccagggctacacagagaaaccctgtctcaaaaaaaa

SEQ ID NO: 124 - DNA, 204 bp, Mus musculus
aggagcacatgaaggataaggataaggaccgggaaaagtctaagcatagcaacagtgaacataaagattctgaaaagaaacacaaagagaaagagaaaaccaaacacaaagatggcagctcagaaaaacataaagacaaacataaagacagagacaaggaaagacgaaaggaggaaaagattagagctgctggggatgcaaaaa

SEQ ID NO: 125 - DNA, 292 bp, Mus musculus
  misc_feature (1)...(292): n = A,T,C or G
gggttccaagactgccctttggtattcaaacgggagccgctttggactgactggctgccttcctcctcctccccctcctcccccttctgcttcagatttatttattatatgtaagtacactgtagcagttgtaggacacactagacgagggcaccagatctcattgtgggtggttgtgaaccacccaccatgtggtngcngggantcgaactnaggancnncataatagcacncanggctcnaaatnncttgagccntctcttcnnnctntctgnattccttgttatggcat

SEQ ID NO: 126 - DNA, 175 bp, Mus musculus
  misc_feature (1)...(175): n = A,T,C or G
ggttccaagactgccctttgggattcaaacgggagccgctttggactgactggctgccttcctcctcctccccctcctcccccttctgcttcanatttatttattatatgtaagtacactgtancagttgtaggacacactagacgagggcaccagatctcattgtgggtggttg

SEQ ID NO: 127 - DNA, 158 bp, Mus musculus
  misc_feature (1)...(158): n = A,T,C or G
tgccctttgggattcaaacgggagccgctttggactgactgnctgncttnctcctcctcnccctcctnccccttctgcttnanatttattnatnatatgtaagtacactgtancagttgtaggacacactagacganggcaccagatctcattgtggg

SEQ ID NO: 128 - DNA, 283 bp, Mus musculus
actgtactgactgctggtgagactgctgcaatggctcgtcagtgtaccccgctcctccagcagccagcgcactgcctcaataccctcgttcaaggtcaccagctgctgcaggatcttcacatcgatagctcgcaggcactctggaggtggaaggaagaggatcagaagttcaaggccattctcagctatgttggcagttcaagaccagccggagcttcatgaaaccctgttttaaaattaatgaaataaaataataaaataaaacaagatttaaaaaaaaaaa

SEQ ID NO: 129 - DNA, 176 bp, Mus musculus
ggttccaagactgccctttggtattcaaacgggagccgctttggactgactggctgccttcctcctcctccccctcctcccccttctgcttcagatttatttattatatgtaagtacactgtagcagttgtaggacacactagacgagggcaccagatctcattgtgggtggttgt

SEQ ID NO: 130 - DNA, 156 bp, Mus musculus
ggtttcttctctgtgtagccctggctgtcctggaactcactctgtagaccatgctggcctcgaactcagaaatcctcctcgtgtgccaccacttcccggccgggtcatagcttttttaacctttaaataaagagcttttgttgggaaaaaaaaaaa

SEQ ID NO: 131 - DNA, 251 bp, Mus musculus
  misc_feature (1)...(251): n = A,T,C or G
ggagttccaatgaattcactcaggtttctctttgaaggtcagagaattgctgataatcatactccgaaagaantgngnntganaaagtatganggctggagagatggttcagnanntaanancactgactgctcttctggaggttcaattcccagcaaccacatggnggcttataaccatatataatggagnctgatgcactctcctgggtgtgtgnctgaagacagctacagtgtactcatataaatata

SEQ ID NO: 132 - DNA, 220 bp, Mus musculus
gatcacttcatggatttgcctcacgtacctatgtttgttaatttcctgtgtcgttcccaagatcacgtaaaaccctctgagcattattgacctccctcccagcaaccacatggtggctcacaaccacctgtaacagagatctgatgccctcttctggtgcgtctgaagacagctacagtgtacttacatataataaataaataaatcttaaaaaaaaaaa

SEQ ID NO: 133 - DNA, 269 bp, Mus musculus
  misc_feature (1)...(269): n = A,T,C or G
ggtgcatgatgagttcattgttgcagntgtacacgactcactcatgaaaaggatgtcagagaccccattacagatggttgtgagccaccatgtggntgctggaattgaactcaggacctntggaccttaacctctgagccatctccccagccccaaaacaccaaatgactctaccaaatcatcaagaagagaattcctctggcttcggctcaaggtccatgcttgttcacctctggatattaaatgggcgataattccaaaaaaaaaaa

SEQ ID NO: 134 - DNA, 322 bp, Mus musculus
gttgggttaacggcatggagaacagtcacccccaccaccaccaccagcagcccccgccgcagcccggcccttcgggcgagaggaggaaccaccattggagaagttacaagttgatgattgacccggctctgaaaaaggggcaccataaactgtaccgctacgatgggcagcatttcagcctggccgtgagtagccggcgcgccttcccgccacccccacccacccccggtggagcccccagacaagaggcgaacggagggggacccctcttccaggacagccagggctatacagagaaaccctgtctccaaaggaaaaaaaa

SEQ ID NO: 135 - DNA, 186 bp, Mus musculus
ggttccaagactgccctttggtattcaaacgggagccgctttggactgactggctgccttcctcctcctccccctcctcccccttctgcttcagatttatttattatatgtaagtacactgtagcagttgtaggacacactagacgagggcaccagatctcattgtgggtggttgtgaaccaccca

SEQ ID NO: 136 - DNA, 406 bp, Mus musculus
  misc_feature (1)...(406): n = A,T,C or G
gtctactaacccaactctactgaactggcctgcccctgtatgcatgaagtgtctctgcgagtggatcgagctggcagtgcctgctgacctgtgaactgaactgctggtttccagacaacacagatgggagttgctccaaagaacctttctgaacatgtacacttcccccgtatcctttcttttctactatctcaggtgggtggngggctataagggaggttaagacatttaagaaccgtcattaaaaataaggattgaggggctggtaagatggctctgcaggtgagagcactgactgcttttncgaaggtcctgagttcaaatcccancaatnacacggtggctcacaaccatctgtaatgggatctgnannccctnaatctggtcntttgaagacagcttacaa

SEQ ID NO: 137 - DNA, 490 bp, Mus musculus
  misc_feature (1)...(490): n = A,T,C or G
gaagctgcagtgcttggttcgggtcntatgttcaggggaccantatttagataaagaagaaaatagcaaaattatggatgttgtgttccagtggcttacaacaggagacatccacctcaaccagatcgatgctgaggacccagaggtnggagactgcatcatagaaatggangatggctcannttcttagaaccatttncaaagggacaganagatctntttcaactcacccctaanacnnttggaanacagnttggaganntggctcataggctaaaaatactggctgttcntccagaaggcccaacaaccacatggtggnttacaaccatcctttangggatcctgggccctctttnggggggccccccgtctnttaatttaaatnancggtttttttttnttgtttccggggggngggcgncccctccccttggaacttntttnncctnanttgaacnttttttcttttggaaaagggcccccttgg

SEQ ID NO: 138 - DNA, 290 bp, Mus musculus
ggttccatggtgacggccacacccaccccgctgcggccagatgtcatgcgggtcacctcactccagggctggggatgagaccttaaaccctggattcaagaaagagaccagaggactggagagatggctcagtggttaagagtaccaacggaaggtcctgagttcaaatcccagcaaacacatggtggctcacaaccacccataatgagatctgccgccctcttctggtgtgtctgaagactgctacagtgtacttatacacataaataaaatctttttaaaaagaaaaa

SEQ ID NO: 139 - DNA, 256 bp, Mus musculus
  misc_feature (1)...(256): n = A,T,C or G
ggttccaagactgcccttcggtattcaaacgggagccgctttggactgactggctgccttcctcctcctccccctcctcccccttctgcttcagatttatttattatatgtaagtacactgtagcagttgtaggacacactagacgagggcaccaggtctcattgtgggtggttgtgaaccacccaccatgtggttgctgggatttgaactcaggatcttcanaagagcantcagggntctaaaccgctgagccat

SEQ ID NO: 140 - DNA, 327 bp, Mus musculus
gtctccatatgctcagtcaaatctccataactgtaaatcaacactaagagcactggatagttaaggtcaaggagactctcacaaaggatccagaaggtgtagtccagattaaggtgtgttccaccacacctttaatcccggatgaccttgaactcggagatttaatcttctggaatccatagccactatgcctcaagatctccataccaagatccagatcagaatcttctatctccaagcctccagataagggtacctggtgagccttccaattctggattgtagtccattccaaataaaatcaagttgacaaccagaaaaaaaaaa

SEQ ID NO: 141 - DNA, 301 bp, Mus musculus
  misc_feature (1)...(301): n = A,T,C or G
tccaagactgccctttgggattcaaacgggagccgntttggactgactggctgccttactgctcctccccctcctnccccttctgcttcagatntatntattatatgtaagtacactgtagcagttgtaggacacactagacnagggcaccagatctcattgtgggtggttgcaaacnnangacannntagacganggcaccaganctcattntgggnggangagcatncanggctctnaaccgctnagcnatntntccagnctcctgcattncttcttaaggcatgaatgatccagcata

SEQ ID NO: 142 - DNA, 141 bp, Mus musculus
gccactcgcaaacgacctgtcttctcacgctctctctctctctgtttctcactctgtcctctctctgtctctatcctatactaacttgtttcaaattggaagtgaggatatgtattgcccaagggattaaataaacaaaaa

SEQ ID NO: 143 - DNA, 349 bp, Mus musculus
gtgagccctatttctagagacagctgctgctgaccctgtaactcaaaggacaaactagctggctaaactcattcttggtactggtgttggattattctagaggttggatatgagcctaggtggcctgctggcctaggaggaggattgaggtcactttcttgcagttgcctttaagaatgtgtggaatcggggctggtgagatggctcagtgggtaagagcacccgactgctcttccaaaggtccgaagttcaaatcccaagcaaccacatggtggctcacaaccatccgtaatgagatcttgactccctctttttggagggtctgaaaacaagctacagttgctcttac

SEQ ID NO: 144 - DNA, 238 bp, Mus musculus
cctgaaacctggctgctcaggtttcactgttatcgggaagagagcatgtggtggagcctgccgccctatcattctgccactcccactgctgctgcctgctgcctagctgttccggagccaaacacgtggtcacctgcaactggactcgtaggatgatttggcaggaatgggccccttccccttcttcataacccagtgtctggaatagtaaaatcgaaccttgatcagaaaaaaaaaa

SEQ ID NO: 145 - DNA, 304 bp, Mus musculus
ggagaaactcactcgccaaacaacgtctcgtggtatcctggctgactctggccgcttcagcagagcctggctgttcctgctggattgtgctactgctacagattcatgtttggtgtttgctattgaactgggctgcttatatcctgacacggaagagtggaatcgcccaaagcaagtacttctaatcaggtccacaactccctttgcctttaaccttgctttttccctacctctggttggtgggctaaaagggagggttaagaaccccaaataaagtcagttttgaaaaacctaaaaaaaaaaa

SEQ ID NO: 146 - DNA, 250 bp, Mus musculus
  misc_feature (1)...(250): n = A,T,C or G
gaccttgttggaagaagtgtgtcattgtggtcatggactttaagacctgcancctagctncctggaagccaatctttacctagccgcctntgaaacagctcctnctgcaccatgcctgnctggacgctgncatgcttcnccttgatgataatggactgaatctntgaacctgggcaaagagccnatggggagaagagaatcttnangctctgancatccaggagggtgaagatgttaccatgaactgcag

SEQ ID NO: 147 - DNA, 334 bp, Mus musculus
atctaaaaaaactcaaagcaatgtccctaagaagccacttgaaaattcctagatggcctgggagttagaagcatgaaacacagcctcaaaaccaaaaccaaaaagcgggctacaggtataaaatgagtgttaaagaagtttgaaatagggctggtgagatggctcagtgggtaagagccgactgctcttccgaaggtccgaagttcaaatcccagcaaccacatggtggctcacaaccacccataatgagatctgatgccctcttctggtgcggctgaagacagctacagtgtacttacatataataaataaataaatcttttaaaaaaaaaaa

SEQ ID NO: 148 - DNA, 182 bp, Mus musculus
ggttccaagactgccctttgggattcaaacgggagccgctttggactgactggctgccttcctcctcctccccctcctcccccttctgcttcagatttatttattatatgtaagtacacttgtagcagttgtaggacacactagaccgagggcaccagatctcattggtgggggtgggttgg

SEQ ID NO: 149 - DNA, 337 bp, Mus musculus
  misc_feature (1)...(337): n = A,T,C or G
gtgctgggattgcaggcatgtggccactgtgacaggtttcctgtgctgtgccgaggcccgggcccagcgcctcgtgcacactagagaaaattctgcatactgacccacatccccagctctacccgtgtatatttctagtcattctttaaacccaagcaaatgtgtagagtctcctatgctgagatgttttttgccactaccagacatgtaggctatcatttgacgtctttattccctgagagaaaacttcttttctgtatttatgaactttattattgagaatcactgntaacctttctgccaaattaaagacacttgtgtctctgtttacacggac

SEQ ID NO: 150 - DNA, 377 bp, Mus musculus
  misc_feature (1)...(377): n = A,T,C or G
actctagaccatgtgcctaggtagnttttnccttctccgcagctctgcttccccagcancgctcaccacacccttgttttgagaacctcactaagtctggagctcanagaaatgangaaggtggatggagactgactcatgngcttgaaaccatcaaattgttcaagatcatcaagggactgagcctaaggctctgtcggaggagggagaagccactgaaacagggggacccaggactcacatggtggctcgtaaccatctgttactctaacatccaattctgacctgtaggcgccagtcatctacaagatgcatagacacatgtaggcaaaacacttatacacataaaataaaactgaaaacaagaatttaaaaaa

SEQ ID NO: 151 - DNA, 354 bp, Mus musculus
  misc_feature (1)...(354): n = A,T,C or G
gtctttttccccttcctttctgtggacacagnacagctcttcggttncaggattgtgctggtcaattattttctcctacttccatgttatcaagactacttctacatctttttcactgtatgtatatatgagtgaaattatttagagcattatttctaaaaaatgtatttccagctctaaaataggttgagattatcatgaacatttgttgccgggcagtggtggcacacgcctttaatcccagcacttgggaggcagaggcaggcggatttctgagttcgaggccagcctggtctacagagtaagttccaggacagccagggctacacagagaaaccctgtctcgaaaaaaca

SEQ ID NO: 152 - DNA, 669 bp, Mus musculus
  misc_feature (1)...(669): n = A,T,C or G
aagcctgaggttgagctggaaccggcttcgaatctgccgcttgggagagatgagagggacgggggctgactgctgctggaccgactcgctcagctccttcaggtccatctcggccttgcttcgatctggaagacaccagtatagcctcaatcagaccaggttctcacactccaccacccaccactcagggtttctctgtggagccctggctacactggaactcactctgcagaccaggctggcttcagactcagatacgtgtctgcctctgccttctgagagctgggattaaaggcctgtgccaccaccacccagttcacatgaaatttaaacacgtccgcccgtaactctagttctgggcaatctgacatcctcttctggcttccgtgggcaacgcatgcacgtggtgtgcctatccacgcattggatctagatgttcgtgtgcataaatgcctttaaaaataagctttacacataaataagagcggaggagtttgcgcctggcctcaaatgctgctcctgacgagaagcacctgactcctggagtaaacatggattcaagtcctggagtggacgcccctttccttntgggaaatggacccggagcagcaattagcntacctatctgctaggtcaagcacacaacaccncaaattaaggaaaaaaaaa

SEQ ID NO: 153 - DNA, 336 bp, Mus musculus
ttgagggtcatctgtctttgttgtctgagatgcaagagtcacaggagaagtcatcaccacggtctgcgggagagacgtggcagacggcgtggtacggatctgatctacaactgatggccaactagtgacacccagatgactgtcacattcacgactcaggtatcgaaggatgttccagcccctccctactacctcccaccacagggtatcactatgtagcctggctgtcttggaactcactacatagaccagcttggtctcgaactacagagatctacctgcctctgcttccggagtgctgggattaaaggctactgagccagtcaaaaaaaaaaa

SEQ ID NO: 154 - DNA, 582 bp, Mus musculus
  misc_feature (1)...(582): n = A,T,C or G
agctgtttcccaagccgaagttagagttctcctccagtcgtctggaccaaggggagttgttggacctgncctgctccgactcgggcacacctgtagccaacttcaccatccagaaggaagagacggtcttgtcgcagtatcagaatttcagcaagatcgccgaggagagtgacagcggggagtacagctgtactgcaggcatcggcaaagtggtcaagagaagcggcctggtaccgatccaggtgtgcgaaatgctctcgaagcccagtatttttcatgatgccaagtctgagatcataaaaggacatgccataggcatcagctgccagtccgaaaatggaactgcacccatcacttaccaccttatgaaagcaaagagtgacttccagactctcgaggtgacctccaatgacccagcaacattcacagataagcccaccagagacatggaataccagtgcagagcggataattgccatggctccggtaggtccagagttcaaatcccancaaccacatggtggctcacaaccatccgtaacnagatctgactccctcntctgggagtggcctgaagacagc

SEQ ID NO: 155 - DNA, 258 bp, Mus musculus
  misc_feature (1)...(258): n = A,T,C or G
cctctacatacgagacagctctgacaggctgagataggaatgcctttttttgntatctattttttttaatgtttttattaggnattttcttcntttacatttccatgagctatcccaaaagtcccccaatnncccctccccaggaatgccttttngtggccaanaatctngntgggnnctgttagtgtnatgattntatncactgtaaagatatggctctgtatttcttgcatgcctaaagcaccttctgattgatta

SEQ ID NO: 156 - DNA, 275 bp, Mus musculus
  misc_feature (1)...(275): n = A,T,C or G
aagggagaccctcactcaagcactcaagncnnggnacngccgcggacccangangacnctgagaccgtactcgaaatgtataangagatagctctaatnttanagcgagtnntcnacctnctganggacccgatattgttgncttagtaccatancctggactgcgtttatcctttttctaatanaagctactaacttattaaggatgcagggcccaaaagtaagcnaaagcatnagtggaatttaaaggtcctgntatattggaaaggaaagtg

SEQ ID NO: 157 - DNA, 288 bp, Mus musculus
  misc_feature (1)...(288): n = A,T,C or G
gactgagatgatggatcacatgggtggtgatgaggatgtagatttaccagaagtagatggagcangatgatgattcacaagacagtgatgatgaaaagatgccagatctggagtaagggctgttgtcatcgcctggattttgagaaaggaaaataacttctgcaagatttcacaattgagaaaacccctgagtcgatagctctaaaggcagatatgctgtacttgcctcctctaacccagttgtcaacctttttttttctttttcttttttttctttttttttttttg

SEQ ID NO: 158 - DNA, 190 bp, Mus musculus
  misc_feature (1)...(190): n = A,T,C or G
gaactgagggagcccgaacctcagctgncatggcgtcacagcatngggctgcgcatgcatgctcatgcgggagcaggcccagcaggaggagcatgcgagagcgcatgcannagcaggctgtcatgcattatatgcaacagcagcagcancagcagcagcagctgggtgggccccccaccccagacatcaa

SEQ ID NO: 159 - DNA, 226 bp, Mus musculus
  misc_feature (1)...(226): n = A,T,C or G
ggtgtcccataggtctccgtgcgactaggaggggcagnggtggcggcggtggcagcagcggcagcggcggcggcggcagcagcagcagcagcagctagcaacctggtcagggtacgctctccatccacagagatgagccgtcaggctgctacagcattatccactggcacctcaaagtgtccaccatcccagagggtacctgccttgactgacacaactgcatgca

SEQ ID NO: 160 - DNA, 401 bp, Mus musculus
  misc_feature (1)...(401): n = A,T,C or G
gactgaggcctgaagggaaagatttcagcattcatcggcttgtcctgggtaagctgaacaaagtctggtttttgttgtgtgtggnttttccttccttcctttctttttctttctttccttttttctttctttctttttttaaacttgggaccaccaagttgtaaagatgtatgtttttacctggcagttgttccactggtgaactgtcaagttgagaaanagtggattgctagnttgtgttngnttttaanattaaattaatccttgataaaagttgcttttttangggttagnctttgaccactatagtttgatgccatctccattttggttgacctgtctcaccatcaggcctgntactctccatgactaactgngtaagtgcttataatggaataaat

SEQ ID NO: 161 - DNA, 100 bp, Mus musculus
  misc_feature (1)...(100): n = A,T,C or G
gacttcaggatgcattngacagtgactgngaaactggnaggatcatgccnaacaattatnaaaatggaaantacagtgactaccaggaaacataaaaaaa

SEQ ID NO: 162 - DNA, 195 bp, Mus musculus
gaactgaggtgaagcactggtggaaggaaaagaacacgcaggagcgaccagggactgagcgagtggagttgaccggagcaagaagaggtcctaaaaattcaattcccaacaaccacatgaaggctcacaaccatctgtacagctacaagtgtactcacatacataaaataatgaataaataaatatttagaatga

SEQ ID NO: 163 - DNA, 216 bp, Mus musculus
gactgagactctcagcgttctcatctctcctccttcaccatgaagctgatggacaaattccactctcccaaaatcaaggtgagatggctcagcaggtaagagcacccgactgctcttccgaaggtcctgagttcaaatcccagcaaccacatggtggatcacaaccatccctaacgagatctgactccctcttctggtgtgtctgaagacagctag

SEQ ID NO: 164 - DNA, 348 bp, Mus musculus
cttcaagaagctctactgcctgagggccactggctggaagaaccaatttctcttacacaggttccaggtgtgcatgtcgggcatccctgtgggtgtttgtggtcggctgtctgtccatgctgctataccccagtgtgctctttgatccagtcccgaactacagggagccttgtgtacacgcctggcttgttcctctgagcgcagccttcaccccagctcaccacaccagcctggaacattcgcccatctttctccgcgcttgacaaggggccaccagagtcaccctacgcaaaataaataaataaataaataaataaataaataaataaaatacccgaaaaaaaaaaa

SEQ ID NO: 165 - DNA, 448 bp, Mus musculus
  misc_feature (1)...(448): n = A,T,C or G
gactgagctacggagtcaaggccaagtgagggggaccagtgcggcctgtgatgactgaagccaccagcgtctcagtgttggacgtttccagtgtgtgtctgacgattcgagcacacgtgtgcacacacacggacggacacacacagacacacacacacacatattctcacacacttatgcgtgtgtgaatatgtgtgtggatgtctgtagctgtatgtaaagcacatatggctacaggtctagatacacacatgagtgtatatatatatgtatgtccatgctagatacacacacgagtgtatatatatgtatgtacatgctagatgcacacatgagtgtatatatatgtatgtacatgtaaagatcgaagggagcagggcgttttctccaaagtactggacctggctttaaccaaaattcttaaggaaaatntttcaatataaaaccc

SEQ ID NO: 166 - DNA, 462 bp, Mus musculus
  misc_feature (1)...(462): n = A,T,C or G
ctgagccagcaagcacatctgttggagcctacttcttcaggattacagtttatacacgaagaccagctgaaacacccagactgcagactcacgcccactggaagcctacctcttcaggattgcagtttatacagagggccagctgaaacacctagcctcgtggaactagactcttggacttcccatttacagctgtccattgttggnncctgtttccangtccctgcatgggttactgtcccgantncccttaaggatatgctgtgatatngcatngtnggccanataaaccttttcctgaccaatntgcanatagntgnnctagttctgnacttcctctttggtctggctgttggattatggtccacagtgaggaccanacnnagacacagtgaaaggctacttnctggctggaangggacatggtgtggtgggtcanntgggggcatcctttgttttcca

SEQ ID NO: 167 - DNA, 391 bp, Mus musculus
  misc_feature (1)...(391): n = A,T,C or G
ctgcttcagcaggatggagcatcantgagatggntcagcaggaacgcaagcagcagcaggagctgcgcttggccttgaagcaggagcgccgggctcaggcacagcagggccatcggccatacttcctgaagaaatctgagcagcggcagctggccttggctgagaagttcaaggagctgagacgcagcaagaagctggagagtttcctgagtcgaaagaggcggcggaacgcaggcaaggacaggaggcacctcccgctgagcaaggagtagtaagaactgtcccaggagacctggatctgtgggaacggggcttggcttgcccggctctggatctttnccttgctcaaaaatgagcttgatgactaccactggcctaagctgacttggaa

SEQ ID NO: 168 - DNA, 574 bp, Mus musculus
  misc_feature (1)...(574): n = A,T,C or G
aagacggatgctccccattctgatgtgaaggatgangaagggacngnagaanagncnnccncnnnntttanacngactancanganaancttgaagagaaacacnngagtgatgctgaagaagatggtgttactggcagtcaagatgaggaagatagcaagcctaaagcagaggaggatgaaattttgaacagatcgccaagaaacagaaagccacgaagagagtgaaacaatctttaagaacttgatctgtgatttcctctccctccccttcccctgcaaatgtggtcctaggagagggcctggtgtaccttangtgggagctcaaaacctcaagatgtcaccatcaacaggttccagtgaatactagcctgtaattttaaacatctagcagtaaataattgcagttgtaatgtaaaggaccctgtttctgtagaaaggaaaacatttaacataatggttgngaaatgtaccatgaaacactaactagngttttggttgnttttaaaanttttccagggnggctttttttttttttcccttttccctttttttttttttttttttaaaaaaaa

SEQ ID NO: 169 - DNA, 203 bp, Mus musculus
  misc_feature (1)...(203): n = A,T,C or G
actcactatgaagccnnagatgaccttgaacttctgatcctctcacctccatccctgagtgctgggactgctggtatgtgccgttatgngtgtgctgttgggagattganctcaaggcctctcccatgctatgcaagcaccctaccaactgagctatatccccagccttcccaccttanagnggtatattgtactgaggatta

SEQ ID NO: 170 - DNA, 270 bp, Mus musculus
  misc_feature (1)...(270): n = A,T,C or G
gactgagattctcacaaacacaaagataaacacaaagatcgagaacaccggcataaaggagcacaagaaggataaggataaggaccgggaaaagtctaagcatagcaacagtgaacatanagattctgaaaagaaacacacagagaaagagaaaaccaaacacaatagatggcagctcacgaaaaacataangacaaacataaagacagagacaaggaaagacgaaaggaggaaaagattagagctgctggggatgctaaaataaaaaaa

SEQ ID NO: 171 - DNA, 342 bp, Mus musculus
  misc_feature (1)...(342): n = A,T,C or G
ctgagagagattcagcctcaaagacatcaaagccataagacactatgtgtaaggtcaatgggatctttgggaacaaaactgattcggaaaagacacctcatcttatgggagctgggcctctgtgtcacctggaagagcaaacacatcatttaagaatccatatgccaacaaggttttgctgaaaggaccccgatatagctgtcttgtgtgaggctatgccagtgcctgncaaatacagaagnggatgctcacagtcatctattggatggaacacagggcccccaatggaggagctngagaaagcacccaaggagctgaaggggtctacaaccctattggttg

SEQ ID NO: 172 - DNA, 331 bp, Mus musculus
  misc_feature (1)...(331): n = A,T,C or G
actgaggtgactccanngaatgggtcattccatctccanaggggtnagnnacccaccagctgaaaactgctttactaaattggtctgcatggctggcctacaattcaaagatctgcctgtttctgcctcccgagngccgggatttaaaggcatgtgttgggctggagagatggctcaacggttaagagcacagactgctcttccagaggtcgtgagattctgactccttcctcangcatcctgataaagatgttcgtttactggtggcttgctgccttgctgatattttcaggatttatgctcctgaggctccttacacgtcgtgagatcc

SEQ ID NO: 173 - DNA, 353 bp, Mus musculus
  misc_feature (1)...(353): n = A,T,C or G
tcaaacgggagccgcttnggactgactgnctgccttcctcctnctccccctcctcccccttctgcttcagatttatttattatatgtaagtacactgtagcagttgtaggacacactagacgagggcaccagatctcattgtgggtggttgtgaaccacccaccatgtggtngntgggattnganctcangatctttanaagagcagtcngggntctaaacngctgagccatntttccagnctcctgcatttcttcttaaggcatgaaatgatcncanaatggnacgacagtcttgnnttnttttcctttttagaananattttttccctttttncactcattatactgtatt

SEQ ID NO: 174 - DNA, 327 bp, Mus musculus
  misc_feature (1)...(327): n = A,T,C or G
tgagatcaacttgtgataatgctgccattgagagaaactcacccaacagctgctcatgaggtcctgcagaagtngcctcttccattgccttgcctcaggatggntggtaaacctggcnattncttcangattgaactacagctgcctggngtctgcctgccaagaggactggtctgcagctgctgaattgtatttgatgtttgctacaggactgacctgctgcccaagaagatcaagcttgcccccaaagaactactgatgaacaggctcacttcccctatatcctaataaattttctcttctactacctttgatgggtagtgggct

SEQ ID NO: 175 - DNA, 235 bp, Mus musculus
  misc_feature (1)...(235): n = A,T,C or G
actgagaaagtctcgggctggtgagatggctcagtgggtaagagcacccgactgctcttccgaaggtccgaagttcaaatcccagcaaccacatggtggctcacaaccatccgtgagatgccaggacttggaaggtggannccaaactggntcctgaacaaacagaaggatgtgagngatgggaagttcanccaggntctggccaacggtctagacaacaagctgcgtgagatga

SEQ ID NO: 176 - DNA, 325 bp, Mus musculus
  misc_feature (1)...(325): n = A,T,C or G
atggcacaattcaatgaaactgactttctanagaanccaagagttggggctgnagagatggctcagtggttnagagcactgactgctcttcnatgacgtcctgagtgtcaattgccagcanccacatggtggttcncancagatncatncctgnagatcgcacatgcccnctnctggtgngtctgaagncantctccnnctnacttgcatannntnaaaccttaagtgtttggcatgaggctttaacagacacaatctggagtgnctatgttttacccaagangcccaaaaaagtancatttgattgcctagagtggatcataaa

SEQ ID NO: 177 - DNA, 280 bp, Mus musculus
gctggcgacatcggccctcgtctgacccaggaacgctaaggaaggcacaatgcggcgctgggtgaagatgcttcatagaggagaggagtccctgaagatgcagagcactgtggcagcatcagaagctgacaatgaccaattgagagcggccagcagagccagtcctcttacagctgcagaactcacggtgtgccagtcaatggccatttggcttttgaagcaaattgggaaagtgtaaacgcttgataaatagttgcctcttgagtgaaaatgaaaaaaa

SEQ ID NO: 178 - DNA, 410 bp, Mus musculus
  misc_feature (1)...(410): n = A,T,C or G
gtggctgccaatttgatgtaaactccctgctatgacgcttaactgtctggaggagaagtacaagttggactaggaacagattgatccctccacgtcagcctcctggccacccgagactcacatcttgtcagagaaacacaatgggctcatttagctgtacattgaactggtggtcttctaagagaatgtgatgataataagactttaaggtaaaagttaagaactggggctgggggctggagagatggctcagcagttatagagcactgactgctcttctgaaggtcctgagttcaaatcccagcaaccacatggnggctcacaaccatctgtaatgagatctggcaccttcttctggngtgtctgaagacagctacagtgtacttacatataataaataaataaatctt

SEQ ID NO: 179 - DNA, 290 bp, Mus musculus
gactgagatttaccttcaagactgctgaaattttaactacccaagatgcctgtccctcccccaccagcacccccaccgccaccaacatttgcactcgctaacactgagaagcccaccttgaacaagacagagcaggctgggaggaatgctcttctctctgacatcagcaaagggaagaagctaaagaagacggtcaccaatgacagaagtgccccaatattggacaaaccaaagggagccggcgccagtgctggtggctatggtggaggcggcggaggtggtggtggagg

SEQ ID NO: 180 - DNA, 287 bp, Mus musculus
  misc_feature (1)...(287): n = A,T,C or G
agactgagagagaagcctgccgatgaatgtctcgtggaattccanccgattcttcccacttncgatgggtnaanccaccgntactgattcncatttggngtnngctatnggactgaacctgctgatatcctgacatgaagcttagagtcgatctaaagaactacttctaaacaggtccacaatgcccttgncctagtaaccgtatctntcccctacctctggtcggtgctctagaagggagattgatgcattgaagaaccccaaataaaataggctttgaaaaatca

SEQ ID NO: 181 - DNA, 273 bp, Mus musculus
  misc_feature (1)...(273): n = A,T,C or G
tatttgcacttaacagcaacctcagcttnttgccaggcggngagtaccaagagctgctgtggagtgcagatgctgacaggaagcagcagtgcagcttcaagggcaaggacccaaaggtgagactgagggatctgggcagaacactgagcagatatggctgttatgggaaggacacagtctggacatcgccagcactgggcttcagccccagccccagccccagccccagccctgtcctntatgngatcctggataaatagattcatctggtca

SEQ ID NO: 182 - DNA, 453 bp, Mus musculus
  misc_feature (1)...(453): n = A,T,C or G
ttaccttaaagatctncctgttttgcctgccgagngtcttattannggctgctccatcacaccagggatggcaagatgccttcnagggtaatagcactgactgctcttccnaaggtgcctgagttcanatcccagcanccacatggncgctcaanaccatctttaatgagatctgatgcctgtttctggtgtgtngtgacaacagctactncgtacttatatatnacnaatggatatctctttcaagttttacctgccaaagttacacatgaaggggacactgtgggagangagtctgtctcctgcanaatgtctctaanaagcccaggtttctccttaacctangaaccctgcagctgtaggacccanaagtggggtctcttctctatactaactatcttaacccttatctcttgcccactgaatattaaataaaatatcattagttaatcc

SEQ ID NO: 183 - DNA, 308 bp, Mus musculus
  misc_feature (1)...(308): n = A,T,C or G
gcctccaggatccagtccggctgtcgcgttgcctgatcctgttcaagttgnaggcttgggaggaactgantggtaggtcaaggcttctatcttttttccatgctaagggctgtaccactgccaaaggttangaaaagataagaggggctggcgagatggctcaacgggtaagagcacccgactgctcttccgaaggtcctgagttcaaatcccagcaaccacatgggggctcacaaccatctatnangagatctgactcnctcttctngantgtntgaagacagctacagtgtacttacatataataa

SEQ ID NO: 184 - DNA, 440 bp, Mus musculus
  misc_feature (1)...(440): n = A,T,C or G
agactgagaaaggccaacttccatcaatgggtggatgnnncntgggactntgctncacgantgcntgggctgatagagcctnanattcaanntggctcattntaccctatggaaagaggaggtggagtgngagagccaattgnaatggaanacagngaggaaacaaaggccttcnagacacaanaggactgctgagctcacaaagactgaggcagcaccctcaggacctgcacaggtctaagcttgatgcgatctgagtactgagagggcaactggacacaattctatattgataacccagaagctttctccagttgataaactctttcaaaggaaaaacttgttttctaccatggagtgacactggatatngaaaccttcccgttaacaggcagacttccatgggctcaagacttccaatacaatatgaactcagttgc

SEQ ID NO: 185 - DNA, 207 bp, Mus musculus
ctgagggttccaagactgccctttgggattcaaacgggagccgctttggactgactggctgccttcctcctcctccccctcctcccccttctgcttcagatttatttattatatgtaagtacactgtagcagttgtaggacacactagacgagggcaccagatctcattgtgggtggttgtgacccaccctgcgggtgctgggattt

SEQ ID NO: 186 - DNA, 318 bp, Mus musculus
gtctcctatgctcagtcaaatctccataactgtaaatcaacactaagagcactggataagttaaggtcaaggagactctcacaaaggatccagaaggtgtagtccagattaaggtgtgttccaccacacctttaatcccggatgaccttgaactcggagatttaatcttctggaatccatagccactatgcctcaagatctccataccaagatccagatcagaatcttctatctccaagcctccagataagggtacctggtgagccttccaattctggattgtagtccattccaaataaaatcaagttgacaaccagg

SEQ ID NO: 187 - DNA, 438 bp, Mus musculus
  misc_feature (1)...(438): n = A,T,C or G
tccagactgagaaaangagaagaaatggcagnggttgatgacaaccacccntcntccccaaaccaaaaggancaggnaggaaccccntcttccaggactcctgggacacagagtcttcaagcagcgacagtggtgggagcagcagcagcagcagcatcaacagcccagacagggccagtgggccagagagcagcctgagccacaccatccccggatcctgccccagcaccccccagccgatgcctgagcagtctggcactatgcccaagggcccttactttccacatcaaccagacccttgaangaggctcactttcataagcctacagcacccgangccggcccgccgacatgatgctccttccggcaagtttcttgccttctgtgaanggacaagcactgtgcaaaatggatatttcaacttaatgatttggtttt

SEQ ID NO: 188 - DNA, 357 bp, Mus musculus
  misc_feature (1)...(357): n = A,T,C or G
agactgagggagatcatagacaacacaagcaaagagaacgggatcgacatcatcctggccgacaggaccttccatctcatcgcggagtcccccgaagatgccagccaatggttcagcgtgctgagtcaggtccactcgtccacagaccaggagatcagagaaatgcatgatgagcaggcaaacccccagaatgctgtgaggtgctggagagatggctcagcagttaagagcactgactgctcttccgaaggtcctgagttcaaattccagcaaccacatggtggctcacaaccatccgtaacaagatctgatgacnccctcttntggagtatgcttacagctacagggtacttacat

SEQ ID NO: 189 - DNA, 100 bp, Mus musculus
  misc_feature (1)...(100): n = A,T,C or G
gagcacggcagtggtgacccaacagctggtgtctaagcccagccgggagaccccgagggccanggccctgtcgtgttagcaccgcagatcggaaaaaaaa

SEQ ID NO: 190 - DNA, 361 bp, Mus musculus
agactgagggagaagagatgggtgactgtgggtgatacctccctgaggatattcaaatgggtgcctgtgacagatagcaaggagtgtaccatattcatgcctggtgcccaggaaggtcagaagagggcttcagatccctcagaactggagtcctggatggctgagtcaccatcgaggtactggaaatcaaatccagatcctctgaaagagcagcagacactccttagctgcagaaccatctctccagctcctccacagtttaaaaatattatgtatttttatcttatgttcatgtgtgttttgcctgcagaggccaaaaatacatatctgttctcgtggaactcgaattacaaaaaaaaaa

SEQ ID NO: 191 - DNA, 241 bp, Mus musculus
cgaagaaatgctgtacgtgcccttaaatcactctctaagaaataccgcttagaagtgggaatccaagctatggaacatcttatccatgttttacaaacagatcggtcctgagttcaaatcccagcaaccacatggtggctcataaccatccgtaacgaaatctgatgccctcttctggaatgtctgaagacagctacagtgtaattatatatagtaaataaataaatatttttaaaaaatg

SEQ ID NO: 192 - DNA, 262 bp, Mus musculus
  misc_feature (1)...(262): n = A,T,C or G
cttttccctggtcccangacttncccacccctcctccaaataccaagactatgggcttaaaantttcaaatgggngctggagagatggntcatcnagttaagagcaccgactgctctttctaaggtcctgagttcaaatnccancanccatatngnggctcacaaccatctgtaatgnaanctgatgccctcttctgntgtgtctgaagacagctacagtgtactcacatatattaaataaataattctttaaaaaaaaaaa

SEQ ID NO: 193 - DNA, 224 bp, Mus musculus
  misc_feature (1)...(224): n = A,T,C or G
tgaggtccgcatagccgctaaattcatcacccatgcacccccaggggagtttaatgaagtattcaatgttccaaccctgggacagccctgaggtgctgaacaagggagattgctgcaagctccattacaacctggacgaaaggntgtctcaaaactnaaatgaantgaatagaacccggncgggggcggtgcacccctttattcccaccacttgggaggcacag

SEQ ID NO: 194 - DNA, 402 bp, Mus musculus
  misc_feature (1)...(402): n = A,T,C or G
ctgaaggcagtttacattcggagttccgcggactcttcanaggctgtaaaaatgccggagtcacgaaagcctgcaaagagctgttgcttgccttgctgctgctgctgctactgccgtccctgcccgtgctgcagctgctactcctgctgctgctgctgctgcttgttgctgattgttgcggttgctggttgctgtttactgtttgctgccggctggattgctggattgctgattgccagtcggcgaaatcctggcgatgaagatccaacttgccccaagaaacaagacgaccctaagaagtagtctaatgatttcaatacgccctttccccctctgaccntttttctcttaccttatgctgggggattggaagggtggcagaaaaaagaacccaataaagta

SEQ ID NO: 195 - DNA, 413 bp, Mus musculus
actgaggggagtacagttctccaacactgccccagctaaatcatctaatgccacaggtgacaaggggcaagaccacctctcctgttctcatgacctagggccggctctcccacctgacagaggcagcaaaaacgtaggtggtgggagggcacctctccctcactcatgctaccacgtggcagacaagggacatgcagacaagggtcatgtgctcatgacctctgtcaggatcacccaagtctccaccatcagtgccaactctactgtgctgctcagatcagtttcttcctctgaagatgcgctgtcttttgcagatttcattgattttttctttatacctggttcagcaaaacaacataggaaggttaaaagacaggctcaacagtgaaaatttggtatactttgaacccata

SEQ ID NO: 196 - DNA, 420 bp, Mus musculus
  misc_feature (1)...(420): n = A,T,C or G
acctgcttgctcggggtggagctncctgctcatncatnctaccacgcccactggnggaacntgaggagccacanacgtgcnccnttctactggancaatanantattatggtgggaatngggtcccctnccccttccttcataactanccgnnanagcaataaaanttgagccttgatcatnangantgaaaaaagaaggggggggggnannancccaacatggacacaggaatcttcttgatagttcagtcaagttctctcttctttatttccccccaacccccagcttatgttcctcagcaaactctcaattacagnggggctatattctatttttctttaagggnnatggataccatnnantttcctaannacccttnanggggataaaagggntttacatattacagggggaaaaa

SEQ ID NO: 197 - DNA, 376 bp, Mus musculus
agactgaggataattgaaataaatcaaaaccaaaagcaggtggaacaggatattaaagttgccatcttcacattgatggtggagataaacaaaaaagggaaagctctgctgcaccagcttgagagtcttgcaaaggaccatcgaatgaaactcatgcaacaacagcaggaagtggctgggctttctaagcagttagagcacgtcatgcatttttctaaatgggctgtttccagtggcagcagcacagccttgctgtacagcaagcggctgctcctgcttcctgacctgcttgagttccagtcctgacttcctttggtgatgaacagaatcgtggaagtgtaagctgaataaaccctttcctccccaaaaaaaaaaa

SEQ ID NO: 198 - DNA, 248 bp, Mus musculus
ttgacacccttagtctgcaaaccaaactcagggccttctgtatgtggagcatttgttgtaccgctgcctgaggacgaggaaggggaagttactgggctggcttcgagagatatgtatgtatgcctgatgcctgtggagttccaaaggtgtgtcccctgggattggagttacaggcagttgtgagccatttgggttctgggatgctaacccaggttctctgcaagagcaacgagtgcttcaaccactga

SEQ ID NO: 199 - DNA, 100 bp, Mus musculus
  misc_feature (1)...(100): n = A,T,C or G
ctcatagtaaaaagaagcgatccgggtcncgcagncgncattcnaagtatctcacctgtcaggcttccattgaattccagcctgggagctgaactcagta

SEQ ID NO: 200 - DNA, 249 bp, Mus musculus
actgaggattttggctttagagaagcttggtgctgtcttcaatcaagtagccttcccattgcagtacacacccaggaagtttgtcatccaccctgagagtaacaaccttatcatcattgagacggatcacaatgcctacactgaggccacaaaagctcagagaaagcagcagatggctgaggattgtaaggaattgggctgccgactgtaagtcatcaataaattcctttactatctagaaaaaaaaaa

SEQ ID NO: 201 - DNA, 260 bp, Mus musculus
  misc_feature (1)...(260): n = A,T,C or G
cctgagactgcaagtggcaatgtggaagcaaaagtcgtctgcttttacagaagacgggacatctccaacacgcttatcatgctcgccgacaagcatgctatcttncactannagcctttggangaanacataaaactcncanntcctnntgcgccntgcctgcctggatactgccatgctctcacctggatgataatggactgaacctctgaacctgtaagccagacccaattaaatgttgttttttataagaaaaaaaa

SEQ ID NO: 202 - DNA, 442 bp, Mus musculus
  misc_feature (1)...(442): n = A,T,C or G
gatgaggtaggcacggctggccantcctgggtgcaccccaaaatacagtttgctttaattgaacaatcactcaaaaactggcncaaggaanananntgatngnngnnccnganctgcgggtcgcggtaataaaggttcccgtaaagnngactgttaagaaggattcaactgtatgaattcagaacttttcagctggggaacgagagtaccagtgagtacagctttacgaggtaagtccgatcttgaactttctaacgaaattcaagacagtctatcagaagtaaagtggaaaatggctttacaagttatgtttggccttgaattttttctagtgttagaancccttttgttccttttcacatgttatcaagtggttaaagcagggcggattctaaaggaaattcannacaagctntcanaagtaaaacggggagaaagagtt

SEQ ID NO: 203 - DNA, 197 bp, Mus musculus
gactgggatgtcctaacttcaggatggaaggaaggaaacatgatgccatattgaagaggttttgtctttgtttcaatgggagaagaagagctacggataccatcaaattaataaagattagatttggccaggggagacctcctgaaaatcttggctacagacatgaaggaaaaaattcaagaagaccaaaaaaaaaa

SEQ ID NO: 204 - DNA, 301 bp, Mus musculus
  misc_feature (1)...(301): n = A,T,C or G
gactgagggagaaactcactcgccaaacaacgtctcgtggtatcctggctgactctggccgcttcagcagagcctggctgttcctgctggattgtgctactgctacagattcatgtttggtgtttgctattgaactgggctgcttatatcctgacacggaagagtggaatcgcccaaagcaagtacttctaatcaggtccacaactccctttgcctttaaccttgctttttccctacctctggttggtgggctaaaagggagggttaagaaccccaaataaagncagttttgaaaaaccta

SEQ ID NO: 205 - DNA, 267 bp, Mus musculus
  misc_feature (1)...(267): n = A,T,C or G
atgacttggataaagaactaaacaactacattatgcggtgtagggaatggtactgaaaagaaagcaaaaaaagccaagactaaaattaaagctgaagtataggaggagatggaggaggaggaggcggaggaagaacaggtagtagaagaggagccgactgtaaagaataaaaagaagaaggataaaaagaaacacataaaggaagagccactttccgaggaggagccatgtnccagcacagcagttcctagtccagagaaaaaaaaa

SEQ ID NO: 206 - DNA, 370 bp, Mus musculus
agctgtagctgtccctccaaacaaggctattcagagagttggagacaacagaaaaggcaagctagacacgttcagcatgagtctaggactgggctccgtaatttcctgtgcgtcttcagaacaatctggcaaccacacagtttgtaaactgtacacctgaacttccaaactttacatattatacatacatacccatacatatggatagatacacgtatatgtatatatgtatattgtgtatatgtatataaatatatatgtgtatatacatatatttataaaatgttaagacatatctgatttactttcaccactacagaattatgtatattactggaaaaatttagaaatacttgcttcttaatagaat

SEQ ID NO: 207 - DNA, 256 bp, Mus musculus
  misc_feature (1)...(256): n = A,T,C or G
acactgagagaccctgttgactgactgagctggcttggccacccacccttgaaccaacgatcatcctgtcttggtttcccaagtgctgggattacagatncccngngactngcatgacacccctgtnctcttntctcggagacccngtgacttgcctgncccacagaacacactnagcgaaaanctgctgnctgaataancctnctgcccccgtgcgtctcctnctccgtagncgtnactgacggactgtggtgac

SEQ ID NO: 208 - DNA, 544 bp, Mus musculus
  misc_feature (1)...(544): n = A,T,C or G
ctgagccccctgggatgatacccaacaactcatgagaataatcttcatgaccgaagagtgagagaggataatgcaggaagccaaaaagaatgtcccttcagacacgagggcatcatcgattgaccaggctgttatctacagcgggtgtctcctgattagacctggctgggactttaacacagcagaaggtaagtggcacctgaaggtctaccgccaggctccattggcaggtctcaaggagggctgtactacaacccacaaatttgaccaaggtacgtgaagttagacaaggatcagatgagtcacctgctgcctttctagaatgactcatggaggcattccgtcagtatacaccctatgatcccagcagcaaggagcacaaagctactgngactatggcatttattagaccagcttgttagagatatcaggaaaatgcttcagaggctanagggatgacagggtaagtcactgaggggtttaatgcaggttgcttgaggaagtctaccataacagggagacagggggagaaaaaaccatattg

SEQ ID NO: 209 - DNA, 525 bp, Mus musculus
  misc_feature (1)...(525): n = A,T,C or G
gcaactgaaggaaaagctgccctatgtggtaatgaaaaacttaacactgtcctgcatgactgcctatggaagaccgatgacctctgccctcaagcccaatggagaatcctaggggcgctagctccatccatctgtaagtcccagaacccaagtctcaggagcggcgggagaccggagacaaagtgagagaggtggatggaagcgaaagacaagcaggagagagagaagagaccacgctgactctcactccctcttctttaatacaacaaagttgcaacttgggctcctggatctaaataccgcaggtccagagaagacaaaacaccaggcatcactgcatctcttagggggtacccaccaagacctccaggtccttcactccaggaagggccctgttgaatatctgcagagagatgggttggagcacagcttggtgctcagtcctggccctgtggctcctctggtgtgctgtctgctccaacgcaacgtcagggggacngcaatgcgtttcctttttgacatcca

SEQ ID NO: 210 - DNA, 352 bp, Mus musculus
  misc_feature (1)...(352): n = A,T,C or G
agaactgagggttccaagactgccctttggtattcaaacgggagccgctttggactgactggctgccttcctcctcctccccctcctcccccttctgcttcagatttatttattatatgtaagtacactgtagcagttgtaggacacactanacgagggcaccagatctcattgtgggtggttgtgaaccacccaccatgtggttgctgggatttgaactcaggatcttgcagaagagcantcagggctctaaaccgctgagccatctntccagnctcctgcattccttcttanggcatgaatgatcccagcatgggacgacagtctgccctcccttctttttgagggagat

SEQ ID NO: 211 - DNA, 433 bp, Mus musculus
  misc_feature (1)...(433): n = A,T,C or G
actgagcataaacagaggcggcatgacaagaaacnnaggctctggaggttttggtggtggtggcaccaggagagggacacgtggaggcagccggggaagaggtagaggcaccggcaggaactcaaagcagcagctttctgcagaggagttggacgcacagctggatgcttacaatgcaaggatggacaccagctaaacaactgagcaaatccacacaagtaacaggacccagaagcctcgcctgtgatgcctagagggagggttggcaactgaactgtgcagacaatggnggatttggtttggttctttttatttcttttctttttttttttnttcccggtttaaaaaaggatntaaaaacncttntaaaggnttttttttttttttttttggggttggggngggnttttggttttttttttaaaaaaaaaaa

SEQ ID NO: 212 - DNA, 100 bp, Mus musculus
ctgatacatctttagatggtggaagatttctctgagaagtgagtatcggtcagcaaaacctggagaaacaaaggaaagccatgaacacttgctagataac

SEQ ID NO: 213 - DNA, 372 bp, Mus musculus
  misc_feature (1)...(372): n = A,T,C or G
gtgacaggaggccatantgactganttgtgganctctcacaggtgctgcaggaggcntgnntggtgccntactgnnnaaactgnanaanggngnnggatgatcatctcactgacaatttagagacagagaaactgtgtgactacttctccacacacctgggtgtctaccggcccgtgctggctgcgctggagtctctgaaccgtgcggtgctcactgccatggacactacaaagctgggaggagatgnagtctcccctnccccancctgaaacctgcttgctcngggnggagctncctgctcattcgttctgccncgcccactgttgtancctgnngaatcacacncgtgcacctttctactggaccagaaa

SEQ ID NO: 214 - DNA, 203 bp, Mus musculus
  misc_feature (1)...(203): n = A,T,C or G
agaactgagggttccaagactgccctttgggattcaaacgggagccgctttggactgactggctgccttcctcctcctccccctcctcccccttctgcttcagatttatttattatatgtaagtacactgtagcagttgtaggacacactagacgagggcaccagatctcattgtgggtggttgngaaccacccactcatgtg

SEQ ID NO: 215 - DNA, 100 bp, Mus musculus
  misc_feature (1)...(100): n = A,T,C or G
cactgagaccaacttttacaagtctacctgttcgagaagaaatgatggcgaaatactcaaacctttccttaaagagctgtnatatctcagtgacggaaaa

SEQ ID NO: 216 - DNA, 430 bp, Mus musculus
agactgagtatccctggaactgatgatgtcatccagccagctgctcctgttgacccgggacacccacccttagctgactcgtcccaccatggtgacgctgtcagctcagtctctacacatctgacagtccagagcgcctccccatctgctgcccgtgcaagtcctgctcctttggccccagaacatacagcctcggctccctccgcagccggtcctggagtagaagtcacccccactgcctcccgagtgctgggattgaaggtgtgtaccaccacacccggcgagtctccctttcttaaccccatgaggataatccttcgccaccatgatgtgctctcctaagtcattttgggcactgttagctacgcacgcacgtcctcttttcatagacaaagaaactgagaacaaacaaggtacaaaacaacaaaaa

SEQ ID NO: 217 - DNA, 377 bp, Mus musculus
  misc_feature (1)...(377): n = A,T,C or G
cactgagaggctcacaggacacctgcacaacaaaggctgtccccgtatgtaatcaacctggacccagctgtgcatgaagtcccctttcccgccaatattgatatccgagacacagtgaaatataaagaagtcatgaagcagtatccttttnctcatctgctgcgagactgactcggtcatggcaaactgctgnctttaattgntttgggggagnggctttgcctgtcangnngngacaatacaagnagaaaactcatgnccttttaggnaatnntattaagaggataagatgagggctggtganatggctcaacaggtnagaacactgactgctcttccaaaggtcctgagttcaaatcccagcaaccacatggtgg

SEQ ID NO: 218 - DNA, 495 bp, Mus musculus
  misc_feature (1)...(495): n = A,T,C or G
cactgaggccaagttcacagtggagactgaccccacatgtgagtttattttgcctcaccagttcaggtcacagttcacggtggccccatggtgtccacagtcaggaagcaaagagagatggatgcttgtgctcaacccaacctctcctttttatccagccttggatcacagcccacttcatgatggagcaacactgggtcatcacccaagtgccnnggcacatgcagccacccagttttggactttcagcttctaggactgtatcccatgtaagcctctttataaattatccagcctcaagtgctctgttatagcaacagtaaccaactaagaccccataactccagcaactaatacttaatgaataattgctgacttaatctctcatcacttggattctataaaggtctttggcaatcatttcttatnggaanaaatattnttnttcccaatttgggcccccncctactttggggnaagggggggttagaaggt

SEQ ID NO: 219 - DNA, 230 bp, Mus musculus
  misc_feature (1)...(230): n = A,T,C or G
gaaactcctgcctatttntgtgtctttgctcctgctccttaccccttatttgtaaggcccanccttgaagaattgcttgagggctggagagatggctcagtggttaagagtactgactgcacttccagaggtcctgcaaccacgtggtggctcacaaccatctgtaatgggatccgaagcctcctggtgtgtctgaagagactgacagtaaaactcacataaaataaata

SEQ ID NO: 220 - DNA, 295 bp, Mus musculus
  misc_feature (1)...(295): n = A,T,C or G
gactgagagaaaaaagcagagatcgtgaaagggaacgggagcgggaaagagagagagaacgagaacgggaacgagaacgagagcgagagagagaacgggagagagaacgggaacgggaaagagagaaagacaagaaaagagacagagaagaggatgaagaagatgcntntgaacgaagaaaactcgaacgaaaactgcgagagaaagaggctgcttatcaagagcgccttaagaattgggaaatcagagaacgaaagaaaactagggantntgagaaggaggcagaaaaaaaaaa

SEQ ID NO: 221 - DNA, 229 bp, Mus musculus
  misc_feature (1)...(229): n = A,T,C or G
aattgaggcttccaagactgccctttggtattcaaacgggagccgntttggactgactggctgccttcctcctcctccccctcctcccccttctgcttcagatttatttattatatgtaagtacactgtagcagttgtaggacacactanacgagggcaccagatctcattgtgggtggttgggaaccacccaccatgtggttgctgggatttgaactcaggatcttca

SEQ ID NO: 222 - DNA, 127 bp, Mus musculus
  misc_feature (1)...(127): n = A,T,C or G
gaaattcaatagcagtacagcatttgccttgcatgtgtcagncccttgtttcaaactctggtaccagagtttagatctggttttgataaataaataaataaataaataancaacaaaatataaaaaa

SEQ ID NO: 223 - DNA, 100 bp, Mus musculus
gtccacttcttcaacagctttttccatagacagctggtaaccaaaggctataatggagttaaaagatggacgaagaaggaaatctggaaaaagaagaaaa

SEQ ID NO: 224 - DNA, 195 bp, Mus musculus
  misc_feature (1)...(195): n = A,T,C or G
ttaggaaaaaggtcacaggaactgaaggggatgaaaggagctaccccacatgaacaccaacagcagtgncnactngcctggacccctgaatactcccagaancaacacccaaagaacatacatggacttattcaaggcctgtggcacatatctagcaaanaacttgantntccccagtgggagaggatgtgccca

SEQ ID NO: 225 - DNA, 316 bp, Mus musculus
  misc_feature (1)...(316): n = A,T,C or G
ccaactgaggtcttgccccctgctactctcccaaaactactgaactggactgctggtatcatccatggagtgtttgtgagtggactgagctgctgctgctgacctgtgaactgaactgctgatttctaacaatgcagataggatttgctccaaaaaaccaatttctaaacaggtacacttcctccatatcctttcttttccactacctctggtgggtggttggctagaagggaggttaaagcctttactaaaaagagggttgagaaaaaaattaaagcctacanccttttattaaataaacttttgtaatgggtaa

SEQ ID NO: 226 - DNA, 227 bp, Mus musculus
gaggactctcctgttcttgccacatttgactgaagaaggacaacggggtcaaggtttgaggactgacaggtaagttttcctggagattcaaaactgtgcttttcaacaagcattggggctcaaagttctgtcagtttgagggcagcctagtctacaaagcgagttccaggactgccagtggtctgttacacagagaaacctgtcttggaaaaccaaaccaaaaaaaa

SEQ ID NO: 227 - DNA, 182 bp, Mus musculus
  misc_feature (1)...(182): n = A,T,C or G
attcttgagtttcttggggccangaagatgacacgantagatctagacnctggcctagcgtgacctccanagtttggnccttgcntaaaggagaaaattaatnccttcagttgntctctgacctccacatgcgtcctgtaacatgggtgtatgcatacacataaacacacatccacacgttg

SEQ ID NO: 228 - DNA, 140 bp, Mus musculus
  misc_feature (1)...(140): n = A,T,C or G
gaagaattttcatcaacaaacatcattttnaaagaaattnanggcgaaaatggggcattgggnnnccntgggggaagggggagactganagaatgagaatgaatnatggccaaataaataaataaataaataaataaaaa

SEQ ID NO: 229 - DNA, 204 bp, Mus musculus
  misc_feature (1)...(204): n = A,T,C or G
attatatacaggagtgcntaaattttgtctgttacagccncggccattagaagctggggcaaagcttttctgcctacttaactctgattttatntatnngttatatgtaagtacactgtagctgncttcagacactccagaagagggagtcagatctcattacaagatggttgngagccaccatgtggttgctgggatttgaac

SEQ ID NO: 230 - DNA, 235 bp, Mus musculus
  misc_feature (1)...(235): n = A,T,C or G
actgaggcagggcgtgagtactcgcctgctgccaccactgcggaaaatgggggtggcnagaagaaacagaaagagaaggagctcgatgagctgaanaaggaggttgccatggatgaccacaagctgtccttggatgagctgggctggcaaggaaaggttggggtgtgctggctgaaggagagaaaggagagaaaggagagaaaggaaagaaggaaggagagaaagaaagaaagaa

SEQ ID NO: 231 - DNA, 141 bp, Mus musculus
  misc_feature (1)...(141): n = A,T,C or G
gttgtggaggaggcagagaatggaacgagatgcacctgccaatgggaacgctcaaaatgaggaaantggggagcaggaggctgacaatgaggtagatgaagaagaggaagaaggtggggaggaagaggaggaggaggaata

SEQ ID NO: 232 - DNA, 104 bp, Mus musculus
tgaggaaagaccgggaacgagatcgggatagagaggaccgatctaaagatcgtgaccgagaacgtgatagaggagacagagaacgggagagggaaaaaaaaaaa

SEQ ID NO: 233 - DNA, 740 bp, Mus musculus
  misc_feature (1)...(740): n = A,T,C or G
caggcttgaccaagaacctgaagcctggtnaggatgacttggannntgacctgnaactggatcccatgctgncttttgaacagagcaggnggatggaccttaatntggacctagacttggacttggaccagncanagctggaccctggatctgagctggattctgagatccaagccaaacctgagctggaactagagtcggagcttgagacagatgcccagacagagccagagacagaggaagagccagagctggagccagagccagagccagagccagagacagagccagagccagagccagagagagagaggaagaccaggacaaggtctcagagcccctggcggaactacttcactgcttatcgctttggttcttcaaactcccagtctagagctccacctcagacacgcccagaaaagagaaagccacataattaccccaattctccctaaaacaataaggatccttggtaggtgactgcaaaatgcaggctgagtatcttggcaaatcccttctgccctacttggcatgacatctagactgggtacaaagccaccggtcctggctctgtgaccccttcattgctgacttncttcttattggattcttgggatcacatgcccatcatatgcccangctatgtgacttcaaggntggcaantggcttttggggtctggcttcaggctttgcanaatgnatttttaacagatttcagtgaaggcttcctggacgtact

SEQ ID NO: 234 - DNA, 291 bp, Mus musculus
ggttccctaaaatcagactctgtcatcggcaagtggagactgtaacacgactgggaccctcacatgccgataagcagtcaagaatattttctgggagctggagagatgggctcagcagttaagaacactgactgctcttccggaggtcctgtgttcaaatcccagcaaccacagggtaactcacaaccatctgtaatgagatctgatgccctcttcgggtgtgtctgaagacagctacagtgtccttacatataataaataaataaatctttttttttaaagaataaaaaa

SEQ ID NO: 235 - DNA, 306 bp, Mus musculus
agggagaaactcactcgccaaacaacgtctcgtggtatcctggctgactctggccgcttcagcagagcctggctgttcctgctggattgtgctactgctacagattcatgtttggtgtttgctattgaactgggctgcttatatcctgacacggaagagtggaatcgcccaaagcaagtacttctaatcaggtccacaactccctttgcctttaaccttgctttttccctacctctggttggtgggctaaaagggagggttaagaaccccaaataaagtcagttttgaaaaacctaaaaaaaaaaa

SEQ ID NO: 236 - DNA, 600 bp, Mus musculus
  misc_feature (1)...(600): n = A,T,C or G
acaatctggagcaaaagatcctgcaggtgttgagcgatgacggcggccctgtgaagattggccagctggtgaagaaatgccaagtgcccaagaaaaccctcaatcaagtcctttaccgcctgaagaaggaggacagagtgtcctccccagagcctgcaacatggagcataggcggggctgcttctggagatggggctcctgcaatccctgagaactccagtgcccagcctagccttgatgaaagaatattaagattcctggaagccaatgggcctcacagggccctgcacatcgccaaggctctgggaatgacgacagccaaagaagtgaacccactcctgtattccatgagaaataagcaccttctgagctatgacggacagacgtggaagatctaccactcacgtcaggaaggccaagacatagtcttgccatgtagccctggatgtcctagaactcaccatgtagaccaagctggtcttgaacccacagagatcttcttgcttctgcctattaaattctgggattaaagcaccactacacctggaaaaatatcttttaaagncngtaaaataaatgaacaattaaagaatgcattct

SEQ ID NO: 237 - DNA, 266 bp, Mus musculus
  misc_feature (1)...(266): n = A,T,C or G
gtggaactagactcttggacttcccatttacagctgtccattgttgggactagcggctcattcagcaccatggagagtgccaccatcagccctcagcccccacagtctgactccctggaggcctttccccagaagagcatggagcctgcagacatcgctgtcctagttctgtacttcctctttgttctgcttgttggattatggccacagtgaggaccaaacgaagacacagtgaaaggntacnttctggcttggaagggacatgg

SEQ ID NO: 238 - DNA, 459 bp, Mus musculus
  misc_feature (1)...(459): n = A,T,C or G
atctcagcggttctatagcacgctccagatgtcaaattaaacctcggtgtaagcggagatttcatcaaagaatctacagctactacatttctgagacaaagaggctatggctggcttttagaagttgaagatgaggatcctgaagataacaagccacttttgtatgtgaaagancatgtaatttgngtnttaagacctaatntgnaggatgcttatgtnnnttttanagacagatgaagaaaatgtnagcctttntggngaaaaatantanagatgaaatcganaaaacttttggtgaaaaacagagatggctcancagntaagagngcttcctgctgtttgagagagcttgngtttggtccagcactgtatcaggcaatgcagctgcctatagctccacatctgacttngtacacttatagaaacacatacacaaataaatcttttttaaaagtaaaa

SEQ ID NO: 239 - DNA, 277 bp, Mus musculus
  misc_feature (1)...(277): n = A,T,C or G
gagtgatcgaagaggagccatgatggntaaacttctttaaaagaaagaacgacaaactggctttccttgatgcctttgaacctgagtccagaagacttagggacaatggatagctaaaatttatttatatctgaagttgggtggcagggtgcactccagaggcagaggcagaggcagggggatctatctccgagtctgaggtcagcctggtctacagatcgagttccaagacagccagggctacaaagaaaaatcctgcctcaaaaaatggaaaaaa

SEQ ID NO: 240 - DNA, 346 bp, Mus musculus
  misc_feature (1)...(346): n = A,T,C or G
aactgaggaaggtgacanatctctggaaanggngctacatatgattcnangacaccatgnggatgctggnnatgaaantggggccctctggaanaacannggaagaacggccaatgctcttagcagacgaagcctctccagcctncccttcacaacccctagtgttatcaagagactagcaaaccttcanggggtcttcaactaaataatacaatgagttttgcctttaaagagcacactgctgggctggtgagatggctcagtgggtaagagcaccggactgttcttccgaaggtttgaagttcaaatcccancanccacatggnggctcacccccccccccccc

SEQ ID NO: 241 - DNA, 282 bp, Mus musculus
  misc_feature (1)...(282): n = A,T,C or G
ccactgagcttaaacacaactgaagtctgtctgtctcaagtgtattaatgacgcctgcccaangtggtgggaaagtccgtgaaactgcaatcgggggctggtgtgatggctcagtggttaanagcaccgactgctcttctgaaggtcctgagttcaaatcccagcaaccgcatggtggctcacatccatccgtaacaaaatctgatgccctcttctggagtgtctgaagacagctacagtgtacttacatataataaataaataaatctttaaaaaaaaaaa

SEQ ID NO: 242 - DNA, 100 bp, Mus musculus
  misc_feature (1)...(100): n = A,T,C or G
gatcactagacgggagatgctgccaagngccccagacgggatcaagactgcaatgacaaccataagatnccctaaagctaggacactgcaaaaaaaaaaa

SEQ ID NO: 243 - DNA, 398 bp, Mus musculus
  misc_feature (1)...(398): n = A,T,C or G
ggtcctctgaacaatctacaagaacaactacagcngacacctcagcgcaggaaaaccntaatngntnnnnccagcctttcaaaaccaaaatntttaggttttaccttttagcatggtacattactgngaaaacataacggaatgcagaagaatacagcttttagcttactttggtgaaaaaggattcaaccctgatttttgtaagaaatacccagatgtttcttgtgacaattgctgtaaaacaaaaggnaattacttaagtggngatgcaggcctttaatcccagcacttagaaggcagangaaggtggatctctgaaatcaaggccagnctggtctacacagagagttccaagacagccaaggctacattatgaaacccaatctcaaaaaaaaaaa

SEQ ID NO: 244 - DNA, 457 bp, Mus musculus
  misc_feature (1)...(457): n = A,T,C or G
agctgctggtgcttgtgctgttcccaagccaaagggaaggagagtgcnaggaagaactagtggtggagtcccggtatctgcaatatgacaagaaagcnaanaagggtccccctggcctntganaaagaacatcaaagatggcttaacctctgtagtggctattcctgggtgtcaacttgactatatttgggaatgaactacaatccagaattggaaggctcaccantgaccctaatctggaggctgggagatagaagtntctaanctggatcttggtatggagatcttgaggcatagtggctatggattccagaanattaagacagggagatctccgagttcaaggtcatctgggattaaaggnatggnggcacataatctgggctacaccttctgctggagaccatataaggacattggaagaagggagnctcactctcgctcctttgcctgcttg

SEQ ID NO: 245 - DNA, 445 bp, Mus musculus
  misc_feature (1)...(445): n = A,T,C or G
actgagattgggacaaaacacccatgggaaggagttacagaggcaaagtttggagctgagacaaaaggatggaccatctagagactgccatatccaaagaaattaaagatctcagaagatgcctctcacagaagccagttgtcatcctctgaactgctctaatgaaaagcaaggaccaaaggatggctgagggccaacagccacggagaaaatcaatcctgtcattgcgagtgggtctggacgcaccctcttctttccagtggagccagaggtggcaccagcttgggctaagcctttgaatgtggacttgcaaggactcagccaagttgtgtctagattaccggccaaaagacacctgtaagagttaagttgggngttggtagttgctcacttttggagnaatttattactanttcatagataaccaataaagagtcttttatcg

SEQ ID NO: 246 - DNA, 169 bp, Mus musculus
  misc_feature (1)...(169): n = A,T,C or G
tgcggccataattgttcctattgccaaaccaagttctgtgctcgatgtggaggncgagtgtccttacnctcaaacaagctattgtctattggcatctttattcaccattcanaattaacttgggnatgggtcccaaaagctatgancctattccaaaacttgaaggcca

SEQ ID NO: 247 - DNA, 459 bp, Mus musculus
  misc_feature (1)...(459): n = A,T,C or G
tgaggtgatccctgactaacagcgtcntcaatgcntctgtcgcattcaagacatctgnggggtttctgntctttttaagttttaacccnctggnaggatganggtttcgtcacacctgcaaacccgngccgntcacggtttctcaccggagtggcttgnttctagctctggatctgtgactcatgcagaaacccaaatggaatcatcttgcantagttgaaagtgcttcctctgaagataccaggttttcattgaggnggaagcaaggnacctgactctcaaggnnacanngtgacccatttagaggcntatgnntaatgtataccgnnaatgctatnangcctgtcgagatggcncancaatnaagagcactgactgctcttccagangtcctgagttnaattnccagnaaccacatagtggctcacaaccatctacaatgtgatctgatgccctctt

SEQ ID NO: 248 - DNA, 225 bp, Mus musculus
  misc_feature (1)...(225): n = A,T,C or G
ccgcccaccatgggagccctggctgtnttggagctcacagaaaaaccgccaactntgncgacccctacttaactctggttaaaggtgtgcaccaccatgcccggctntncacaganaatnnttattcttagcatggtaggctcttccttctggatcattttattcattntactacacagnanagnttttactaaaagactttttnccccatttaagaaaaaaaaa

SEQ ID NO: 249 - DNA, 385 bp, Mus musculus
  misc_feature (1)...(385): n = A,T,C or G
actctcaaagagaaaaaggctgtgctcaccaacntcctgntgcgcctgcngtcatccangggaggtagacaagagaacctggagctttggggttaaattcttttactgaggagggattaaaagcacaacaggggtggggggtggnatggggaaagnnnctcaaggatgctgttcancatgagccngacctggatgtcnctcatgatttgcgannttaanttactgcnntgacantnctcacattgtgtcctctaanaaccggttaagccnttgtananagancagcacagctggccatcagagtcaccaatcccaacgccaggctacgcancgaagaaaatgagtagatggcttgtgtgcatgtttttatgtttaaataaaaatg

SEQ ID NO: 250 - DNA, 430 bp, Mus musculus
  misc_feature (1)...(430): n = A,T,C or G
gagcctagcatggctgtcttctgagaggtctaccagcagctgactgagacagatgcacataattacggtacagataattggactaaggtcaaggacccctattgaagagttaggaaaaaggtcaaaggaactgaaggggatgaaaggagctaccccacatgaagaccaacagcagtgtcaactaacctggacccctgaatactcccagaaacaacanggnnngaacatacatggacttattcaaggcctgtggcacatatctagcagagaacttgagtatccccagtgggagaggatgtgcccagttctgtagagactatataccaggtagatacctggggaggtcaccctctcagaggtaaaatggagggaagacagagtggggaattctgggagggggtaatgggagatggggcaatatttgggatga

SEQ ID NO: 251 - DNA, 351 bp, Mus musculus
gactgagcgttgttgcagccagcacatggaagcaactcaggcgctcaggaggctgagaggaggaagcagcgtggaggtggctgaggatggagctcggtcggtcggatgcttgcttcacagacacaaaacaacccagggctgcagagatggcccagtctttaagaacatttctgttcttcccaaggacctgagtttggtgccagaatccacagctggtggtccgtaatagcctgtaacttcaacttcctgtcactccagctccaggggatccaatgccctttcctggccttcctgagcacctgcatacacatcacagacagacagacagacatataaataaaagtaaaaaaa

SEQ ID NO: 252 - DNA, 265 bp, Mus musculus
aagaaagtctcgccaacagatggaatgaaatggaagagaaaatgtcaagtcttgaagacaaagtagaaaaatggatcgatcatccaaagactatgtgaactataaattaaaaatctaggcattcagggaatctgggacagtatgaagagaccaaatctatgaataattgatatagaggaaggagaagaaactcaagtgaaagccacagaaaatatcttttaacaaaaatcatagaataaaaaatttcaaatctaaagaaaaaaaa

SEQ ID NO: 253 - DNA, 147 bp, Mus musculus
  misc_feature (1)...(147): n = A,T,C or G
agactgaggaggaattttctcagcaaacatcatttaaaaggaaattaatggcgaaaatggggcattgggaaaccatgggggaggggggagactgagagaatgagaatgaataatggcaaantaaataaataaataaataaataaaaa

SEQ ID NO: 254 - DNA, 498 bp, Mus musculus
  misc_feature (1)...(498): n = A,T,C or G
gactgagaatggcctacctgctcctgtccggctggcgaatcttaccacntctgnntcttctcccagagacctcccccantcagatcagaagtcccaccttccntttgctgcccagctaccagacattaaatccttattangncagaagcataancagnacnnatagncncantncaccctcatactcagcnntnngctggaacagaantaacaantgaataacacaatnggcnaccttcaagtagtgcnccccaacacacatgacaagggtgctttgagtaggtttggcctccatagattcatgtattncagtgcttggccataggaatggcactagtaggaggtatggtcttattgaagaaggggtagctttgttgaaggaagtgtctcactgtggggatcagctttgaagactcttatgctcaagcaatgcagaagacagtcccctagctgccgtcagatcaagatgtagaactcttagctcctccagcaccatgt

SEQ ID NO: 255 - DNA, 389 bp, Mus musculus
  misc_feature (1)...(389): n = A,T,C or G
tgtgggtctccacggcacanggctgagtggngctggacngggnggngtgctnagtccatgtcaggacacacaggacactggtgcagcttcattctagactgaatcaccaaagagcaagcacngggctggacctaggccccctgcatatatatagcatgatgagccacttgatcttcatgcaggtctcccaaacaactggaggnggggctgtccctgagtctgttgcctgcttgtggatgctgcgcccctaaatggacagncttgtctggcctgggtgggagaagatgtgcctactnctcctgcagggacttgatgtgcagggggcgggggtgggggagtgacatttganatgggggcttccccttctgtttgagaagggggatggggga

SEQ ID NO: 256 - DNA, 416 bp, Mus musculus
gactgaggaaggaaatgaagtgggctagtcttgcgcaccggactcgaccagcaagaacgacgctgcaacaggatccttctgcacacgtttattgggagagcttgattgcagaggcgaaaagaccctgagcccagaactggtgctgcttatataggcctaggagaggcgtgtctcacacccggattggttatgcactatgcctcatttgcatgttcctcatctgattggctactctctctctctgtacctcacagagcctcgttatcatacctcatttgcatgtctcacatctgattggttatactctcaaagcctcattatcatgcccgggccaggcagtgtctttgcaaaaaactttactgcatatgtacacattggttgttgtccaaacttatgcgtggggggccccacaaaat

SEQ ID NO: 257 - DNA, 305 bp, Mus musculus
  misc_feature (1)...(305): n = A,T,C or G
ccatattaaaaatgcacatggttaatcngaacaagggctgaccattttgaaggacctttatttccttgatgctcccggtctttgtctccaccgtctatcgtgctctcatcgaatggctgagctctgtgtgcgtcacgaagggaattcacntntcagcaccnacacagcancttacanctgcctgtaactccagcttcanaggacccaacattctcttctggcctccgaggncatctgcacccgtccnccaccccgncnncatgcatagccaagattaaaataaanctttttttcttttttaaaat

SEQ ID NO: 258 - DNA, 177 bp, Mus musculus
  misc_feature (1)...(177): n = A,T,C or G
gagttgctgcaaacagctctcctggngccaagggatgctcatctcatatttgaatcatcccgaccctcactgagcagacagctcccagagtaacactgactcagagagaaggaattcgagtcacccgggatatttaaaatattctagtagccacgtagtttaacagaaagaaaaaaa

SEQ ID NO: 259 - DNA, 173 bp, Mus musculus
gggttccaagactgccctttgggattcaaacgggagccgctttggactgactggctgccttcctcctcctccccctcctcccccttctgcttcagatttatttattatatgtaagtacactgtagcagttgtaggacacactagacgagggcaccagatctcattgtgggtgg

SEQ ID NO: 260 - DNA, 311 bp, Mus musculus
  misc_feature (1)...(311): n = A,T,C or G
agatctgtccagaaggancagggccaacctgcaaagaagatcagaccacacagccgtctagaagaacagaaaccagctgagctatctggaagagctttagaccaactgagacactgaggaggacactctccagcctgttgagctgcctgcaggctgtgcagtgtgctacaagttcccagcgttgtgagctgtcacccatgctggggtgggcctccgggatgcagctgtctttgagtcatttctgctccgataagtaacccctcacccatattcttccaagtaaccccaataaaactcatggcttaccaagt

SEQ ID NO: 261 - DNA, 100 bp, Mus musculus
  misc_feature (1)...(100): n = A,T,C or G
agtctcgaaattcgataaggtatcttgcnatatattcagaactctccctcctagtgacngcaatgaattcgacccagaagaagatgaacctaccctaaaa

SEQ ID NO: 262 - DNA, 210 bp, Mus musculus
aggtacctgagaggaccagaagatggtgtcagatcctcgggagctggaattgctggcagttgtgtgttgcctgacatcatgagtactagaaagttaactcaggtatctatctataagagcagtaagtgctcttaaccactgagcagtgtctctaacccaacatgatagatctgtatatataaatacatatagataggaaattaaaaaaaa

SEQ ID NO: 263 - DNA, 247 bp, Mus musculus
  misc_feature (1)...(247): n = A,T,C or G
gggttccaagactgccctttgggattcaaacgggagccgctttggactgactggctgccttcctcctcctccccctcctcccccttctgcttcagatttatttattatatgtaagtacactgtagcagttgtaggacacactagacgagggcaccagatctcattgtgggtggntgctaccnnntntccatgccgaangagggggatcacanatagatatagaggtgaaaataaagatctcagttac

SEQ ID NO: 264 - DNA, 152 bp, Mus musculus
  misc_feature (1)...(152): n = A,T,C or G
gatgtcccaggctccggacaacctgcaagccagagacgctacagaaactggaaccanaccctggtncncttccagaaaganngatngatcgaaagaatgaaagaaagaatggcaaaaattaagggtcacccngggggctccaagcctatgag

SEQ ID NO: 265 - DNA, 176 bp, Mus musculus
gggttccaagactgccctttgggattcaaacgggagccgctttggactgactggctgccttcctcctcctccccctcctcccccttctgcttcagatttatttattatatgtaagtacactgtagcagttgtaggacacactagacgagggcaccagatctcattgtgggtggttg

SEQ ID NO: 266 - DNA, 128 bp, Mus musculus; identical to SEQ ID NO: 98

SEQ ID NO: 267 - DNA, 426 bp, Mus musculus
cccggaagcagaagagagaagagattgagaagcggagctgcaagttgtgtgtcaagccttggccccaccatggactcctccccgactctgccccactgaaaacgtcacatggttattttagcaaatggacgttcaggtagaccccctttccagcccttatcccaggagcccccacgagtaacatatgagaagcttctctgttcctttcttaaaactaacttatggggctggagagatggctcagcggttaagcgcactgactgctcttccgaaggtcccgagttcaaatcccagcaaccacatggtggctcacaaccctccataatgatacctgatgccattttatggagtgtctggagtcagctacggtgtacttaagaaataataaataaaacttaggggggaaaaaaaccctaacttaaaaaa

SEQ ID NO: 268 - DNA, 379 bp, Mus musculus
  misc_feature (1)...(379): n = A,T,C or G
ggctggagagatggctcagtggttaanagcactgactgctcttccagaggtcctgagttcaattcccagcaaccacatggtggctcacaaccatctgtaatgggatctgatgccctcttctggtgtgtctgaagacagcgacagtgtactcatataaataaaataaataaatatanntaaaaagncnnantnncatnctnnnannttnntggactcanctacagtttgcctcccaaaatccagggtcaacactgcaccactgaactgtgtcccccagggtaccatcacagagaatggcaaaaaatgagcccacatatgcatgccaacagagtctgatattataggcttgntgttttggaatcttaaccccctggatctg

SEQ ID NO: 269 - DNA, 390 bp, Mus musculus
  misc_feature (1)...(390): n = A,T,C or G
gccctgcacacagacaaatgtatacattcttggagaagactaccttcttccaggtgatgaccggggtgggcacagccttcacctcacaggaaaggaatacttgagtcccagtgacattgtgaatatctcgagggggcatgaggaccacgggagctggaagacaaaggaacaaccctgtgatggtttgcatatgctcagcccagggctttaagacgctcctcctggctgcctgaagttagtattcccctagcagcctgcagatgaagatgtagaactctcagctcctcctgcaccatgcctgcctggatgctgccatgctcccaccttgatgataatggactgaacctctgaacctgtgaacaattaaatgctgnccttataagaaaaaaa

SEQ ID NO: 270 - DNA, 290 bp, Mus musculus
  misc_feature (1)...(290): n = A,T,C or G
ccagcctgaaacctgctngctcggcgnggaggcttcctgctcattccttctgncacgccgactgntggaacctgaggagccacacacgtgcacctttctactggaccactanataatatcggcagggaaatctgggttgccctcccccttacttcataantgggnagtttcaacnataaaatgaagagccttgatcagaaaggtaaganaatgagggccagntnaagatcgnnctgatccacggacaccaagttattccgnggggagacatggctagcttagccctgctg

SEQ ID NO: 271 - DNA, 355 bp, Mus musculus
  misc_feature (1)...(355): n = A,T,C or G
cgagcctgggctagaggccatanattacttcttgaggaactctnanangncgagacagggaaggcaaagaacaaagacccnaaggaaannctgcagnctgtttatgagtgtatagagaaaacgtgtgagaaacttggacagctcaatattgatatttccaacatcaaaggtatttttattgagaattttcctctatagtccagctctgccaatcccattcatgaattcattctttcagcttataataattgagtctttataagctgcaagcctttttggtttttgtttgtttgtttgtttgtttgttggtgagactttgtgggaagaacaataaacaagattctcagttcccgtt

SEQ ID NO: 272 - DNA, 234 bp, Mus musculus
agccaggctggatgaagaaaacttgaagaagattttccgggaggttcagataatgaagatgctttgccatccacacatcatcagactctaccagaggtcctgagttcaagtcccagcaaccatacaatggctcacaaccatctgtaatggaatctgatgccctcttctgctgtgtgtgaagacacctacagttactcatatatataaaataaatattcttttaaagaaaaaaaa

SEQ ID NO: 273 - DNA, 283 bp, Mus musculus
cctggcccttcagaagtggcgccccgacttgtgcagcaaggtggcccagatccatggcattggaggagatgtagtctcccctcccccagcctgaaacctgcttgctcggggtggagcttcctgctcattcgttctaccacgcccactgctggaacctgaggagccacacacgtgcacctttctactggaccagagattattcggcgggaatcgggtcccctcccccttccttcataactagtgtcgcaacaataaaatttgagccttgatcagaaaaaaaaaa

SEQ ID NO: 274 - DNA, 249 bp, Mus musculus
  misc_feature (1)...(249): n = A,T,C or G
gaggcaccctaggatacatttccacacaggccttgtggatgcccatctctactgcctgaagaagnacgntgtggatttnttaatggagaatagcagaacagncttcctaaatgaccancnncaccttanancncanttcgcaganactatccccagctggcnnaagncacgcctctggtagagcacaaggaggatcaaatccccaaacctgggattaaaatgaangcacattcttatattaaaaaagaa

SEQ ID NO: 275 - DNA, 410 bp, Mus musculus
actgaggtcctactctcgctctcccagctattcgtccaagtctggcaaagaggagcccacctagcagaagctccaggtctcgtcgaagtcccagctactctcgctacagccccagcagggaacgggacctaaagtatggtgagaaagaaccacagccccgagaacgtgcgcgcaggaggcgacggtcctactcacccatgaggaagcgcaggagagactccccaagccacctggaggctcggaggataaccagtgcccgcaaacgccccatcccctactaccgacccagcccctcctcgtccagcagcctcagcagcgcctcctcctggtacagcagcagcagcagcagcagcagcagtagcagccgctcccctagccgcagttactcccggagccggagtccctggcct

SEQ ID NO: 276 - DNA, 389 bp, Mus musculus
  misc_feature (1)...(389): n = A,T,C or G
ccaattgaggccgacttgggttgaggtagagctcngggcatgaggccataattacntgttgtggaactctaannggtcggacaggaagaatcnatgcnttaaanaaagacatcnctncattgtgatttgttcctgccccaccctaactgatcaatcgaccttgngacattccttccctggacaatgagtctcatgatctccccaccctgcaccttgtgacctccacttctgcccaaaagagataaccacgtttaactgtgcttttccactgcctacccaaatcctataaaactgccccgcccctattctccctttgctgactcctttttcgtactcagcccgcctgcacccaggtgattaaaaagctttattgctcacacaaaaaaaaa

SEQ ID NO: 277 - DNA, 100 bp, Mus musculus
  misc_feature (1)...(100): n = A,T,C or G
tggtgggcccacctancacgcgtgcaaaangctccncgggtgctcaacttnttctagtcccaactgcgtacgtgcgacccacggaggcaataaatatcca

SEQ ID NO: 278 - DNA, 422 bp, Mus musculus
  misc_feature (1)...(422): n = A,T,C or G
actgaggctcattctccacnaagtgctcaccngtctttgcgngggatgagagancacagcgcngngnangctnagagattnttcatccntcctcggaagtgctngancancatnttcagcttgtcttcctgcttgaccacgataagctacaggcagaaggcagatggtaaggaagggcaaatggtaaggaagaacgcatacacgcgtatgctccctccccagcttccttcctgatgacctgacaagaaatccaggactcagagcagggctactgcgctgagcataagagtacttgtctggagctggagagatgatggctcaatgggtaagagcactgactgctcttccgaaggtcctgagttcaaatcctaacaaccacatggnggctcaccaccatccttaatgaaaactgatgccctctt

SEQ ID NO: 279 - DNA, 303 bp, Mus musculus
  misc_feature (1)...(303): n = A,T,C or G
aggttgtctcggagcaaagctcagatacgaccctggaattgatttacagaattgccagctgcccaacatggattcttggaaccanacctttagaattcaacaaacaaacnacccagggggctggagagatggctcagcactcaagagcactgactgctgctcttccagaggtcctgagttcaattcccagcaaccacatggtggttcacaaccatctgtaatgagttccaatgccctcttctggtgtgtctgaagacagcgacataaaatgaataaatctttttaaaaacccaaaaaaaaaaa

SEQ ID NO: 280 - DNA, 238 bp, Mus musculus
  misc_feature (1)...(238): n = A,T,C or G
caagactgccctttgggattcaaacgggagccgctttggactgactggctgccttcctcctcctccccctcctcccccttctgcttcagatttatttattatatgtaagtacactgtagcagttgtaggacacactagacgagggcaccagatctcattgtgggggcgcttctggatagaggctgtggcagaagtttgacgggcccttnntttgcttgcaagggatgggatgtgggct

SEQ ID NO: 281 - DNA, 339 bp, Mus musculus
ggcactccagttaagagattacctcaaatctgaaataaggctttggacttgtgaacagtattggaattgttgaagaatatggggacttttgaagttgtcctggacatatgtttgcattataagatgacaatgaaggctgccaggaaaactcagaaagtattgtattaccttttgacttgagttcaatccccaaagccaaagtgtggaaggagagagctgattcctgcaacttcttctgacttctatagcactcacatatatcgggcacatgcatgagtatgcacacacacacacacacacacacacagatcgaaaatagttgtaataaaaaaaattaaa

SEQ ID NO: 282 - DNA, 427 bp, Mus musculus
  misc_feature (1)...(427): n = A,T,C or G
gagcctctggtgggcagtcacaagcccacggattccaccagtgaccccaggcctgctcacatgagtgacgcgttcctccccatggccagcagcagagcggactgaatgctgggtgacaggctggcctcagactccggtgctcaaggatattcttgcttcaggttcccaaatagcactgcacactgcacacgtgaatccccatgcctgatggtggatnnctgagttngaggccannctggnntacanagtgagttccaggacanncngngctacacanatnaaccctatcttgaanaacacattaacaaaactgacaaataaatgttatcctttttaacaggtaatagagaatggccccanggggaccctgnaaaagggacttntaatccatcaacatggagcttgagtcttcttgggaagaaaaaaa

SEQ ID NO: 283 - DNA, 327 bp, Mus musculus
  misc_feature (1)...(327): n = A,T,C or G
ctgaggggactggagagctgaaggagcgccagcttctccagaattggacttctcagaacctttaatatgctaatgtgcattgtgaatctccaagatggggatatgatatgcagcattcttgaatacttctaatgacagggagcccactacctcataagtaaacccagacaaacagagataatttaacagaaaagaaacatatatcgggggctggagagatggctcagcanttaagagcactgactgctcttccanaggtcctgagttcaattcccagcaactgcatggtgactcacagccatctacaatgtgatctgatgccctctt

SEQ ID NO: 284 - DNA, 386 bp, Mus musculus
  misc_feature (1)...(386): n = A,T,C or G
agactgaggtatctgagcgtnngagtcaanctggattggcaatngggttagcatggactcccttgggtggcannatnatgtttggggaancangnttaatggncggtgaangccaactancactgaccggncannttggggacgtnntgaaggantctgnccatctnancntnagntgcnttgaaagcnatgcaaacaaataccacctgactaatgcttttggaagttttgatcttnttgacaacacagacatcgtgagatgccaggacttgnaaggngaangngngtntcactcctgggccactgccaaggcttangcatgaaaacttgacatgcgggactggagagatggcttantggttaanagcacttgctactcttgcaga

SEQ ID NO: 285 - DNA, 448 bp, Mus musculus
  misc_feature (1)...(448): n = A,T,C or G
gactgagcttccctgggcccccgagatggctgctcttctgggcgtcccctcccctggccggggccgaggacactgctgctgcgcaagagtcttcaggatggctttggtttcaccctgcgccacttcatcgtgtacccacccgagtcggccgtgcactgcatcctgaaggaggaagagaatggaggccgaggaggaagaggaggaggaggaggaccctccccacggcaccgcctggagcccatggacaccatctttgtgaagaatgtgaaggaatgtggccccgcccacagggcggggcttcgcacaggagaccggctggtgaaggtgaatggggagagcatcattgggaagacctactcccaggtcataggtctgatccaaaaaaagngacnacacccttggactntntattatgcccaaggacgaaggacatcctccagctggccta

SEQ ID NO: 286 - DNA, 100 bp, Mus musculus
  misc_feature (1)...(100): n = A,T,C or G
ccctcaaagctcttgctcgctctgngatgaagattgggcggatngaggatgtcacccccattccctctgatagcactcgaanaaaagggggtcgtcgcgg

SEQ ID NO: 287 - DNA, 268 bp, Mus musculus
gcttaagtttaaaacactgaggagttggcatccattggaagaccttgagatcctctcagctcagaactccaggaccgatgcatcttcccaccaccttgaagcactgaaacaacaagaaaagccgggcgtggtggcgcacgcctttcatcccagccatcgggaggcagaggcaggcggatttctgagttcgaggccagcctggtctacaaagtgagttccaggacagccagggctacacagagaaaccctgtcttgaaaaaccaaaaaa

SEQ ID NO: 288 - DNA, 239 bp, Mus musculus
  misc_feature (1)...(239): n = A,T,C or G
agaactgaagcccctacgatacatttnccacaggccttgtggatgcccatctntactgcctgaanaagtacgtngtggatttcttaatggagaatagngaacaggcttcctacatgaccccancaccttacancacattngnaganactatcccnagctggcataagtcacgccnctgcttgagcacaaggcaagatcaaatccccaaacctgggattaaaatgaaagcacattcttat

SEQ ID NO: 289 - DNA, 219 bp, Mus musculus
  misc_feature (1)...(219): n = A,T,C or G
tacgacactcaaaccttgggggttgcggnctttggcnggnttatggttgannctgccntcnncntcttnctgntgtcancattctccatgaaggagacgccctctgaaaaaacccttgccaaccagcgcaaggagatggcgaaaactcaccaccagaaaggagagaagaaaaagaaggagaaaaccgtggagaagaaaggaaagaccaagaaaaaaaaa

SEQ ID NO: 290 - DNA, 312 bp, Mus musculus
  misc_feature (1)...(312): n = A,T,C or G
gaccatgacatgagcagacngtcggacagctactaataacacctctgatggaacgctttttgcaggatgttctgcgacaccacccatataattaccaagacaacagatctgcaccaaatgagccagaggcggcggcagcagcggcagcagcagcagatcctggatcacctgaagtggtggtggttttggatgattctgatgagaaggaggatgatactgcagatagcggtgcagagaggaactccgaggattcaggatctgttgaagagatagattatagacctggtacttctcaggaacacgcagaagttt

SEQ ID NO: 291 - DNA, 208 bp, Mus musculus
  misc_feature (1)...(208): n = A,T,C or G
cgtcctgggagagagatcgcanaangagcccgctcaagatctcccacccgaagacgctccaggtctccaagacgacatagatccacatctccctccccttctcgactgaaagaaagaagagatgaggaaaagaaagaaactaaagaaataaagaacaaagancggnggatcactgnggaagacctggagggtaaaacagagggcaccg

SEQ ID NO: 292 - DNA, 337 bp, Mus musculus
  misc_feature (1)...(337): n = A,T,C or G
acaatctctctccttaaggaacttaatcaccctaatatcggggggctgctggatgtcatccacacagaaaataagctttatctggtttttgaatttctgcaccaggacctcaagaaattcatggatgcctctgctctcacgggcattcctcttcccctcatcaagggctgacaagatagctcagcaggtaaaagagcctggctncaagcctgatgacctgatggcctgaactccacccccaggttccacacaagaagaacacggnacttctgcaaattgncctctgacttccacacatgcactgtggcatgcccctgcccgctctaaatacaagaaa

SEQ ID NO: 293 - DNA, 417 bp, Mus musculus
  misc_feature (1)...(417): n = A,T,C or G
actgaggtctgccctgctgctgattcgtgtttgctatcccagcactactgaactggactgctggtatatcgtggngtagctgcaagtggattgagctgctgctgctgctgacctgtgaactgaacggctgatttcctgacaatgcagattggatttgctccaaagaacaatttctaaacaggtctatttcccctgtctcccaataacctttcttttctactacctctggtgggtgttgggttagaaggaaggtcaaagcatttaagaacaattattaaaattagggctgggaaaaatgtaagcatatacacaatgcactgtggcatatatggtcatgtgacacacatatacatgtaataatcatgtgacacacatatacatgtaataagtgaatatgnttttttttaattaaaacag

SEQ ID NO: 294 - DNA, 190 bp, Mus musculus
  misc_feature (1)...(190): n = A,T,C or G
tgatacacacaggcctntgaggaaattntaatatgttctggaagctctaaagaaaagcaagagtgcctggcagtggtggaggacacctttaatcccagcacttgggaggaagaagcaggcagatttctgagttcaagaccagcctggtctacagagtgagttccaggacagccagtctcaaaaaaaaaaa

SEQ ID NO: 295 - DNA, 464 bp, Mus musculus
  misc_feature (1)...(464): n = A,T,C or G
aaccctagattcacggacatgccggaggagttcaacatggncacacacgactgtcacagacggcagattctgaacctacatcgtgagcggaagatgtagcctttctacaggacgcatcgagatgcaaggcaataccgtgtcatagaatgtcgtaccacccaggacgagggtgtccccggggccgaggaggacacggagccagaccttcagcaccagctttcagaccccaaaacctgcgacttcttcatccccaacagccgcctgcgcagtatcaatatgagcctccgagcgccccctcttcctcctactcgaactctcaggcccccagcactcaggaggcagaggcataaagatctcctgagtttaaggccaggtctacagagtgaggtccaggatagccagagccacgcggtgaaaccctgtctcaaaaagaaccaaattaaaccaaatcaaaccaaaaaaaa

SEQ ID NO: 296 - DNA, 389 bp, Mus musculus
  misc_feature (1)...(389): n = A,T,C or G
ctcttgtggcctctctggacacagtatgcgcatagtgcagagaaatacancctgaaagctggncnnggtggctgtatgcctttaatcccagcacttgngaggcagangcaggcagatttgngagttcanggncnncctggtctacaaagtgagttccaggacagccagngctatacnganaaaccctgtgtcgaaanccagcatacatannancganngacnagaanacagnttcaggattcttacntataataantaaatcttttnttcaaggatggaactccaccctttacttgggtntaagtatgtatggaatgttntgataggacatannaatagnggncagatgtggaaatggnagggagacaaatatacatgtgaaataaact

SEQ ID NO: 297 - DNA, 609 bp, Mus musculus
  misc_feature (1)...(609): n = A,T,C or G
gagccttgctgacccaggctgnaacatcactgaaaagttccaccccaacatgatttacaactntgaatgcctgcgngngtngnggcccctgnacagtaccgngcagncagcctacaccactcaatgnaagagtcctctcctccattgctggnccctgatcacatggcttaaagctaatattnacctttgnttcctacgccaaccaaaggngaatnccacatctntgntcacatcngggnnnctactgaaaactganccttcaagcttactgaccgnaaaggatgacttatcggatatatgaggaagacacattctgnntgagctctgcntgcctcttccttgtgtaactggttcttnttgaatatcattgnatggagacgccnccatgagcattcttcaacttccagacagggtttccttgannagctctagctatcctggaactatatntgtagnccaaggaagcctcnaacccacagngatctgnccgcctctgctttctgacatgctaggaataaagacacatgccaccactaccactcancactcaaataatatatccttcttccccctgtaattcctttccaactcccctcccatctgaatctt

SEQ ID NO: 298 - DNA, 407 bp, Mus musculus
  misc_feature (1)...(407): n = A,T,C or G
tgagtgttaggagaattcatgcatccctgtgaagatgacatcgtgtgtaaatgtaccaccgaggagaacaaggtgccctacttcaacgcccctgtttacttagaaaacaaagagcaagtcgggaaagtggatgagatatttggacagcttaaagatttttatttttcagttaagttgtcagaaaacatgaaggcatcttcctttaaaaagctacagaagttctatatagacccatacaagctgctgccgctgcagaggtttctgcctcgtcctcctggngagaaaggacctcccagaggtggcggcggnggcggcaggggaggtcgaggaggaggaagaggaggcggtggccgaggtggtggaagaggtggtggttttagaggaggcagaggaggaggtgggggctt

SEQ ID NO: 299 - DNA, 330 bp, Mus musculus
  misc_feature (1)...(330): n = A,T,C or G
acatgaccttgcaagttccntaggnggggggccattatcatnatggcattaantgcacagnaggatccaggcgggcatagctgctgganangctgagagttcnatatcttgctctgaaggccaacannagatgtcttccgntcaactaggnggagggnctctggangcccncccncacantgacatacttccttcaacaaggccacaccttccancaggagccncctcattggcggagcatantcaaaccaccacaagaagtcagactcccaccnangggatgaggcataagcctctataactgcatgagaaataaaagacagaggccgg

SEQ ID NO: 300 - DNA, 356 bp, Mus musculus
  misc_feature (1)...(356): n = A,T,C or G
ttgcctcctcactttcctgggcattcctncgagatctcctgcctctcaaccttngctagtgctattctctgtgcctgggacgatacctttgactcatcttcgactgacctaatggagagagcttgggctttggaggcagagagacctgggttcggattttggcttcacagcttattatttggacaagtcctttggaaaagtatttaacattctggagccccagcttcctaacctgtaaaaatggagatgataacacctaccttgcagggttgttgtaaggattaactgaaattatatatgtatctaatgcaattaacatatagtcaataaatgacacgtcgctgtaaaaaaaaaaa

SEQ ID NO: 301 - DNA, 323 bp, Mus musculus
  misc_feature (1)...(323): n = A,T,C or G
tgccaggcccactgctggancctgtggagccacacactgngcacctttttactggnccanagattattnggccgggaatntgggtcccctcnccnttccttgataactgnnngnnagnagcagnanaantggagntttgatcacgaagangatnangaaagaggtatncacatggcccttaggctaaagtcacacagccagctcttgctggctgtcaaaggctcttgattttctctcagagcattttatttcactttgatttgggggaaacaaaagtggaggtacaatcaatccttgctgattgtgaaggtcaaaaaaaaaaa

SEQ ID NO: 302 - DNA, 317 bp, Mus musculus
  misc_feature (1)...(317): n = A,T,C or G
gactgagtattactgcgactatcaaaagtaggntgttgacatgggagacacccgggcgggcaccaaggctggattncatcacggcatggaatgagcctcaccaaggcttttgaaaatgtccttccgnnctggagagatggctcannggttaagataaacaccaacaacttttccagaggtcctganttcaaatcccantaaccacatggnggctcacaaccatntgnaatagggtctgatgccttcttctggtgtgtctgaagagagcaatggtgtactcatatacattaaataaataaatcatttaaaaaaaagaa

SEQ ID NO: 303 - DNA, 175 bp, Mus musculus
gtagtagctggcaaccctgtggaccttggcgacctaatgctaacagctctgacagactagtgctaacaacttagcaacaaagcatcaagaagcctggggtggcaggggatgggcctcccccctttataagagcagactcttagtaaactttgggccatgattggaaaaaaaaaaa

SEQ ID NO: 304 - DNA, 261 bp, Mus musculus
  misc_feature (1)...(261): n = A,T,C or G
cttcttgtggaaatctgaanncacggacgggnaacactgcctggtctccatancaggaactacagccaggtcctctgaaagagcagccagtgctcttaacccccaagccatctctccagcctgagagtgcgtgttcttatcatgagaagacaaacagcagtggagttggcaagctgcctcagtgggtaaaggtacttgctgcaaggcttgcagcctgatttcgattcccagaacccagagaatagaagtccgaaaaccaac

SEQ ID NO: 305 - DNA, 322 bp, Mus musculus
  misc_feature (1)...(322): n = A,T,C or G
tccttctcttaggaggctngcttcactggggccccctnagnnagggngttgaaaccctctgnaccccattatttcntttnncatncnctttnatgcttctnttcggaacagtcagcggnagtngaaaccttnttccattnggtcctcgtggcaanggcagagattgctccagcagctccacacagtatgcaagacatcttttataattnttgntttttgaaatggggnctcactgnatagctttggcttacctgtaacattctcttgcttttacctcctgagtgctgggataaaaggcatgnaccacctatactcaagaata

SEQ ID NO: 306 - DNA, 201 bp, Mus musculus
  misc_feature (1)...(201): n = A,T,C or G
actgagggcaggtgagatggctcagcanntataacagnttgttgcacaagcctcaggacctgagttcaattcccagaacccatggaaaagatggaaggagagagctgacttgacaaaactgtcttctgaccactgtacacggactggagccacggactgcattgtacaggaaaataataacaatttgtatgaaaaaaaaaa

SEQ ID NO: 307 - DNA, 200 bp, Mus musculus
gttggatctcctggaaacatgaatcacagaaaggtgtgagctgccattcgggtcccaggaactacagccaggtcctctgaaagagcagccagtgctcttaacccccaagccatctctccagcctgagagtgcgtgttcttatcatgagaagacaaacagcagtggagttggcaagctgcctcagtgggtaaaggtacttg

SEQ ID NO: 308 - DNA, 247 bp, Mus musculus
gactgagaatgttggcttccagagcgctgagcctgattggcaaagagagccatttctacttcggtgtgccttcgagcacatggtgagcaggactttcactcccttccctgcccttttctttctcctccctttggtttttcatgacagggtttctctgcatggctctggctgccttggagcgcagagacttaacctgcctctgactcccatgttctgggattaaagatgtgcaccgcaaaaaaaaaaa

SEQ ID NO: 309 - DNA, 185 bp, Mus musculus
  misc_feature (1)...(185): n = A,T,C or G
agtttaactctctggagaccttcttgcaacaggagacaaaggtggcagagttgttattttccagcgggaacaagagcacccacatggcggctcacaaccatgagatctgacgctcttctggtgtgtctgaanacagctacagngtacttacatataataataaataaatgtttaaaaaataaaaa

SEQ ID NO: 310 - DNA, 183 bp, Mus musculus
ggacggtgtggaaaacaagaacagagagatatgccaaggaatgctgaccataatcagaagctaaaagaggcaaagaacagtctctctcctagagcctccagaggggcctcaatatcttgattgcagacttctgggctccaaaattttgagaagaataaattcctcttgttttaaaaaaaaaaa

SEQ ID NO: 311 - DNA, 601 bp, Mus musculus
  misc_feature (1)...(601): n = A,T,C or G
actgagggaggagtacgtagtatcccntctccntggtcngctacctnnttgataggaantggnnntganngntgattncacctgncncnctnantggggnaacctgnggagccanncacntgcnnctttctactgnaccagagattattnggnggnaatcgngtcccctcccccttccttnataantgangnagcancaatnaaatttgagccttgancagaagntaaaanaacacttgntgctgangagggactgtnntccantgaatgccnangtcacagagtttagaaatgcgactttcagctcattccttccattgttggacaaggatcccctgccccaggacttctctgtgaagatggctccactcttcaaggacttcatgaccacctataaccggacttatgaatcaagggaagaagcccagnggcgattgactgtctttgcaagaaatatgatacgagcacagaagattcaggccctggaccgcggcacagctcagtatgggatcaccaagntcagtgacctcacagaggaggaattccacaccatctacctgaatcccctcttacaganggagtctggcaggaagatgagtcc

SEQ ID NO: 312 - DNA, 322 bp, Mus musculus
  misc_feature (1)...(322): n = A,T,C or G
cagtgtggacttcccacgcactgcngatggtgacctgnctggcnctgtgcacnctnaactgcnggaccatnactttnagccantgaggcctggngaacccatnttcaagcttttcancggagaanacgtnctgtatganggggactccattgngtaccctgtgtgnattantgaggcnagccantnnngaacatncctccatggcctntgcatcagctcctgctccctgacctgcttgagttccagtcctgacttcctttggtgatgaacagcagcatggaagtgtaagccgaataaaccctttcctccccaaaaaaaaaaa

SEQ ID NO: 313 - DNA, 289 bp, Mus musculus
  misc_feature (1)...(289): n = A,T,C or G
gagcctttgttctctaggctcaaggtgctccagtgagtgagacaagcgagcgaaaaagcagctccagaaggcctactcaagaaaagaagacctgggctggtgagatggctcagtgggtaagagcacccgactgctcttccgaaggtccgaagttcaaatcccagcaaccacatggnggctcacaaccacccataatgcgatctgatgccctcttctggtagctctcttcacacagtgtacttacatataataaataaataaataaataaataaatcttaaaaaaaaaaa

SEQ ID NO: 314 - DNA, 313 bp, Mus musculus
  misc_feature (1)...(313): n = A,T,C or G
gcagaggcctggctgtctctgttggtacgngccactgctgctgattcctgtctgctgtcctgactctatggacctggactgctggtgtacccatgaagtgtttgtgagtagacggagctgccactgctgacctctaaacgaaactgccgatttccagacaacacaaatgggagttgctccaaagaacctttctaaacaggtccacttgccccatatcctttctttgccactacctctggtagatggtgggctaaaagatgttaaaacatttaagaaccatcattaaaaatagatttaaaataattaaaatgac

SEQ ID NO: 315 - DNA, 441 bp, Mus musculus
atggagcaactatccgatgaagagattgaccatggtgctgacaaggaagatcaggacctggataaaatgtttggggcctggcttggagaatccagctccagcattaaatccggatccagttcttccagtatcccggagtctcagtcaaaccactctaatcagtctgacagcggagtgtctgacaccctgccaactggacatatccgttcccaaagtaccgtgagctccatcttctcggaagcctggaagcgaggcactcagctggaagagtccagcaagaggaccagagttcaattcccagcaccctcgtcgggcaactcaaaaccacctataactccagctccaaaggatccaatgtcctcttctggcctccaaggcacctgaacatgtgtggcatacatacacttaaataaataaaagtagatttttaaagaaaaaaaa

SEQ ID NO: 316 - DNA, 294 bp, Mus musculus
  misc_feature (1)...(294): n = A,T,C or G
gctgccatggcgtcacgcatcgggctgcgcatgcagctcatgcgggagcaggcccagcaggaggagcagctgagagcgcatgcagcagcaggctgtcatgcattatatgcaacagcagcagcagcagcagcagcagctgggtgggccccccaccccagccntcaccncccctgtacaccccnanacntncctntntgngcctntnggcttctctgtgctnngtgacccccaaatgatgggaaacagttttcttgtttaaagaacagtaaagccattctatatacaaaaaaaggg

SEQ ID NO: 317 - DNA, 219 bp, Mus musculus
cccaagtcctgccttcatgccaccatgctccccactatgataagtggactacacctatgaaaatgtgagcaagccccagtaaaatgtcttctttggttgtggctcataatcatctgtaatgggatatgatggactcttctggtgtgtctggggacagcaacaatgtactcatacaatcaatcaatcaatcaatcaataaataaataaatgcagattcag

SEQ ID NO: 318 - DNA, 449 bp, Mus musculus
  misc_feature (1)...(449): n = A,T,C or G
aactgggaaacgtaggtctgaatggagatgggctgcagaccgagcagctattgtcagcccgctggaactggcttcaggcccatgtttctgacttggaatatcgaattcgtcagcaaacagatatttacaaacagatacgtgctaataagatatttgtgcttggaatatagcctggatcatctgcaagagcagtaattgcacttaacccctgagccgtctctccacactgcatgctgtgttttaaactgttagtatggggctggtgaggtaatttagtgggttagcgtgcttgctgccaaccaagcatgatgacccaaattgaatcttcaggacttccatgggtggaaagtgactcatgtgagttgncctctggtgtacttatangcatggggcgtgcacatgccnacataaataaagggaaaataaaatttaatcttttcctatcttac

SEQ ID NO: 319 - DNA, 424 bp, Mus musculus
tgaatcgaccagttttgagaggagaagatggggacaaagcggctcctcctcctatgtcacgcacagctgtctgggattcctatgccaccaccacctatgggacttccccctctacagcctcctccaccacctccaccacctccaccaggccttggccttggctttcctatggctcacccaccaaatctggggcccccaccacctcttcgagtgggtgagcctgtggcgctgtcagaagaagaacggctgaagctggcacagcagcaggcagctttgctgatgcagcaagaggagcgtgccaagcaggcagctgtgttaatggagcaggaacggcagcaggagatcgccaagatgggcactgcagtccctcggccgcctcaggatatggggcagctgggtgtccgtactcctctggggcctcgcg

SEQ ID NO: 320 - DNA, 558 bp, Mus musculus
  misc_feature (1)...(558): n = A,T,C or G
tgagcatcttgccaaagtccaggcactccctacacccagggacagctccagagaagcaacctccagatgctccaacctcctgggncggncncagacacttcaactggacttgttccttctgatctatccacagaccctggacagcaaggaaagagccaactcctaagactgaacaaactcccccatatccattcttctaggttccctagagacacatggccccaaagccagcaggaagtnggcagatatcacaatgaccctatttctgcttcaccatcacccctttctaatcttttcctttttaattctggctaagctccgccccacagttacctggcaacaggcaggtgtgcctgacccactataaaaaggggccgcttgccccctcctccctgtcttgctcctctcttgctctcttgctttctgctacccttccccccaccccccacccccaanccccgngctcatggtcaagcctcttctctacttctctctctntctctctccctntntctgnctttctttgctntactaccctctaaactcccatccccatgt

SEQ ID NO: 321 - DNA, 489 bp, Mus musculus
  misc_feature (1)...(489): n = A,T,C or G
gactgagggcccacctgtctggaaatggatcgttcttactgaacttcgcaggctgcgcagtgtgcagtaagcgggactttatgctgatcacaaacagatccctgaaagaggaagatggagaagaaatagttacctatgaccatctgtgtaagaactgtcatcatgtagtagccagacaccgagtatacattcagcatcatggatgaatttcaggaatataccatgctgtgtctgttgtgtggcaaagctgaagatactatcagtattctccctgatgacccccggcagatgactctgctgttctaagcctcccgtggttctcttgtaactgtgttggcttggtttaaaatagagcaagcctagggctggagagatggctcagtgggtaagagcacccgactgctcttccgaaggtccgaagttcaaatcccagcaaccacatggnggctcacaaccatctgtaacaagatctgactccttcttctggag

SEQ ID NO: 322 - DNA, 448 bp, Mus musculus
  misc_feature (1)...(448): n = A,T,C or G
tgaggagctggtgagatggctcagcagttaaaagcactgactgctcttctgaaagtcctgagttcaaatcccagcaagcacatggtggctcacaaccatctgtaatgagatctgacacactcttgtggtgtgtctgaagacagctacactgtacttatttgtaatcataaataaatcctgggccggccggagctagtggagtgtaccagagcgagcagaagtcctaaattcaattcccagcaagctgatgaagcttcggtacagctacagtatgtactcatctacataaaataaatgattaaattcttaaaataaataaataaatctgattctattatnaaaatttgctactacagnttaaacttctaatggactctngatcttagtgccttattgggataaatactttgcacagatttttntncctttggnagctatgtctcatttc

SEQ ID NO: 323 - DNA, 360 bp, Mus musculus
gtggacatcgaggccctgtgggtggccctacagcgtggaagggatgaccgagctttgggacatgtcctggaaacctaaggtgctggagctagaagagacttgatagcacttcagagaccatcaggctgtttcgtggaacttccaccaaagagatgcagaaatggagactccagagctggagagagagagatgactcaactgttaaaagcactgactgctcttccagaggtcctgagttcaattcccagcaaccacatggtggctcacaattatctgtaatggggatctgatgccctcctctggtgtgtctgaagacagtgatggtgtactgacatatatgaagtaaataagtaaattaat

SEQ ID NO: 324 - DNA, 530 bp, Mus musculus
  misc_feature (1)...(530): n = A,T,C or G
atctgaggaccccggcgagacgcagnggccgccaagagcttcatcttggacatgtatgcgcgcgtgtacgcgagctgcgcagagccccaggacggtggcaggaaaggctcccgcgcgcgccgcttcttcgcacacttcacctgtgccacggacacgcaaagcgtccgcagcgtgttcaaggacgtgcgggactcggtgctggcccggtacctggacgagatcaacctgctgtgacgcgggacagggaaccccaagcgcgacgcggtcgtggcgaggacatacctccccctggtggccgcgcgtggaactgcaggtccaggagctgccaagtggggaagccagcccacagggagagagtccctgcttcctactgggcccccaagcccagctcccctgtaatttattccctcgcccttcttctagtttgttggagaaaggacatgagccgggtctttaaccccagcgctccggaggcagaggcaggaggattttctgtgagttccaggaccatgttttcaaaaacaaacaaa

SEQ ID NO: 325 - DNA, 350 bp, Mus musculus
  misc_feature (1)...(350): n = A,T,C or G
agactgaggctgtttcccttgcaatttgtgcagttgtttgtgcatgatgagagccgncagcagctcaaggtcaagttccgaactggccgcgccttctacttgcaactgcgctctcctncagagacccgcgactgcgagtttgnccgctggntgcggntgctctaccgcctgcgattccactcgcccacctgcgcagtgcccttcacgcacgaggacactgctgcctgangaagaggaggaggaggaggnggaggagggtcanaantcaggcgagatgagggaggacagccctcagtggcatcacagggtcaggatggnggctgtttacacagngctttctgctttccagg

SEQ ID NO: 326 - DNA, 315 bp, Mus musculus
ccccctccaaggaaacagcccacctggaaacctcctccttggaatggtgtagcctcccataagcctgaagcaggctgagccaggcttgactttgttgccactaaggagatcacctggggtggagcctgcctccctggatcactctattgttctgccactgccactgctgccacctgctgcctagctgttctggagcctacacgtggtcacttgcagctgggctccaagaatgatttggccgggaatgggctttcccctctcctttataaagcgtgtccgccattaaaaatttgaaccttgatcagaaaaaaaaaa

SEQ ID NO: 327 - DNA, 422 bp, Mus musculus
  misc_feature (1)...(422): n = A,T,C or G
gaactgaggaagaagatgagcctgaagtccnaacgcaggggaattcatgtggatcaatntgagctcctgtgcaagaaaggatgcggttactacggcaaccctgcctggcagggtttntgctccaagngctggagggaggagtaccacaaggcccggcccgaggcagatccaagaggactgggaactggcagaacggatatttgatcacactgtgaacctcangattgggntatttactgaaantaactggtgggctggcaagatggctcaccctgtaanagtactgatcgntctttctgaacatcctgagttcaaagcccttgcaaccacccgtaatgagatctgacgccctcttctggagtgtcagaaaacagctgnagngnacttatgtataataattaaaaaaattttaaaaaaaaaaa

SEQ ID NO: 328 - DNA, 294 bp, Mus musculus
  misc_feature (1)...(294): n = A,T,C or G
tgaggtggggtctttcattgtcagcgctcatttccctgctgnaagcttggcgnagccggnnanagctgcggggtgaacattgagaggnaccggcatagcatccgctccggctcctcacggagaagatggccgagggcgagcggcagccgccgccaggtccggagttcaaatcccagcaaccacatggtggctcacaaccatctgtaacaagatctgacgccctcttctggagtgtctgaagacagctacagtgtacttacatataataaataaataaatctttaaaaaaaaaaa

SEQ ID NO: 329 - DNA, 460 bp, Mus musculus
aactgaggctggcattgaacttgctatgtagctgagatggccatgaactcatgatcttcttgcctcttcctcccaagtgcctgggttacaggcctgtgctactatgcccagtttgtgtagtgctgaaggtcaaattcagcatttccccgcccgagaggctagcactgtgccaactgatttgcatcgccagccacaaagtgaagctggagccctgcgtcagaagtctagttttataggcaactgtcctgtgatgtcagcggtgcagcgcgtgtgccaccttatttagctaagcagatcgtgtacttcattgggatgctgaaggagatgaatgggcttcgagtgaatgtggcagttaaacataccttcattttttggacttgcatatttagctgtttgaacagagttgtttcctttctgaatttcaaagataagactgctgcagccccatcacaatatccag

SEQ ID NO: 330 - DNA, 495 bp, Mus musculus
  misc_feature (1)...(495): n = A,T,C or G
aactgaggtgaagcatggctaaaagcctgcggagtaagtggaagaggaagatgcgggccgagaagagaaagaagaatgcgccaagggagctcaacagactgaagagtatcctcagagtggacggggatgctctaatgaaagacgttgaggagatagcaaccgtggtggtagccaaacctcgccaggagaaaatgcagtgtgaggaagggcnggtgtgacggcgcagatgaggaaaaggatgacatgaaaatggaaactgaaattaagagaaacagaaagactcttctagaccagcatggccagtacccagtgtggatgaaccagaggcaaagaaaaagactgaaggccaagagagaaaagaaacgagggaaaagcagagcaaaggccgcgaagggcctggcctggtagaccttaaagactggcaagggagacaggcgtggtggtgcacccctttcatcccagcactcaagaggtggaggcaggtggatctctgtg

SEQ ID NO: 331 - DNA, 445 bp, Mus musculus
  misc_feature (1)...(445): n = A,T,C or G
gttgatgggaacaaacacagagacccacagctgcacantgtgcagagactgagaaaccttggaactcgcagncctaaatgggatgtgtccatnagatntctcntgctanngactcagggaactctggcagaaagactgtaggagccaacgagnatgaanactgccacaccacctccngtnagatctgngtgacangttccacagactgaggcgattgacctcaaaagggggaggcctcatggacccagttgtggaactcttcggcggaggaaaaatattgagccgtagntctcatggcaatatatctgttttgnttnctnttattccttttcttatttacatgtncctggnagctgaggccaaggtcttaagcgagaataatgggtantaaaccatccntanagatggggccactancatgancttctncccagggaatcaaagc

SEQ ID NO: 332 - DNA, 382 bp, Mus musculus
  misc_feature (1)...(382): n = A,T,C or G
actgagcctaaattgtatagatctgtgattgaagatgttattaatgacgtacgagnacatttttctggatgacggagtggatgaacaagtcctgatggaactaaaaactttgtgggagaataaactaatgcagtctagggcagtagatggatttcattcagaagaacagcagcttttgttgcaagttcagcagcagcatcaaccccagcagcagcagcatcaccaccaccaccaccagcaccagcaagcgcaacctcagcaaacggtacctcagcaagcacagacccagcaggtcctcattcctgcatcccagcaaggtgagaaggctggaggtccctgttgtgccttacaagttacacttgtcttttgaaagtaaaaaaaa

SEQ ID NO: 333 - DNA, 382 bp, Mus musculus
  misc_feature (1)...(382): n = A,T,C or G
tcatctacaaccacatcaccttctncatcatgatcctctacctnactgaagctgantccacagtgctgcctgatccaanattgtcatcggaggaggctgaaccgtttcaccccntgcgggnganactcccaggcngnactcatcgtanctggnaccccatcggggatatgccaaacacactggcagttctcctcaaacgtgacaccagcagncttcacccagcannaagaccaagcagcgccactccagaacgcagagccccctccaggccttctcccagattacctgattttgattnaactttgggaaggggnatctgtctaaaanaaaatcntccatttcatctagattttccagttttggcnaatataggcttttataa

SEQ ID NO: 334 - DNA, 222 bp, Mus musculus
cgggcagcgatgatgtgacgaacgcagggtattctgcagccaattctaattcaattttcgccaactccacgggtttctctgtagccctggctgtcctggaactcactttgtagaccaggctggccttgaactcagaaatctgcctgcctctgcctcccgagtgctgggattaaaggcgtgccgccatcacgccagcaaataaataaatcttaaaaaaaaaaa

SEQ ID NO: 335 - DNA, 100 bp, Mus musculus
  misc_feature (1)...(100): n = A,T,C or G
ggaactcactgcgtanatngggctggccttaaactcactganattngtctttntcctgcctgctgaaattaaagtngngcgtcaccctgaaaaaaaaaaa

SEQ ID NO: 336 - DNA, 100 bp, Mus musculus
tgaacaagaagatgtccacgtccaagaggaaagccaggatgggcaagaggaagaagtcacctgttgaagatgttggacagagagagtcagaaaaaaaaaa

SEQ ID NO: 337 - DNA, 483 bp, Mus musculus
  misc_feature (1)...(483): n = A,T,C or G
actcctctgagagacttgcagcagttggatttagccggggcaccgtgtgagcagtgacggatggctttacttcatgaatgtgtttgagaatctgaagcagttagtgttttttgactttagcactgaggagttcttaccggatgcagcactggtgaggaaacttagtcaagtgttatccaagttaactcttctgcaagaggtaaagctcacgggctgggagtttgatgactatgatattagcgctattaaaggcacctttaaactagngactgcttaatgcacctgtgccaccaaatactccaggactccctcacttccagcaaatcattaaaagctacgcagaagtggggccacagagctggctcagaggttaagagcactggctgctcttccagaggacctgggattgattcgcaccacctacaaggtggctcacaaccatctgtaactccagttccagggggatccaacattttctgatct

SEQ ID NO: 338 - DNA, 406 bp, Mus musculus
  misc_feature (1)...(406): n = A,T,C or G
gattcttcgacttcacatccacagctgcccattgttgggttagctggactacaaattggcactgtctccagtccgctgtcccttcctaacagaggcacctgttcctggtggcacagttcctggtggcattcagagtgccagggaaagcaacaggaggaggaagaggggagcaggtctgagaagcagaagcgcatctggtggtgaccatcgctaggaagagaaggaggcaaggatcggnccaggagaaatgatgtccaaagtcaaacnagtcatacaagatctcactgtggagaaagacaaaggnggggaggcctccagacncagtgaagaanaatcccaccttctggaaagaggnanagaccaagaatcctggaggttatgtcaaanggaaagtcaggccgacttg

SEQ ID NO: 339 - DNA, 253 bp, Mus musculus
tttgagccctgaggcctggtgaacccatcttcaagcttttcagcggagaagacgtactgtatgagggggactccattgtgtaccctgtgttcattaatgaggcaagccagtaaagaacatccctccatggcctctgcatcagctcctgctccctgacctgcttgagttccagtcctgacttcctttggtgatgaacagcagcatggaagtgtaagccgaataaaccctttcctccccaacttaaaaaaaaaaa

SEQ ID NO: 340 - DNA, 379 bp, Mus musculus
  misc_feature (1)...(379): n = A,T,C or G
tgagtggcagcccagcagcagcagcagcagcagtgggagcaggaacagcagtaacaacagcaacagcagcacagccgcctcagagctttggctcctgagccccctgtgggctgaaggcattgcaggtagcccatggtctcagaagaagtgtgcagatgggattaccgtccacgtggagatatggaagaggaccagggattggcactgtgaccatggtcagctgggggcgcttcatctgcctggtcttggtcaccatggcaaccttgtccctggcccggccctccttcagtttagttgaggataccactttagaaccagaagatgccatctcatntggagatgattgaggacgacacagatagctccgaagacgttgtca

SEQ ID NO: 341 - DNA, 303 bp, Mus musculus
  misc_feature (1)...(303): n = A,T,C or G
tatgctagtcactctcctaactgtaaatcaacactaagagcactagaaggttaaaggtcaaggagactctcacaaaggatccaggaatctccagattgatttccagaatggttgaccagtttgcaattccacaagcaatggaggagtggtcctctttctccacatcctggccagcaagtgctngtcacctgagtttttgatcttanccattctgattggnggaaggtggaatctcantgttgctttgatttatatttttctngatcactaaagactttgaacatttcttttgaaaaaaaaaaa

SEQ ID NO: 342 - DNA, 376 bp, Mus musculus
  misc_feature (1)...(376): n = A,T,C or G
gactgagtaccgttccccagctgaagagttctgaatccacgccggatccttctcaacagtctgttttacgggaacctttattaaccactccttccccgtgatgcagttctgaatcctccctgtagcagggggtcttcgctcgtgcctgaaagatgtttcttgtcccgggtctcagcaccacttgttgcacgcgcccccaactcgccagcaagaacgacgctgctacaggatccttctgcacacgtttattcagtcctatttctttcttgtntatatctcccttgtttatatctcccctgnttatatcttccttgtttatatctcccaataataacaatcctctctcgaataataatcctccctctaagaaaagaaa

SEQ ID NO: 343 - DNA, 440 bp, Mus musculus
  misc_feature (1)...(440): n = A,T,C or G
tatctcacaccacagctatgcaatacctaggaanangatactgatcgcctggaactanggaaacaagtgcganagntnngctnanngtaaatntacttangnctanagganccctcgggcgagctatacctggactccttggtggngatgacagacgacaggctgattggntgctagccaggaccacncttctccttgtgctggttctnctccaaactgcnacttaatgcacctgcaatgaaaanggccccacaccagtaaccagatgctgcantgagccgntttattttgctattacaactgggcanatnagacaattttaagccangtggnggngatntatgtctttgattctagcactcaggaggcagaggcaggtggatctattgagttagaggccagcctggtctatagagtgagttccaggacagtcctgtgtt

SEQ ID NO: 344 - DNA, 428 bp, Mus musculus
  misc_feature (1)...(428): n = A,T,C or G
ttgctaggggtggagcttcctgctcattcgttctaccgcgcnnactggtggancctgaggagccacacacgtncacctttgtnctggannaganattattcangagggaatcgggncccctcccccttccttnataannagttgncctnncannngantnnganccttgatcagantactnacanaaagaaagactgntngnnnaaaaatggctgttaccttgagaagaattttggatgcccaggctggtgaanaagggactgacantggaccatcttcctagaactcccanctaaatacttnaggacacatcttntggaatgnatttnacttttgagggggagggagaaattgacttattgattcctaaattctttgcaggatcagaangncngcctttgttacagagtnaacacagaaccaacact

SEQ ID NO: 345 - DNA, 325 bp, Mus musculus
  misc_feature (1)...(325): n = A,T,C or G
gactgagattgacaattgccttttttgcactctctgggctggatatgctggactccttggatgtggtgaacaaagacgatataatagagtggatctactccctgcaggttcttcccacagaagacagtgaactanataatnnggtgcttgctcanagnnatnnattgnatggatnncacnaccnnnaatggnggagccanagaaagnagccaangngctgaaggngtctgcntccctataggtcnaacaganagtgcnaactaaccantacccncanagctngtgtatctagctgnatatgtancaaangatggcctagtcggcc

SEQ ID NO: 346 - DNA, 391 bp, Mus musculus
  misc_feature (1)...(391): n = A,T,C or G
agactgccctttggtattcaaacgggagccgctttggactgactggctgccttcctcctcctccccctcctcccccttctgcttcagatttatttattatatgtaagtacactgtagcagttgtaggacacactanacgagggcaccagatctcattgtgggnggttgagtacnnccnngcatngcngntagggncgatnggnactcagggataanannnntgactagctcttggctntnanccggaanatacantatgtgcnggctantgcacttcatatattaaggcaatgaatgatccnntcatggnacgacagtctgccctccctcctttttgagccattttccctctttcaccatatactcaataaaataagtaaatgaaaaaaaa

SEQ ID NO: 347 - DNA, 294 bp, Mus musculus
  misc_feature (1)...(294): n = A,T,C or G
gactgaggataacgcgtatggagtgaaactccccnctgcagggttgcctgttagcttcctgaggtctcagcggagatatggactgtgggagtgacacttcanggactgggaggctgtggccttgctggcagaagtgcatcacagggacaggcccagtagtttctctccagtctcctctccagcactacttccgtcttcctgccacaatgttccctgaaatgaagataatgaactgaaccttagaaattgtaagccagccccaattaaatgtttccatttatttaaaaaaaaaaa

SEQ ID NO: 348 - DNA, 432 bp, Mus musculus
  misc_feature (1)...(432): n = A,T,C or G
actgaggtgatgtacaactttttgacctccagaaaagctcacagcaaacccacagtttctataacacaaacccaagatggaagtcctgtctactgtctcgagtttaacagccagcagacacagctcttagccgctggtgatgctaagggcatggtcaaagtgtggcagctgagcacagccttnacagaacaagggccccgggaggtggaggacttggatcagntanaancagaaatcactacctgagggctggtgagatggctcagcgggtaagagcactgactactcttctgaaggtcctgagttcaaatcccagcaaccacatggtggctcacaaccaaccgtaatgagatctgatgccctcttctggtgtgnttgaagacagctacagtgtacttacatataataaataaatctttttaaaaaaaaaaa

SEQ ID NO: 349 - DNA, 462 bp, Mus musculus
  misc_feature (1)...(462): n = A,T,C or G
cctttattaaccgctccttnccangatgcagntntgaatcctccctgtaannataggggtctttgctcgtgcctgaagatgttncttgtcccgggtttcngcaccacttnttgcgcgcgctcgactggccangaagaacgacgcttcaacaggatccttntgcncactgtttattgngagagcttnattgtanaggcnaaaagactttttgcccagagctggcgctgcttttataggcctaggagaggcgtgnctcataccctgattggntatgcactaagcctcatttgcatgttcctcatctgattggctactctctctcagtacctcacagagcctcattatcatatcncattngnatgtctcacatccaattggctactntattagnaccttacagaanctgattatcatgcctgggccaagcagtgtttttgcaaaanncttttcctgcatatgtat

SEQ ID NO: 350 - DNA, 335 bp, Mus musculus
  misc_feature (1)...(335): n = A,T,C or G
aagagcccactcccacccgctcaacacaaccctccacatcagcatgttcgaagtggtccaagagcactccaacagggagtctgacttgttctttttggatcttcagacagctccgatctggggacgagggctggctggtgctggacatcacagcagccagtgaccgatggctgctgaaccatcacaaggacctgggactccgnctctatgtggaaaccgcggatgggagggagatgtagtctcccctcccccagcctgnaacctngcttgctcggggnggagcttnctgctnattcgttctgncacgcccactgctggaacctgaggagccacac

SEQ ID NO: 351 - DNA, 262 bp, Mus musculus
agatgggccgagagctggggggtgcgcccagacgcctcgccagccaccagtctgagttggtctttcgaagcagtaggaacctgtcaccttcagccagagacacctgccggccatctgccccagtgtcctgagttcaaatcccagcaaccacatgatgactcacaaccacctgtaatgagatctgatgcactcttctggcacatctgaagacagctacaatgtacttacatataataaatagataaatattgaaaaaaaaaaa

SEQ ID NO: 352 - DNA, 161 bp, Mus musculus
tggcccaaccaaagtcttttggataaagcaccacaatggacaatgtaaggaaggcagccaaacgcttacgcttcccaaggcacagatctttcccagcatgggaaagatcctccctccctccctccctccctccctccttccaccatacactttattaaaat

SEQ ID NO: 353 - DNA, 526 bp, Mus musculus
  misc_feature (1)...(526): n = A,T,C or G
gactgagtggataatcttcctgtcctacacctgaccaaggcatcccctaaaagccccagggaggctggaccatggtacaagaactgccttcatccatcaccgtgagcaagtgtggaagagatgcatcaatgtttggnnagncatagcccacttntggcttaccataggccatgngctaccgancctgnttngggcatctnttgaatggctgcttattgcagtntgtgccttcatattnccacaaancatattatcgnnatccatttttatngtaaaactcaagggtgtaccanaaacctaatttatccctaatagctnggcttgattttacaaacctgctntcagcaccgacctcatttgcttttatctgaagctcaggaaagttaggaaaaggntgaattgattcgttaatatgtaggacatgtanccgggcgtggtggctcacgcctttaatcccagcactccanaggcagaggcggncggattnctgagttcnaggccagcctggtctacagagtgagttcca

SEQ ID NO: 354 - DNA, 311 bp, Mus musculus
  misc_feature (1)...(311): n = A,T,C or G
gcccaagtggatgcaggancccgggcagccagaangggttggtatctcctggaancatgaatnacataaaggtgtgagcngccatncnggncccagganctacagccaggtcctctgaaagagcngacnntgctnttaacccccaagccatntntccagnctgagagtgctgnnntcttatcatganaagacaaacagcagtggagttggcaagctgcctcagtgggtaaaggtacttgctgccaaggcttgcagcctgatttcgattcccagaacccagagaatagaagtcgaaaaccaaaagaaaaaaa

SEQ ID NO: 355 - DNA, 212 bp, Mus musculus
  misc_feature (1)...(212): n = A,T,C or G
tgaggtgacccgaatggagctgtggcccggggccnggacggcgctgctgctgctgctgctcctgctgctgnccaccctgtggttctgcagctccagtgccaagtatttctttaagatggccttctacaacggctggatcctcttcctggcgatcctcgcaatccccgtgtgtgctgtgcgagggcgcaatgtcgagaacatgaaaaaaaaaa

SEQ ID NO: 356 - DNA, 233 bp, Mus musculus
  misc_feature (1)...(233): n = A,T,C or G
tgaggcattttgtgaaagaaggaagatcctgtctgctgcatggataatggactggaacacagaaagaccatgcagggttcggctgtagaaggccggtatctccanaggccagaagacaccatcagatctcctgngactggagttatagaggttgtgagctgccatgttgaaccaancaggtnactgagggacacaggcatgtggatggaaaccctgctgggagaaaaaaaaaa

SEQ ID NO: 357 - DNA, 225 bp, Mus musculus
agagttctgaatccacgccagatccttctcaacagtctgttttacgggaacactttattaaccgctccttcccagtgatgcagttctgaatcctccctgtagcagggggtcttcgctcatgcctgaagatgtttcttgtcccgggtttcggcaccacttcttgcgcgcgcccaactggccaggaagaacgacgctgcaacaggatccttctgcacaaaaaaaaaa

SEQ ID NO: 358 - DNA, 305 bp, Mus musculus
aagggcattcagatctgggagaagtggccccagaagtaaaaccttcagacagccaaacatctgtggcaattgcagatttggaatggagagaaatggaaggagatgactgcgggttccattatggagatggtacaaacgaggctcaggacaatgacttccccacaggtcctgagttcagaaatcctggcaaccacatggtggctcacaaccatccttaatgagatctgacgccctcttctggtgcatctgaagacagctacagtgtacttagatataatagtaaataaattatttaaaaaaaaaaa

SEQ ID NO: 359 - DNA, 273 bp, Mus musculus
  misc_feature (1)...(273): n = A,T,C or G
actgagtgcatggagctgtttgagaagacagaggatgtgcccttgttggaggagatgtgtccctgggggaaggtcttgaaacttcaaatgcccactcactccattctcagcacctctcttcctcatgtctatgaatcaggatgggaagttctcagctactgctctagcatcatacctgcctgccagatgccgtgctccctgccatggatggtcatgaactcaccctttaaaactgnaagccctcaataaagtctttcttctacaaaaaaaaaa

SEQ ID NO: 360 - DNA, 231 bp, Mus musculus
  misc_feature (1)...(231): n = A,T,C or G
tgcaggctggttagaatgaacaggacacgcccacacccacagtactgtaccttnatattctcgccatccagaaggtctatgaagccatcatcctcatcggacaaagtggcttttacagggtttcttcctggagccgtgactgtcctggacctcagtctgtacaccatgctgcccttgnccacctgcctgtctcccaagtgctgggattaaagatgtgcaacccccacaaat

SEQ ID NO: 361 - DNA, 231 bp, Mus musculus
agcatgaaatcgggcagcgatgatgtgacgaacgcagggtattctgcagccaattctaattcaattttcgccaactccacgggtttctctgtagccctggctgtcctggaactcactttgtagaccaggctggccttgaactcagaaatctgcctgcctctgcctcccgagtgctgggattaaaggcgtgcgccatcacgccagcaaataaataaatcttaaaaaaaaaaa

SEQ ID NO: 362 - DNA, 460 bp, Mus musculus
  misc_feature (1)...(460): n = A,T,C or G
tcggctttgggatgggttgcgacggaggcaccatccccaanaggcacgagctggtgaaggggccgaanaaggtcgagaaggaggccagaagaaagcatcggacccccgggagctggagctacaggtttttgtgagccattaatcttggctgttagaaccaaattcggatcctttggaagagcagtacacaggcttaaccacggagccatctntccagcctgaactttgtgtattaaagtctccccaaaccattcccccttgtgaagtcacacccttnttaaactatatnagctcttagcactctgaattggtggttgagcccagctgatggtcacgtgactgcaagctcctcctggtgaactctgtgcactgnttttcttncagaactagcacagtttctgccacagtgaatatttttgaataaatgcgtgggcnttctgtaaatcttacaaaaaaaaaa

SEQ ID NO: 363 - DNA, 100 bp, Mus musculus
cctcattggctctagtggctcctccccgagccggaccagcccggagtccctctacagtgacagctccaatggcagcttccagtccctgactcaaggttgt

SEQ ID NO: 364 - DNA, 266 bp, Mus musculus
  misc_feature (1)...(266): n = A,T,C or G
tattgattctgcctcccaacaagacaanctgaggaagtgatgagccacgnagccgnngacccttanagttttagatgngcggatcccttcatgtatactttacagaaaaccatgntaagccgggcgtggtggcgcacgcctttagncccancactctggaggcagaggcaggcggatttctacaaagtgaggccagnctggtctacaaagngagttccaggacagccccagggctatacagagaaaccctgtctcaaaaaaaaaaa

SEQ ID NO: 365 - DNA, 520 bp, Mus musculus
  misc_feature (1)...(520): n = A,T,C or G
tgacgggaatttgaacccccatggccccgatgacagcatcattgtacttgatgatgacgatgaagatgaagctggtgntcaaccagggccctccaacctaccccccaatcctgcctcaacaggacctggtcctggcctgtctcaacaggccactggtctctccgagccccgtgtggatggagggagcagtaactccggtagtaggaagtgctacaagttggataatgagaagctctttgaagaggtaagcttttgagataaaagggtcgccctttgtggcccatgctggccttgaactcacagagcttttacttgcctcagcctcctgacagctaggattaaaaatgtgaaccactgggcaggagagatggttcagcggttaagagcattgagcatccagaagtcctgagttcaattaccaccaaccacatgatggcgtggctcacagccgtctgtaataggatcttgatgccctcttctggtgtatctgaagacagctacagtgtactcatatatagaa

SEQ ID NO: 366 - DNA, 346 bp, Mus musculus
caggcggtgctaaattttgtctgttacagcctcggccattagaagctggggcaagcttttctgcctacttaactctgattttatttatttgttatatgtaagtacactgtagctgtcttcagacactccagaagagggagtcagatctcattacagatggttgtgagccaccatgtggttgctggggatttgaactctggaccttcggaagaacagtcgggtgctcttacccactgagccatctcaccagccccatatatgtcatttttctgttcccacattctctgtaagattattgtcataagttaaaatctgaattaaagcatttgaatgtcaaaaaaaaaaa

SEQ ID NO: 367 - DNA, 487 bp, Mus musculus
  misc_feature (1)...(487): n = A,T,C or G
ctgaggttgagaacatttccaagctctgactacaacattaaacctcagaatggaagagatgggaagtcgcatcgaggacttacagagaaacgtggatgacctcatgactcaggccgggatcgagaattctatcaaagaaccagcgacctgaanatgttaaacngagtcacgtgggattgctaaaacaaaaactacaccatacgtctgagtaataataatctatgcagtatgaattntgatgttcatttaaattttttttggacacttaaaattaaacccagggcctaaactataggcaagtggctaccaacgagctataccctaacccactgaaaactaaaattcactttaacaaatgaagaacaaagctgggccataatggcacatgccttcaaccccagcacttgggtggcagaggcatgcagccagtctggcctacagagggagttccaggacagccagggctacacagagaaaccctgtcaaa

SEQ ID NO: 368 - DNA, 150 bp, Mus musculus
  misc_feature (1)...(150): n = A,T,C or G
gcacctctggaggtggaaggaagaggatcangaagttcaaggccattctcagctatgttggcagttcaagaccagccggagcttcatgaaaccctgttttaaaattaatgaaataaaataataaaataaaacaagatttaaaaaaaaaaa

SEQ ID NO: 369 - DNA, 100 bp, Mus musculus
  misc_feature (1)...(100): n = A,T,C or G
ttccaaagtctgaccaatcattgcaccagtcgagctgacanccagtgctggctgtttgcctgtaccactattaaaaaataattcagtttaaaaaaaaaaa

SEQ ID NO: 370 - DNA, 394 bp, Mus musculus
aacagcggacacccagagagctaaatgtctcctaccggctaagacaaggaaacccactcagatcgacctctcgtggatcaaacagactctatccaggtaagtacacctgccaatcactaagttcctgtaggaggctgtagcctcccctttaggctgcccgagctgcaccttaaagatggcgcctgccagatgcagagctggtggtgtaaacaagtccatattcggtggagacgtgcccctgccgccctgattggctgaagccatgtacctggtgaggtgacgtggcctgccgtgagtggatgggggctgagagtatataagagtgagaggcccggggttcgagggagatgaagactgaaaatggctgaataaactgctgatagaagaaaaaaaa

SEQ ID NO: 371 - DNA, 321 bp, Mus musculus
  misc_feature (1)...(321): n = A,T,C or G
aacatcttacctccaagaactgcttcagacgtatgaaagagcccatctggnctgagctngtgatggcttcaattctatntagngganagacngagagacttgtgantacntcctntgaggaatctcaacactccatcangngangctcactggttaacancaccgactgntcttccaaaggccctnagttcaaatcccagcaaccacgnggggggctcacaaccatncgtaatgagatntgacnccctcttntggngtntttgaagacagcgacagtgtnctcactataataataataaataagtcttaaaaaaacgaaaa

SEQ ID NO: 372 - DNA, 247 bp, Mus musculus
  misc_feature (1)...(247): n = A,T,C or G
tcacctgtaggctgtcttcacaacacntccagaacagggagtcanatctcattacngatggntgtgagccaccatgtggttgctgggatttgaactctggaccttcngaagaacagtcgggtgctcttacccactgagccatctcaccagccccatatatgtcatttttctgttcccacattctctgtaagattattgtcataagttaaaanggggaattaaagcatttgaatgtcaaaaaaaaaaa

SEQ ID NO: 373 - DNA, 400 bp, Mus musculus
  misc_feature (1)...(400): n = A,T,C or G
gctaaggcagttacattcggagttccgcggactcttcagaggctgtaaaaatgccggagtcacgaaagcctgcaaagagctgttgcttgccttgctgctgctgctgctactgccgtccctgcccgtgctgcagctgctactcctgctgctgctgctgctgcttgttgctgattgttgcggttgctggttgctgtttactgtttgctgccggctggagngctggattgctgattgccagtcggcgaaatcctggcgatgaagatccaacttgccccaagaaacaagacgaccctaagaagtagtctaatgatttcaatacgccctttccccctctgacctttctctcttaccttatgctgggggattggaagggtggcanaaaaaagaacccaataaagta

SEQ ID NO: 374 - DNA, 259 bp, Mus musculus
aactgaggatcggctcctggagcaggacagcctctgtggcctcactggcgctgctttggtttctgggacttccgtggacctggagcgcggcggcggcgttctgtgtgtacagtgggtggcggcggctggcgctttctgcgtatcgtctgcaagacggcgaggcgagacctctttggcctcgaactcagaaatcctcctgactctgcctctgcttcccaagtgctgggattaaagacgtgcgccaccaccaaaaaaaaaa

SEQ ID NO: 375 - DNA, 205 bp, Mus musculus
  misc_feature (1)...(205): n = A,T,C or G
catgatggttgtgagccaccatgtggttgctgggatttgaactctggaccttcggaagagcagttgggtgctcttaccactgagccatctcaccagcccctcaacagatttctgatcngtgagaaaagactggaacatagatgctttctctcctacggcctgaccttcagggtgacaaccatctctgaactgaccgggaaaaaaa

SEQ ID NO: 376 - DNA, 275 bp, Mus musculus
agcacgacagagtcacctgggagaaagacatctggagcacatcgatttgaggtgggaagatcagcccactgtgggtggcaccatcctgtaggctgggaaactgcattgtgttaaatgaagaaaggatctgagcagcaatgcgctcatcgttgtttcctgattgtgggcaaccagctgctccaagctcctgctactctgacgacttccccactgtgatggattctaaccttgaaccatgaaccagaataaaccctttctcccttcaaaaaaaaaaa

SEQ ID NO: 377 - DNA, 182 bp, Mus musculus
aacgacaggaaaagacctgtggtatatgaaactttgttgacaagagaggatccagagactttaaaagcaaaactctgctgggcatggtgactcctgtctgtaattccagcacttggcagcccaaggcagatggaggaggatcagagttcagaaagtgaagcaaattaatggaaaaaaaaaaa

SEQ ID NO: 378 - DNA, 215 bp, Mus musculus
  misc_feature (1)...(215): n = A,T,C or G
tgtcttgcatcgtggttatagaccaagggaataaaacaagatcttataaataggctgcaggcctatcttgaagaccatgcagaagaagaagcanatgaagangatgtactgggagatganactgaggaagaagaaccanngcccatanaactgcctgttaaagaggaagancctcctgaanaggctgttgatatggcatcggaaaagaaggtggt

SEQ ID NO: 379 - DNA, 315 bp, Mus musculus
  misc_feature (1)...(315): n = A,T,C or G
catggcttcctgtgcgtgcttggcaactngacagnaatctanagtcatctgngaagagggaacctnaattganaaaatgcctccaccacaccggactaagngggcnagancatctacctgctgctgctgctggcgacggcancancancagcnttgtttgctatcatcagagcccaccttctttggccttncaatgtgagctaaagaccagnagntctccagaaagtgtntncaagnttgggggncnagnctggggntggtggngngcnnaagctggtgggggggggnggntggggggggggggggggggtgcgg

SEQ ID NO: 380 - DNA, 384 bp, Mus musculus
  misc_feature (1)...(384): n = A,T,C or G
aaatggcaattcagcaagctgataaactgnangaanaaacacactgatcttcantgtcgcgnaggttctcaaggccattggtttacagagaactggcaagcacagaagtaaagtggaaaatggctttacaagttatgtttggccttaaattttttctagtgttagaagcccttttgttccttttcacatgttatatagtgcttaagatagggnngaaaattctagaggaaattcaggacagtctatcagaagtaaagcggggagagagagtaggagcaaggagaaacggtaagtatacaggcctttccaagggtcttgaacccgaggaaaagttaaggtcaggtaggaatacctggggagagattagaaggaagaaaaagaaaa

SEQ ID NO: 381 - DNA, 100 bp, Mus musculus
  misc_feature (1)...(100): n = A,T,C or G
gggaaaaggggacggttttctgaagttatgaagatgcagagnctgagagatggganttactatgcgtgcanacaaatgaagcagcactttgaaagtattg

SEQ ID NO: 382 - DNA, 199 bp, Mus musculus
  misc_feature (1)...(199): n = A,T,C or G
gactgaggatgtcctaacttcaggnatggaaggaaggaaacatgatgccatattgaagaggttttgtctttgtttcaatgggagaagaagagctacggataccatcaaattaataaagattagatttggccaggggagacctcctgaaaatcttggctacagacatgaaggaaaaaattcaagaagaccaaaaaaaaaa

SEQ ID NO: 383 - DNA, 234 bp, Mus musculus
  misc_feature (1)...(234): n = A,T,C or G
agacacccgggcgggcaccaaggctggattccatcacggcatggaatgagcctcaccaaggcttttgaaaatgtccttccgggctggagagatggctcagtggttaagataaacaccaacaacttttccagaggtcctgagttcaaatcccagtaaccacatggtggctcacaaccatctgcaatagggtctgatgccttcttntggtgtgtntgaagagagcaatggtgtact

SEQ ID NO: 384 - DNA, 214 bp, Mus musculus
ctcctgtctgctgatggataatggactggaacacagaaagaccatgcagggttcggctgtagaaggccggtatctccagaggccagaagacaccatcagatctcctgggactggagttatagaggttgtgagctgccatgttgaaccaagcaggtcactgagggacacaggcatgtggatggaaaccctgctgggagaaaaaagaaactggtga

SEQ ID NO: 385 - DNA, 100 bp, Mus musculus
aagactccctcacagttgtttttgaaaataattggcaaccagaacgccaggttccgtttaatgaagtgcgattaccaccaccacctgatataaaaaaaaa

SEQ ID NO: 386 - DNA, 334 bp, Mus musculus
gcgggaattgacaggaggactacgccgcaagggataggcccagaatagcaaccaggaaacaaaatctcatcatggccaatgtccaccaggaaaacgaagagatggagcagcccctgcagaatggacaggaagaccgccctgtgggaggaggtgagggccaccagcctgctgcaaacaacaacaacaacaaccacaaccataaccacaaccaccaccgaagaggccaggctcgccgacttgcccctaacttccgatgggccattcccaacaggcagatgaatgacgggttgggtggagatgggagatgatatggaaatgttcatggaggagatga

SEQ ID NO: 387 - DNA, 127 bp, Mus musculus
  misc_feature (1)...(127): n = A,T,C or G
aatggtgacgaggctccctaaagcagcctcgaccatccacagacagcctcacgtttttctcctactttttccttatgcttttaaatggcagactcgccgggcgtggnggcgcacgcctttaatccca

SEQ ID NO: 388 - DNA, 199 bp, Mus musculus
  misc_feature (1)...(199): n = A,T,C or G
ggcaaacttcatgtgcangctgatacacataggcctctgaggaaatttgtantatgtcctggaagctctaaagaaaagcaagagtgccgggcagnggtggaggacacctttaatcccagcacttgggaggaagaagcaggnngatttctgngttcaagaccagnctgggctacaagagtgagtccaggacagccagtct

SEQ ID NO: 389 - DNA, 237 bp, Mus musculus
  misc_feature (1)...(237): n = A,T,C or G
ccttcaagtctgctatggaattgaaagtgacctggagcttccggtcctttctacctccccggtgctgggatggcagaagtgtgtccgcctgcgcattantcatggttaggaataaaacccagggcttcaagcatgcttggcagatgttctgccaactgagacacanccccagtccccaaaccacatagttntgaaatacaggtcacattnaaaaataaatcaacctgggatggacgg

SEQ ID NO: 390 - DNA, 295 bp, Mus musculus
  misc_feature (1)...(295): n = A,T,C or G
ccctccaagagctgagatcacaagaatgtaccaccataagcggttttgtgtgtgtgttggagagatggttcanaacttaagagcatatgttggtcttctagaggataggaattcatttcccagaacccaagccaggtggctcacaaccacatgtaactctaaccccccaagatccagtatctcggcctcttcagacacctttactcacatgcacatatcaacacttaaatgcacataatttaatttatttccaggaagaaaaagttcctaaaaatcaagacacctgctcaccaga

SEQ ID NO: 391 - DNA, 146 bp, Mus musculus
  misc_feature (1)...(146): n = A,T,C or G
agtgtgtatcatgcggttattgtcatctttttggagttttttgcttggggactactgacagcacccacattggtgaacaatgttctaggacagccagggagacacagagaaaccctatcttgnagaaaggaaggaaggaagaaaaa

SEQ ID NO: 392 - DNA, 193 bp, Mus musculus
  misc_feature (1)...(193): n = A,T,C or G
gactgagagttgtgctgaagacaacagacacctccatgacaacccaaagtatcacagaggaaatgaaaagctgtggactcattcanagttaattaagattaggtatgaccatggaagacttgtgtatgaccatggaagacttctagagaatcttgactggtgatgtggaaaaagagacccagaagaacaaaaa

SEQ ID NO: 393 - DNA, 484 bp, Mus musculus
  misc_feature (1)...(484): n = A,T,C or G
ccattcttgcccaggagagagcgaggtcctggacctggagagtgagctgtacctgggcggtctncccgaggggggacgagtggacctgccactgccccctgaggtgaggacagctgctctccgggctggctacgtgggctgtgtgagagacctcttcatcgatggacggagtcanagatctccggggcctggctgaggcccagggggctgtgggcgttgctcctttctgctcccgggagaccctgaagcagtgtgcgtcggccccttgtngaaacgggggcatctgtcgagagggctggaaccggttcgtctgtgactgcatcggaaccggctttctgggtcgggtctgcgagagagaggccacggtcttaagctatgacggntccatgtacatgaagatcatgctgcccactgcgatgcacacggaagcagaggatgngnccttacgcttcatgtntnanagggcatatggactcatgatggc

SEQ ID NO: 394 - DNA, 167 bp, Mus musculus
  misc_feature (1)...(167): n = A,T,C or G
agatgtggtggtagtaggcggttatgaaactatctgcaaggaccgtctgatgggccagtgtggcccagcgtctnaactaccnanntggcaaaancattggntccttgctacgctctcactatgaacgcattgtttatncctatgaaatgtaccagtctgggagccaa

SEQ ID NO: 395 - DNA, 298 bp, Mus musculus
  misc_feature (1)...(298): n = A,T,C or G
actgacaacagcccttctggggacagtgactgctacatgaactacaggagtgtcgaccaggactctcagcccgtctattgcaacctggagtccctggggcgatgaggagagtgtggtctcctggcgctgaaccagcctccaacagcccccaggatccagcacgctcaacatcacagggggtagaggacaccccaccccacccagagcagaaggagggtgagaaacaaccctccggtcagngngcatgcatgtgatgctcaataaaagctctgggagcagctgactctaaaaaaaaaaa

SEQ ID NO: 396 - DNA, 383 bp, Mus musculus
  misc_feature (1)...(383): n = A,T,C or G
gactgagctttttcctgcaatacagtggntacagtttgctgtggctactctgagtgttataggctccagntcactcctcacctacatcgcgttccagagcccacagaagtctgcagagataaagccactcttgtatctgagcttctctgacctgctcctgggaatttgctggctcgtaaaggcacttctctatggaactgcagcagcccacaaggacagcatctgctataacctacagaccgtgggggagnttnttctacnttgnctcactcctctanacngcannctacantgnggcgcnaggttntnanangnngangaagacagagcngnctnagtcgtagatggagcacttgtnganccaggtgacagattatgttatg

SEQ ID NO: 397 - DNA, 126 bp, Mus musculus
  misc_feature (1)...(126): n = A,T,C or G
ggtgatgccgggaggccccccagccctcaatcagccaatcctcatccccttcaacatggngggacagctaggtggtcagcaaggcctggncctcacactgcccaccgngaatctcacaaacatcca

SEQ ID NO: 398 - DNA, 160 bp, Mus musculus
  misc_feature (1)...(160): n = A,T,C or G
aagtggcaaggagagactcaggagttttgccccaatgngngggnggtgctggttggctgtaagctggacatgcggactgacctggccacactgagggagctanccaagcagagactcatccctgncacacatgagcagggnactgggctggccaagcaag

SEQ ID NO: 399 - DNA, 368 bp, Mus musculus
  misc_feature (1)...(368): n = A,T,C or G
tgtagccactgaggaccagcaaaaacacctgtccaaggcagcacagctgggaccagaancaagtccccaattgtctactctnctgtgcccatctgcttctgctcagtcccctagaccttggaacagagggtcccgcactcacacaggccatgattgaagctgaagcgaagagattcctgagacctgctccaccagcagtcagaagactccacccgagagacgggccccaggcccgntccgacctttttaactggatcagacccgggccctgcacttcnnattgaaggatgaagactgctgnaggtcaccgccctgcggaangctgagncctgtgatgtgtgngaaangtatcctacgtggcagagaag

SEQ ID NO: 400 - DNA, 195 bp, Mus musculus
  misc_feature (1)...(195): n = A,T,C or G
gcgacgcacactgctcancanctatgaggtgttccagttactaacggatctcaaagagcagccggagggngagtgggaagaaacaagcatacccctctgcaaccaaaactaaaaccgccntcaccttatgagacccttaaagnccttatcaaaacccccctcgtgggnncccagagnccagcggatttgtcccaa

SEQ ID NO: 401 - DNA, 232 bp, Mus musculus
  misc_feature (1)...(232): n = A,T,C or G
gaactgatttgggaaggncaatgtgatcctatcagtacataaggaagccccctgctcatgtcttccacagtggccngnggtgggnntgccaacatcttggattacaagagcctccgtgagatcgtggtgaaccaccagtattcanctggctgttttcactacaagtgccctgctgangtgncggnggnanaagncaaaatnnnntccttaacnagatatgtgaaaataactg

SEQ ID NO: 402 - DNA, 177 bp, Mus musculus
  misc_feature (1)...(177): n = A,T,C or G
ccgctgatagagcagcanaagaatatncagangnccaacttgatgantcnngcaaggctnacaggcctcagagcaagggatgacggggngnctgatctgctaaatgaggcaangcaganactcagtnggggtggtaaaagatacnaccccncnaccaagngctgctggatgggctgg

SEQ ID NO: 403 - DNA, 203 bp, Mus musculus
  misc_feature (1)...(203): n = A,T,C or G
gactgaggcataatagtggatccttgatgtggaagacgtccttaccagctttgacaacacaggaaatgtttgnatgtnctntgctcagnatgaantatatccactagctttatcaaaccaaaaaaatatataaaaggancttnntntgtggnacccagggggtggncttgacatgcncggcaaggantcatagcttgttattt

SEQ ID NO: 404 - DNA, 248 bp, Mus musculus
  misc_feature (1)...(248): n = A,T,C or G
aactgaggtggtgggncttctcttgcctaaccagancgctggcatccactgtcttctggcagtgnctgaaccagagccataangnctgnggnaactatgcaaaccgcattgctaccaagccttcacctgcatccaagttcatccagggctacctggncgctgtcatcagtgctgtttccattgctatngggncaatatcctctccctgacngagcagggggtccttacnccaaaataccatggggggg

SEQ ID NO: 405 - DNA, 320 bp, Mus musculus
  misc_feature (1)...(320): n = A,T,C or G
ttacaggagaattagaatatgctacgaaaatctccaggttttcaaatgtctatcatctttccattcatatttcaaaaaactttggagcagatacgacgaagatcttttatattggcctgcggggagagtggactgagcttcgccggcatgaggngggnntctgcanctatgaagcgtnagccaacccagcagaccaccgggtgcnttnaggtcaccccncacacacactttnntttcttaagggccagccggggctccctnanatgcgctgttagngaaaatgngcgaccacctgctggggaaggacagaggatgctcca

SEQ ID NO: 406 - DNA, 411 bp, Mus musculus
  misc_feature (1)...(411): n = A,T,C or G
actgaggatccaaactactggatacaggtgcatcgcttggagcatggagatggagggattctagacctggatgacatcctntgtgacgttgctgatgacaaagacagaatacacctgccagatgattaaaatgngcctaatttantcagcatgggtacttgtncaccagagatantttnaagctaatgtcangngacntcttcntctatcctgcanaaaatgctaaaancattgaacagagcngggnnncactttcaaaaaaatgtttgnaagctnccctnggcttnccccccaagnggatatttaaaancccnccccccccccnttttcttttccttttngttntgcaanttaatnntgatantccgttctgnaaatggnaccatggggccttaaaatttttaataaaac

SEQ ID NO: 407 - DNA, 238 bp, Mus musculus
  misc_feature (1)...(238): n = A,T,C or G
ccactggtcatatgaaggtgnnattgnaactgcacatggattgtcactcctttcaccccaggacctccgcacctggcatgaagacttgtcctgggggagggaaagagcttttcctgagcttcacccactagctcctctttttctccctacacacacagaccctggggcttacacaagtggctgctagaccctgctgtaatcattaaatcccacatctcctgattcagcaaaaaaaaaa

SEQ ID NO: 408 - DNA, 137 bp, Mus musculus
  misc_feature (1)...(137): n = A,T,C or G
agagaccgcacaggaaaagaagctaccgcttggncaanctctaccttgaacaactcaggcngcaagaggagganaaggctgaagcccgtgcattcnaggaggatcaggnggccgngcgcttgaaggaggatgtgctg

SEQ ID NO: 409 - DNA, 425 bp, Mus musculus
  misc_feature (1)...(425): n = A,T,C or G
tctctcctttattgcctactagtnntttaaattggacttttaatatcctaccagcgtgctcttcagacacacatggtgcattgctgccattcccgggattgcatctttgagacacacactcttaggagccttcagcaaacaaagaggccacctcccggtacggctaccggggagggagngatcctgaccgccctctagccttggccagctctggatatgaatcctaccatggaaccgagantggagtcctgtctggcccaggtgctgcaagaaggatgtgggaaancaggttgcnggttgaccaggaactcatagactatttctcagacagacnagaaagtccggctgaccttgagcacnaccaanaccctngttnggataagctttgagnatgggactccgntancttcttggggtgaactcta

SEQ ID NO: 410 - DNA, 196 bp, Mus musculus
gagaagctcgaagcggtctgggggcctctccgctacagtctgcccgatccctgccaggcaacgccccttgcctcaagcacttcccgcttgacctgcgcacgtctatggatggcaaatgcaaggagatcgctgaggagttgttcagccgctcactggctgagagtgagcttcgtagcgccccttatgagttcccaga

SEQ ID NO: 411 - DNA, 284 bp, Mus musculus
  misc_feature (1)...(284): n = A,T,C or G
ggcctgtcccagaagttcctctgnngaccaggctggcctcggactcagagatccacctgcctacnctgagaattgttaaaatggacggtgctacacaggaagagaccattccagtatggactggccattggaaaggacggcacagtatctggactgttctgtgtcctnaggatgctgcggaatgaacacacatctgcgcaagaatacagtagcagaaaccacatctcacacttgataggggagttggaactatgaatattacagtctataataaataacacata

SEQ ID NO: 412 - DNA, 199 bp, Mus musculus
  misc_feature (1)...(199): n = A,T,C or G
gactgagatatcttgataatggaaataataaaatggcaattcagcaagctgataaactgttgaagaaacacaaagatcttcactgtgcaaaggttctcaaggccattggtttacagagaactggcaagcaggaagaagctttcaccttngnnaacttggaggatatagttggnttgaatgngctttaaagaaaaacaaa

SEQ ID NO: 413 - DNA, 366 bp, Mus musculus
  misc_feature (1)...(366): n = A,T,C or G
ctgaaagccggtctctaccgtctaaccaccacgtttggngaacatgtgaacagctgngcaggcatctgcagaacatcagaaaagacttcagcaattcttggagttcaaagaatagtgaagtagntataaacctgtgttcattacccctgctgatacaactacagatgggcacaagnnaatgtcgagcatttcttgggatcagaagagagccggactaattagacgctnncttttgtcgatgntgctntgctttgaaaactatactnncntggggaggantncatngganagaaattctncancngantaancccgnaaaactgcccttctctngtacccangtgctttttttgtggggtgttggta

SEQ ID NO: 414 - DNA, 547 bp, Mus musculus
  misc_feature (1)...(547): n = A,T,C or G
gactgagagaaaaggaaagcaattcaggttcagacgactagaatctttcgtgcatgattcctggcgacagcaacgtgacaaggtgcgtgtccagcggctggaggtgaagcctcgtgctttggaggtgcctgataaacatcccttggccttcgttatacgcatggaaaggattgaaggagtgagtttattgggtaaagagcaccatcatgaaacttggcctgaagaaattgttcagtggtgtctttgtcaaggtcacccctcagagtgtaagaatgctgcgcacggtggagccttatgtgacctggggatttccaaatctgaagtctgtccgggagctcatcttgaaacgtgggcaagcaaagattaacaataagacagtccctctgacggacaacacggtgatcgaggagcacctggggagatttggtgncatttgcctggaagacctcatccatgaaattgccttccctgggaagcatttccaggaggtctcttcattttctgngccctttcctnctctctgnggcccgncatgctaccaggacac

SEQ ID NO: 415 - DNA, 393 bp, Mus musculus
  misc_feature (1)...(393): n = A,T,C or G
agcagcaagtgatccagtctcgagtgaatgaccaggaaggccgagaagaattctatgttcactatgtgggctttancnggcgactggnccnaatgggtggacaaganccgactcgnactgaccnngacagtgaaggatgccgagcagaanaactactgananatncctgatgtgagctggccgagcagccagagctcangatcactcgaaaccaatngcgaangcatgatganatnanccntgtgcnganggcnctggcccctcccttctcagnacnatcacngcttanccatngcnactcncatgtgannggcagcccncagggaaggagatctntcnatntgagcancatcnttgtgnatgacgtggctggcaaagaccacaagatatact

SEQ ID NO: 416 - DNA, 416 bp, Mus musculus
gactgaggtagccttcatggctgtgccacatcattcgcaggagacttcctatctgctgcctccagatccagaggattgggagaaacaaggcatcccggactttgtctatggccaggaggatctcgttggcaaggaagttcagtggcccagggattcacctagtgccgtggacacagtaccactatcccgtttcgactctgccctccgctctgcgtggaggcagcggctggaactgggcctgtttcggtaccgcctagaagatctgcagacccaaatcctccccggttccgtggggtttgtagctcagctgaatatagatcgagggatacagaggaggcgcccccagaatatcagaagtgtgaggcaggagtttgacccggaacagtttaacttcaataaaattcgacctggagaag

SEQ ID NO: 417 - DNA, 333 bp, Mus musculus
  misc_feature (1)...(333): n = A,T,C or G
ttcggtgctacagagtctctaatcttaaaagctgaagggaaagnggagtctgatnncttttgncaattaggtcacattcancctcttatnggaagatnatncaaaagcnttatnntgcataccatgnggtacntacagtntacagtctgattactggaagaatcgctgcctttttatataggtcttggtttggtctacttccattacnatgcattncagngggctatnaaagcattntnaggaggtgctcctatgtncgancccagctttttggcgagccaaggaannttnatttacgactcnnggcttatcgttcnaagcgaacacagacta

SEQ ID NO: 418 - DNA, 398 bp, Mus musculus
  misc_feature (1)...(398): n = A,T,C or G
aatacaggcttgagcaagagcctgtgagaagacggatngngcaagagcgagccagagcgagacgaagatggcaggaanttgccatgtggtcaagagtagcatgaagaactgctgcagagccctcatttcattcttcctcgtacacaatgaatcacgttgggatttcattccggggctatgtacacaaaagtgncangaggaaaatgttctctactcttgcataaaaatgtgtccagtgcttcaggaatggaacttctgctctgccgggcagggcttcagggattctgccgccagagatgaaaggctcctcctcctgttttatngntctgnaattcccgaggngttaatcatccanccctggncngctccccttgnngggatcctcggtgctattctga

SEQ ID NO: 419 - DNA, 376 bp, Mus musculus
  misc_feature (1)...(376): n = A,T,C or G
tgagggagacaggatggtgntgtcaaaacttccaagttnttctcnnagatntncngcaacncgggggncacnatccagaagnnctaccccntngtncccntcttcctcctggnccactncatgggcgggtgccatctccntcctantggctgnanagaggccaacctacttttctggcatggtcctgatttcacctctggtccttgccaatccggaatctgnatcgactttgaaggtccttgctgccaaactgctcaattttgtcctgccaaatatgaccttgnngcgcattgactccagcgtgctgtctcggaacaagtcggaggttgacctgtacaactctgacccactcntctgccgagcagggctgaaggtg

SEQ ID NO: 420 - DNA, 140 bp, Mus musculus
  misc_feature (1)...(140): n = A,T,C or G
ctcctccgaaaaaaactgttcggtttggcggaactgngacagaagtcttactgaaatacanaaagggtgaaacaaatgacttagagttgntgaagaaccagctgtctgatcctgatataaaggatgaccagatcattaag

SEQ ID NO: 421 - DNA, 290 bp, Mus musculus
  misc_feature (1)...(290): n = A,T,C or G
actgatattgcagtaggtgaanaaactataaaggatatgtcagagaataaacaagaagtacatccagatactattacagatgtggaagctgttatagatacagaggaagaacttattaaagaatgtgaagaaatgtggaaagacatggaagattgtcagaataaattatcacttattgggaactgagacactcacgaatgcagatgctcagctctcatttacttatttatgcaaatggagggggntggactggctgaaccttgggncaatggaaaaaaagaaaccccgaa

SEQ ID NO: 422 - DNA, 268 bp, Mus musculus
  misc_feature (1)...(268): n = A,T,C or G
tggcgttttttgatgaagaataanaaatttaaatttcaaaccactttcaccttggaggagctgactgcggtccccttcgngaacggggtcctcttttgcaaggtccgnctgctggatggcggngattttgtcagcttgtcatccagtggtcttcaaatgaagatgatgaantcttatactctacntgcnccatgcctgcctggatgctgccatgtnnctgccttgatgataangaactgaacctctnatcctgtaaactaaccccaat

SEQ ID NO: 423 - DNA, 261 bp, Mus musculus
  misc_feature (1)...(261): n = A,T,C or G
aagtactgaaggctttgtggtgtgtgggggttgnactcacatggctgcagccagtgtgacttcccctggctccctagaactgggngggnctggtttctccaaaaccctcctggggaccaggttaaaacccaagtacctctgttcancctgcaaaaaacatcctgcgggaggccctttccaggcccagtggggncaccnnntactggntccttctgcctgaccaggatncctnanggtgtgcatcccattggggggggggtg

SEQ ID NO: 424 - DNA, 245 bp, Mus musculus
  misc_feature (1)...(245): n = A,T,C or G
tggccctggtggctcaggtactgatgctggagaatgggctactgcggactccacccatgggctggnnggcctgggaacgcttccgctgtaacatcgactgtgtggaggacccgaagaactgcatcaggnnaactcttttttatntaaagntnggnatcnatgacnnaatggngnatantgaccnctggggtntacnttcctacncctctaaaantggtggaagnggggggcggaaacccttcggg

SEQ ID NO: 425 - DNA, 297 bp, Mus musculus
  misc_feature (1)...(297): n = A,T,C or G
gccctgattgagcagactgaggaagcagnggccgcacttaactccctccccgaggtgcctccctggactggggaggtcaacccacctctgagagacggcngtgcttctagggcgcanctccttgggacccctccgatagatctgccctcttgaaaagcaaactgagggctctgngnggntggccctcggagcctacnaagatccagctgcttcgggggtaggattgacaggattgnnccccanagtggactagggttgaacagnttccggnaccgaaaataacagccaagggggaaa

SEQ ID NO: 426 - DNA, 294 bp, Mus musculus
  misc_feature (1)...(294): n = A,T,C or G
cttttcaccggtccctaacctgtaccanntttgggannaggatggaaattnaccanagactcatcaagtccataccactcctgctccactgcagcaggatcaaancangaaggggaatgggagatgcatagaaaattggaaccacagaaaccaggaagtctcctttctgggacagccagngacgctccagcacctcctccagnccctggtccttggcacagcccctccctcatggctggatcctcagatcataatagggccttggagcagggtttgagggtggggatggggatt

SEQ ID NO: 427 - DNA, 167 bp, Mus musculus
  misc_feature (1)...(167): n = A,T,C or G
acctctgcagtgtcaacanggctcaaggangcaggatggatcncgatgtgnctaacatgttngngattgcgggngggaccctggcnattccaatcctggcattngttncntctttcctcctgnggccttcagcactgntaagaatctattattggggggtgactcag

SEQ ID NO: 428 - DNA, 248 bp, Mus musculus
  misc_feature (1)...(248): n = A,T,C or G
tgacttactaccaaatgtgtggtggatgttgaagcgaacaaagttattctgaatcctgtaaatacaaatctttccaaaggagatgcccgtttnntaacnaagacannntngttanganactnntcatnccccacatcctngaaaaccatnnttcnggatgtttgccaagtatggactnnggtnattatatnnntntcaaaaanatttattcaaacaaaatcttaaaggcatccctaagttaaactgac

SEQ ID NO: 429 - DNA, 276 bp, Mus musculus
gagacataaaattatgaaggatatcaccagaagctatgtaaacatttcatctaaggaccaagagaagttcaaggtgttttcacaaggaaaccaaagagacaactgatatgaaagagctgaccgcagtcagagaaatcttgggaggagaactagagtgaaggacaaggcgatgagcaggtgggagagaaaaagcaaacagctcaaacagatcctgtgaggcgggcgacccaggacgaaggctggctttggcgttatctaggtaaccccggtttgttg

SEQ ID NO: 430 - DNA, 105 bp, Mus musculus
  misc_feature (1)...(105): n = A,T,C or G
gagtctggggagctcctgcntaagtcagactgagggtacaaaaatgcagagcaataaagcctttaacttggagaagcagaatcatactccaaggaagcgtcatgc

SEQ ID NO: 431 - DNA, 434 bp, Mus musculus
  misc_feature (1)...(434): n = A,T,C or G
ggtatggcagctggcgagaactggccaaggccctagccagcaggaacatcccagccgttgatccgaatctccaattctaccgtccccagcggctgagcctcaaggaccaagaaattgcccgaagtaggagtaggaacagtagctacctgaagtggaacaagcctgtcccctggctctcagaggtacaatgtcgctgtcttcagacactccagaagtaggagtcagattttgttacggatggttgtgagccaccatgtggttgctgggatttgaactcnggaccttnngangancanncgggtgctcttacccactgagccatctcaccagcccacctcttgtgatcttgactagaagtaccttcctaacccagctcttccactgtttttaaataaataaacaaacaaataactcactgaccttaaaaaaaaaaa

SEQ ID NO: 432 - DNA, 238 bp, Mus musculus
  misc_feature (1)...(238): n = A,T,C or G
tatggatatcaattctcctggaagaaaaataagaaggccggaggggnnctatctgaagaggaagtgtttggctctttctnggnaanggntatgcaaatactggcaagaatgagcggctaatgagccactgctaggtaaacatagtgggaggaaaaccaatanccnctggacccctcagcacnactgnacgagcccagnctcntccaatcaacggaacccagcaagaaaaacccctacc

SEQ ID NO: 433 - DNA, 406 bp, Mus musculus
  misc_feature (1)...(406): n = A,T,C or G
ctcctgcattaagtcagactgagtgtggttttgcttgtggccatttccagtgcgtttgaatttggacccgttgctgggagagaccttgccatcagagataacagcatttgggaccagaaagaacctgccgtacngcgatcggtctttccagttcgtgccttccgtggggatacagaacagtaagtcgcttaataaaacttgctgtctgaatggagggacttgcatcctggggtccttctgtgcctgccctccttccttctatggacgcanctgtgaacatgatgttcggaaagagcactgtggggtctatcctcngtnggtacctggctgcccaagaagngttcccttgtgcagatgctggcacggcccagctccantgncttcctcaagaccttttctacctggc

SEQ ID NO: 434 - DNA, 199 bp, Mus musculus
  misc_feature (1)...(199): n = A,T,C or G
agttcctccatccacaaatnatgcantcgagtttcctcgaatttggggtcaatacacacagagtgcaatggatgaacctcgctctggagaaatcncctgcatantcatggnatttccccttncaggatttatacagaagcagtcttccaaattctgaaacctgatgcccatcacatactttgggctgagaaaagaaaat

SEQ ID NO: 435 - DNA, 114 bp, Mus musculus
  misc_feature (1)...(114): n = A,T,C or G
ctgccttgctccatgaagtccaggacantntgacatgcacttgtttcttgtctcttcatttgaagaatagtcttcgtccactgtgtacatttttttttgttaatagggaaggag

SEQ ID NO: 436 - DNA, 312 bp, Mus musculus
  misc_feature (1)...(312): n = A,T,C or G
aagactgaagtccgtgcccctcgcctgcagctgccatggaagctccttggaccctttagaagcttctcgccgccatcacaggagccccagaggatgtgcagcggggtctccccagttgagcaggtggccgcaaggggacatggatgacacggcagcacgcttctgtgtgcagaagcactcgtggggatgggctgcgtaaccattatccacnggcagtcgcaagtcctcggccctcattgtcagcgnggggngcccncgactttccagtttgtgcagaancctgacaagtctgccccccatctcaccgtctct

SEQ ID NO: 437 - DNA, 251 bp, Mus musculus
  misc_feature (1)...(251): n = A,T,C or G
aatatcttttgcacatgantgttctgcctgcatggatgactgtacagcangctcatgcagtgatgaaggagaccagaagaggtccttgggaatanagttacaaacaattttgaaccaccatgtgggtgctgagaattgaacctatgtcctgtggaagatcagccagtgctcttaaccactgagaaatctctccatcacctattcttgtggtttggtttttctttttctttgngngtttctttttttttttt

SEQ ID NO: 438 - DNA, 226 bp, Mus musculus
  misc_feature (1)...(226): n = A,T,C or G
atactgaaggcactattgctgaggtagaacagatgtgnttgagatcgaggctaaactggacaagctggtcaaactgtgcagnggnatgatcgaagccggcaaatcctacgtcaccaccaacaggctctttgtgagccggcgtccgagacctgtctcagcagtgccagggcnacaccgtcatctcggaatgtctgcagaggtttggagacagcctacaggagatgga

SEQ ID NO: 439 - DNA, 255 bp, Mus musculus
  misc_feature (1)...(255): n = A,T,C or G
atactgaagaataggagggggagccccgaggatgacctgattggaatcccattcccagaccacagcagcgagctcctgagctgnnngaacgagcagngccagctnanccatctgtgcgacctcaccatcaggacgcagggtcttgagtaccgcacccacagggctgtgcttggctgcctgtanccactacttnaagaagctcttnncgggaggnaggagggggaaannnggaatgngaactgggggggggggggg

SEQ ID NO: 440 - DNA, 271 bp, Mus musculus
  misc_feature (1)...(271): n = A,T,C or G
gaactgagttctggcaggtgatctgtaagacagtgactgantatggatcatttgaacgaggcaactcaggggaaagaacattcngaaaggngntaacaatgtgagtgatccgaagggtccacccgccaagattgcccgcctggagcagaaccggcagccctctaggaagagganggcttgggagcacaggttggaaagatgcagggagtgcctttaaancactcgggccatctcatgaaaacngggggtaggaaaggaaccatgtttccca

SEQ ID NO: 441 - DNA, 272 bp, Mus musculus
  misc_feature (1)...(272): n = A,T,C or G
ggtctatgtgcactagnagtaattctgncacaatgcctgaaataactaccagccatcttgnngaaaaacaggntcaggntctagcggngatgtgtattcttattgatgaaaatgacaataaaattgggggctgacaccaagaaaaaactggtcaccttaatgaaaacatcgacaaaggattattacatcgagcttttagggggcttcttgttnaatactgaaaanaagctcctgttgngggggngatcagatgctaaaattcctttccaggg

SEQ ID NO: 442 - DNA, 285 bp, Mus musculus
  misc_feature (1)...(285): n = A,T,C or G
atgcagcagctgccccangccctgtgcttggcaacattccccccaatgatgggatgcccggaggccccatcccgnnaggnnnctttcagncttttatganaccacaatatcncagngcgacncaaagcctccaantaaaatgggaaaccatatctatnggaggagttcctgggacacagatcattgctgccnaatnccanggatcccanacgacaacaaggacacnccaacatgggnggnggaatgcanannatgaaccctcccngaggggatgggggcccatgg

SEQ ID NO: 443 - DNA, 286 bp, Mus musculus
  misc_feature (1)...(286): n = A,T,C or G
aaatttccccagtactgcagaagnacaggnattcaaggacaacctctacagccaacccagttgagggccagcctgttctatcgagatcctgtctngnggacagaaaaagtttcatgagancgtggtgtgttggcacaatcttttatttccgagcacttgacgcattaaaattaagggcagatcttcatgatttgggggcgggccctgggcttgattccaaagaaccttgtttccaaaacaaacaaacgaaaaagttgtatgggggggggggggccaagggttttgg

SEQ ID NO: 444 - DNA, 244 bp, Mus musculus
  misc_feature (1)...(244): n = A,T,C or G
gactgaggtacctcctgnatgatgtcaaccccncaaaggcttcaatctccgcagggatgnctatatccgggtggcatccttgctgaagacgctgttgaagactgaagagngggtgctagtcctgcccccgtggggccgcctctaccactggcaaagccctgacatccaccaggtccggattccctggtccgagttttttgacctcccgagtctcaataaaaacatccccgttataaaaaaaaaa

SEQ ID NO: 445 - DNA, 190 bp, Mus musculus
ttttagggacttcctgaagctgttttgagttgtttacttatggatcttcactgcaagatggactgttccaatctcagaggaactcttccagaaacctgtatagactactttggccttgaactcctacaggtgggcctgcctttgcttcctagtgctgggattaaaggtgtgtatcaccacaaaaaaaaaa

SEQ ID NO: 446 - DNA, 136 bp, Mus musculus
  misc_feature (1)...(136): n = A,T,C or G
ggcttggggagatccgcttgtcaggggnncatcctcttccaggctgagggctcccgttagacactctcacgtttgacatcttcctcctcggggcgggtgtggaccaccgcctcgcctntctccggaaaaaaaaaaa

SEQ ID NO: 447 - DNA, 236 bp, Mus musculus
  misc_feature (1)...(236): n = A,T,C or G
gactgtgcagttgttgctcctggtgatgtggatggctcnaatgtgctcactgccagagctacttcgggnccagnactgaacttntcaatgtctgcatgggatgccanncaccacaaagaaaaanccgggcccctggaggacaattttacacgaccaggggcanccccctgggaagancnaattccctggctgttcccacgaaacaccaagcccaggaagccacattaagggacgtt

SEQ ID NO: 448 - DNA, 177 bp, Mus musculus
  misc_feature (1)...(177): n = A,T,C or G
aggtgatttgccgttgagaagctcgctacttcgcacacaaacaggaggtacggggactcaaaccccacattccctctccaccctcggccttctctctctgctcgtcttcaaaattgtttgaagtttgaaatttcacgaaattcattgtagcanaaattangcccgttgtacaaatca

SEQ ID NO: 449 - DNA, 238 bp, Mus musculus
  misc_feature (1)...(238): n = A,T,C or G
cgatcagtagagggaccagaaacttctctgcaaagtggcctgcacaagctcaaaccagccaacgtctcagtgtggacagggaagggctcatgaagtgctacccctaactgagaagtcactgacagttggaaggacgagttggggttcttcacagatgtggtccctgaaaggcttacccgtgctcctngtcaccagagcacaggaaattgggaggagaatggggggggggggggataga

SEQ ID NO: 450 - DNA, 207 bp, Mus musculus
  misc_feature (1)...(207): n = A,T,C or G
ggctcaataacattgagcggcccctgcttatcttcggcaccaagtttgacctgagacagtggttcctggtgactgactggaacccactcaccgtgtggttctaccgagacagctacattcgcttctccacacagcccttctccctgaagagngcngggacaactgagtgcaccctcctcccctgactatccagactagtgtggaggt

SEQ ID NO: 451 - DNA, 460 bp, Mus musculus
  misc_feature (1)...(460): n = A,T,C or G
gactgaggaaggatttttgctgggagaggtaagacaagaggaaacttttagcatcagtgactcacaaatcagcaacacagagtttctgcaagtaattgagatccataaccatcagccttgttcacaactcttcagtttttatgattatgcgagcaaagtgaatgaagaaagtttggacagaattctgaaagatcgggaggaagaaagttatcggatggtacaggttccggcgaaacacccagcagcagatgtcctacagggaacaggtcatccacaagcagctcacccgcatcctcggngtgccagacctggtcttccttctcttcagcttcatctctaccgccaacaattctactcatgctttanaatatgttctctttanaccaaaccgnaggtataatcaaaggatatcacttgntattcccatctaggcaatactagccagcaggagtccaaaggg

SEQ ID NO: 452 - DNA, 169 bp, Mus musculus
aacagcttgttgcacaagcctcaggacctgagttcaattcccagaacccatggaaaagatggaaggagagagctgacttgacaaaactgtcttctgaccactgtacactggactggagccacggactgcattgtacaggaaaataaaacaatttgtatgaaaaaaaaaa

SEQ ID NO: 453 - DNA, 464 bp, Mus musculus
  misc_feature (1)...(464): n = A,T,C or G
tgaaggtgaagcttcctgttgaagatggcagacccagtatgttctcactgaagttccggcggcattgaagcgggtagccaagtatgtcatccggggcttttatggcattgagcacggtgttggccttggacatcttgatccggaacccctgtgtaaaagaagangacatgctaggagctgntcaagtttgatcgggaagcaacttcgatccgttttgaataatttaaaggganacaagtncatcanatgcanaatgaggntagagactgctgcanatgggaagaccacccgccataactactacttnatcaattaccggactcttgntaatggaagggaaanataagctggaccncatgaaaaggagggattgaaacagangnagagaaattcccccaatcgggcttcccttcaaatgnnctgtctgntgcagnactttcactganctaaaaacccatcaactg

SEQ ID NO: 454 - DNA, 280 bp, Mus musculus
  misc_feature (1)...(280): n = A,T,C or G
acctgactgccgagattgtcacgacttcctggcctgattctgcaccgcctgantctgncagngaccgacaggctttgttcaaaagnatgcccaaacctttttggccctctgtgctacagattacacctttgcgatgnatcctccatcccatgatcngcacagggcagnattgggggcanccgngctaggcctagggnggccngctctatgtctgggggatgaacctnacaaaagttgnnnggcccgggaatcacaaggncnctgaaggccccccaggcct

SEQ ID NO: 455 - DNA, 319 bp, Mus musculus
  misc_feature (1)...(319): n = A,T,C or G
gtcttttgccgcttcatgtcntccatggctcctcaagacatttgtcctcatggtaggacttgccactgttgcgtttatggtgagaanggtgtncctnacaacagactncngcaccttcaanccaaagtttccagannctgnatgggtngacccanggctgaagctttctaccanaggancatctgaggaanctctttacctacagtgacatctggctcttccccaaaaatcagcggggactgtaactctgncaaactgcgantgaaatataagtttcnggatgcctatanccagaacgnccttccagctgggaatgcca

SEQ ID NO: 456 - DNA, 367 bp, Mus musculus
  misc_feature (1)...(367): n = A,T,C or G
cccctgccggagaagaaatgggngactgtgggtagatacctccctgaggatattcaaatgggtgcctgtgacagatagcaaggagggattttgaactttatacaaacgaatcgtagggctggagagatggctcagcagttttgatggctcagcagttttgagcagtggatgctgttccggaggacctgagttcagttcccagcccccacatggnggctcacaaccttcagtaactccagttccaggagatctggtgccctgttctgacatctgctggcaccaggtagatatgtggtgcacagacggtatatgcagccagtgcaccgtacacataaaataaaaattgaaaaactggaaaacaaaaaaa

SEQ ID NO: 457 - DNA, 428 bp, Mus musculus
  misc_feature (1)...(428): n = A,T,C or G
ggcttgcccctgatcagcctgagtctgatctgaagattttggtgtttaaggcattaagaacatagcctgaatngttcatggagtttttcatcagtatgtctgaaaccataaaatataatgatgatgatcataaaaccgctgtttctgaaaaacactggaacgaacagcggcctgggagggagaatttctgcgacatcgccttcgtgggttgaaagatgtaaagttcccgagcccaccgggggngntcctcgcccgccttgcaancacgttacttttnaanaaagctttttcaannaaacctagaaagtnagaacacgccccncccccgtnataagagaatagnatttccctcccgctnctnacaatattttngaagagggtgctgaacttacatngtnncnccggccaggatttccggagaaaaaggg

SEQ ID NO: 458 - DNA, 430 bp, Mus musculus
  misc_feature (1)...(430): n = A,T,C or G
gactgagatgacggcccttcttaccaaccaaacagaatgaggaatttcgtcccttcattcgaaggcttccagaattcaaattttggcacgcagctacaaaaggcattcttgtggctatgatctgtaccttcttcgaggcttttaacgtcccagtgttctggccgatcctggtgatgtacttcatcatgcttttctgtatcacaatgaagaggcaaatcaagcacatgattaaataccggtacataccattcacacacgggaagaggagatacaaagggaaggaggacgtgggcaagacatttgctagttagaagctggactgattctcacacggactcaagaacggttttgagccattgtaacaatgcctttttcttcacataaagtagttgattatgaggaagnngaattntctttttaagaggagccc

SEQ ID NO: 459 - DNA, 122 bp, Mus musculus
  misc_feature (1)...(122): n = A,T,C or G
atactgagtatagtaagaagacatggtttccccccacttccacagcacgtctaagttttatcttttttggggtccttatctgtgggccacantggaacanctgtgaatatcacgctgttaaa

SEQ ID NO: 460 - DNA, 188 bp, Mus musculus
  misc_feature (1)...(188): n = A,T,C or G
gaactgagcatcttccccacccccagcccccangaccaaggacaggctgngtccagacagaaactgttcagcatcccagtgaatggacaattgcactgaactccacaagcatcccctgccccgtgcccatgtcctgagatccttatcccagatggaaaagttaaatcgtggctttgaaaatccaaact

SEQ ID NO: 461 - DNA, 203 bp, Mus musculus
  misc_feature (1)...(203): n = A,T,C or G
gactgagtatcttcagatgaaatggccactgcttgatgtccaagcaggaagcctccagagcagacaaaccctcaaggatgctcgttcgccgtcgccagcacacatcgttgtaagtaacctctcagtgagagccncccctctgttgagggtaaaggatcttttgatgattaaaaactcntatttgtctgttctttaaaaaaaaa

SEQ ID NO: 462 - DNA, 471 bp, Mus musculus
  misc_feature (1)...(471): n = A,T,C or G
gactgaggtcctccttgtggttcatttctgggcaccatgggctccacagtgtgtacagatgaacgatgtcatggcagagttagctaaagaacaccctcatgtttcatttgtgaagctggaagccgaagctgttcctgaagtatctgaaaaatatgaaattagctctgtccccaccttcctgttcttcaagaattctcagaaagtcgaccggttagatggtgcacatgccccagagttgaccaaaaaagtccagcgacacgtgtctagcggagcctttcctcctagtactaatgaacatcttaaagaagacctcagccttcgcctgaaaaagctgactcacgctgccccctgcatgctgttcatgaagggaacacctcaagaaccacgctgtggtttcagcnagcagatggnggaaatccttcacaaacacaatattcagttcagcagcttttgatatcttctcagatgaaa

SEQ ID NO: 463 - DNA, 340 bp, Mus musculus
  misc_feature (1)...(340): n = A,T,C or G
gactgagatttgataatgggctgcattaaaagtaaagaaacaaaagtccagccataaaatacacaccggaaaatcttacagagcctgtaggcccaagtgccagtcattatggagtggaacatgctacagttgccccgacctcttccacaaagggagcattcagtnaattttaaacagtctttcnatgacaccctttggagggtcctcagggntgactccttttggaggagcgtcttcctcattntgagnggtgtcaagttcatatcctacagggtttancagaggatgggcaanaaagtgatactttcctntggaatnaagaagccctaaaagtataaac

SEQ ID NO: 464 - DNA, 238 bp, Mus musculus
  misc_feature (1)...(238): n = A,T,C or G
actgagattcagagtcatggngctcagcgangcgaatttacagacactaacagagtntttaaagaaaaccctggatcctgatcctgccattagacggncagctganaagtttctcgaatccgtagaaggaantcagaattacccactgttgcttttaacattattggaaaaggcacaggatantgtcattaaagtatgtgcttcagtaaccttcaagaactatattaaaaggaattgg

SEQ ID NO: 465 - DNA, 276 bp, Mus musculus
  misc_feature (1)...(276): n = A,T,C or G
gactgagactccagttctcatctggctccgtttttaaactcccaccttgtcactcatctcgcctccttggagtgacatcatgaaggtggtgaacctgaagcaggccattttgcaagcctggaaagagcgatggagtgactaccaatgggcaatcaacatgaaaaaattcttccccaagggagccncctggggactttctcaacctancaaaagcgctcctggagcaggctatgatngggccttcccccattcctttgatcctgtcgtacctgaaga

SEQ ID NO: 466 - DNA, 167 bp, Mus musculus
  misc_feature (1)...(167): n = A,T,C or G
aactgaggtcgcagacactttgtgaaangagtgagtcagagacgacgacntaccccngctctcttcccataccttagcagctgnccaggagttttatgcttgagcagaaaccaatccgtcaaccccagaggggatgataaattataacggttggggtaattagagga

SEQ ID NO: 467 - DNA, 116 bp, Mus musculus
gactgaggttagaaagaatcaaacaatgcagacataattcatctccttaaagattgcgccctagtacttgaaatgctttttatttaaaagagtagtaaaaatggaaaaagaaaaaa

SEQ ID NO: 468 - DNA, 390 bp, Mus musculus
  misc_feature (1)...(390): n = A,T,C or G
gactgagggacagagtcattacagttgaaactacagagtattctgctagcagccttcagatgaagatgtagaactctcagctcctcctgcaccatgcctgccgggatgctgccatgctcctgccttgatgataatggactgaacctctgaacctgtgttgatgtgctgtcgggacagctctggagcacccttaaagtccagctcttccatctccgcttcatagaacttgcggcagaaatctttaaaagactgaaacagggcaacacgaaagtgattagcaccaacagcctcctttccagtgcacaaaatcaaaatatcaaaatgtaatgggngnacagatttgattngcaaagaaaaangnggaaatgtataaaaattcaaaaaaaaaaa

SEQ ID NO: 469 - DNA, 266 bp, Mus musculus
  misc_feature (1)...(266): n = A,T,C or G
actgaggaggtgcccagacatgggtgactggagcgccttggggaagctgctggacaaggtccaagcctactccacggccggagggaaggtgtggctgtcggtgctcttcattttcagaatcctgctcctggggacagcggttgagtcagcttggggtgatgaacagtctgcctttcgctgtaacactcaacaacccggttgtgaaaatgtctgctntgacaagtccttccccatctctcacgtgcgcttctgggtaaaaaaaaaaa

SEQ ID NO: 470 - DNA, 212 bp, Mus musculus
  misc_feature (1)...(212): n = A,T,C or G
aatggaggctgaagagttaattggcagcagtgtgacgattgattccatcatgagcaaaangagagntnttanaaatatgatatgtgaagactgtactgatgagctaagcttgtctaaaatccttgtgccgatcacaccgaaactggntaanccaaaattatgagggttggggaacaccccanagaactggctgaattttcttgctgaaacta

SEQ ID NO: 471 - DNA, 238 bp, Mus musculus
  misc_feature (1)...(238): n = A,T,C or G
ggacctgccctgcctgctacnnnganngtnnnnnacaggaccataggtgnngcaagacctacagcancctgntcanagtggtgggacgttgcattcccancccagatctgatcgtcttactagagcaggagaagngagcccctgnatggcttgggaaaaaagaaacaggcagcccgagcccaaggttttggagacaggntttccnancctgaaaatnaganctccaaaaaatcatgtt

SEQ ID NO: 472 - DNA, 364 bp, Mus musculus
  misc_feature (1)...(364): n = A,T,C or G
actgagagaaatgcaggcagnagntcaggatggnaccgaagattccgatttttctacngatctcnagcatacagacagntnanagagtgatggcacctcgcgccgatctgcccgagtcacccgctcctcagccaggctaagccagagttcccaagattccagccctgttcaaagatngncagtctttngnaactgnggagcctgnctattctaccagaagggtgacccgtagtcagcagcagcccacgccagtgactccaaaaaagtaccctcttngacagactcgctcatctggctctgaaacggagcaggtggtggacttttcagatagagaaactaaaantacagctgatcatgaggagtc

SEQ ID NO: 473 - DNA, 244 bp, Mus musculus
  misc_feature (1)...(244): n = A,T,C or G
actgaggtttaagtgccccatttgttccaagtctgtggcttccnatgagatggaaatgcactttataatgtgtctgancnggnctngngctgtcctacaatnatnatgtccctnactaaanatgccgggtgagtgtgctaatctccctgaagaanctgcttcaggnggncacaatagccagcctgccttgccttgtncatctataacaaaagnctgcntaanctcatggtttagaagtgaacag

SEQ ID NO: 474 - DNA, 368 bp, Mus musculus
  misc_feature (1)...(368): n = A,T,C or G
gactgaggtctactgctgctntgctctacangggttacttcctccaggactgccanctgtnngatcanaataccgtgacactggtgtggcttgaagnatattnacncctcnccngagaatctctaangctacntactcacccacttctganaggcgtcaaaggttccaancgaacnctgcctggntcactagcatcagaatttcacagatttaggttcattctncttgcctcagaaattgatcaaggggcacattcccctgatgttaactgcaaaccttgacgagtctctnaagacagntcaancttcanncncgntttgggctttctgaacctctgggccaccttcagctccatttccttgacaagg

SEQ ID NO: 475 - DNA, 374 bp, Mus musculus
  misc_feature (1)...(374): n = A,T,C or G
gctctggaagctatggaccagcangaccttccagaagagagagaccgatggcagtgggtgaactgataagggccctaggtaggctnaaagtgnccacggaccaagatagcattgaatttggggcatctgggggctggcttgactttataaattggacctgntaagcnaggttcctttggactgggaatgnaaagaaggtggtcacttgnnnaaataaatctggatggcccttggggcntacccncgncccccggggngttttctgggaagcnagtngngaaaaagggggtgcctttttaaaaaagtttttnttgttaaccaactgggcccccacccntttaaattnttgganttttaaaaaaccccctttggaa

SEQ ID NO: 476 - DNA, 275 bp, Mus musculus
  misc_feature (1)...(275): n = A,T,C or G
gactgagatggtggaccgggcaagattactgcttgctgccttgagtganagtgggattantccgaatgacctctttgatgttgatggtggagatgctggccttgccacccntacncccccttcanttcaacagaagcngcctccatccacttcaacagctnggggctgaatcacaataantcaacttccatctttgngatntnccaatanncnatgactcactcccccncctgctaaatacgaaggggganaaccntcacctatgacaaattatc

SEQ ID NO: 477 - DNA, 394 bp, Mus musculus
  misc_feature (1)...(394): n = A,T,C or G
gaaaaccggccattgaatagaatncacngtcaagccgannatncnanccggannatgtgattatncangctanngngcatatgctactggagacnacaaagcctgtttccctcgganccaatccgctggcttaaattttgcagaganaatcacgtcagnccncagaaantcttcctctgtagccatctgnnanncnnngngtntnaantaaatgactgtgnctaatcagnncacagcctcatggagantgactcctacccctctgttcctgaggtccaaaatgnacttanatctgctgtcccanagccagaggaggctcacntagacctcggagggggactcttggaagtcactggctttcctttccccttgctgccaagaccatattctgaag

SEQ ID NO: 478 - DNA, 429 bp, Mus musculus
  misc_feature (1)...(429): n = A,T,C or G
gactgagatgggctggttaaaagaaaagcagcttatcgaaccgaggtgagcgatgaagacatccaaacaatgccatgctgtacaccgggagcactttctatttgcctgggaaactacgagcggagcccctggatgccattccactcgaaggcccccaagtctcctaaggcactgcaagtaagaaggagccgggctcaccatgaattcactttgaagtaggtgggaacttgccacgacgtccctnagctgtgaccgtgtgagaactcggctggctgactgcaggtattccatggctaccttctcccggggggttgagacggncacaaagggttcnacactgcccaggctactcatgtccagcgtaagagatacattggaacctcgnctacatgggggaaagaatgntattacatttaaatattcatgaat

SEQ ID NO: 479 - DNA, 143 bp, Mus musculus
  misc_feature (1)...(143): n = A,T,C or G
tatgctggatgagcagtgggnaccatctanatcgcatagaagaaggcatggaccaaataaataaagacatgagagaggcagagaagactttaacagaactcaacaagtgttgtggcctctgcatctgcccttgtaatagggcc

SEQ ID NO: 480 - DNA, 249 bp, Mus musculus
  misc_feature (1)...(249): n = A,T,C or G
actgaggatgggatgcgtgaagtccaggttcctccgagatggaagcatggnctcnaaaacagagccaagngccaatcagaagggccctgtgtatgtgccggatcccacgtcctccagcaagctgggaccaaacaacagcaacagcatgcccccagggtttgtggagggtgagtatctaggaagcttgggtctcagcatcannatacatncattancttagattctgagttgtaactgatgggggaaagt

SEQ ID NO: 481 - DNA, 407 bp, Mus musculus
  misc_feature (1)...(407): n = A,T,C or G
tgttgggccttaagaggccccaccggtctttctttgagtncttnatccggacactcatcatcgtgggcactggcctgnctgaggntcctttnttcagtcctncatctgcgatggccacntggcctcctaatggaaggatcatctcttttggggcttgnggaactttctgcaccatcgggaaaccacagtggaaccacattgtcttgannagacctgagcccagggccccatatgccccgggcgtgggctggtagnncatgggncctnancncgccaagtggnggncccgccattggcnagnnggtgngccttnccaattttccccccttggaaaatgcttcattggtngncccaaggnggggtaaaaaagtnccgttnacgggacaaggngggcccatcgggttcct

SEQ ID NO: 482 - DNA, 143 bp, Mus musculus
  misc_feature (1)...(143): n = A,T,C or G
tgatgaaaaaggagattntatgagcagatgancggtcctgtnacctncggctnccgccagnanaatggtgccggcgtnatcctccgagntgttgnaagagccggagagancatccagaattccttgggtggaagatgagggcc

SEQ ID NO: 483 - DNA, 171 bp, Mus musculus
  misc_feature (1)...(171): n = A,T,C or G
agacaacgggaggcaccacagaagtctgtggaaagaacagnagatgaantgnccaganctggccttacagggccttcacagagaacgtnccacccgctgnaaactgatgccgggctccttccccncgggacggagtcttcaaacatatnggggataaaaatgtttcctggt

SEQ ID NO: 484 - DNA, 531 bp, Mus musculus
  misc_feature (1)...(531): n = A,T,C or G
gactgagaactgtgcctggtgatcttgnttgcttcagtgcatctacaacatcggcctactcctccctgtcggtgattgagatgatctgtgctgctgtcttacttgtcttctacacgtgtgacctgcactccaagatatcattcatcaactggccttggactgacttcttcagatccctcatagcaaccatcctgtacctgatcacctccattgttgtccttgtagaaggaagaggcagctccagagttgtcgctgggatactgggcttacttgctacgttgctctttggctacgatgcatacatcacctttccctctaaaggcagcaaagacatacagcaggctcccactgaccccactgatgggcccggtgatcgtctttcagctgtctctgctacctggncaataggctcctccatcaaaaaactttcctcctgnnggggcggggggggggctccgcctttcctccaaagctntcaggagggagaaggcagggnggatccctggngaagttgaggccagggctacacag

SEQ ID NO: 485 - DNA, 483 bp, Mus musculus
  misc_feature (1)...(483): n = A,T,C or G
tttaagcccgcgccctccgggtccgctttgcnggatcnccgctagnctggacctgncacgcggaccgcacggngagctncnggnaanggcagaganngacngagtcgaangnctngctctccntacncacatcgcccggntagnctctcctacacancgctgactttcanaaggagctgctctcctcacaccttggacgccgccggaagctncaaaccgcgccgcactaacgccgccgacatgtttccgagccccgcccacacgcggngagctacttccgccctggcggagttcgctcctcggcagaaactggagcaggagctcagggttccgcagcnagcggaagtgctgccttagaacgcangctcaatggctttcctatanccccggaggancggggagctaggggcngtgtccnaagcgcanaaagccacagttgttcaattttctaagcaacaggnctctggtgatccaggcttgtgg

SEQ ID NO: 486 - DNA, 356 bp, Mus musculus
  misc_feature (1)...(356): n = A,T,C or G
caaatccggagattgccaggaggcttctcctcanaggtgctaatcccaatttgaaagatggaactggttttgctgtcattcatgatgctgccagagcaggtttcctggacactgtacaggctttgctggagttccaggctgatgttaacattgaagataatgaagggaacctgcccttgcacttggctgccaaagaaggccacctccctgtggtggagttccttatgaagcacacagcctgcaatgtggggcatcggaaccataagggggacacccgccttcgacttggccaggttntatggaagaaatgaggtcatttagcctgatggaggcaaatggggttgggggagccacaa

SEQ ID NO: 487 - DNA, 220 bp, Mus musculus
tgaggtgggaccccgggggtagagctcaagtccccaggcttggcagcaagcaccgttagctgcagcatgtcatggtttagtggcctcctggttcccaaagtggatgaacggaaaacagcttggggggaacgcaatgggcagaagcgcccacgccacgcgaatcgagccagtggcttctgcgcacctcgctacatgagctgcctcattaataaaaaaagaa

SEQ ID NO: 488 - DNA, 179 bp, Mus musculus
  misc_feature (1)...(179): n = A,T,C or G
cggggcactgcaagatggctcacnagataatactnaactgctttgatgtgacccttgccccttcattnacanacngggggtcctttanactcccagccctgggcacacacaccactcactgaacaccccagnctgaatagnttattttctcantagncaccagagggtgtgttcacttg

SEQ ID NO: 489 - DNA, 104 bp, Mus musculus
gactgagagtcactgttcattttatttcaaaatcggagcatgttcgtcatggagacagatgttctcggttgcacaataaaccaacctttagccagaccattgtg

SEQ ID NO: 490 - DNA, 384 bp, Mus musculus
  misc_feature (1)...(384): n = A,T,C or G
tgagctcttccccaacgcccctacgccaatgtctccancngncacaaacgtgaatganggactntcncantnctcttcaanatncanangggcnctgngnananntttccagggangagggttatgactgattgcgggacatgccagatggtaccttcttagttcgtgatgcctcaacgaaaatgcagggggattatacattgactttgagganggcnggaattgcctgtatctgcaggatctntaatatgtgatggccctcgttggcacctccntanncncnggcataatctaaaatggngnaccttattacnctctaccaccacaagtctctcgctcagtccaatcccaaactcgaccgngaanctgcgtacccagtctcca

SEQ ID NO: 491 - DNA, 546 bp, Mus musculus
  misc_feature (1)...(546): n = A,T,C or G
ctgagggtgacacttcccaaatctcctcagtcctgaccccggcatcatgcatcggaccacacggattaaaatcacggagctgaaccctcacctnatgtgtgccctctgtgggggctattttattgatgccaccaccattgtggaatgcttacattccttctgcaaaacctgcatcgtacgctacttggagaccaacaaatactgccccatgtgtgacgtccaggtccataaaactcgaccactgctgagcatcagatcagacaaaaccctccaggacattgtctacaagttggtgcctgggctttttaaagatgaaatgaaacggcgacgggacttctatgcagcataccccctgacagaggtccccaacggctccaacgaggaccgaggcgaggtcctggaacaggagaagggggctctgggtgacgacgagattgtcagtctttccattgagttctacgaaggcgtcagggaccgagaggagaagaagaacctcacggagaatggagatggggacaaggagaagacaggagtgcgattccccta

SEQ ID NO: 492 - DNA, 426 bp, Mus musculus
  misc_feature (1)...(426): n = A,T,C or G
gactgaggtcttgtccaataaacatgtggctcctgaccacctactacagatatgccagcgcatcggtccgatgctggataaggaggtcccgcccagtatctcaagagtcacttctttgcttggtgcaggaaggcagtctttgctacgaacagcaaaaggtaccttaatttgaagaagtgttgngctttgtattagtagcttacattgcagttgggaagctgtgtaaacaccgtggaaggtttgcagtgccatggactgactatacacttctctgcattagtgnaagatctccgaattttanccatgcgcttgatgggtcctcccntttcanaaaacgtaaaatgttaagcctgaaaagcttgnagttnaacatggggcttggaggttttatcaanggngagtncttaatttttttccattttggaa

SEQ ID NO: 493 - DNA, 478 bp, Mus musculus
  misc_feature (1)...(478): n = A,T,C or G
ctgaggatccactgaccttganccactccaaagctctctggagtcagaatcagctggcttcctctagccccactccaggatcttttccccagggcactgaaagcctcagcccttggcagatangaaacgtctgggcaagggtnnacaggcacactggcaacctgtgtgagtaangagcgcacactgaactgcccatnncaccaacaccctcctttaccctctcctgntcgctctnggccctattcttgctntgtgtgtggaaagaggttttcactcaagcatnagatggagacacattaccgagnccacacaggagagaagcccttgtcctgtagccctatggtcctcagcgctcncgggatttctctgccatgaccaagcacctgaggacacatgggggctgctccctatcgatgccctctgtgcagggatggctgccctagcctggccttcatgnaggngcacatgcgtgggcact

SEQ ID NO: 494 - DNA, 454 bp, Mus musculus
  misc_feature (1)...(454): n = A,T,C or G
attgaggaacagaggttacacctgggccccgagaggctctggaaactcctccccaagcacaaaacccaactgtactaagctttttgtgagctatttcagcccagaatggctgtggaatccagagcaatttcaacctttaaaacctcaggaccatcaagaagaattaatnctagtgaaaatngaagatagtagcttttcttgaagtcaganatgtaaacagaatgggagngcccaaatcttgccnggaattatttcgccagcaattcaggaagttttgctatcatgaaacaccctagggccccgagaggctctggggcgactccaggaacttttgccatcagnggnttaatgccagagttgcacacaaaggagcagatcctanaactgttagtgctggagcnagtccctgagcatcctacctgaagagctgaagatanggggttcaacaacatgg

SEQ ID NO: 495 - DNA, 104 bp, Mus musculus
ggcctctcagccccaagaaagaccgtgtgaatcattgtctaacaatatgtgaaaacattgtggcacagtctctcagaaaacaaagaagaagaagaagaaaaaaa

SEQ ID NO: 496 - DNA, 272 bp, Mus musculus
  misc_feature (1)...(272): n = A,T,C or G
actgattcggtattctaaagaaatggacccttccttccaaatgagaactgcatatggaccacaggacatctatacagtgaaagctgtgcttctggggctggagagatggcttgaaggttaagagcactggctgctctctcagaggacccaggttcaattcccagaaccacatggaagctcacaactttctgtaactccggttccaggggacccggcgcacatgcaggcaaaacgccaacgctttagaataaaantaattaaaagagaaaaaa

SEQ ID NO: 497 - DNA, 102 bp, Mus musculus
gactgaggttagattcagaacaagaaagtggacatatccaaggtctcctccaagtgtgggtccaaagctaatatcaagcacaagcctggtgaaaaaaaaaaa

SEQ ID NO: 498 - DNA, 349 bp, Mus musculus
  misc_feature (1)...(349): n = A,T,C or G
ctctgcacactactgaactggaccgangntatatccaggaaacgcttgtaagtggatcgagctaccactgctgcctcctatgaactgaactgatgatttcctgacaangcagatgggatntactccaaagancaactgctaaacaggnccacttccctggtatcctantaacctttcttttntactcgctctggnggatgatgggctananggaaggttanagcatttaagancctatnanaagagccgggngtggnggcacntgccnttnatcccagnactanggaggcagaggcnggnggatgactgngtttgaggccancctggngtacagagagagtgccaggac

SEQ ID NO: 499 - DNA, 413 bp, Mus musculus
  misc_feature (1)...(413): n = A,T,C or G
ttatctcaggccggtcctgccccgggggacctgctcctcaacgacatcatggtgnccctggnccnaccttcttncgccagcaggatgggctgctacggggtggctacnaggcacaggaaccgctctgcccggctgtgccaccgaggaaggntgtcccaggcantagctttacctatgtcantgaggacttncggacagagnctcctcccagccccagcagagatgttgaagatccccgggaacatcaggcacacaatgcccacctccgtgggcccccaccaaagctcatccctgtntctggaaagctggagaagccttggntgncctgnaactaactgggcagaccagggctgncctgnanctcacagagatctgtttgcttntgcctcccgagggctaggataaaaggcatt

SEQ ID NO: 500 - DNA, 265 bp, Mus musculus
  misc_feature (1)...(265): n = A,T,C or G
gactgagattctccaaacacatagataaacacanagatcganaacaccggcataaggagcacatgaaggataaggataaggaccgggaaaagtctaagcatagcaacagtgaacataaagattctgaaaagaaacacaaagagaaagagaaaaccaaacacaaagatggcagctcagaaaaacataaagacaaacataaagacagagacaaggaaagacgaaaggaggaaaagattagagctgctggggatgcaaaaataaaaaa

SEQ ID NO: 501 - DNA, 377 bp, Mus musculus
gactgagggttcttaaggcccgccccgccccaagccccacccacagcgctggccccgcccaccatgtcgatgaccatcttgcgcaggctctgccgctggcgcttgctgaggttctggaagatgtcgcagttctcttcttgcagcagcttgaatcccacagccaggtggtggttctcaagcacagactcatcgttgtacatcaatgccagctccgaatctgcagagtaccacgtggtaccacctgggtaccaagccccgccccgtgcgcgcccagtctccagttggcttcattttcagactgactagtcccagccccctcctctgccacatcggtgcttcaccaaataaaccaagcatcaggcgcctaaaaaaaaaaa

SEQ ID NO: 502 - DNA, 325 bp, Mus musculus
  misc_feature (1)...(325): n = A,T,C or G
gaactgagagctcttccctgctcttctggncagccagtgtgtgcagaatgccccacaagattggatttgtagtggtcagctcctctggacatgaagatggcttcagtgcccgagaactaatgatccacgcaccaaccgtcagtgggtggagatcaccaaagtaaggctgggctgaggggtgggcatgagagcgcatctctgagtgaatacatggtgggctttacacctcctggtccaaaaacatgaaatgcatgtctagacagctatatggtatatttcagtgaaaactaaattaaatcattaaaattaaagtacaaaaaaaaaa

SEQ ID NO: 503 - DNA, 257 bp, Mus musculus
  misc_feature (1)...(257): n = A,T,C or G
gaggttcctgaaggcttcaaaggaaacctgatagaggaggctcaatggccctgagtgaagatgaggctgaggcagaagtgtctgtcaacacaaaggtccnatcctgtggcaagatggaggaanacaggcgttgctgccttccagganctacatnacagctnttcaatgcnttctggaactataactgtactnntcatgcacaggaggntnnacantgnannacttccntgctaccnctgtgatccatgtgaggtgtt

SEQ ID NO: 504 - DNA, 152 bp, Mus musculus
tgtgtgcacagctgcccacccagtactggacagcatcagcatcattgacactcctgggatcctgtctggggagaagcagcgcatcagccgagatttatttattattatacagtgtactatataataaataaataaatctttaaaaaaaaaaa

SEQ ID NO: 505 - DNA, 395 bp, Mus musculus
  misc_feature (1)...(395): n = A,T,C or G
acactgcagaggaaacccgaattgcttggttggcattggcgagcacatttggttaggagagatagatgagaatagttttcatagcattgatgatcccaactgtgagaggaggaaaaagaattcctttgtgggcttgactaacctgggagccacctgctacgtcaacacctttctccaggtgtggttcctcaacttggagcttcggcaggcgctgtacttatgccccagcacctgcagtgattacacgaagggagatgggatccatgggggaaaagattatgggcntcagacttgnnnnnancatnttaaaaacttgttagctttgttgcaaaacaagtaacaanacgatacatnggatccttcaggatttgttaaagccttgggcttagatactg

SEQ ID NO: 506 - DNA, 475 bp, Mus musculus
  misc_feature (1)...(475): n = A,T,C or G
tattggttaccgtcagagctagantcccatcactgtccttcaaaatgcaatggaatgtaccacggactatgtctcggctggcactcaggacattcgtggaagcacagaaagccaggctctttgatcaccattggcgcataaaagggccgttacttgtgcatagaggtgaatacagagttggcttggacaccacaccttcgaaagcagnggctacatttatctgctgntcagtgccttgcanagnaaaggaatctcttaaatgctcaagccgcgctaagttggggacccttgcgccaggaaacgaagggagnngnacattttattccaanagggtttttatcttccagntccacatcccagganactncatcagaaaangaaggnagaaaccnggacnctttacaanacaacttctatcaggnctgtatcaaccggtntaagaaagacatttagacaattatgggaaaagttggta

SEQ ID NO: 507 - DNA, 487 bp, Mus musculus
gactgagataccattaacaaggacactaaggttcccaacgcctgcttgttcaccatcaacaaagaagaccacactctggggaacatcattaaatcgcagctgttgaaggacccgcaggtgctgtttgctggctacaaagtccctcaccccttggagcacaagatcatcattcgtgtgcagaccaccccagactacagtccccaggaggctttcaccaacgccatcacagacctcatcagcgagctctcccttctggaggagcgattccgggtggccatcaaggacaagcaagaaggaattgagtagcagctgaaagaagcgttgcttagtggctggaaggctggcacatactcctcagggcccttcagtttaccacatggcgacagccactctcagctctctagctgggcacggggctctgccccattctgtacatagactgttgttttcctaataaaagcagagcagaaaatgggaaaaaaaaaaa

SEQ ID NO: 508 - DNA, 455 bp, Mus musculus
  misc_feature (1)...(455): n = A,T,C or G
gctggaattacagtttcccaccgttcgtggctcagattgggtttttgaagttgatcaaggagccacggcctncagcacagccctactgccagcaccatcagaggttctgaaccaccccgggaacgctcaattggggatgtcaccacggcaccatcagagcctctccatcgcccagatgaccacaatttgaccaacttaatcattgaacatggcggtaagccatctnggaaggccttcccagtcctggacattgactaccctcacgtgcgcacaccctttgagatctccctctggattctcctggcctgcctcatgaagataggtaccgagcctgctggcttgccgaagcctcctgcagtcctgcactgaagaaccatctnctccctccaagctgcttttnttttccttttgcctttttcnaaanatcnattttattttatatatatgaagcatcc

SEQ ID NO: 509 - DNA, 514 bp, Mus musculus
  misc_feature (1)...(514): n = A,T,C or G
agaccagtgtctcttcaccagcacccagngtaaggtctgctgngccatgctgatttctgagtctcagaagctggcgcattaccaggtaggcaatttctactgtcttcgtgcgcttaccttccaggcccagcactaaggcctcgctgcttctgccttacacagtanaagctccttgcacagntaccagatgtttccttcatacgtgtttgctagggctngaaattgacttagtttctgtgactgncatctngcccatgcaagacagtatcgttgnatgcanatcttgccggacaacgtgatgcantaggaagtntgagctttggctttacctanaccagaatttgaagccaaccccgttattcattgnaggcaagaaatttcaccatacttggccttagcttcatcttgtacacaatancagtgttntcatagattgtnggaggctaaatttaatagcatgtaaagggcctagcacatggtagggactcaataaataaagttttcttcccccaca

SEQ ID NO: 510 - DNA, 157 bp, Mus musculus
  misc_feature (1)...(157): n = A,T,C or G
atgtgttcttataagcacacacagnggctcacacctgtnggcccgttcttgggaagnagaggtgggaagattgctgtanntnganaccaacctgggctaagnacattanagggaccctcctttaaaaggggaanntatnttcctcattggaagaaca

SEQ ID NO: 511 - DNA, 444 bp, Mus musculus
gactgagcttcctcattgttctggtctgcctcatcttcagtgtcctgtccactattgagcagtatgccgctctggccaccgggaccctcttctggatgaggacccatggaagacttctgaagccgatcacccatggagctcctattaaaagaaaggacatgcatgccttacactattgagacagatgtgggtgttctctgatccgaacttactgcaagggctgctagtgtttgcctggcatacatgaatccctgggttcaatctccagtctgtataaactgtgcacagtgtacacacctgtgatcctagtgctgggagatgaagagagagaggagcactggaaggtcaaggttatccttggctacatagagtttgaggtcagcccaggctccatgagactctgccccagcaatagcaacaacaacaataacaaaaataaataaa

SEQ ID NO: 512 - DNA, 508 bp, Mus musculus
  misc_feature (1)...(508): n = A,T,C or G
gaactgaggccctgancttnatcatcttcttngcttcacggcctccatctctgcctacatggctgcagcgntgctagagttcctcatcacactggccttcctcttcctctacgccacgcagtactaccagcgcttcgataggctcaactggccttgtctggacttcctccggtgtctcagcgccatcgtgcatcttcctggtggtctcctttgctgctgtgacctcgagggagggagctgccattgctgcttttgtttttggcatcatcctggtctccgtctttgcctatgatgcattcaagatctaccgaactgagctgatgcccagtaccactgagggggaccagcagtgattccagggttacctggctcccacgaccagacacaaggaaccgnggagcccatctttgtcatctttgaattcaccgaaccccaagtacccaccatctatattccacgttttcaganatgngcgcttganaagncccaggcacctgggggaactggg

SEQ ID NO: 513 - DNA, 424 bp, Mus musculus
  misc_feature (1)...(424): n = A,T,C or G
gactgagttttaagctataacattaatcatgagtgaccaaatcaaattcatcgtggacagcctcaataaggagccttttaagaagaattataacttaatcacatttgattctttgggaccaatgcanctattgcaagttctcaatgatgtcctggctgagattgaccccaagcannatgttcatatcagagnggnnatgccagagcagacngccaaacgactgatgaatcttcttggtntccttncgnacaaacctccgggaaatgccacngacatgagcnccttccgtcangncttggtgattggaagtaaacctgtnattaacccagngctccactggcttcttcagcggagtantgaactaaagaaaagggnntatttagctngtttcttaataaaaccggaggtgccaagtgagtttctt

SEQ ID NO: 514 - DNA, 310 bp, Mus musculus
  misc_feature (1)...(310): n = A,T,C or G
gactgaggtacaccaggggttggaaaaccacactaggcaaagaacttgcatcaagatcaggcctgaagtacgttaacgtgggagatctagctcgagaagggcanttatacgacagctacgatgangantatngntgncccatnctaaanaaggatagggatgcacnatnagttngancatctcctgcaggatnggcgggattgnttgtngattacccacggagtganacttcttnnccnancgctgggnctatntantggtnagcagtcagtgancagatgatggtgtattgtataacacgacttgctac

SEQ ID NO: 515 - DNA, 353 bp, Mus musculus
  misc_feature (1)...(353): n = A,T,C or G
gactgagggaaagtcatggaggcttcaggatcttcttcccagtctcaagacagtggtggagtccacagggaaacggaagatcactgnnccgaccatggggancacggaanatnaagggaangcccntgagcnatatannanagacaagtcttcctcttcatcctcatnctcntcatcttcttcctcctcttcctcctcatcttcttcctcctcctcagacagcagcnatgaggaccagccctccnnaggtcccagganacacanaaggaggccccncagggacagcttgcgtggtgccgaccatggggaactggaagtgctaaaggacgagcttcagctctgcggaggtggtg

SEQ ID NO: 516 - DNA, 535 bp, Mus musculus
  misc_feature (1)...(535): n = A,T,C or G
actgaggatccttccttgatgacctcgagtctatggaggagtccatgtctcggagcctgggcaagcctgccnagtcctcgaagcagnacctgctggcagntcatcctcgnaatatgaggcgctggatcgggagcttccgtgtatcacnaaaattctcanaaccnccctctgcccancccctctgcctttgcatggagacttaggaagactttacacacgtggaggcgctgcaggctttggaggtcganctgnccggngncatggaaagcgggcgcgttaacagtattcgctacganaatatgaatgtcatctgnggnactgcgggccgnagggacaggnggctnatcaccgtcacagacttccagacgcggtcacgcctgctgcgctccgggctcaccctccgtggaaccgngtacccactggtgcgccacgacgatttactgctgggggactaccgtttgcacctgcgccgctctntggnncnaaaggcgcatgctcgaagccctcggggnggagcctgccgacgaagattgac

SEQ ID NO: 517 - DNA, 353 bp, Mus musculus
  misc_feature (1)...(353): n = A,T,C or G
gactgagactcccttaggtgctaatcatttaactgcaagcatgagtggattaagtctgcatccagagggactaagagttgtcaatcttcttcaagaaaggaacatgcttccttccacaccactgcagcctccagtcccaaatctgcttgaagacatccagaaactcaactgtaacccagagttgttccgatgcacgctgacgagtgtccctcagactcaggccttactgaataaagccaagcttcctctggggctgctgctccaccccttcaaggacctagtgcagtngcccgtggncacctccancacanttgggagangccgntnatgcaggacttacattaaccctttcg

SEQ ID NO: 518 - DNA, 348 bp, Mus musculus
  misc_feature (1)...(348): n = A,T,C or G
tctgagccttctggccggggctccccgggncctcgctgtgggtgatgactataccaaccggactggtgctgtgtacctgtgtcccctcacggcccacaaggacgactgtgaacggatggacatttcagagaaaagtgagacgagcctgagggcagagctaagtcgggagcctggcagagggggcccaaaggggagatacgaggttagcttgttagggaggcaacatttgaagctaaggggaaaagacagcgagatcaaggctagatctactatgggctacatagtgggttgcagaatagcttgagatgtaaaatggagatcctggctcaaaaagcaaaacaaaacaaa

SEQ ID NO: 519 - DNA, 365 bp, Mus musculus
  misc_feature (1)...(365): n = A,T,C or G
gactgagatttgccgagaggctgagttctctataggaacacccacatttgtatggatgagtggggaaagatagctgcagcagacaaggaaaaagcgtcctgacctcccgagaacatgagtatgctaaaacccagcggcctgaaggcccctaccaagatccttaagccgggaagcacagccttgaagacgcctgctgctgctgcagatccagnggagaagacaatacccagtgagaaagcctcgggccctccatcctctnagaangntgatgaaaatgataaattttnctctggagtagaanaaantgggagtgatgagccagccttcgtgtattacatgaatgatggtgtttatggttcttttgc

SEQ ID NO: 520 - DNA, 165 bp, Mus musculus
gaggacctgaaccaggccccccaaccccaagcagatgaggggcctccaggccgctccatcggggggatctcacccaacctgggtgccttggacttgctaggcccagccactggctgtctgctggacgatggactggagggcctgtttgaggacatcgatacctcc

SEQ ID NO: 521 - DNA, 230 bp, Mus musculus
  misc_feature (1)...(230): n = A,T,C or G
ctgggagtgtggcaggaagaagagctactgnatgcatggctacattagggnnngaaaatgagttgngtnaacactaagaccagggtgcctttcngaaggaagatcttnaactaaatatgaatgagggnnnnaantttttttgtaattataaatgggagaanaaaatttatgaaatgtntgtcatnagcacattgattataaatgtctttctgtnggaaaaaaaaccattg

SEQ ID NO: 522 - DNA, 218 bp, Mus musculus
  misc_feature (1)...(218): n = A,T,C or G
ggtgcaagcgtctcctgatgaaatgtattgtgccccactgggctccaaaggcagnatctggcgctgttgatgcccttgttggaactttccagagtcctaagccccccatgctgtaaatagtctggtcgtcagagtagggaggagcccgggcttctgtttatgtatgtatttatttatttattatttatttattacacctaataataaaaaggtgctca

SEQ ID NO: 523 - DNA, 427 bp, Mus musculus
  misc_feature (1)...(427): n = A,T,C or G
gactgagctcggattgggaaaatgaacggaggaagcaagatgaagggcagntntgtnccctgtgcatccgtcccctggcaggatcggancaggagatnantanncatgtgnagcactgcctggctaagagggaaggctcctgcatggctnaagatgatgctgtggacatngagcatgcggacagcanccgctttgaggaatatgagtggtgtgggcngaagcgnatacgggccnccacactcctggaaggtgnnttcccaggtataaagcnnntganaacttgggtggtggnactcnggnnaccaggccacttctgggancnggacannntttccaccacgngggagctcttgctcgtggggctcctttgngaccgngctgcccgcctgtgatgtgcatattaaagnggntgtatttgggtggggaa

SEQ ID NO: 524 - DNA, 452 bp, Mus musculus
  misc_feature (1)...(452): n = A,T,C or G
gaactgagggatagcgtggcccagtcgtcttctttgtggactatgcagtctaactgcttatatatggaagaaatcgaagactcggtgactgttcgggattatatccaatccactatggagactgaaanggacccccagngcctcttggacctggccaggaggatggngcnggttcttggccggaatgcacgancaagacctcattcacggggacctcaccacctccaacatgctcctgaggcggcccctggcgcagctgcacatcgtgctcatcgactttgggctgagctttgtctcaggactgccggaagataaaggcgtcgacctctatgtcctggagaaggccttcctcagcacgcacccccacaccgagaccgagtttgaagcctttctgaagagttacggggcctcgtccaagaagtccagtccagngctgaagaagttagatgagg

SEQ ID NO: 525 - DNA, 466 bp, Mus musculus
actgaggtcccttaggaggaaccccttgagctcccggtctaaaacacgttatcgccagtgtgagggctaaatggtgtgataacacccaagagccaggaaatggcaacagccttggagccagaggaccaagatctttgggaagaagaaggagttctgatggtgaaactggaggatgatttcacgtgcgggccagaatccgccttggagggcgatgatcctgtactggagacctctcaccagaacttccgacgctttcgataccaggaagcatcaagccctcgagaagctctcatcagacttcgagagctttgtcatcagtggctgaggccagagaggagaactaaggagcagattcttgagctgctcgtgttggagcagttcctcactgtcctgcctggagaactgcagagctgggtgcgaggccagcggcccgagagtggcgaggaggcagtgacgctggtggagg

SEQ ID NO: 526 - DNA, 162 bp, Mus musculus
  misc_feature (1)...(162): n = A,T,C or G
tgcaccatacatccgagagtttcgaggaccctccganatgctcactcctanancaagagcagaaacccgggaagaacgcatggagagaaagagacgggaaaaaannggcggcgacagcaggaagtggagacagagctgaaaangtgggaccctcacaatgat

SEQ ID NO: 527 - DNA, 380 bp, Mus musculus
  misc_feature (1)...(380): n = A,T,C or G
gactgagtttgtgaagtgtggatatgcaggctctaattttccagaacacatcttcccagctttggttggaagacctattatcagatcanccaccaaagtgggaaacattgaaatcaaggaccttatggttggtgatgaggcaagtganctgcgttccatgttggaagtgaactaccccatggagaatggcatctgtgcggaattgggatgacatgaagcacctgtgggactacacatttggaccagagaaactcaatatagacnccagaagctgnaagantttacttacagaacctcccatgaatccaaccaaaaacagagaaaagattgtnnaggaatggttgaaacttaccanntttttggggtgtttgaacccttca

SEQ ID NO: 528 - DNA, 314 bp, Mus musculus
  misc_feature (1)...(314): n = A,T,C or G
gctaaacctctaaatgtggncgcacatnctcaattaaanggttanngtttnttnnatanntgnccttgggcagttactatgagctacaggntctgcctcatctagtcctccgcatacagcggggagggtgagacttggggatgcgcccacctcaggcccatgcatcaccagatgggctctcacatgaccattacacacactgatggtgacactgtatcagaaagccactcagctttgccttttaactaaatctggacctgcttgccacaactatgtaacgnaagcctctaactactaatttctgaagagaagct

SEQ ID NO: 529 - DNA, 315 bp, Mus musculus
  misc_feature (1)...(315): n = A,T,C or G
actgagagaagaatggagggcagtggaaagctcgcatggtagaggatgctgtggagtaccatctcttcctgataccagataaagccagaggcacagaagagcacagagagattcttcagaagtacatttgaacggataatgacccagttngnacccatactgggancctttatatctggcagaatcagcctttcaatctcaaatacaagcctgcaaaagggaggtgttcctgctcatatgtacggnatgacaaagttttggagataacattgaggatgaatgggcntattgtttatgtaataaaggcagattaca

SEQ ID NO: 530 - DNA, 278 bp, Mus musculus
  misc_feature (1)...(278): n = A,T,C or G
gactgagatgctgtcacatctggagctgactcttcaggcatccaancaaaatggcacggagacccaggaggtgttcctggtcctcgtttcgaacaaaaatgtcttcgtgaagttccaggccccggaaatnccattgcacttggcctacgactccagcctggtcatcttncaaggacagccaanagtnaacatcacagtgctaccatcccttacctccaggaaacagatcctcgactgggcagccaccaagggcgccatcacctcgatagcagcactgg

SEQ ID NO: 531 - DNA, 409 bp, Mus musculus
  misc_feature (1)...(409): n = A,T,C or G
gactgaggtccaagcatggaaagtcataaaccangnactagcaaagatgatttaatacttaatatcatatccagaaaaattaaacaacttccagantcagacaggaatctgcttgaatatggatcancatatatnggacttaatgctgcttttgggggcctaataccanacagtctatttcggcgaatcttgaacgngacacaagctcgattagcttctagcttaccaatggctgtgatcccatttttgacagcaaacctgtcctaccaaagtcttgtaagtttacctttgagtacaggtgatttgaactgtgaaacctgtaccacaacacggggtgcactagttggtttggttatgggtggtctgtaccctatcctttnggctatacccgnnaatgggggccttgctg

SEQ ID NO: 532 - DNA, 489 bp, Mus musculus
  misc_feature (1)...(489): n = A,T,C or G
gaactgagtgcccaagatgtatgcagtgtgatgccaagtttgacttcatcaccagaaagcatcactgccgccgctgtgggaagtgcttctgcgacaggtgctgcagccagaaggtgccacttcggcgcatgtgcttcgtggacccagtgcgccagtgtgctgactgtgccctggtgtcccaccgagaggcggagttttatgacaagcagctgaaggtgctcctaagtggagctaccttccttgttacttttggggactcagagaagcccgagaccatggtttgtcgtctttccaacaaccagaggtgcttggttctggatggggacagtcatcgtgaaatcgagattgcacatgtctgtactgttcagattctcacagaaggcttcactcctggagcaggcancacccgagccantggcatgctcctgcagnacacagtgccaggggcagaggctgcagcacagctgangctgatggcccggagaggac

SEQ ID NO: 533 - DNA, 292 bp, Mus musculus
  misc_feature (1)...(292): n = A,T,C or G
gactgaggctgactcatgccgatctggctgaagcctggctgntcctgctaggtcgtcgtgccactactactgctatcccgacgctactgaactggactgctagtatattcatgaagtgttttcaagtggatccagctgccgntactgaccagtgaactgaactgctgatttcctgacaacacagatgagatttgctccaaagaaccatttctaaacagggccacnncctccatatcctaatagcctttcttttctacctttggngggtggtgctctataagggagggtaaac

SEQ ID NO: 534 - DNA, 147 bp, Mus musculus
  misc_feature (1)...(147): n = A,T,C or G
gggagaccttctggtagttctgttctgggncccttanccagagttccaccagccacaccggtggntcgaatgtntgagcagagnaacgtgaactccgagcctgaggaggaggaaggaggcctggaagatgaagatggagatgatgat

SEQ ID NO: 535 - DNA, 487 bp, Mus musculus
gaactgagagggttccccttgcaagcatatccaggcggcccttgtcccaccattgcataatataagctacatcgtggatatgatctaggtcctcggagtgtctgttctcatgctctggaccagactgagaatagaaaggagcctggctatctgagagatcctgtcctgcaactggctgttctggggctagaggatgtggacgtgaccatggactacactggtatatatttggtagaggacagctctgctgaaaaatattctactgtgcacaagaactctcaaaagaaagttatctggggctggaacattggcagctaagagcactgactgttcttcggaggtcctgagttcaattcccagcaaccacatggtggttcacaaccatccgtaatgggatctgatgccctcttctggtgtgtctgaagacagcaacagtgcactcacatgtatatgtaaaataaataaatacatctttaaaacaaacaaa

SEQ ID NO: 536 - DNA, 132 bp, Mus musculus
gactgaggtgtgcgccttgaccgagttcgaggaggccgccagaagtacaagcgacggctggattcggagaacagcccctacctgaacctgccgatttccccacctgctaaaaagccattgaaaataaaaaaa

SEQ ID NO: 537 - DNA, 486 bp, Mus musculus
  misc_feature (1)...(486): n = A,T,C or G
gactgagcctagtcattgttggctacgagcaatggtaatacaattatcactcagcttgactactcttctggacaagttctcaaatatttctgaaggcttgagtaattactccatcatagacaaacttgggaaaatagtggatgacctcgtgttatgcatggaagaaaacgcaccgaagaatataaaagaatctccgaagaggccagaaactagatcctttactcctgaagaattctttagtattttcaatagatccattgatgcctttaaggactttatggtggcatctgacactagtgactgtgtgctctcttcaacattaggtcccgagaaagggaaggcantcaaggnntcnntggantactgnnnacagtgagatggaacnntnntanncttnccctgctggatgancngaatgtgtngctanngaaagaantngngggactttntgctacaaaggtctncgcnggctcgggactgtcctta

SEQ ID NO: 538 - DNA, 174 bp, Mus musculus
  misc_feature (1)...(174): n = A,T,C or G
gactccacaggaggcctacanacagngggctantccagccacgctctangaaccatnacaaaccnttnctctttataagaaattggctcangcattaccgatgaaaagctgtgtaacacaggctttgaggcataaaaccgatttctattaaatgtagaaacaaaatgaaaaaaa

SEQ ID NO: 539 - DNA, 339 bp, Mus musculus
  misc_feature (1)...(339): n = A,T,C or G
gactgaggtgacagtacaganaagtaccacaaacgttatccaggcanctgactgacatggtgagacagcttgtagacgatgtcaagagccttnaaacgcanagacccaacttgngatctggacaggacaatgcacaaggcaccccacccacggctggnnagatggctcagcgattannancactaactgctnttctgaaggtcctgagttcaaatcccaacaaccacatggtagttcacaaccattcctaatganatcagatgcactcttctggtgtgtctgaagacagctacaatgtattcacatataataaataaataaatctttaaaaaaataaaa

SEQ ID NO: 540 - DNA, 531 bp, Mus musculus
gactgaggttttaatgtggaaacagtagaatataagaacatttgtttcacagtatgggatgttggtggtcaagataaaattaggcctctctggaggcattacttccagaatacccagggtctcatttttgtggtagatagcaatgatcgtgaaagaatccaggaaggagcagctgtgctgcagaaaatgcttctggaagatgagctgcaggatgcagtgctgctcctttttgcaaacaaacaggatctgccaaacgctatggccatcagtgagatgacagacaagctaggtctgcagtctctccgaaacagaacatggtatgtccaagccacttgtgctacacaaggaactggtctgtatgagggactggattggctgtcaaatgaactttcaaaacgttaaatgaagctggatatctaaccaaggacatgtttgatagaattggtctaggcttgttacaacaaaattagtttgcatcttggttattaaaggatatctgagacaggtttgggcagaaattacagcgtttaa

SEQ ID NO: 541 - DNA, 158 bp, Mus musculus
gactgagttatatccgactttatgggagaaaatttagcaaagaagatcatgttctttttattaagttactgtatgagctggtatcaattccaaaactagaaatcagcatgatgcagggatttgcccgtctcttgatcaacttgttaaaaaaaaaggaa

SEQ ID NO: 542 - DNA, 398 bp, Mus musculus
  misc_feature (1)...(398): n = A,T,C or G
cctcctgggctggggttcgctgctcatcatgctcaagtccgagggcttttactcctacctgtgtacgaagccaggtganatgggcgcctnnntcgggngcaggtttctggagtggntctttgggagagggctgctnggtggtgaanacgtaanccagntatgcctgcttgctgaacagtgcctctangtgccnnanngnccattntgatcctnanccngctatctgtgtgcntnnnatnatcatngtccctgtttggggntggngaaactgaggctgggaccantcttccnttntgcccangctgtgagactaatagactaatagactcctgcgtgcngncangaacgttgntggccttntgntcttccagggcctgcancattgcttgccttgcaaa

SEQ ID NO: 543 - DNA, 228 bp, Mus musculus
  misc_feature (1)...(228): n = A,T,C or G
ctcacagccacccatagaccanttncaatgagaggcatctgacccagcaggaaacgaggcctgctnaacagagccttggatatggacgatanggattacagtgaggcccgatggcctgtcngataagnaccacccccagtaaggcccagnaagtcncctcnagaagaattgccaangaangtttcaannagtagccntatgccngngtcacccttgcttcgaaggctt

SEQ ID NO: 544 - DNA, 253 bp, Mus musculus
  misc_feature (1)...(253): n = A,T,C or G
gactgagattttcagaatgatcaactagntgaaatatctccattgggtactgaggaagtctcagcagttagccaagcgtggtttacaactaaagaagataaggattctctcactaacaaaggacataaatggaagcaggggatgtggtccaaggaagaaattgatattttaatgaacaacatcgagcgctatctgaaggctcgcggaataaaagatgctacagaaatcatctttgagatgtcaaaaaaaaaaa

SEQ ID NO: 545 - DNA, 371 bp, Mus musculus
  misc_feature (1)...(371): n = A,T,C or G
gactgagactaaaatgtcaaaaagaaaatggctctgttagcccaacagttacccagacggttcaactcagttaagttgactagcttcattaaagccaaacaagtcaccaaacgctctgcaagagcaggaaaagccgtgttgcccggcttctcagctcagcctctgctctcctctgacacgagctttctccggcggggaattaagacttacaggactttgttctggaatcgtttccattctgccagcacaaataggacaaagagttctgcccgaaagcactctgcttccttcagnggntgaacagcagggagaggatncttagtcttgaatcanagcttgccttttggaagaagnggacgacctgcctccat

SEQ ID NO: 546 - DNA, 434 bp, Mus musculus
  misc_feature (1)...(434): n = A,T,C or G
ctgaggcacaacccggtctcccagaanaggagaagtgcctggcgtggattacagctttctgactgtgaaggagttcttggacctcgagcagagcgggaccctgttggaagtcggcacctatgaaggaaactattatgggacacccaaacctcccagccagccagtcagtgggaaagtgatcacgacggatgccttgcacagcctgcagtctggctccaaacagtcgacccctaagcgaacaaagtcctacaatgatatgcaaaatgctggcatagtccacccggagaatgaggaggaggaggatgtccctgaaatgaacagtannntttacagccgactntggagaccaggacgagccacntctccaagaagcaccgcntcccacctgtgaatagtagcatcctcgcttgntcccatcacgggaccctttttaa

SEQ ID NO: 547 - DNA, 221 bp, Mus musculus
  misc_feature (1)...(221): n = A,T,C or G
atggatactacaatggtgaatttgtggactctgtttgagcagcttgtgcgccggatggagattatcaatgaaggaaatgaaagcattgaattcatccaggttgngaaggacttcgaggacttccnaaagaagtatcaaangacccaaccaggagctggagaaattnaaagacctattgttgaangcagagactgngctggaaccgccctggacgttaaagc

SEQ ID NO: 548 - DNA, 337 bp, Mus musculus
  misc_feature (1)...(337): n = A,T,C or G
gatgacgtccatatcttntcgnaagagagggaatcagaaggtacttnngggagaatagtgatgcagctttncaccntcacngcaatgagaggattctgaggccttctgagnggaatcagagataccttgccaagtaatatgtaccagaaaaatggcttacatcatgggaaatacgcagtgaagaaatcacggnnaactgatgtggaagaccttactccaaaccctaaaaaactacttcagattggtaatgagctgcgcaagctgaataaggtgatcagtgacctgactccagttagtgagcttcccttaacagcaaggccaaggtcaaaaaaaaaaa

SEQ ID NO: 549 - DNA, 173 bp, Mus musculus
gactgagagctctgcacatcgagagacttaaaaccacctgagatcatcagacaagtcacatacatgggactttttgaattattgccattttgtatttttatcatgttatgtgtaaaagacagtgcctattctgtgttttactgaatcaaattgacaatgtcagaaaaaaaaaa

SEQ ID NO: 550 - DNA, 164 bp, Mus musculus
  misc_feature (1)...(164): n = A,T,C or G
atcttcctgctccagcctcttgaatggaatgctgtacatagactccagaccagctcagctacttcctcctatgctcttgacacaaatgaacactgaccacaaatttctatttgtacaatctgtggatgtnccaaaataaaggaattggagctgaaaaaaaaaaa

SEQ ID NO: 551 - DNA, 105 bp, Mus musculus
ttttttccactattattgcatgcagattaaaacgttaagagcttttctttccccttaaaaattctgaattatgaaaataaacatttcaaatttcaaaaaaaaaaa

SEQ ID NO: 552 - DNA, 203 bp, Mus musculus
  misc_feature (1)...(203): n = A,T,C or G
cacagactgagttttaaggacttcctgagctgttntgagttgttnacttatggatcttcactgcaagatggactgttccaatctcagaggaactcttccagaaacctgtatagactactttggccttgaactcctacaggtggncctgcctttgcttcctagnggctgggattaaaggtgtgtatcaccaaagagaaaaaaca

SEQ ID NO: 553 - DNA, 407 bp, Mus musculus
  misc_feature (1)...(407): n = A,T,C or G
atactgattctctgatttcccagtaatgccactcagttgccaatatcaagcaagcgcatataagacacttgtaatcttttaagaaaagtagtacttctcttcacagtatctggcggatcaacactggagggtgaggtgtcagcttccagaaagatcatcatgaagttatatgtgtttctggttaacaccggaaccacgctgacatttgacactgagctaactgtgcaaactgtggctgatnttaagcatgccattcaaagcaaatacaagattgctattcagcaccaggttctggtggtcaatggaggagaatgcatggctgcagatcgaagagtgtgtacttacagcgctgggacggacacaaatccaatttttctttttaataacagaaatgatcttattgaaag

SEQ ID NO: 554 - DNA, 179 bp, Mus musculus
  misc_feature (1)...(179): n = A,T,C or G
gactgagataaaatcaaagacaaaattaaagagaaagacaaagagagaagaagaaaaagaagcatggnngtnatgannngagatcaagaaagaaaatggagatgtaaagattttactgaaaagtgggaaggagaaaccaaaaacaaatatagaagacttacaaattaaaaaagtaaaaa

SEQ ID NO: 555 - DNA, 219 bp, Mus musculus
  misc_feature (1)...(219): n = A,T,C or G
cgttgaaacatgcccgtccttcaccanactgagcttccacagcgcggtggttggtacaggactctccgtgagactgatgctctatacacangagaaacngctgtcttcanancaaacacaaacaaanganagttttctttccttcctattggcgcaatccatacttattncaggaaattccttaagacgtntagatgtaaaatacaaaactgtgcacaa

SEQ ID NO: 556 - DNA, 750 bp, Mus musculus
  misc_feature (1)...(750): n = A,T,C or G
gcttacaggtggtgcagaaagaagtctggtcacaactggctacagtgaacgagctgggtaccccaaggacatcttaccagttccagccaaagatctgatctacgtacacctgcgtcatgctgagaccctcaagcctcactaaaagggtccctgcctagttctgtttactaatctgccttattctgttgttgttcccatgttaaagatagagtaaatgcagtattctccacatagagatatagacttctgaaattctaagattagaattacttacaagaagaagtggggaatgaagaatgaaaaattactggcctcttgtgagaacatgaactttcacctcggagcccaccccctcccatctggaaaacatacttgagaaaaaacattttctggaacaaccacagaatgtttcaacaggccagatgtattgccaaacacaggatatgactctttggttgagtaaatttgtggttgttaaacttnccctattcccttcccattcccccttccagtttgngggntttttcctttaaaaagcttggggaaaaatttttaagtccgtcgtcgagactcctctaccctgtgcttaaaggggtattgaagtttcgacccccanagctnttggnctgnggnggctttcatggttggctggntttattttcaaacccccaanaacttttgggccnnggggggcttttaatgnttgcctanctttaattaaatcttaccctttttacaatttttaaaaaa

SEQ ID NO: 557 - DNA, 218 bp, Mus musculus
agtcatgcaggaaaacctcagcatttgcttcatcaggagatgacgttaaaatatgggatgcttcgttcctgacattggtggataaattcaaccctcacacgtcaccgcatggaatcagttcaatctgttggagcagcaatagtatcctttaaaaaaaagtaatcactttttgttagttttcaatgatctgtgcaactgttggcccataaaaaaaaata

SEQ ID NO: 558 - DNA, 223 bp, Mus musculus
  misc_feature (1)...(223): n = A,T,C or G
gactgagggtcccttacantcccttaaaaatccattctcccttccttgctccttgcagcagtaacatgtatgagtaccatgcaccaactgatcgtgccattggagctccttaaacagtaatcacaaccaagctaggggaacatttgtatccgataaaggggtgggagtgcaggttttaggcactcctttgggagaacaaagaaattaatgtaaataaaaaaaa

SEQ ID NO: 559 - DNA, 222 bp, Mus musculus
  misc_feature (1)...(222): n = A,T,C or G
aattgtctcttgagttccactgggaatgcccagtctggggctgtgagaaacactgtatcaaagagatcccggctcagcacctgagacacaggcatgaggcctgcaatgggacagagcaaccggaactcctaaagactgtcttccaaagttgtgctgggccttttgnggggaggaacatgactgtgtcatggggaaaatgcatacatctgtttttacttaaca

SEQ ID NO: 560 - DNA, 380 bp, Mus musculus
  misc_feature (1)...(380): n = A,T,C or G
actgaggtagcccagagctcgagagctggcttcctgaccatcagacaagcttcgccaaaagtcttcacctaagcaaacaggagaaagaagggnttctntgtgnnnccccgnctgtnctggntctgactctgtacgaccgncacaccnagctggccttgaacttaanaganctgcctgcctctgtnctgactgctggnatggaagggatgtgccactactgagagcttgttttagtgcttacancttgctntgctgctaagccagatgccctgagtgtgatnctctagaatgacaaggtgngagagaaccaattccccannttatcctcacacccacacatgagntgcgncaagtgctcccacctctgacaataaaaaaaa

SEQ ID NO: 561 - DNA, 149 bp, Mus musculus
  misc_feature (1)...(149): n = A,T,C or G
aggactgaggtaaaatcaaagacaaaattaaagcagnanagcacaaanagtagaagaagaaaaagatgcataangtaatgantngagatcaagaaagaaaatggagatgtaaagattttactgaaaagtgggaaggagaaaccaaaaac

SEQ ID NO: 562 - DNA, 126 bp, Mus musculus
  misc_feature (1)...(126): n = A,T,C or G
gtctcgggctggtgagtanggctcagtgggtaagagcanncgactgctnttccgaaggtncgaagttcanntcccagcaancacatggctggcgtcacaaccntccgtnncgagatctgacgccct

SEQ ID NO: 563 - DNA, 134 bp, Mus musculus
atggattttcctgtaaactgcaagataaccgggacaccagtctcactcaatcctttttgccgggaaccccttcctccctattaatgcatgataatgaataataataataagaatttcatctctaaaaaaaaaaa

SEQ ID NO: 564 - DNA, 424 bp, Mus musculus
  misc_feature (1)...(424): n = A,T,C or G
aactgagcctatcctctactcctatttccgaagctcctgttcatggagagttcgaattgctctggcgttaaaaggcattgactatgagatagtgcccatcancctcataaaggatggcgggcaacagttcactgaggaatttcagaccctgaaccccatgaagcaagtgccagcgctgaaaattgatggaatcaccattgtccagtcactgancatagnnctngaccgngganaaaactctgactnngngncccgttnctnccttagnnacctaacntacgagcnatcnngcgcatganngntnanctcntnnctggtgganngcagncccttngaaacctgtctgtcctgaagcaagtggcacaggagaaccagatgcaatgggcccaaanggtattacttccggntttaacgctctggaaaa

SEQ ID NO: 565 - DNA, 341 bp, Mus musculus
  misc_feature (1)...(341): n = A,T,C or G
tttcagctcagttacttatggcttcccctaaaattgcttccctctcttggggacaaatgaaagtgcaaggctctacnttaacctataaggattgcaaagtatggccaggaggcagtcgggcttgggactggagggaaacaggaaccgagcattctcctggagagcagccagcagatgnaaaggaagttgctgagaaaggggtacagactcttgtgattggcagaggaatgagngaggccttgaaggtgcctccgtcgattgtggagtacctgganaaacaaggcattgatgtgcgcngtcctccaaacagaagcaggccgngaangagtataatgccttgg

SEQ ID NO: 566 - DNA, 439 bp, Mus musculus
  misc_feature (1)...(439): n = A,T,C or G
caccacagacttccaggagagctttgtcacgtcaggtgtgttcagtgtcacggagctgatccaagtgtctcggacacctgtggtgaccggaaccggacccaacttctcactggggagagctgcagggtcacctggcatatgacctgaacccggccagcgccggcatgagaaggactctacccagcacctcctccagtgggagcaagcggcacaagtcgggctcgatggaagaggacgtggacacgagccccggcggggactactacacgtcggccaactcacccacgagtagcagccgcaactggacggaagacatagaaggaggcatctcatccccgggtgaanaagacagagatggacaaantctccattnnacagcccgtccccccaggactcccccnggttntccagcttnacacaacaccacaggccggtcatt

SEQ ID NO: 567 - DNA, 469 bp, Mus musculus
  misc_feature (1)...(469): n = A,T,C or G
actgagcctcctggtgcaagctatatttaatggtgatccagatgaagtccgagcactaatatttaagaaagaagatgttanctttcaggacaatgagaagagaacccctttgcatgctgctgcctatcttggtgacgcagaaatcattgagctgcttattttatctaggagctagagttaatgccaaagacagcaaatgggttgacacctttacacagagcagtttncatcgtgtagtgaggaagcagttcagatacttttgaagcattctgcagatgttaatgctcgagataaaaattggcagacccctttacacatagctgctgctaataaagctgtaaagcgccgctgaatctttggtacctctttctgagcaatgtggaacgtgtcccgaccgagcanggaagaactgnattacatcatgnagcttttcagtgggcatgggcganatgggcaaactactcttgtc

SEQ ID NO: 568 - DNA, 385 bp, Mus musculus
  misc_feature (1)...(385): n = A,T,C or G
gtcttctagtaagaacaaagnctgangacntgatctctcagctgcacnctaacctcctgggctccatgctgacctgtaaagctgccatgaagacantgattcagcagggagggtctattgttaatgggggaagtattatnggtnagaaaggcacccgnnggccaactctgcatacagtgccaccaaaggaggactcgttggnttntcacgctcggttgctaaagaggttgcncggaagaaaatgccngtgantgtgntgncaccaggatttattcgcacggatatgacnntgacactggaaggaagancactncnnganaaacattcctcttgggaggttnngagaaactcntgagggancacatgccgttgcgtttttttttag

SEQ ID NO: 569 - DNA, 303 bp, Mus musculus
  misc_feature (1)...(303): n = A,T,C or G
gactgaggtggggtctttcncccaagaggaggaagaagagaacctacggcatgacaagcccccctacacctacttggccatgatcgccttggtaattcaggccgcacccttccgcaggctgaaactggctcagattatccgtcaggtccaggcagtgttccccttcttcagggacgactatgagggctggaaggactccatccgccacaacctttcctntaatnggtgcttncataaggggcccaaggaccctgcaaagccccaggccaagggcaactttttggncgggggatgggagcctga

SEQ ID NO: 570 - DNA, 532 bp, Mus musculus
  misc_feature (1)...(532): n = A,T,C or G
gactgagatggtggctcgctgatttctccgatccctaatccgggtcacagaatggttaaagcagctttaagtanagagtggattattttcccctcagtttgtcttctgtttgcggctctgaaagaatgttgtgggcgctctttttcctggtgactactattcacgctgaactctgccatccagatgcagaaaatgcctttaaagtaagacttagcatcagagcagctcttggagataaagcatatgtctgggacacggatcaagaatacctcttcagagcaatggnggcattctccatgagaaaagttcccaacagagaagcaacagaaatttcccatgtcctgctttgcaacataacccagagggtgtcgttctggtttgtggtcacggacccttccaacaattatactcttcctgcagctgaagtacagncggccataagaaagaataggaaccggatcaacagngcattttttctggangaccntactctggaatttttaaaaannccttntactcttgctccccccga

SEQ ID NO: 571 - DNA, 256 bp, Mus musculus
  misc_feature (1)...(256): n = A,T,C or G
tatttacttcttgtggacctctcacagatcangcngnnaaggngnatagngccttgnttgcccaaggggtnatggaaggaggtgtcntccattccacctgctganggcgcctacagaggagaataattcagaccncncagncnngggtgactaccagtccttggcctcagttcctgcctccagatttctgccttgagggggctaccctgacttccctggatgatgaactacaagntgtaagataaaataaaccctt

SEQ ID NO: 572 - DNA, 240 bp, Mus musculus
  misc_feature (1)...(240): n = A,T,C or G
actgaggtctcctgctcaccagntgncttgacagtttttcttgcaacattggccattggttttcactgccttcaaaagatcanaattactccagaaattggagagttggatttaaaagaaaaaacttgaacaaatggacaatatgtctataacaaatacaccancaagtaacgatgcctgtctgagcattgtacatagtttgatgtgccatangacaaggtggggaaagtgaaacctttg

SEQ ID NO: 573 - DNA, 350 bp, Mus musculus
  misc_feature (1)...(350): n = A,T,C or G
aatgggatacgacacgggncttacacagcctctcggaaacatatctatattgatanaaatacngaagattatnttgcncagggtttcacaaggcaaacagggtacctttcacagccagnanggcttttggagtncggcaccaaactcgtggnaggaaccacttccnggggaaggggtggccaagaagcacctgggcttgcccgntctttaataccggtgaagggaggcccaaaagangaaaacagggagcnaacgggtttctggcattnatggcccctcctccttttgctggnngctgncattaantgaancannccgcnncggaanattcccttagggttggggggcat

SEQ ID NO: 574 - DNA, 523 bp, Mus musculus
  misc_feature (1)...(523): n = A,T,C or G
catgaacgtgctggagccagtgtggttcaactcacctggaatctgagcactcaggagtctgaggcaagattgccaagagttcaaggccaccctcagctacatgggtctcaaatactcaagagttcactctgcggctgaggatgacctcaagcctctgatcttcctgctgggatcactgagagctgggattacagcattggacgcgttctcacgaccggccaggaaagacgcaacaaaccagaatcttctgcggcaaagctttattgcttacatcttcaggagccagagtgcaagaagcaagagagagagaaaacgaaaccccgtcccttttttaggagagttatatttcgcctaggacgtgtcactccctgattggctgcagcccatcggcccgagttgacgtcaccggggaaggcagagcacatggagtgganaaccaccctcggcatatgcgcagattatttgtttaccacttanaacacaagctgncagcgccattcttgtaacggcgaatgtggggcgc

SEQ ID NO: 575 - DNA, 274 bp, Mus musculus
  misc_feature (1)...(274): n = A,T,C or G
gaactgaggctttatggaaggcagtggtcggancagggcagaatcctaccctgacagcagcacactggtcattgccgtggcangngaggagacgactctggtgtgtacaacataaatcttgaaaaatggaggctggagaagcacattccaggcatcanagattaaggttgtggattatccctggtccctccagnggcanccaaangtgacanaangtggaagantgactgggngcatcntgaanctgggaagccgtgcggggcctatgatggga

SEQ ID NO: 576 - DNA, 420 bp, Mus musculus
  misc_feature (1)...(420): n = A,T,C or G
tatttgtataactttgcnagtgctgcaacanagaagataactgaatctgttacagaaacagctcagacaatcaagaaatccgtggaagaaggaaaaatagatgatatccttgacaagactattttaggagattttcaaaaagaacagaaaaaatttgttgaggaacagaatacaaaaaagtcagaagcagctgtgcctccgtgggttgagagtcatgatgaagagacaattcancagcagattttggctttatcagctnaaaaatnngtgagtcgggctggcgaganggctcagnaggtangagctntgactgctcttccanaggtcctgngtncanancccannntccacatggtggctcacanccacccatantaggatctgactccctcttctagagagtctgaagacagctacttg

SEQ ID NO: 577 - DNA, 492 bp, Mus musculus
  misc_feature (1)...(492): n = A,T,C or G
cactatccgatgaagatatgaccatggtgctgaagaagacagcgacnnggangatcaggacctggataaaatgtttgnggcctggcttggagaactggacaggcttactcagagttgngatnctgacaagcccaaggaacacantaataangatctnctcttnnccnggaannaantatnngcaacttttnttnccgcttntccatatacaacttgnatgaanctntgaatnatngngagactgtgnacctggatnccctgatggctgacctctgntctatagagcaggagctcagcagcattgnctcaggaaatagtaagcntnacgtcacagaaccntaaagttcctcagaagctacctgctgnccgacatacgtcacaacatggcaccctgagaggaccgtcatcttcctctaatagaacaactaaaccttcccatgctaactactccctggatgacatcactgctcagttagaacaggcttcctcctg

SEQ ID NO: 578 - DNA, 165 bp, Mus musculus
  misc_feature (1)...(165): n = A,T,C or G
agaagtctgtggaatgttgagcatctgaggggnaacatatggnggcaccatcccaggcattccaaatggatttggtcgggctgttntatcaatacnngaaganaataatatggacaactggtnaagagaatgcaatanncncagtncagccccagtagcccaggg

SEQ ID NO: 579 - DNA, 357 bp, Mus musculus
  misc_feature (1)...(357): n = A,T,C or G
aggctccctgctgccccatcctggccacaactttgtgcggcaccatcttagaaaccgtcccgacctatatggccccttctggatctgtgctaccctggcctttgtcctggcagttactggcaatctcactttggtgcttgcacagagacgggacccctccattcactacagcccccagttccacaaggtgaccatagcaggcatcaccatctactgctatgcgtggctagtgccgctggcgctgtggggcttcctgcggtggcgtcagggcacgagggagcgcatggggctgncaccttcctgganacagtctggngtctacgggctactcactctttggnttcatccctactgggg

SEQ ID NO: 580 - DNA, 437 bp, Mus musculus
  misc_feature (1)...(437): n = A,T,C or G
gactgagatgagcagtttgcagatcaaggtaagacaaagtcaaagcagacaccaatggtgttatcaaaaccagcaccacttgcagnaaaagacaggaggaagaggaganagagggacagagcttgctcantctttacttcaacnnagctgatcanaagcaatcttggnngnataacacccanccaagtcgggaaggtccttgcaaagggancccaagccttccccngctctacttcangtngaagnncctttngaangagcnttcgccttgaaaacctnnagcttctccnanggangtntacngccnaggggcttcaatcggaccgtcccaggatcnanggaagaangctnttgcccatagangnttgctggacccccncagnaaactngattnggcaagtcncagntttggtncttgggaagaacagctggccttt

SEQ ID NO: 581 - DNA, 345 bp, Mus musculus
  misc_feature (1)...(345): n = A,T,C or G
agactgaggtcaagtggactccagctaantactgtcaatcaacagcctgtttcccttccacctgcctattcctgagggtgaggtcttttccctggtcccaggacttccccacccctcctccaaataccaagactatgggcttaaaagtttcaaatgggtgctggagagatggctcagcagttaagagcaccgactgctctttcgaaggtcctgagttcaaatcccagcaaccatatggtggctcacaaccatctgtaatggaatctgatgccctcttctggggtgtctgaagacagctacagtgtactcacatatattaaataaataattctttaaaaaaaaaaa

SEQ ID NO: 582 - DNA, 397 bp, Mus musculus
  misc_feature (1)...(397): n = A,T,C or G
ccactgagctgttgaataagtgctncagctncgncttgggagtttgncttgccagacngctctgctnttgcacctgaacagcccangggccanngtggaccctgnnacantcatngagaggggcaggtttatgctaancctatgtntacaacagtnttttggggggtgtagntccaccacntgccacattcacttgnnatganggcnctgtagncaagctagcacccagagccccttncaaggctacacaattacaggttcctcggccactctggaactgaaggggcaagcctgctccnangnnaccctanccnggngtgctggccgacagnctaggcccacatctctgntttgctggcttccnttgtactctcaacccctgccttgttctgggatc

SEQ ID NO: 583 - DNA, 122 bp, Mus musculus
  misc_feature (1)...(122): n = A,T,C or G
catgatcccacctccccccagtctcccgggccctcctcggnctggcatgatgcctgccccccacatgggaggccctcccatgatgccaatgatgggcccccctccgcccggaatgatgcccg

SEQ ID NO: 584 - DNA, 254 bp, Mus musculus
  misc_feature (1)...(254): n = A,T,C or G
actgagggttccaagnctgccctttggtattcaaacgggagccgctttggactgactggctgccttcctcctcctccccctcctcccccttctgcttcagatttatttattatatgtaagtacactgtagcagttgtaggacacactagacgagggcaccagatctcattgtgggtggttgngaaccacccaccatgtggttgctgggantngancnnaggatcttcaaaaaagcagncggggggtctaacccg

SEQ ID NO: 585 - DNA, 274 bp, Mus musculus
  misc_feature (1)...(274): n = A,T,C or G
gactgaggtactacataaatagccaagnntattccatttttagtaccaggagttctattaaattagagtatgaaggaaactcatttgtatcatggaaaataccagaaagctgtaaagttgaaaacactacatcacccannacaacactacnctgcntnagggctgganttcacacantnaanccaatngctggannccaanaagtggaacggcctctgactgtggntaattcttatatntgctacttgcgggatttcacagacgggtaagtgga

SEQ ID NO: 586 - DNA, 467 bp, Mus musculus
  misc_feature (1)...(467): n = A,T,C or G
gacctgaggccaggccatctgccttcacccgctctatgtcatgcttccctgcaccctggctgcctccctagctttcatgctacccgtggccactccacccaacgccattgtcttctcttttggaggcctcaaagtgtctgatatggcccgtgcaggattcctgctcaatatcatcggagtgctgactatcacattatccataaacagctggagtatccctatcttcaagctggacacatttcccacctgggcctactccaacacaagccagtgccttctcaatccgcctaattccactgtaccaggccactagactatggtacaacctagctctgctaggggaaaagccgctggtgccactcctcggagccaggagagggcgctccaaactccaagtcccgggacagaggtgcagaatcggntgggcgcatgcgaaatcatgtattcgngtgtttgcctacatcctg

SEQ ID NO: 587 - DNA, 477 bp, Mus musculus
  misc_feature (1)...(477): n = A,T,C or G
ggggaaacgccgacctgantacnntccctaagcccaacagacactcacactggttcgtcatgaagtcggagaaattccatgatgagctctccgaccacgtattctttacgtntctgatccagaactgaatggtaattctccagaacagccttntggtactcctcactgaacatgcgaggcggggtcctgggattcaacgcaaaaggagtatcagagccgggcctgacgtgagatccactcgaccaccactaaaagatctgtcctgatgcggggctgtactaactgcaggactcaactgatgctggctcagagaccctgctgtntgatgtcagctagcattagagagactttaaaggacacgcctggcagggggggctggcgtatgccnttagtcacagccctggannaggcnggaggagctcttgagtttgaggccagcctggnctacagaacgagttntgggacagcctgggctgg

SEQ ID NO: 588 - DNA, 477 bp, Mus musculus
  misc_feature (1)...(477): n = A,T,C or G
gactgaggtgtgcatgcctgcgtttccatgaatgacgcataccaggaggacagaagactgatacaccaggctggagttacagacagttgtnctcnngttgactnaggaggnngcannctncttngnctgctgngccatcattgctggatgcctttanntactgaganangnttctactgtgnntccnnggngtttnntanntcnacctcaantaacatgnggttggnngctaacnntttatgtntttnagaagtgactttnancttctggncctcctatctnctcctcttcctcctcttcctcttcttcatcctcatcctccccttcctctncctnctcctntncctcctcctnctgagagctgggaccacaggtatgtaccacacgcccagcttatacagcacactctgcagactgagctcaagtgcggtttcatgggtgtggatggcaaaaggtgtttaagctaaaaggaattca

SEQ ID NO: 589 - DNA, 305 bp, Mus musculus
  misc_feature (1)...(305): n = A,T,C or G
actgagaacacccatcatggtagttggaagaaggatcgtcggctcacccctcctcnccttcgttcaggcacacctgccagaggcaagacctcngtggntnggatgaagtcntcttctnctatgtgcttgggggtcctggaaggacctggcccccttcaagggaccatnagagaagaannnttgttntgggaggcctttactagaaatgaagggangcttatgngcctggcttttnncnacatcccnagaggtatgnggngggaaantntgntgcnanaatcttnttgggggcagttgaaccgatg

SEQ ID NO: 590 - DNA, 358 bp, Mus musculus
  misc_feature (1)...(358): n = A,T,C or G
ctgccatcatgctgtnacattcaccatagatagaanacttncntntntaggggaganacngacaagacatcntatcgacagcaactataaaagtgcatatccncannnnngatgancctcctttnatcgaccagctgggctttggngnggccccaggcttccagacgtttgtgtcttgccaggagcagaggctcatctacctgccctccccctggggcacctgcaatgctgttaccatggactcggatttcttcgactcctacagcatcacggcctgccggattgattgngaaacgcgttacctggtggaaaactgnaactgccgtntggtgcacatgccaggggatgccccatactg

SEQ ID NO: 591 - DNA, 517 bp, Mus musculus
  misc_feature (1)...(517): n = A,T,C or G
tgttacctttagtgaacaagagcatgttcccaaactcaattttgggtcgtccacccttcactncaacccaccaacagcataataacttctttgctctctcaccaactctttattcacaccagcaacttatagatgcacaattcaactttcaaaatgtagacttgtctagagctgtgtccttacagcctctgacatatggaactgtcagtccaatacagaccnggancatctccatttatttcgaggaaggaagagaataagcgatgaaaaaagcttttcctcttgatggnaaacggcagcgtttccatttcaccccaccaagagccaactataatncaaccaggctagngcctttatcaagggtgacagaaggatatttccatgcccaccggntgtttcacacgcactatataccagaatatagtncagatggtggtcccaccctnntngnagaaattnccacctgnanagaaaccctccgagaaaatnacaactngccctggagggccaaagataa

SEQ ID NO: 592 - DNA, 307 bp, Mus musculus
  misc_feature (1)...(307): n = A,T,C or G
cacatttttggtcacngacagcgatatctnaataggaggaactgggcaggagcagggcattctctccagcaggngagtggtcacgactactacaactccaacccgaaggccatcactacgggcttaaatggccgattcggctaccgccggaacactccagccctccgccagcncacatcggtgtttggagaagncacaccatttcccattttctgaagacattgcgtcacaaggcttgacctgcgtcttcaagacagatgtttagatgaactgtcaacgatattctatatttttaaagattgattcg

SEQ ID NO: 593 - DNA, 275 bp, Mus musculus
  misc_feature (1)...(275): n = A,T,C or G
gctgttgaagctgcggntcagggtgnggccatcaccgggaccagactttcacaactggaactggcccaatgcaatgatttttgntgncacagtcatcaccaccatcggttatggcaatgtggctcccaagaccccagctgggcgcctcttctgtgtcttctacggcctcttcnggggtgccgctngngcctgacatggatcagtgccctgggcaagttncttcggggggacggngcaaagaggttagggccantttcttaaccaaggaagaggag

SEQ ID NO: 594 - DNA, 334 bp, Mus musculus
  misc_feature (1)...(334): n = A,T,C or G
aaatggcttccaaaagagctctggtcatcctggccaaaggagcanaggagaatggagacagttgattcctgtggatgtcatgcggctagccggtattanagtcactgangcangcttcggnttgnnaagngaccccggggcacnngtaaacccgtgatgtaantggattagtaccaaaatacccagccctngaaaagatgcaaaaaacgcnttggaccattnccaaangnaggggggtnccttccngggnaggaaatcntggntgccnaaaaatttnatctcattcgccttannggggaatgnaaaaccctcaagnggcccgggaaaagaagga

SEQ ID NO: 595 - DNA, 493 bp, Mus musculus
  misc_feature (1)...(493): n = A,T,C or G
gaactgaggatccttttgtggcattccacatcaacaagggtcttgtgagaaagtatatgaactctcttctgattggagagctggctccggagcaacccagctttgaacccaccaagaataaagcgctaactgatgaattccgggagctgcgggccacagtagagcgaatgggcctcatgaaggccaaccacctcttcttcctggtctacctgcttcacatcctgctgctggatgtggctgcctggctcaccctttggatctttggaacttccttggtgcccttcatcctctgtgcagtactgctcagtacagttcaggctcaggcaggttggctacagcatgactttgggcacctgtcagtctttggcacctcgacatggnatcacctgctacntcattttgngattggncacctgangggggcccccnccagctggnggaccacatgcatttcagcacnatgccnagcntagctgcttccgcaaggaccccg

SEQ ID NO: 596 - DNA, 115 bp, Mus musculus
  misc_feature (1)...(115): n = A,T,C or G
ggattgaaaatgaattngattcctaaatgagaagctcgagggtgaggcaccgtttcgaactctgcngtgtgcaangaagacgaggaagttccagcatggcctcggggggatgttg

SEQ ID NO: 597 - DNA, 205 bp, Mus musculus
  misc_feature (1)...(205): n = A,T,C or G
ctgaggtgtacagagccaatgggccagatggagctgtgggccgaggccagagaatccacctgcagttatgggacacggcgggggcaggagaaggtttcgtagcttaacccactgcattcttcagggacgcttttgggtttcctgctttcttgttcgacctggacaaatgaancaaagtttcctcaattgtccgaaactggataag

SEQ ID NO: 598 - DNA, 166 bp, Mus musculus
  misc_feature (1)...(166): n = A,T,C or G
tgaggatgagttccacccgttntnngaggcgctgctgccgcatgtgcgcgccttcgcctacacctggttnaacntgcaggcagcgcaagcgcaagtacttcaagaancncgagaagcgcatgtccaaggacgaggngccgctnttttttatgatcaatatgagaga

SEQ ID NO: 599 - DNA, 325 bp, Mus musculus
atagaaggtggtgatggactacagctcagaaaggaacatactctcaaaatatttgcttatatcaattcctggactcagaggcaatgtctatgctgcttcaaggaatataagcatttggagatttttaaccaagtagtatgtgcacttattaatttagtcattgcccaagttcaagtgctccgggaccagctttgtaaacattgtactaccattaacatagattccacgttggcaagatgagagtaatcaagcggaagaaccactgagtatagatagagaatgtaatgaaggaaatacagaaagacaaaaatcaatagaaaaaaaa

SEQ ID NO: 600 - DNA, 500 bp, Mus musculus
gactgaggtatttacataaggaatttgcagagtcctggtcagttccaaggttggcagaaggctttgatgtcagcactgatgttatccgaagggttttaaaaagtaagtttgtacccactttagagcagaaactgaggcaggaccaaaaggtcttgaaaaaagctgggtttacccgagagattggtcagctccccgtttctgaagataccttgaaggcgctcagtgcaggccgctctgtgtctggcctgctgatggcaggggatgaagtctcctctaaaagtcagaatcacagcacagctttgaaagtggcgaagtcgcaccctcacagcacagacgcacagaagaaacgggaagggagggataaaagaatccaagtcttggaagagagcctagtgcctgccaccacagccttgggtcatcagcgagagcttcagaaatcccgccaccagtgacttccgaggccaccggaagagctggcagtgatacgttaccaagtgctg

SEQ ID NO: 601 - DNA, 149 bp, Mus musculus
gactgagaggaagacgaacagctctccccactggagccataactttacccatgagccttcactctcagccagggcagacaagagcccagcgcgtctgagactgttcctgctctgacccagatatccaacctgctagtgtgtgaggtcat

SEQ ID NO: 602 - DNA, 339 bp, Mus musculus
  misc_feature (1)...(339): n = A,T,C or G
gtcccatactacaaggaacccactgatctggtngaanaagatggccaagattactacnttngcgaccaggngagatgatgcagcagtgatatngtagcanggnacttcattgagcangctganttntcngggnacctgtncgggacatgnanggaagntgtcttggctgagcangccatnaacctcatctgcgtgctannngtatgacctacnnggggggcgcngcatcnanaagactgntcttgtgtncgattctacatctttgggcagcctncctcgctggacgtgctggagcaacgactgcnacngcgcaacactgagactgatgagagtctggca

SEQ ID NO: 603 - DNA, 452 bp, Mus musculus
  misc_feature (1)...(452): n = A,T,C or G
gaccccattgtgatagctcatcgcttgtctaccgtncgtantgctgacgtcattgctggggntgatgntgnngtnattnngnagcaaggaantcatgatnagctcatgagananaagggcanttacttcaaacttgnnatgncncanacagcagtgaaatganattganttatgganattgaancttgtaaatctaaggatganattgataatttagacatgtcttcaaaagattcaggatccagtctaataagaagaagatcaactcgcaaaagcatctgtggaccacatgaccaagacaggaagcttagtaccaaagaggccctggatgaagatgtacctccagcttccttttggcggatcctgaagttgaattcaactgaatggccttattttgtggttggnatattctgtgccataataaatggaggcttacagccagaaaaaaaaaa

SEQ ID NO: 604 - DNA, 471 bp, Mus musculus
  misc_feature (1)...(471): n = A,T,C or G
actgagaatggtccagaactacccatgagtcagtttggctggagctcgtgagcattcaataatgagtggtgcggctttaggacttganattgtttttgncttttttctggcattgntcctcctgcatcgctatggagactttaagaaacagcacagactggtaatcatcggcacactgcttgcttggtgtctctgctttctcattgtcttcatactgccgctggatgtcagtacgacaatatataaccgatgcaggcatgctgctgcaaattccagccctcctgaaaataccaacgttacgggactggatgcatctgtcactccagctccaagacagcatccatgtttcaagccgnggagttacattccagatggaatcatgccaattttctggagggtagtttattggacatcacagtttttgacgtggatcctgtnaccttttatgcagncatatgcaagatctggagg

SEQ ID NO: 605 - DNA, 408 bp, Mus musculus
cactgaggatggcctcaggcaaagtacgctgcacccgaaagctgcggagctggatcgtggagcaggtggagagtggccatttcccaggggtgtgctgggacgatgcagccaagaccatgttccggattccctggaagcatgcaggcaagcaagacttccgagaggaccaggatgctgccatattcaaggtgagagttcctgaaggcaccccaaagcacttttgtctgtttggggtttggataaaggaaatactagaaagtagaacctgggctggtgagatggctcagcggttaaaagcaactggctgcttactcttccagaggtcccgagttcaattcccaacaaccacatggtggctcacaaccatctgtaatgggatctgatgccctcttctggtgtgtctgaaaa

SEQ ID NO: 606 - DNA, 279 bp, Mus musculus
ctattggaagaggcaaccatttcagtttgcaagtctttggttgaaaacaaccctcgaacaggaaatcttgctgcgctaactaaggtcttcctatctagaaccagggagctcaggctctcagccgagtgtcagaaccacatcttcatctggcagacacacaatgctctgtttattatttgctgcttgctgaaagtgttcatccgtgagctgtctgaggaagagctgcagcttcattttacatatgaagaaaagtcccccggcagctacagttctgactct

SEQ ID NO: 607 - DNA, 535 bp, Mus musculus
gactgagtataggtactgcttgctttctgcactaccaccctcaagaaactcagctatacaagttgggggaatgaatgagaaacgtcatttcactcattatgtacagactacacgggactgcagcagtcagagcccaaaggacccactgatgaagaggaaaatatcgctaagagcaaaggacaggcaactcgaggtggaacagtgtaaagacctagggtgttccatttggccacacctaaggaaacacctccttcttgtctgaagaatggttgagtgctgatctaaggatcagcatcgaggcacttacttgaattaaaaaacctactataaaatgcattccgaatagtaaccaggccgggtagaggctggcgacggctaaacccttctccgtggtgtctgtttttgtacacaacgctacgaagggggaaagccaggcttgtgggtattcttacactcacggctaagacccagaagccggggcagcatcaggaccagatcccaatttcccaaatgactggcactgaagagggggaag

SEQ ID NO: 608 - DNA, 458 bp, Mus musculus
  misc_feature (1)...(458): n = A,T,C or G
gactgagacagctctggccctcctgctgctcccatagcccgaattanaaccaccaggaagaaagaatcttatactgattctgtggaggacctgagatcccggggatgtggccactggacagtgcttcggtctcttcacctgaggggagcctccccctaaagaagaaactgagtagagccgtgacagggagacgccattcacagacgaatctgaagtcactgcaccttgaagggtagatgggtgcacaaggaatgggcggcatgctgtcccgtttctcccagcactggacagaaccaagctgtgctggtgcaaccctttaatcccagcacctgggaggcggagacaggtgcatctctgagtttgaggccaggctggtctctagttccaggagagccaagaatccttaaatttaaaatatatgatatataggtcaatcacanagtgcttgcctagcatcg

SEQ ID NO: 609 - DNA, 531 bp, Mus musculus
  misc_feature (1)...(531): n = A,T,C or G
actgagctgccaatggtgtccttaccatactggaacttaccaagaccggctaacaaagctgcaggaccaccttcggcaactttctattctcttcaggaagctgcgactggtctatgacaaatgtaatgagaactgtggtgggatggaccccattcctgttgagcaactgattccatatgtggatgaagatggctcaaagaacgacgaccgggctggtccacctcgttttgctagcgaagagagacgagaaattgtagaagtaaataagaaactcaaacagaagaatcaacagctgaagcagattatggatcaattacggaatctcatctgggacataaatgccatgctggcaatgaggaactaaagcgatatttaaatctcctgctctactcatgtgatgaagttgggttttttcccccctcttgagtattcctccctttgaaaaacgttatttatgtctttattttaacagctagcactaaagnttctgttttcactttaaaagtatttactagcttttttttaatactg

SEQ ID NO: 610 - DNA, 441 bp, Mus musculus
tgagggggcagctggcatcctctgctacgtgggagcctatgtcttcatcacctatgacgactatgaccacttcttcgaggatgtgtacacagctcttccctgccgtggtgatcatagctgtaggagccttgcttttcatcattggcctgatcggctgctgtgctacaatccgggagagccgctgtggacttgccacgtttgtcttcatcctgctcttggttttcgtcacagaagtggttgttgtggttttgggatacgtgtacagagcaaaggtggagaacgaggttgatcgcagcattcagaaagtgtataagacctacaacgggaccaactccgacgctgccagccgtgctattgactatgtgcagagacagttgcactgttgtggaattcataactattcagactgggaaaatacagattggttcaaagaaacaaaaa

SEQ ID NO: 611 - DNA, 400 bp, Mus musculus
  misc_feature (1)...(400): n = A,T,C or G
gaactgagtaacaggatctcggtgactggctcattgtggaaagtacagtctctggagtagaagaggaggactgagaactacaggctcagatctctattnagacatccaacagaattaatactatcattggctataacatgaagctattgngttaaagganagaagtcgagtgntaaaagacttcctactccaggatatcctactgaagaagtccagaanaagcatggtggccctgactgacagcagacatgctgnctgatgctaggctgcnggggctgcgtgtgctntcccaccctttgtgnctngaagnngacngtgctcaaanngatgngganaggancggagtccgganatcactgggactcggacatcgnctacacngagggttgaacccacaggt

SEQ ID NO: 612 - DNA, 393 bp, Mus musculus
  misc_feature (1)...(393): n = A,T,C or G
catgcgctgtggatatggagacagcccttcaaaaggagcagttccatatgtgcaagcatttgactcgctgcttgccaatcccgtggcagagtacttgaagatgagtaaggagatcgggggagatgtgcagaaacacgtaaggacacggnggccctttctttctcctcctatttaaggactggangcttttcttcacatctcttcaccctcatcataggggcgtccggnagccccctgactccttttcttcttcctttattggtccaggaacagaaatggggttcatttttctaggtctcctgttttattacatattctgtgtgtttcttttatgacaaagcttgnntggtcataaaatacaactgngntcatctttgtgggggaaaaaaaaaa

SEQ ID NO: 613 - DNA, 465 bp, Mus musculus
  misc_feature (1)...(465): n = A,T,C or G
actgagaccagatcactcctggntgaagtgacagctgtggtgaagattgaaaaagatgttggtggcaataatgggggtagcggcaatggtggcggtgccgccttttctcagactcgaagcagcagcacaggcagtagcagcagcagtggtggcggaggagggcagcagaaccagacatcaggaggctcgctgcaaggaagtcagcagaaagagggagagcaaagtcagcagacacagcaacaacaaattcttattcagcctcagctagttcaaggaggacaagctcttcaggcccttcaagcagcaccattgtccggacagaccttcacaactcaagctatttcccaggaaacccttcagaacctccagcttcaggctgttcaaaactctggtcccatcatcatttcggacaccaaccgtggggcccaatggacaggtcagttggcagacccttcagctgcaaat

SEQ ID NO: 614 - DNA, 234 bp, Mus musculus
gactgagcctacacagtccagacatccgagggtgtacgcccaacagccgcttctgaggcccgtttgaaagccttggctgtttgccacgggcctctggaccactacgactttctgatcaaatctcaggagctgagggaggatgaacatcaaagaagagtcgttcagtgtttgcagaagttacaggaggaccttaaaggatacagcatagaggaaggagggctcttttcaaagctt

SEQ ID NO: 615 - DNA, 194 bp, Mus musculus
  misc_feature (1)...(194): n = A,T,C or G
gaggccataataacttcntgtggnactctcagaggccngnacacngaagcatggcttggtnccataacacangaacnngcatcgaaggcgngngcngangggcccacaggccnacagnaggagagggccccaaagctaccaagggagnctctggccacanatgngactcgttgntcctcctgggactgcgatgg

SEQ ID NO: 616 - DNA, 128 bp, Mus musculus
  misc_feature (1)...(128): n = A,T,C or G
gactgagcctgtgcctactgcactagcccaagtggacagagagaagatctatcagtggatcaatgaactgtccagtcctgagnnagntntccatgatgtctcaaagnataagctttaagaaaagaaca

SEQ ID NO: 617 - DNA, 484 bp, Mus musculus
  misc_feature (1)...(484): n = A,T,C or G
tctggggagctcctgcattaagtcagactgaggctacgagggccccggttctgtactggtgtctccggtgccgcaccagatgctgccctacatgaacgtgggcacgctgtcgcgcactgagctgcagctgctcaacccagctgacttgtcggcggaagcggcggcaccgcaccatcttcaccgatgagcagctcgaagccctgggagaacctcttccaggagacgaagtacccagacgtgggcactcggggagcagcttggcccaggaagggtgcaccttcggaaggagaagggnggaggtctggttttaagaaccgccgagccaagtgggagacgacagaaagcgatcctcntcggaggagntcagaaaaacgcccgaagaagtggaacaaagaacgtcctnaaaaagccttcgccggagaagaggggaagaaggaaggtaaaagcgatttggactnnggacagcttgacgggccgcggcccg

SEQ ID NO: 618 - DNA, 464 bp, Mus musculus
  misc_feature (1)...(464): n = A,T,C or G
tgttcaatccaatgactccaccacaagtcaatagctatagtgagccatgctgtctccgacccctccacagccaaggagtccccagcatgggaacagaaggactttctggtctgcccttttgccaccaagccaactttatgtcagggtcccagggttatggagcagccagagagaccagcagctgcactgaaggatctctctttcctcctcctcntcctcctnggagtanagtcanattaacaaagaggcggactctntccatntcgcccctttnngatgccagcctggacctgcaaaccgnaatccggaanctnacccaactnacntgggaggntntnatnaactntcgctgtacatannngggcggttnctacggncatctntccattgnnaccatgagcccttcttaaggattcccacctnagatgagncatcaaaaaggaacttcacctcccttatggagg

SEQ ID NO: 619 - DNA, 495 bp, Mus musculus
  misc_feature (1)...(495): n = A,T,C or G
gagccgttcagtccaatgcattgatagttaatttgacagattcaaagggtacttgcctttatgcagaatgggagatgaatttcacaataacatatgaaactaccaaaccaaaccaataaaactataaccattgcagtaccctgacaaggcgacacaccgatggaagcagtttgtggggatgaccggaatagtgccaaaataatgatacaatttggattcgctgtcctcttgggcctgngaattttaccaaggaagcatctcatttattcaattcatgacatcgggctttcctacaacacttagngatagcacaagtattttcctgggngctgnagctaaagggagttcatactggttaaaaancctggagaattttcaaagntncctttgnaaggccatcctttaaggggcaatagnggttttaaacttaacaaaccctgacttccctggccgttcaaaaaatattggggggnatttcnccctgcaggctttttg

SEQ ID NO: 620 - DNA, 274 bp, Mus musculus
  misc_feature (1)...(274): n = A,T,C or G
tctggggaacctcctgcattaagtcagactgagggcngnacccacagtccagtaagctagatggcagngttactatngatctacnggcnngatggctatcccagcangttgntcctggnctacaaagtcttgnatgacnngangaaggccatccctggntttcctcttgngncgcacaanacccgnctgcacattgcanaggacccctctcttggactatgagtacttgcccntgntgnncctgaaatcattcatccngtcttctttggaactc

SEQ ID NO: 621 - DNA, 501 bp, Mus musculus
  misc_feature (1)...(501): n = A,T,C or G
tgcttactgagcgtcanancttncacatggaacangttgaaatatgctgaactacgtgcccggcagcaaatggagcagcagcagcagcatggccagacacctcagcaggcgcaccagcacacgggagggccggggatggccccacttggagccacaggccaccctggcatgatgccgcatcagcagccccctccctacccactgatgcaccatcagatgccgccaccccatcctccccaaccaggtcaaataccaggccctggctccatgatgcctggccagcccatgccaggtcgcatgatccccgctgtggcagccaacattcaccctactgggagtggccctacccctcctggtatgcctccaatgcccggaaacatcttaggaccccgggtacccctcacagcaccaaacggcatgtgtaagtagcacagtgggacgacattacccagcatcctgctttggaagctacagataaggaagaaatgctttcttcgtgaa

SEQ ID NO: 622 - DNA, 323 bp, Mus musculus
  misc_feature (1)...(323): n = A,T,C or G
gactgagaatcctacnattcctcatcatgacnccttctcaggncacttttgaaataagaggaactcttttaccangagaggnctttgcaatatgnggaagctgcgatgccctgnnaaactgnaatcctcaaaatgctgcggctcttattantgaanacgacacaggagacagcgngttgtggaaagcagcgattgctctcaatanaggagngncagngaagnaccgcnacttcagaggcngctttttagaaccannggaaggatgtctttatatattnttttattactgnaggacttngcatagtancaactttaagtttgaa

SEQ ID NO: 623 - DNA, 138 bp, Mus musculus
gactgagaaagagtagttgagcagctgcgcagaacctgatagtcaaacaagagcagccggacaagttccagatacagccattgtcacagtctgaaaacaaactacaaacagcacagcagcagccactacagccactgc

SEQ ID NO: 624 - DNA, 383 bp, Mus musculus
  misc_feature (1)...(383): n = A,T,C or G
gactgaggtttgatctgtggatgaaatgaatcatgattttcaagctcttgcattanaatctcggggaatgggagagcttttgcctaccaaaaagttttgggaacctgatgattcaacaaaananggacaangaggcgtatntcttggcnangatgaangganagagactgcatggggaacttctcatcattcaatgtcccagcctattatggtgcagagaagatctggacagagttttcatggaaacagtgaagtaaatgcaattctttctccacgctcagaaagtgggaggccttggtgtgagcatnggtagaatatgtattaagttcctctcctgctgataaatgggattctcgatttaggaagggaacttttggaactat

SEQ ID NO: 625 - DNA, 363 bp, Mus musculus
  misc_feature (1)...(363): n = A,T,C or G
gactgagagtctatgatggaagtcaaagggaagaaaaagtttacaggaaagagtccacagacatcacaaggaaagaataaatttcataagaacagtgagtctagttcttcaaagacattcccaaggaaagctgttaaggaaggcggacctaaagtcacatctaagaactttgagaaaggtgccacaaaacctggcaagaaaggtgtgaagcagttcaagaacaagccacaagggggcaaaggaccgaaggacaaatttcagaaggcaaatanattcagcaagaagcggaaattccagccagatggtgaaagtgatgaatcaggagccaagaaacccaagtgggatgactttcaaaaaaaaaaa

SEQ ID NO: 626 - DNA, 540 bp, Mus musculus
tctcccgactctcgagagactacattcgtcgtcctcctcttgctgcttcttgccgtgcctggagttactgtccgtctcttaagaagtctgccaggccggttacatttctctttccacgagctccttacccataagtatcttccctgagctgtcagtcgctgacccggttgactttcgacgccccctccccaactcctcgcaagcctgatctctgacctttgaaccctagacaccctcatcaacactcttccctcagtgtatccccacatccaacagtcgggatgccgggtgctcccacatcctctctgttccaaataaggcccaccccaggtaggttgtggctgaggagtacgagcctgaggacagcaagagtttgtagtatatagtccactgagatccctgacgcctcgctccacatcaggaaggtcccaccctgaaattccggctccacatacaggatcttgagtctccgaacacctggatccttcgagatgtgaggtcctgtgtccaggtgagcagtgtgctcttcaaaaaaaaaaa

SEQ ID NO: 627 - DNA, 639 bp, Mus musculus
  misc_feature (1)...(639): n = A,T,C or G
ggcggtttgaagcctctgtgatcgaggagcggcgcaagggcgcggaggacctgctgcgcttcacggtgcccatccctgcactcaacaacagcccccaactcaaggagtttttccggggcggggaggtgacacggccctctgaggtgtccagggacctgcgaatcctgccaccccctctcatccccacaccacctccggatgaggcccggctacttcagccgctgcctgcggaaaggaggggccaggaggagttggaggtgccagtggatcccctcccgtccagccctgcccaggaggccttggaccttctcttcagctgtgacagtacggaggaagcttccagctccctggctcgaggtcccctctctgaggctgaacttgcccttttcgacccttactccaaagaggaaagtacaggccccagccctacccacacaggtgagctggcagccattgaggtagagtccaaaaggctggaccaggaaccctgggagccaggaggacaggaggaggaaaaagctgaggatggagagcctgccccagcttaccttggccaggccacagagctcatcactcaagccctgcggaatgaaaaggctggcgcctacgctgctgntntncaaggctaccaagaagggg

SEQ ID NO: 628 - DNA, 770 bp, Mus musculus
  misc_feature (1)...(770): n = A,T,C or G
gtggggtctttcagtgctggaccctgactgccaagaagaactacagggcagatggcatcgggagcagcccactctctaatcaggatgctttccgtctatggtgcctcttcaacttcctgtctgaggacaagtacccgctgatcatggttcccgatgaggtagagtatctgctgaagaagctccttggcagcctgagtttggagatgggcctgggcgagctggaggagctgctggcccaggacgcccagtcagcccagaccgccgtggggctcagcgtctggcagtttctggaactcttcaactcaggccgctgccttcggggtgtggggcgcgactccctcagcatggccatccaagaagtctaccaggagctcatccaagacgtcctgaagcagggctatctgtggaagcgagggcacctgcggaggaactgggccgagcgctggttccagctgcaacccagcagcctctgctactttgggagtgaggaatgcaaggagaaacgaggcaccatttcccctggatgctcactgctgtgtggaggtgcttccggaccgcgaaaggaaagcgctgcatgttttgtgtgaagactgccagccgcacctatgagatgagcgcctcagacacccgccagcgccaggaagtggacgggccggtcacnngcncgccntnngtttanctgggcctgttcaccgaaantgaccttttggggccccttttncggtaggngactaanaacaaggcttgactcccgtccggctctgccctta

SEQ ID NO: 629 - DNA, 521 bp, Mus musculus
  misc_feature (1)...(521): n = A,T,C or G
gacgtctggggagctcctgcattaagtcagactgaggtcccttcactgatgttgtcaccaccaacctaaagcttggcaacccaacagaccgaaatgtgtgttttaaagtgaagaccacagcacctctgcangtactgcgtgcggcccaacagtggggtcattgatgccggggcctctctcaatgtgtctgtgatgttacagcctttcgattatgatnccaatganaaaagtaaacacaagtttatgngttcagnctatgtttgctccacctgactcttntnanatggaggcagtatnncaggnggcaancccggaagaccttatggattcaaaacttagatgtgnttncnantcngccaccatnaaangctagtccacatgntgnagaaatanataaaatcatacctacggagtgcntncaanacanaagcaccggcagccgcaaagtccctgacatcgcccctcgatgacacagaagtaaagaaggngatggaagagtgcaggcggctgcaggggggagg

SEQ ID NO: 630 - DNA, 800 bp, Mus musculus
  misc_feature (1)...(800): n = A,T,C or G
gacgtctggggagctcctgcattaagtcagactgaggtggggtctttcacgctggcttcgcgagcagtccggacccacgatcgcttttggcaaccagaaccggcgcttcagcccccggggtgacgtgcagcccgccgcccagacacatggccccgagcccaagaccccagcatgtcctgcactggagggacgcccacaacttatatatantgtacgaantgatgntnttgctcagtcgggcctggagccgcctgaggggcccagaagtcccagaggcatggctggcaaaaacagtaacaggagcagatcagatagaagctgcggctctgctgacacctacccctgtctctggtaacctcctccctcatggggagactgaagaaagtggatctcctgaacagagtcaagcagcccagaggctctgccttgtggaagctgaaagttcccctcctgaaacttggggactttcaaatgttgatgagtacaatgcaaagccaggacaagatgaccttagagagaaggaaatggaacgcacagctggcaaggccacactacagcccgctggcctgcaaggggctgataagaggcttggggaggnggtggctagagaagagggagtggctgagcccgcttatcccacatcacagntggagggtggnccagcttgagaatgaagaggatggagaaacagtgaagacttaccaagcttctgctgctttcatagctncgggatacaaacccagcacccctgtgccttttttggggaggcanaacatcaagccacggaagaaaaaggaaca

SEQ ID NO: 631 - DNA, 658 bp, Mus musculus
  misc_feature (1)...(658): n = A,T,C or G
gacgtctggggagctcctgcattaagtcagactgaggtacccacccggcngcatggcaaccctacagctgntcatagctcccttgctgtgnntcctgcttngggtcttttgngctccaggtgccagancccatgaccatgnggctgatgnccatnatggnagcntggncctggataagancacagtgcacgaccaagagcacatcntggaacatntngaaggtgncatcgaccaaccagaggcggagatgtccccacaggaactgcagntccattacttaaaantgcatgattacgacggcaacagtttgcttgacggcctanagctctccatagccatcactcacgtgcacaaggaggaggggagtgagcaggcgccagtcatgagcgaggatgagctcgtcagcatcatagatggtgtcctgagggacgatgacaagancaattgacggctacatcgactacgctgagtttgcaaaagtcactgcagtagaccgttggctcttttccttttgtgcacatgtgacccttgctaatgngatggacgtgtctggtaatgcgaaacaacttatttccgtctactgctcaacacttttggtaaaaaccctgtggcannctgntanaatggggtggaggaaanaaaccacatgactggggaa

SEQ ID NO: 632 - DNA, 247 bp, Mus musculus
  misc_feature (1)...(247): n = A,T,C or G
gggtgagccatagactatcctaggaaaccaaaccagggcccngcnttgttcagagaggggacagataatgnactccctganccgnngnatcnaggatagcaccccacagtgcatncctgnctattnagaggcacanactctcagcacgtgggagaccttgctacctacggacaatncttgatntacagntgtattgccacttncttgctcaacaaacctgttactttnttggaaataatatttaatt

SEQ ID NO: 633 - DNA, 378 bp, Mus musculus
  misc_feature (1)...(378): n = A,T,C or G
ccatggtaacatgcttatnctgtntacacttctatatggacaagaagtctaaactaggaacctacctactctggacaaccattcccatctccacccanaggacaccaatcatcgtgacaccacgtccatcttttactaagttcccccaacctcttcctcctctcactagtagacttaatacagaagcaaggatctaggccgggtgttgctactctgaaacttattgcacgagatattgtcaatttatcccctaactgacctcccagagaagaaagaagtgggaagaaacggcgaatagaaggcaagagacactgatcagttagaggggaaactctacatcttctctggttgagagtctggnaactgaagaaggcaaaa

SEQ ID NO: 634 - DNA, 248 bp, Mus musculus
  misc_feature (1)...(248): n = A,T,C or G
ttatcatggggcgcaagttggatcctacgaagaaggagaagcgtggacccggccgaaaggcccgaaaacagaagggtgcggagaccgaattggtcagatttttgcctgcagctgncgatgagaattccatgaggctgtcnagtcttncccgaatncgggcanccaagaggagggcagggtctgttgacgtccctaagccaaataagtctcctgggatcaaaacattgcctggagagttatccaaagga

SEQ ID NO: 635 - DNA, 683 bp, Mus musculus
  misc_feature (1)...(683): n = A,T,C or G
ggactttgaatatgtcggggatcgccctcagccgccttgcgcaggaaaggaaagcctggaggaaggaccacccttttggctttgtagctgtcccaacaaagaaccctgatggcacaatgaacctgatgaactgggagtgcgctatccctggaaagaaggggactccatgggaaggaggcttgttcaagctacggatgcttttcaaagatgactatccgtcctcaccaccaaaatgtaaatttgagcccccactgtttcatccaaacgtgtatccttctggcacagtgtgcctgtccatcctggaggaagacaaggactggaggccagctatcaccatcaaacagatcttattaggaatacaagaacttctaaatgaaccaaatattcaagacccagctcaagcagaggcctacacaatttactgccaaaacagagtggaatatgagaaaagggtccgagcacaagcgaagaagtttgccccctcataagcagcggccctgggctccatgacgaggaagggattggcttggcaagaacttgtttacaaccttttgcanatntaagtcgctccgtacagntactantcncctggganggntgagcgggcnccattttccttttccgccattggatattcagncttttgatttttgantattaannaaaacttgcttttattttaa

SEQ ID NO: 636 - DNA, 665 bp, Mus musculus
  misc_feature (1)...(665): n = A,T,C or G
tctggggagctcctgcattaagtcagactgaggtcctcagtatggcagcctggagcgggcgtggggcgccatgatgacagaagcagataaggtcagcgagctgcaccaggaggtgaagaacagcctgctgaatgaggacctggagaaagtcaagaactggcagaaggatgcctatcacaagcagatcatgggtggcttcaaggagacgaaagaggccgaggatggcttccgaaaggcccagaagccctgggctaaaaagatgaaggagctagaggcggccaagaaggcctatcacttggcttgtaaggaggaaaggctggccatgacccgggagatgaacagtaagacagagcagtcggtcacccctgaacagcagaagaaacttgtggacaaagtggacaaatgcagacaggatgtgcaaaagactcaggagaagtatgagaaggtgctggaagatgtgggcaagaccacaccacagtacatggagggcatggagcaggtgtttgagcagtgccagcagtttgaggagaagcggctggtcttcctgaaggaagtcctgctggatatcaaacggcatctcaccctancggaaacagcagctacttgcatgtctaccgagaactggagcaggccttccggggggcccgatgccccaggaggacctt

SEQ ID NO: 637 - DNA, 233 bp, Mus musculus
  misc_feature (1)...(233): n = A,T,C or G
acttgcaaaattatgaaggantctanaagcagagganaacataaaagnccancnccttngnnccncttatccncaccctccctgagtcttggcatggacacacctatnaangaccttcctggngcagatacatnctcgnatgtgaagggagaaaagcttactcgctgnaaacttgccagnctctacaggctngcagacttgnttgggngggcgcacctggcaaatacgtacat

SEQ ID NO: 638 - DNA, 343 bp, Mus musculus
tctggggagctcctgcattaagtcagactgaggattacaacattccagagtcccactggggaagaacaagatacttcaataaatgaaggagaacaaagaaaactcaagcccttcagtcacgtcagcaaacctggaccacacaaaaccatgttggtactgggataagaaggacctagcgcacacaccctcacaacttgaaggactcgatccagccactgaggcccggtaccgccgagagggggctcgcttcatctttgatgtgggcacacgtttggggttgtatcctgaatctctgggatttgttgtgaataaactccatcaccaagaattagattaaaaaaaa

SEQ ID NO: 639 - DNA, 596 bp, Mus musculus
  misc_feature (1)...(596): n = A,T,C or G
tctggggagctcctgcattaagtcagactgagnattaaatctgagagaatctgacatcagagtgtgtgatgaatcatcttgtaaatatggaggtgtctgcaaagaagatggagatggtttgaaatgcgcatgccagtttcagtgccatacaaattacatccctgtctgtgggtcaaacggggacacctaccagaatgagtgctttctcaggagggctgcttgcaagcaccagaaagacataaccgtggtggcgcgtggaccttgctactctgataacggntctggatctggagaaggagcagaggaggaaggctcaggagcaggngctnacaggaancattccaagtgtggaccgtgcaagtacaaggccgagtgtgacgaggatgcggagaacgttgggtgagtntgtaatatagnctncngtggcttcacnannnnacnctncccgcgntganganngancntcctanaatgnccgntgtgtcntccgaaaggcgacttgcataaagcactgaacngatcgacanantagcnngtcngccnctggncatacacagatgangtggagattattgnnaaagaaggacgacgggctg

SEQ ID NO: 640 - DNA, 578 bp, Mus musculus
  misc_feature (1)...(578): n = A,T,C or G
cccagttgtgacatgatggaactaaggcgccgttatcagaacttatatattcctagtgacttttttgatgctcagtttacatgggtggatgctttccctttgtcaagaccatttcaactgggaaattactgcaatttttatgtgatgcaccgagaagtagagtccttagaaaaaaatatgggctgttcttgatccacctgatgctgaccacctgtacagtgcaaaggtaatgctgatggctagccctagtatggaagacttgtatcataagtcatgtgctcttgctgaagacccacaagaccttcgtgatggttttcagcatcctgctagacttgttaagtttctagtgggaatgaaaggcaaggatgaagccatggccattggaggccactggtctccttcgctggatggaccaaacccagaaaaagatccctctgtgttgattaaaactgccattcgttgttgnaaggctctgacaggcattgatctaagngtatgcacacagnggtaccggtttgcanagattcgntaccatcgccctgnggagacccacaaggggcgnacagttccagctcatg

SEQ ID NO: 641 - DNA, 716 bp, Mus musculus
  misc_feature (1)...(716): n = A,T,C or G
tctggggagctcctgcattaagtcagactgaggcctgaaactcagcaccgaggctctgcaccccactccgagagcgacatcccagagcaggaggaggagattctagggtctgacgatgacgagcaggaagaccccaatgactactgtaaaggaggttatcatcttgtgaaaattggagatctatttaacgggagataccatgtgattcgaaaattgggctggggacacttttccacagtgtggttatcatgggatattcagggaaagaagtttgtagcaatgaaagtagttaaaagtgctgagcattacacagaaacagcactagatgaaatccggctgctgaaatcggttcgcaactcagacccgaatgatccaaatggagaaatggttgttcaactactagatgactttaaaatatcaggcgttaatggaacacatatctgcatggtgtttgaggttttggggcatcatctactcaagtggatcatcaagtccaactatcaggggcttccgctgccttgtgtcaaaaaaattattcagcaagtgctacagggcctggattacttacacacccaagtgccggatcatccacaccggacatcaaaccagagaacatcttgttgtctgtgaatgagcagtacattcgaaggctggctgcagangccacanagtggcagcgctcangagctcccccaccatcggggtcttgcaggctg

SEQ ID NO: 642 - DNA, 276 bp, Mus musculus
  misc_feature (1)...(276): n = A,T,C or G
gtctggggagctcctgcattaagtcagactgagtgctccactccagctacgggaactggtgaactgtcgttgggcggaagaagtgacacagcagcttgacactctccagctgtgtagtctcaccaaacatgaagaaaatgagaaagacaaatgngaaaatcaccatgaaaaacttagtagtgttttgctggacttgtnagaagtgtatctgccatnagtgngcnctttggggaggaatggngngcngancaaatgttntntttggtttacatacta

SEQ ID NO: 643 - DNA, 102 bp, Mus musculus
gaacggtcatcaaagctccgctgtgcttggttactggtgttcctggcaggttcctccgtcctcttatattacacctttcattctcagtcacttcattacagg

SEQ ID NO: 644 - DNA, 781 bp, Mus musculus
  misc_feature (1)...(781): n = A,T,C or G
gtttatcaatgccagcaagaagaatagtgcagcccatgattgaccagtcaaatcgagcagngagccaaggagcagcntatagtccanagggtcagcccatggggagctttgtgttggatggtcagcancacatgnggatncggcctgcaggacccatgantggaangggcatnantatgggcatggatggccagnggcactatatgnanncttngccatgtnangcaatctctnggcaaggggaagtttgcanagcatgccnggggactacnttnctcagggtggtccnntgggaatngntatggcccanccaagttacactcctncccagatgaccccacaccctactcagttaagacatggaccnccaatgcattcatatttgccaagccatccccaccacccagccatggtnatgcacggaggaccccctacccaccctggaatgactatgncannacatagccccacaatgttaaatnctgtacatnccaatgttggcggacaggttatggacattcatgcccantattttaanggaactcnnggaanaaggaaacacacncaaaaactattttaagactctctgaactttgaccanatgtgacacttaatatgaaactccagacanctgtgattattttttactttnntggnatttntcatcnagcaacagaggaccantgngacangaacacanatgtgaaagcgacgggctcatcgtgacaattttgnccatgcaaagatcctttggagaaanactncgagagttataactact

SEQ ID NO: 645 - DNA, 158 bp, Mus musculus
  misc_feature (1)...(158): n = A,T,C or G
gcctgagccgggctgggctcccatttgggttgatgcgccgggagctagcatgngagggctaccccatcgagctgcggtgcccggggagtgatgtcatcataggtggtggcaaacgggcaggatgagtatttaataaaagttcgcaagtttccaaaaaa

SEQ ID NO: 646 - DNA, 117 bp, Mus musculus
  misc_feature (1)...(117): n = A,T,C or G
acttttcctcccccccttgatctcgctgcctgtgaaagccngggnntagntcaactaagacgcctcctgcgagaaagccagagaagagctanggggctgggggaaggagtctaaaat

SEQ ID NO: 647 - DNA, 105 bp, Mus musculus
  misc_feature (1)...(105): n = A,T,C or G
cattagtcagactgaggcgncaagcccatctgngtgngctcgcatccaactgtgatgagcccatgtntgtcaagctggtggaggcactttgtgctgaacaccaaa

SEQ ID NO: 648 - DNA, 212 bp, Mus musculus
gaaagcgatttcgctcacgttttattagcacaaggaagacagccggctccccgaggcagaagatcgcaggcaccattcgctcgccacccagactctgcgcgcagccccggagccagagaggaggatcgtgaaccgaagccagcttcgcagttctagttttgaaagcgccgctggaaaggggcttaaatccgctttttttttttttaaagggg

SEQ ID NO: 649 - DNA, 299 bp, Mus musculus
  misc_feature (1)...(299): n = A,T,C or G
ctttggggagctcctgcattaagtcagactgaggaagccagagctatgcagttctccagctcatccaggacatcagatgagaatgtggactatttgttcaaggtcatcctaagagcttgtccggatggtgctgaatggctgangacattctgtatcccgagtctgttccttgcccagtgtgatttctgtgtggctccagaggctcccctgtcacagcaccttgcccattnggtttctttgaggatgatgnctgccttcaccaaataaaatttgctntctttgccctcaaacaaaaaaaa

SEQ ID NO: 650 - DNA, 238 bp, Mus musculus
  misc_feature (1)...(238): n = A,T,C or G
caggaaagaaaggaaaggaaaagaaaaatagagagggcaaggacaaggaaaatggctctgtatctaccagtgagacaccaccacctccaccaccaaatgaaattagtcctccacatgctgcggaagaagaggaagatgntgactggtngggaggatactaattgtggaatcntcnaaggcnctngnatggttgngatcnntgaccatnctnanaggtctgacacttaaatgttgattt

SEQ ID NO: 651 - DNA, 359 bp, Mus musculus
atgttccccttctctggctgctggaggactgaactgttattgttgctactcctggctgtggctgtgagagaatcctggcagatagaagaaaaatcgtgtgacctggtaggagagaaggataaggagtcaaagaacgaggtggctctcctgggagaggttacggccactgtttaacaaaagttttgagagtactgtgggccagggctcagacacatacagctacatattcagagtatgccgggaagctagcaaccactcctctggagcaggcctggtccagatcaacaaaagcaatgacaaggagacagtggttgggagaatcaacgagactcacatcttcaatggaagtaatagaatca

SEQ ID NO: 652 - DNA, 771 bp, Mus musculus
  misc_feature (1)...(771): n = A,T,C or G
gacgtctggggagctcctgcattaagtcagactgagtgccaccatggcaactgcaccgtacaactactcttacatctttaagtatattattattggggatatgggagtaggaaaatcttgcttgcttcatcaatttacagaaaaaaaatttatggctgattgtcctcacacaattggtgttgaatttggtacaagaataattgaagttagtggtcaaaaaatcaaactgcagatttgggatacagcagggcaggagcggttcagagcggttacacggagctactatagaggagctgcaggtgcgctcatggtgtatgacatcaccagaagaagtacatataaccacttaagcagctggttgacagacgcaaggaatctcaccaatccaaacactgtaataattctcataggaaataaagcagacttggaagcacagagagatgttacctatgaagaagccaaacagtttgctgaggaaaacggcttattgttccttgaagcaagcgcaaaaacgggagagaatggagaagatgcttttcttgaggctgccaagaaaatctatcagaacattcaggatggaagcctggatctgaatgctgccgagtctggtgtacaacacaaaccctcagccccgcagggaggacggctaaccagtgaaccccaccccaaagagaaagctgnggctgctagtgacctctttgctctggcccctcatttgacctttcacctntgnctggtggaagcagactttttactggctccttggctttg

SEQ ID NO: 653 - DNA, 396 bp, Mus musculus
  misc_feature (1)...(396): n = A,T,C or G
actgaggggacaaacaagtgactcctgtcacccctctgggtgccccttanaggccatccagcccaacctgaccaatgtccacagccagggaacagccaatcttcagcacccgggcgcacngtattccagatngaccccactacaaagcggaactggatccccgccggcaagcacgcacttaccgtgtcctatttctatgatgcaacccgaaatggtgtaccgcatcatcagcatnngggggngccaaggccatcatcaacagcactggtcactcccaacattgaccttcaccaaaacctctnagaagttcgggcaatggggcagacagtcnagccaacacttgtctacggcctaggctttgcctctgaacagcagctgacccagatatatttgtat

SEQ ID NO: 654 - DNA, 384 bp, Mus musculus
gacgtctggggagctcctgcattaagtcagactgagaagatggacgacatgtcgcccaggctgagagcctttctctctgaacccattggggaaaaggatgtggcctgggtggatggcatcagccgtgaacttgccatcaatttggtcaccaaaggtttcaacaaggcctatgtcctgcttgggacagtttcttctgatgcacaagaatgaagcagagttccagaggtggatcatctgctgctgtggagctaccggagtgtgaggcccgccagagctccacctgcctgaaggagtggtgctcctgtttcctatagatatctctggcctggcagctctacctcagataaaggctacccatgctacctacagacaaccaaaaaaaaa

SEQ ID NO: 655 - DNA, 443 bp, Mus musculus
  misc_feature (1)...(443): n = A,T,C or G
gaactgaggttgcttctataactggatcacgagtttgctgcacaaagtaccatgtctcgatcccggcagcccccacttgcgtgagctgtgncntccatagnanggatgtctaggaatcagatcccanagagncccgggaaggacccnancacnctgaccacnatncanacctgctgtngctgntcntngaccttnatcaaatcctatgtnaagagggcgtnttgtaaggacggaaggaanattaccctggganaagcnaatgnaanttgggctttantattgtcacaactaaggttgggntgtaaaggaanctcaactgtctcttttaagctactcaagcctgaaaaaataggcattttagatcagtctgcagtgtgggtggatgaaatgaattattatgatatgcggactgacaggaacaaagggattcctcccttgtctct

SEQ ID NO: 656 - DNA, 398 bp, Mus musculus
  misc_feature (1)...(398): n = A,T,C or G
gactgagatcctgatggcngttgtacagcgtggagcctgtccaanatgaagagacccctgcctgtgccgatgagtttgacgactttgtcacntatgangccaangangngacngacagngcatatntggggtntgagagcncctacagngagcagtgagaccttcacanangaggacaccagcaccctggngcacccctgagctgcngcctgaaggggacgtggacagtgctggtggctcaggggtgccctctgagtgcctggacaccatggaggagcctgaccatggtgcattgctgctgctcccaggcagntcngtgcttnacgctccnagngtaggtgtttgtgnagtgcgcagtgnanccttntagttcnccantcttgggctaggtgagccat

SEQ ID NO: 657 - DNA, 107 bp, Mus musculus
  misc_feature (1)...(107): n = A,T,C or G
actgaggctcagacaagagaaaagctctagaagagaccaaagcatatacaactcaatctctagctagtgttgcttatcaaatnnatgcattggccaacaaaaaaaaa

SEQ ID NO: 658 - DNA, 462 bp, Mus musculus
  misc_feature (1)...(462): n = A,T,C or G
ctgagggttccggcgctncccgtgaaaaactngaatggcactggccctgtncacccagccttgctcagggntgaccgngatnctgctgtgtgcagcgtagctgccgangtgcctgacacgagcacccaaacccatcctgcacccaccaccagtangcaaaagcnactgtngaagcctgtgcctgnagtgcccggngtgtgccgaaagaccaanaaaaaacacctcaagaaaaagcaagaatcctgaggatgtggttcggaggtacatgcagaaggtgaaaaaccccgcctgatgaggactgtaccattttgcatgggagcggctgntcacagnatctgggctatgagggcgtgctccgaaaacaagagtgtgcggnccgagcttgtgggccgnctgggccgtnggggncanatgtatcacntggntctgnctggnggccatgtactccaatggcaaaaagga

SEQ ID NO: 659 - DNA, 416 bp, Mus musculus
  misc_feature (1)...(416): n = A,T,C or G
ctgggagatgtgacgccacacaatattaaacagttgaagagactgaaccaggtcatctttccagtcagctataatgatgaattctacaaggatgngctagaggttggcagagctagcaaaacttgcatatttcaggngatatagctgtangtgcagtgtgctgcangggntggatcattcacagnaatcaaaagagacctttncntcatgacactagggatgccttgcaccttaccgaagactaggaataggaactaaaatgttaaatcatgtcctaggcatctgtgagaaggatgcncncctttttgacaanatctatctgcatgtgcaggatcagnnagttgagnccagcngattgacnttttacccgggaaagtttggctttttgagattatccgnagacaggagaacaactt

SEQ ID NO: 660 - DNA, 125 bp, Mus musculus
  misc_feature (1)...(125): n = A,T,C or G
gaagagtggcagacagaaagaanaggctctggtccaaagagagaggatgtgtctctggaacatggatgccattancaaggatgtttttaataagagttttattaatcaagataaaagaaaaacaa

SEQ ID NO: 661 - DNA, 349 bp, Mus musculus
  misc_feature (1)...(349): n = A,T,C or G
ctcgaccctgcctgaccggnctctanngaactgncagtacagttcagncaccttcancncangtgagcgcaagcgaaggcctcatggagaccggaagtcttgtgagatggggctgcagctacgacagaccttcnaggcaggcatnctcacacagctgcacccccgctctcagatcgacatctacgtgcaggtgctgcaagcagatggtgggacctacgcagcatgtgtgaatgcagccacgctagcagtgatggatgctgggatacctatgcgggactttgtatgcncatgttcagctggctttgtggatggcncancactancggacctcanccacgtggaggaagca

SEQ ID NO: 662 - DNA, 156 bp, Mus musculus
gactgaggtgtggaaatgtaaactttgctaggagaccagctgtaacagatgtggtcgagttgcagtaatgtgaattgggctagaagatcagagtgtaacatgtgtaatactccaaagtatgctaagttagaagaaagaacaggtaagaaaaaaaaa

SEQ ID NO: 663 - DNA, 513 bp, Mus musculus
  misc_feature (1)...(513): n = A,T,C or G
gactgaggttgaagctggaatttgccccttacgagcaagaagatgatgagtgacgctagtgacatgctggccgcggcgctggagcagatggacggaatcatagcaggttctaaagccttggagtattccaatgggatttttgattgccaatctcccacctccccatttatgggaagcttgcgcgctctgcaccttgtggaagacctgcgtggattgttggagatgatggaaacggatgagaaggaaggattgagatgccagatcccagattcaacagcagaagtgcttattgagtggcttcaaaatcaaatgacaaatggncatctacctgggancgganatgtgtatcaagaaaggttggcacgtntagaaaacgacaaggantccctngttcttnangcaagtggtacttgagnccattcctttntnangacangtggnccttncaactttgaatctcatgtatgtgcaacacctgtattaanncacctgaatattacatgccatctaagc

SEQ ID NO: 664 - DNA, 252 bp, Mus musculus
  misc_feature (1)...(252): n = A,T,C or G
acgtgttcgaggatgatagggaagggacgatcgggagagatttccatcgatgacattcggataagcactgatgncccncnggagaactgcatggaacccatatcagcttttgcaggggncnccctcncagnnaggnaccgagcccncagggnacncangngcccgttctattncatnccagnctactngtattacgttatggcggccggggnccnccgtgctggtgcttggcgggggggggcctgcccttgg

SEQ ID NO: 665 - DNA, 322 bp, Mus musculus
  misc_feature (1)...(322): n = A,T,C or G
gcccttgctgtcagtgcnnagtgaaangaatgcggagttganangtgagganntggctnctacttnagacngacgagctgaatgatgcccctgnttgcgcccncgnaatcanatgacagcgaggacactcccagcangctcttcggggccagaggaaacacanncttaccagatccaggaactnctgagcagnaccaggtctgtcagncccatcccanctttccagttgngccacagcctcttctgacggcacaacagttagccctttgctgtgncccggggcgatgccgnnaggccccccatccctcaatcaggcaatcct

SEQ ID NO: 666 - DNA, 348 bp, Mus musculus
  misc_feature (1)...(348): n = A,T,C or G
aatcaggaatgattctccatcccagagagatgaagtaattcaatggttggccgaactcaantaccagttcaacctctatncagaancatttgctctggcangcagncttttggataggtttttagctacagtaaaagcccatccaaaatatttgaattgtattgcaatcagctgtttttttctggctgctaagactgttgaggaagatgagaaaattccagtgttaaaggtattggcaagagacagtttctggggatgttcctcatctgagattttgagaatgggagagaattattctggataaatngnattgggatcttnacacggntacaccattggatttttctt

SEQ ID NO: 667 - DNA, 162 bp, Mus musculus
  misc_feature (1)...(162): n = A,T,C or G
ggctctggcggccactgggaaggaggagggcctgagcgcccggctgctggccctggtgggntctgntctttatcgntggnggcattatagggaagantgccttgnangggccgatgcgaaggatggcaggntggattganggatctaccacaccatgggatt

SEQ ID NO: 668 - DNA, 295 bp, Mus musculus
  misc_feature (1)...(295): n = A,T,C or G
ctttggaagaagaaccaggatgactttgagtgtgtgaccactcttgagggccatgaaaatgaggtcaagtcagtggcttgggccccatctggcaacctcttggctacctgcagcagagataagagcgtgtgggtctgggaagttgatgaagaggatgagtatgagtgtgtcagtgtcctcagctctcatacgcaggatgttaagcacgtggtttggcacccgagccaggagcttttagcttntgctagctatgatgacacagngaagctataccaggaagaaggggatgactggg

SEQ ID NO: 669 - DNA, 219 bp, Mus musculus
  misc_feature (1)...(219): n = A,T,C or G
gactgaggttgtggagcatggcctgtttgtcangcactgcacngtggaagtgtatttgctgganctgaagctctgtgagatcagtgaccccaccantgtgctaagntgccattttagcaaagcagacaccattgcaactattgagaaggagatgaggaagctcttcaacatccctgcagaacgtgaaacacggctttggaacaaatacttgagcatcac

SEQ ID NO: 670 - DNA, 519 bp, Mus musculus
  misc_feature (1)...(519): n = A,T,C or G
gtctggggagctcctgcattaagtcagactgagtcctaacaggggctttacagaaatgcactgtatgagcggccactctaattttgtgncttgtgtgtgtatcataccctcaagtgacatatatcctcatggactgattgccactggaggaaatgaccacaatatttgcattttctcgctggacagtccaatgccactttatattttaaagggtcacaaagatactgtttgtagtctttcttctggaaaatttgggacattacttagtggctcatgggacaccactgctaaagtctggctgaatgacaaatgcatgatgacattacagggtcatacagcccgcagtatgggcagtaaagattttacctgaacagggcttaatgctaactggatcagcagacaagaccattaaactatggaaggctggaagatgtgagaggacttttttagggcatgaagactgtgtaagaggcttggcaatcttgagtgagacagaatttctttcctgtgcaaacgatg

SEQ ID NO: 671 - DNA, 431 bp, Mus musculus
  misc_feature (1)...(431): n = A,T,C or G
tgaggtgatgctgggagcagaactccaaaagnccagaagcttcgtgaagctatggctgccttaagaaaatcagctcaagatgtccagaagttcatggatgctgtcaacaagaaaagcagttctcaagatctacataaaggaaccttgggtcaggtgtctggagaactgagcaaagatggggacctgatagtcagcatgcggattctgggcaagaagaggactaagacatggcacaaaggcacccttattgccatccagactgttggnctaggantanaatacagagtgaangactgacnaccaaggaccnagtntgctatccagggaaccatagtgnctatnatnaccacccttccgctnactatgctgnttgaggacanccgantggtgnccaagtacaaagatggaaatcangcctggctttatgctgg

SEQ ID NO: 672 - DNA, 386 bp, Mus musculus
  misc_feature (1)...(386): n = A,T,C or G
tggcactctcttttctgctcttaccctgcnncaacattcctttccagggacagccngntgananctnttcacggagtgntgnaccntgangcgangantttaaaagttgnggcttgaaagacaggaagatacggatggcttcctcgtctggcaacagacgatgatntcactatccccagagctgntntnaatatgatgatcaaagagactcttnctaatgtccgggtggccancgatgcccgagagctggtagtgaactgctgcacggaattcattcaccttatatcttctgaagccaacgagatttgtaacaagtcagaanagaagactatttcaccagagcatgttatacangcactggaaagtttgggctttggctcttacat

SEQ ID NO: 673 - DNA, 253 bp, Mus musculus
  misc_feature (1)...(253): n = A,T,C or G
ttcctaacatgcttgagttncgaaccagtnttncttggagatgaacnctntttngcaagagcgaggnncnnaaancnttnnctttcaggacatggaatctgaattggagaanacagaagctnagctcaatgcagnagtcagagagaaancatctctctctgcgagtaatgcttcactnggaaaaacggnttactgaattaaccagagccaacgagctactaaaggctaagttttctgaagatggtcaccaaaa

SEQ ID NO: 674 - DNA, 127 bp, Mus musculus
  misc_feature (1)...(127): n = A,T,C or G
gactgagatgggagatctttggtcagcncnncctatgaaggtgaatgcttcccaggctcgtcagtttaaaccttgtattaagcaaataaatttccctacaaacctcatacgactggaaaaaaaaaaa

SEQ ID NO: 675 - DNA, 408 bp, Mus musculus
  misc_feature (1)...(408): n = A,T,C or G
gacatggtgttactgtggagtttatgataatgccttaaacaatgacaactacaaccttgctagaggcttcaattaccaccaaggacctgagtggctgtggccaattgggtattttcttcgtgcaaagttgtatttttccaaaatgatgggcccagagactgctgcagaagactgtatngtttggttaaaaatgttctttctagacattatgttcatcttgagagatccccatggaaaggccttccggagctcaccaatgagaacggccagtactgccctttcagctgtgagacacaggcgtggtcaatggctgntgttctggaaaccctctatgatttatagttggctcataaatagtaattgtgtgaacacttgacattattgaatgcaaggtcataaagaaatgta

SEQ ID NO: 676 - DNA, 433 bp, Mus musculus
  misc_feature (1)...(433): n = A,T,C or G
actgagtctccatgaagtgccaggagcacccgtgcgtgtcccctgtctctaatcatgtgtatgagcggcgactcattgagaagtacattgcagagaatggcacagatcctatcaacaaccagcctctctcagaggagcagctcatcgacatcaaagttgctnacccannncgacccaancctcnctccgccaccancntcccagccatnctgaangccttgcacanatgagtgnnatgcaggnnatgctgcacaggtncactctncgccagnaactgctgacaacccggcaggagctgnnccatgctctgtaccaacacgatgcttgcctgccgagtcattgcccggctnacnaaagaggncactgcngctngagangctntggctactctnaanccacangctggncttatttgtacctcaggctgtgccaa

SEQ ID NO: 677 - DNA, 111 bp, Mus musculus
aactgagatccggacatggtggcagtgttggaagtgatctccagtctggagagataccccatcaccaaagaggcactggaggagacacggactagggaagctcatcaatga

SEQ ID NO: 678 - DNA, 495 bp, Mus musculus
  misc_feature (1)...(495): n = A,T,C or G
gactgagagactgtgaatcctgcaggatgactgaatgaacccttgattcacggacatgccggaggagttcaacatgggcacacacgactgtcacagacggcagattcngntnctacttgngggngnngaaganntagcctntctacangttncannnngatnnaagggagnacntngntgntggcggaacacctgacttccaggcaactagggtgaggatcttatacccatacccactgtgacacaaccattccaaccaggtcacacctattccaacaaggccacacctccagatggtgccactccctggtccaaggatatacaaaccatcacattccactccctggtccccatagacttgttcaaaaacatgagtctatgggggccatacttaaacaaagcataatgcaaaatgcatttagtccaactttcaaagtcctcatagtctatagcagtccgcaacaacattaaaagtccaaagttcaaaaaaaaaaa

SEQ ID NO: 679 - DNA, 423 bp, Mus musculus
  misc_feature (1)...(423): n = A,T,C or G
gactgaggaagccagagctatgcagttctccagctcatccaggacatcagatgagaatgtggactatttgttcaaggtcatccttattggggactccaacgtggggaagacatgtgtggttcagcatttcaaatctggagtctacagtgagtcccagcagaacaccattggggtggacttcaccgtgcgctccctcgagatagacggcaagaaagtgaagntgcnagggtncacacacggnaggncangagcnctnccgnaccatnacccaaagctactaccggagtgcccacgcagctatcatcggttatgatctcacacggcggnccacattnaaatctgtccctcactggatccacgagatagaaaaatatgggagctgccaactngntcatcatgctgatcgggaacaaatcagacctg

SEQ ID NO: 680 - DNA, 166 bp, Mus musculus
  misc_feature (1)...(166): n = A,T,C or G
taagagtgagctggtacagaaagccaaactggctgagcaggctgancgctatgatgacatggctgagctatgaaggccnnaacagancagggacacgaacttttccaatgaagaagaggaattctactctnttgttgcctacaanaacggtggtaagggggccccg

SEQ ID NO: 681 - DNA, 206 bp, Mus musculus
  misc_feature (1)...(206): n = A,T,C or G
tcgtacgctctcaaccagagactatagcatgtgctngnatctaccttgcnngtagagcacttcaaattcccttgccnactcggncccantggnttcttctgtttggnaccacagaagaggagattnaggaaataatgcatagagacgcttagactntataccatggaaaaagcctaactatgaatgagctggaaaaggaagtagaa

SEQ ID NO: 682 - DNA, 277 bp, Mus musculus
  misc_feature (1)...(277): n = A,T,C or G
aggccgaaggactgaatnttccataatacatncttaaaaccttcaagaatggttctagcagatcttggaagaaaataacatcagcattacngctcattgagcantgccaccattatcaatgaagaggtatttaaatngctattgctgaaagaaagtatgtccagcatttattggaagcagatgttattattaacntagggaagcacctcagaaaaatgttaaagcctggcatttggttcttgaaaanatggccttcngggggggggccaaaaaaaaa

SEQ ID NO: 683 - DNA, 151 bp, Mus musculus
  misc_feature (1)...(151): n = A,T,C or G
cgcagttactcttttcatgtataaagcagggatgctgnatctacctcacacgggtactaaaagcaaaacacgggaatccaaagccctggggcacatgcccgacatctgaaaagctggnggtttctcccacctcccttccgagtgcatgctg

SEQ ID NO: 684 - DNA, 471 bp, Mus musculus
  misc_feature (1)...(471): n = A,T,C or G
gatggaagcgtccagactccggcagaaggcagaggagctggtcaaggacagcgagctgtcaccaccgacatctgccccctccttggtctcctttgatgacctggctgagctcacaggacaggatacaaaggtccaggtacatcctgctaccagcactgccgccaccaccaccgccaccgccaccacgggaaactccatggagaagcccgagccagcctccaaatctccgtccaatggcgcctcctcggactttgaagtggtccctactgaggagcagaattcacccgaaactggcagccaccctacgaacatgatggacctggggcccccacccccagaggacaagcaacctgaagctccacctgcagcgcctggagaccacccttagcgtgtgtgcagaggagccagaccacaagccagctnttnacccacctgggccgcatggcctntagtttaaaaaaggtgggcccc

SEQ ID NO: 685 - DNA, 217 bp, Mus musculus
gactgaggaaaggcctggaagaagaggccaccaaacaacccaaccctctctccagtcccttgatctactcgtcgcctgcatctggagacccaatggcccaagcctactggcagtgctacccctggcttattctcctctgtgcatgtgcctggagctacccagagcccaagtaccttggaagggaagatgtgagaaactgttcaaccagccctgaggc

SEQ ID NO: 686 - DNA, 168 bp, Mus musculus
gactgaggctccagccttcccccagccctggttgaaatcctcagtggttattcatgaagacgcacccaccatgttttatgggactcattgcattactagtgtaagataaacctagttgtattcatttcttgtggctgatgtaataaatttaaaacaacaacaaaaaaa

SEQ ID NO: 687 - DNA, 408 bp, Mus musculus
  misc_feature (1)...(408): n = A,T,C or G
ccagcacctaaagaaggtcctatgccagttntggcacttatgtagtgtttagaggcaggcagcagggggcctatngtccatgtggacacacacttcatccaggctgncctactctcccgcccttgggcttccttggcctntgatganttnggctgatggccaacacctcacagccatgagaccgagcaaaaagagactggggantttctcagctggctttcgcccttcctcanaggagtnggcccttgggaccttgccctgaaagctggtgatcgctgnnaaccccaccatncctccccaanggccttnatgggcacgggcccatgatcttnacctggtttnagccctgagcgccntgagtgcccgggcgncngganctacctggtttttagggaggattcttggatg

SEQ ID NO: 688 - DNA, 452 bp, Mus musculus
  misc_feature (1)...(452): n = A,T,C or G
acacagcacctgcctttgagggccgcncggagatctgataatgccctcaaggctggntgnctatgctgntgaaagctaatgaccatgagctggctcaggccatnctganngnagccagcatcaccctgcctcatggaacnctttnnnaatgctatagacgagcnggccaaccgcttataaacctgcaccatctattgncctgtccccacctnntngaacttgctgctgcancacacaggaggagganagcctgtatcctccccnagcccacccctagtgngcgccgngaattcccactgaaagtgcgcctcttccacaggcaaggacgngaggntnaatgccagcctgcctgacaccggggggggcaactnaagaggcagctggcccagccnggagggcatttgagccnttcctggcananatggttnttttttgggaaagctgcttacaac

SEQ ID NO: 689 - DNA, 428 bp, Mus musculus
  misc_feature (1)...(428): n = A,T,C or G
ataggagcagaatgttcagcagaggcctacatacnnaanactctcgagggaacaggcttgccatttcacagttgatgagagttctgtggctgggaagagagtcgacagtaacgaaacagtctcgccatcttcactgccttcatctgtgagaactgctgggctaaagcccgaaactggacccctcatgttttggtcacagcaaaatatcacagaacagggttactctgacaactctctgtccagagaacttacacttctgtctgcctgcaatgctgatagttgtcagatgccaaaactccactgccaccgttcaccaccccctttgccaccaaagaaatactctacagccagngcaccaaggttagagagagttggacttagccctgatgtcggggttaccgaggcgtttaacactaatccctccagcc

SEQ ID NO: 690 - DNA, 306 bp, Mus musculus
  misc_feature (1)...(306): n = A,T,C or G
gaaagcttgaggccttccccanctggcaaaaatgaagaaacagatccaccccgttcctcctcaggggngggnnactttntctttancaccatttcccacaatgccaagccacacgggnccccttgccntnatcccnaccttatgattctttatccttggcttgggaanaacagccttccttttaagcctcccccaaagtcccctctttggaacccaaggtttnaagggnggggggggntcctaaaangtntntccnaagcctcataaactcttaatggaaactcaggaacccttggattggacccc

SEQ ID NO: 691 - DNA, 474 bp, Mus musculus
gtgtatgtgtcttacaactatggaaaatcattcagtaagatttcagagaagctgaactttggcgtgggcaacaacagcgaagctgtgatctcccagttctaccacagccctgccgacaacaagaggtacatctttgtggatgcttacgcccaatacctctggatcacgtttgacttctgcagcactatccatggcttctccatcccattccgggcggctgatctcctcctccatagcaaggcctccaacctcctcttgggctttgacaggtctcaccccaacaagcagctatggaagtcagatgattttggccagacctggatcatgattcaggaacacgtaaagtccttctcttggggaatcgacccctatgatcagccaaacgctatctacattgagcgacatgaaccctttgggttctccacagtgctccgcagtacagacttcttccagtcccgggaaaacccagccggg

SEQ ID NO: 692 - DNA, 126 bp, Mus musculus
ggaagaaagctcgcacatttgacttggaggccatgttcgagcaaactcgaaggacagcggtggaaagaagccggaaaacactggaagcaagagaaaaagaggaagaaatgaacagaaacaaaaaaa

SEQ ID NO: 693 - DNA, 283 bp, Mus musculus
  misc_feature (1)...(283): n = A,T,C or G
agagaagccagccaaagaatgtctcgtggtattccagccgattcttcccacttccgatgggttaagccaccgttactgattcgcatttggtgtttgctattggactgaacctgctgatatcctgacatgaagcttagagtcgatctaaagaactacttctaaacaggtccacaatgcccttgtcctagtaaccgtatctctcccctacctctggtcggtgctctagaagggagattgatgcattgnagaaccccaaataaaataggctttggaaaaaaaaaaa

SEQ ID NO: 694 - DNA, 434 bp, Mus musculus
  misc_feature (1)...(434): n = A,T,C or G
ccaggtcccagtgataagaaatcagaactactgccagctaagtggaacagcaataaagaactgtatgccctccggtatgagtctaaggatggagccagaaaactccttctgaaagctgtctctgtggagaacggcatgatcatcaacgtgctggaacttggcacacagcaggtggcagacttgaccctgaacttagatgattatattgatgcagaagacctgagtgacttccacaggacctataagaacagtgaggagcttcggtctcagatcagatctggaattatcacacctatccatgagcagtgggagaaggctcgtgcaaacagtcctccgagggagttcccacctgccactgccagagaggnggacccactccagatttcctcacaccgncctcatactancaggcagcctgcttggcgtgatcccct

SEQ ID NO: 695 - DNA, 405 bp, Mus musculus
  misc_feature (1)...(405): n = A,T,C or G
ggaagaaagctcgcacatttgacttggaggccatgttcgagcaaactcgaaggacagcggtggaaagaagccggaaaacactggcggaaccagtatttggaaggctgggccagaggtggctgtcaccattgcttgttgacccagacagtatagaacattgtgacacacactgatgtggctgctgctgttgaatccctgctttaatgagctgtaggagatacttaatgaagccttacttccagatggcttttccagatggttggcagttctaggaaccaccttagggtctccagagaacactggcagtgngctttttttggctctaactcagatctctcttgtactagaggacggtctggagtctgggaacagctggatttaaatcaagtgctgtgatagtacttg

SEQ ID NO: 696 - DNA, 101 bp, Mus musculus
ggaagaaagctcgcacatttgacttggaggccatgttcgagcaaactcgaaggacagcggtggaaagaagccggaaaacactggaagcaagagaaaaaaaa

SEQ ID NO: 697 - DNA, 433 bp, Mus musculus
  misc_feature (1)...(433): n = A,T,C or G
gtcggatcataaggcagccttctgtcagtctgctctgccccttccctcgaggagcgccctgccttgctgactttctctgcctctggcttcctgtgataggtggtgtcattcccctccaacctgagtgctggcaggtacactgctggccaccagcagatgcccatgaagatgctgacaatgaagatgctggccctgtgcttggttcttgctaaatcagcctggtcggaggaacaggagaaggtggttcatggaggcccgtgtttgaaggactcccaccctttccaggctgcctctacacctcaggtcacttgctgggngggggggcctcattgacccacagngggtgctgacagctgccactgcaaaaaaccgaatctgcaggngatcttggggaaacacaaactacggcaaacagagacttttcaaaggcaaa

SEQ ID NO: 698 - DNA, 436 bp, Mus musculus
gtggggtctttcacccgtagccgactcttcaccttcccgtaaccgcgtctcttaccggtgatggcggtgaactgctggattaaccccttcagcgccgacgacgccgcggagcccccgtgggcagccatcttaccgccgccgccgccgccgcggaacaacaacacagacacacacggggctccctcccccactccgcggcgtgctgtgcgcaggcgcactggcaggcccgaagctccgccctctccctgtctgtgcgcgttttgcgcaggcgtactctccgcgagccgccgctttggtttgaagcctgtgctgctggctggtagctattctttgaaacatttggtgccttctctgaattccggagatatcctcctaagtaccgcattgaatcccaacatcccaaagaatcttcggacctgaaataccaaactcttgg

SEQ ID NO: 699 - DNA, 177 bp, Mus musculus
aatgggagctgactgacatggtggtgtgggttactggagcgtcaagtggcattggcgaggagctagctttccagttatctaaactcggggtctctctggtgctgtcagcccgaagggcgcaggagctggagcgggtgaagagaagatgcctggaaaatggcaatttaaaaaaaaaaa

SEQ ID NO: 700 - DNA, 352 bp, Mus musculus
  misc_feature (1)...(352): n = A,T,C or G
gttaatcctgccatacgaaagatttattaagggagaagaagataagcctctgccgccaatcaaggcctcngcaaactngaatgccctgcgaggccnaaatgagantnaancaaaagtctcgggaaacanacgtancngngnggactacgcccccttttgagnaagaaaaaactggctaagtccaagctgaattggcctcgctggccatttaaatggcgcgccctcgaggaattnggatccggcgcgccatttaaatggccgctatggccaattcttttggtttttaccctggaagaaatactcataagccacctctgttatttacccccaatcttcacaagaaaaactgtaa

SEQ ID NO: 701 - DNA, 434 bp, Mus musculus
gtttattgaaaatatggcataacagattaaacagctccacgtggtgttttgaaattcatgccaactgtaggctgagtgacctgaaggttagagagactgccaaagtccaggagcttcagcatctccttggtgtcgggatccacctcaatgatctcctgatctagggctgagacctcaggaacgtagttatctctcctctctctctcttcttcctgcaacttgatagagatgcctctcacaggccctctctgaatccgcttcatcagatgcgtgatatagccggctatcttgttccgaagcttcttgctggggatgatggcgatctcctcgcacacgcgcttgttggtggtgaaagcattacccagacgcgtgtagtacttctagatgatgacccgggcagccttcttcacggtcttggtgcgaaccagcctttt

SEQ ID NO: 702 - DNA, 363 bp, Mus musculus
  misc_feature (1)...(363): n = A,T,C or G
gcccgtgggattgacgtgcaacaagtgtccttggttataaactacgatctacctaccaatcgtgaaaactatattcacaggagtcgatagcggcagttgatgacgagatggcactcagaaacggcgttgacgtaatttaggacgtggaancataagcgaaacagcacactgtttgaacaaagagcgagtcgaattggcagagggggtcgatttgggaggaaaggtgtggctataaactttgttactgaagaagacaagaggattcttcgtgacattgagactttctacaatactacagtggaggaaatgcccatgaatgtggctgacctaatttaatccctgggatgagatagttttgaatgc

SEQ ID NO: 703 - DNA, 333 bp, Mus musculus
atgatttcctgctaaatgggagcctgggactgggagggctcctcctcctgcttgtcatcatcctgttcatctgcttgtgccggttcagccagagagtgaagagactggaaaggaatgcccaggtctcagggcaggagccccactatgcatctctccagcagctgccagtgtccagtagtgatatcacagacatgaaagaagacctcagcactgactatgcctgcatcgccaggagcacacccacttgagtgcccccaaaagctctcctcgccctgggtgcgtgctcagtcacactatggtcatctagtaaagtctggttccccacttctgaaa

SEQ ID NO: 704 - DNA, 528 bp, Mus musculus
agtttgtgagaagcttctataatggtctgcttggctctcgtcagagctgcctatgagcacatctacctggtgaggaaggtcagtcatgcctgcagatgccatcaacatcgggcttggagctcaaagccagctgcatcacagtctgctgtccagggtgctccaggcagtgtgctggagatacttggtaaatcctaccctcaggatgaccacactaacctcacccagaaggtcctttccaaggtgggcaggaacctgcacaaccagaagttccaccctctgtggctgattaaggagcgggtgaaggagcacttctaccagcagtatatggtgcgatccaggacacctctattctccgtctatgaccagcttcctccagtggtcaccacctggcagaactttgatagcctgctaatcccagctgaccaccccagcaggaaaaagggggacaactattacttgaatcgggcacacatgctgagagcacacacatcagcgcatcaagtgggacttgctgcatgcgggacttaa

SEQ ID NO: 705 - DNA, 513 bp, Mus musculus
  misc_feature (1)...(513): n = A,T,C or G
ggccaagctcagacattacggagcccagagccagaggcgatggagctgagccaccgacaaggtacaaccacgttgacaaggacacacccgaacgacaaggagggccagcaggacatgaactccttcagagctaaccatagctccttggacaactccaaatttaagtaccatgcaaggctctcccagagccccttaggcagctccctgggccagggctacctggagactccacctctaccaccaactcctacctgcaggacttctctgnctatgaactcccatcctgaggacctgaagaaggggcatccagatccagctcgagagatgccagggagaccttccgagagggatgtgtcgggggaggaaggncaagacttcaggtcccaaaacaaangacaggggcctttgaccaagaaggacagggtcnttcttgaaacatccgccccaaanttgggagcaagatgggggaccaactgatctttgaggatnagctcgacaggccattggtgaaaa

SEQ ID NO: 706 - DNA, 518 bp, Mus musculus
  misc_feature (1)...(518): n = A,T,C or G
aaaactagaaaagagaagacaaaatcaaagaccgaagaagcaacagaaggaatggaagaggccgtttcctccaaagcaaagaaaacaaacaagaaagggccttctgaggatgatgtggatccccctaaatccaggaaggccaagaagcaggaggaggagcctcaagacgacactgcttctacaagcaaaacctccaagaagaaaaaggagccccttgagaagcaagcagactctgaaaccaaagagataataacagaggagccttccgaagaagaggcagacatgcctaagcccaagaagatgaagaaagggaaagaagcaaacggagatgccggagagaaaagccccaagctgaagaatggactctcccaaccttccgaagaagaggcagacattcctaagcccaagaagatgaagaangggaaagaagcaaacggagatgcttggagagaaaagccccaagctggaagaatggactntcccaaccttccgaagaaaaaggtagacattcctaagcc

SEQ ID NO: 707 - DNA, 278 bp, Mus musculus
tgcccccccaggagacaaagagcctcatgaaggcttcctacacgcccgaggtgatagagaagtcggtgagggatgtggaacactggcacgggaggaagacggatgacctaggccggtggcaccggaagaatgctatgaacatgaacttgcagaaagcgctggaagagaaatacggagagaagagcagatccaaggccaagtagttgaaaggacattgggaggaagcttgagaatgttgccaataaagaaataaaggctgtggtctgcaaaaaaaaaaa

SEQ ID NO: 708 - DNA, 423 bp, Mus musculus
tgctgaagacagcgcagaatcaccccatggtaagcactctgggccacctcccttcatgtgcccacagctcagtcttttgcagttcatgattgaaggcctggcaagatggctcagtgggtaaaggtgcttgctgccaagccgaaggacctgagttcaatccccaggacccatgtgatgatggaggggttcatgacttccctgaagttgttctctgccctgtctgcacacagacagaatggcaaatgcaatacaaaggaaaacgttcacaaagaaatctatagccggcatggtggtgcacgcctttaatccccgcacttgggaggcagaggcagcgaatttctgagttcgaggccagcctggtctacaaagtgagtgccaggacagccagggctacacagagaaaccctgtctcaaaaaaaaaaa

SEQ ID NO: 709 - DNA, 198 bp, Mus musculus
  misc_feature (1)...(198): n = A,T,C or G
cttgttacctaaaaccccgggcagaggaggagaggagantagcagcggaggaaaagaagagacangggngggggggncaatttngtttggatttgacggggcgcncannntgacanaanngncaagtgaggcgtcagcgagcttgcttttctctagtcgttgagaacgaataaagcttcattgtgcgaaaaaaaaaaa

SEQ ID NO: 710 - DNA, 277 bp, Mus musculus
gtgtataggtgtgaaccaagtgcacgtctagttcccttggaggccagaaagaaggcggtgtcagatccccaggaaccatagttacagatggttgtgtagtctcccctcccccagcctgaatcctgcttgctcaggggtggagcttcccgctcaatcgttcagccatgcccactgctggaagctgccgctttgctgttcggagccggagccgtacacgtggtcaccctgctattggaccccaagactaattggcgggaatcggccccttcccctgctt

SEQ ID NO: 711 - DNA, 410 bp, Mus musculus
  misc_feature (1)...(410): n = A,T,C or G
gtgtataaggtgtgaaccaagtgcacgtctagttcccttggaggccagaaagaaggcggtgtcagatccccaggaaccatagttacagatggttgtctcctgtagctcaacctggcctggaattctctatgtagctgcaagtgaccctgaccttctgagtcttctctaccttccagtgtagtctcccctcccccagcctgaatcctgcttgctcaggggtggagcttcccgctcaatcgttcagccatgcccactgcnggaagctgccgctttgctgttcggagccggagccgtacacgtggtcaccctgctattggaccccaagactaattggcgggaatcgggccccttcccctgctttataattgcgtgtggaacagtaaaattgagctttgatcagaaaaaaaaaa

SEQ ID NO: 712 - DNA, 521 bp, Mus musculus
  misc_feature (1)...(521): n = A,T,C or G
gggtggtaagggtggtggaaacccangtgccncgtcttaagtttccctttgggaaggcccagaaaagaaaggcgggnggtcaagaatcccccaggaaacccataagtttaccagnatgggttggatgttttaaaccctcaagacagggatgggggcttgggggcttgggccaacaacttgctcaacattcttctgagtctgacgggctggtggagtgtgagcgtggctcctaaatttcttgggtaggactgaagaagcacttttaagatgtggaaactcagagaggatgccactgttttgaactattttgaaggcacagccaccagagctttaggtcagaagaggtccttggaactggaattacaggtggctgtgagcctccagggacagggtattttatcacagcaacagaaatgaaaccaaaacaactccacaataaaattcttgggtttcttggggccaagaagatgacacgataaggataaagacactggccaagcatgaccttcagagtttggccc

SEQ ID NO: 713 - DNA, 508 bp, Mus musculus
attgactcgtggtgcaaagacaacagctatgtgatcgctggctattaccaagctaatgagcgtgtgaaggatgccagcccaaaccaggtggcagaaaaggtggcctccaggatcgcagagggcttcggtgatgccgcactcatcatggtggacaatgccaagttcacgatggactgcgcagcgcccacgatccacgtgtacgagcagcacgagaacaggtggcggtgcagagacccacaccacgactactgtgaagattggccggaggcacagaggatctcagcttcactcctggacagccgctcctacgaaacactcgtggattttgataatcatctggacgacattcggagcgactggacaaacccggagatcaacaaagcagttctgcacctgtgctagacaaggaccacactacagcgaagacggaagcatatttttacatttatagaggaatagtaggttagccggaagggaagctgcaagtgtgccttggagggggtgccgg

SEQ ID NO: 714 - DNA, 532 bp, Mus musculus
  misc_feature (1)...(532): n = A,T,C or G
gactgaggaatagaagacatcttaagtaggggctcaagagctggatagcgtcatcaagcaagggtacctggagaagaaaagcanagatcatagcttctttgggtcggagtggcagaagcgctggtgtgttatcagcagagggctcttcctctactatgctaatgagaaaagcangcagcccaaagggaccttcctcattaagggctacagtgtccggatggcccctcacctgcgaaaagattccaagaaagaatcctgctttgaactgatctcccaggataggcgcagctatgagtttacagcntctagtncagctgaagccagagactgggtggatcaaataagtttcttgttaaaagatcttagctccttaaccattcccttcgaagaggaggaggaggaagaggaggaagaagagaaagaagaggaanaaatgnacaatgacgtcgatggctttgactccccaaggtctggctcccagtgcagagccatggccttgcctgagcctactgagaaagaggaggacatttat

SEQ ID NO: 715 - DNA, 326 bp, Mus musculus
  misc_feature (1)...(326): n = A,T,C or G
gcttctgcaaagagagtcccagccttgccacgaggcggaagtgactgagctgccacatccttcanngtggagacatccagaagacagacaacttggccagagggaaatgaaggtgaagagaagccgaggagattggaatctagtgccgaatttgcatccgatgccgagcctaggangtcanagaagacctagggtacaaaagaggaagnagcaaaggggaagntcttccttctgatcccctctattaggtggaggacctcggnggngagacctccgccttctgctctgtatcgctgcacttgantaccataagggggagccctacc

SEQ ID NO: 716 - DNA, 210 bp, Mus musculus
gccgcccgtgcgtggattgccacgcatttgaattcatgcagcgcgcccttcaggacctacggaaaaccgcctacagcctggacgcacggacggagaccctcctgctgcaggctgagcgccgggctctgtgtgcctgctggccagctggacgctgaagatctttggccagtgatattccttgtcaataaatgcctgctagcaaaaaaaaaa

SEQ ID NO: 717 - DNA, 495 bp, Mus musculus
aaaagcttcactggcctgaacatgaacttgctaagaagtttgttctaaatgcagaagaggcattgatcactgacagcaaaagaagcttttcgagtttgtcctctgggattctcaaggacactttcacaactggaaccagtagttacaatgttctactacagagcaaggaggagaaaaagcaccgttcacaaaagcggttttcctctgccagctccaaacaacataggaagcccagcaaatctcctagctcctctcatagcaaagatcctagcaggatgacagccctggtgcctgggacaggcgctggcacttggtactgcctagacgggcagtctgctgtttttgttactagctcagtaccaagtcctgtaaaggtcacccgtgatatctctattacaggaagcggcctagcactgccacctaaaccaaatagcaagggcaaacgacgcaacctgacccggcttccaaagccaaagcaacagcctcagctgtgta

SEQ ID NO: 718 - DNA, 147 bp, Mus musculus
  misc_feature (1)...(147): n = A,T,C or G
atgagaagacgagaacaaagaaagttgataatgaactcaaccctgtctggaatgagattttggaatttgacttgagggggataccactggactctttcatctttccatggtggattgttgtcaanagattttgagacaattggacca

SEQ ID NO: 719 - DNA, 303 bp, Mus musculus
  misc_feature (1)...(303): n = A,T,C or G
gctctgggtgctctaggatgagcgctacacatctgtggcttttgggtataaagactgtgcggcttattaccaagccgcaagcccaagaaccaaggtagatgccatccacacccctgttctctgcctcaatgcagcagatgaccccttctccccagtccatgccttccctctgcaggctgcccagaagtctccctatgtggccctgctcatcacaagctcggggtggccacattggcttcctggaaagggctgatgccctggcaacactgntacatgaaccggggactaaattaatatgcccaa

SEQ ID NO: 720 - DNA, 391 bp, Mus musculus
gtgttttgtctgcatgtatgtcagtgctccacaggcatgcttggtgcctacggaggccagaagaggacatcaggtcccctgtgagttacagatagctgcgagctgccacgtgggtacttagaattgaactcaggttccctggaagaacagccaatgctgttgacttggtcttgcctcaccacctgtctttcctcagtccatgagtgttgccttagcaaaactccacgtgcgtctttatctgacatcgttctatcggtcagagtctttggaagaggcaagaagccgcaggaatctctccatttatttctctctatgagtcttgaccattggagctcaacaacgcatgtaaagtgaaccaatacatgtgtgtcattatcgaagaagaaaaaaa

SEQ ID NO: 721 - DNA, 525 bp, Mus musculus
  misc_feature (1)...(525): n = A,T,C or G
gtaaaatatgaagttacagaggatgtatatacttccaggaaaaaacaacaccagaccatgatgcattatttttgtgcactaaatactcttcagtacaagaagaaaatagctttgttagaacctctactgggatacatgcaggctcagataagtttctttaagatgggttctgaaaatctcaatggacaactggnagaattcttagctaacattggaacaagtgtacagaatgttcgaagagaaatggatggtgatgtagagaccatgcagcagacaatagaagacttggaggtagccagtgaccctttatatctgcctgacccggatcccaccaagtttcccatcaatcgaaatttaacccgaaaggctggatacctaaatgctaggaaatcttcaatattgcaagcagagagtaaaaaggaccatgaagagtggatctgtacaataaacaacatatctaaacaaatatatttgagtgaaaatccagaggaaactgctgcacgagtgaatcaagtctgctctaga

SEQ ID NO: 722 - DNA, 324 bp, Mus musculus
  misc_feature (1)...(324): n = A,T,C or G
attttatcatgtgggcatcctgctgcaactggttctgcttggatggacagcctgnggaggcaccaccaccacagggagccaggacacaagcctattccaaccctgggtacagctccttcccttcccccgcaggctcggaacctagctgcaaggcctgcggggtccatttcgcaagcacaacccggaagcagacctgcttggactgnaagaaaaacttctgcatgacttgntcgagccaagaggggaatgggccacgcctctgccttctctgcctacggttccgagccacggccttccagcgggaggagctcatgaagatgaagg

SEQ ID NO: 723 - DNA, 352 bp, Mus musculus
  misc_feature (1)...(352): n = A,T,C or G
cctacaaggagaagatgaaggagttatctgngctgncgctcatttgctcctgcttntactntcancctcacccgacaccatntacnagcatggcaanntgganntaaccangctgaacntgntcaaaaggtgggnactacccaccttttgagcaagttcagcctggttaagtccaagctgaattggcctcgctggccatttaaatggcgcgccctcgaggaattcggatncggcgcgccatttaaatggccgctattggccaatttttttgctttttaccctggaangaaatacttcnataagcncctntgntatttanccccaatcttcacaagaaaaactgtaatctgtc

SEQ ID NO: 724 - DNA, 288 bp, Mus musculus
aatccagcaactatcacgagaatactccttagccacttcaattgggataaagagaagctaatggaaaggtactttgatggaaacctggagaagctctttgctgagtgtcatgtaattaatccaagtaaaaagtctcgaactcgccagatgaatacaaggtcatcggcacaggatatgccgtgtcagatctgctacttgaactaccctaactcgtattttactggccttgaatgtggacataagttttgtatgcagtgctggagtgaatatttaactacaaaaaaaaaa

SEQ ID NO: 725 - DNA, 108 bp, Mus musculus
  misc_feature (1)...(108): n = A,T,C or G
ccggctggacccccattatccngggggggggggatcgaaacttgggcaanggaggctccaccccctcccantgatcttctgctccncatnccggaactctacccgctc

SEQ ID NO: 726 - DNA, 457 bp, Mus musculus
  misc_feature (1)...(457): n = A,T,C or G
gtgacggtgttcgagtgaccatgncatagcgccttgactgcccgacaccnngtaggggtgcaggactttgtcctgctggagaatttnccagnagaggctgccttcattgagaacctccgccggcggttnngggagaacctcatttatacctacatcggtcctgncctactntnngnncaatccctaccgagacctacagatctacngcccggcatcttatttnaacgggnnncgtgntagnnagtannntangacngtnccancctcatttngttngcacnggcttgacactgtataccgggcacttcgtactgagcagtcgagnacccggcagtgatgattntctgggnngagagcgggggcaaggcaagacagaggcacacaagaagggagggnaagactttgcctnagtanganagacttgaagcaaatcctaaagtgaagccccgcccctcag

SEQ ID NO: 727 - DNA, 478 bp, Mus musculus
  misc_feature (1)...(478): n = A,T,C or G
gaactgaggaggctgtgcataggtgttctgcagaccatgaactgatcactagtccccagacattcatgagcacagtgtgaggctcctgacccacntccaagntctctnngnnctctntgactaagcnacctccngngnntacnnctanangccacgatncnaatcctgccntntngttactgnnnttgcnaatagagacctttcacatgtnagaggtgtaaccngacaanaaacggctncgancggagtgggnngatcgatatggaantgagatnctatttttntcaaanactatngcaanantgntcgactnagncggccccggactgggacaangtgcntgctcaagtacacccgagtacaacgagccacaacgagagccgcaccaaatgctgacatcatcgagaccctgagggaagaagggttttcaacgggctgtgacagcccagagccggatggggaggactcactggaatta

SEQ ID NO: 728 - DNA, 389 bp, Mus musculus
  misc_feature (1)...(389): n = A,T,C or G
ggagatgtctgcttgnccatgactggctgncttcgnggactgaggntcatacgtgccngnaaacaatcctaangctgccgtncctannaatcacatcccangctagactngaccntcanacctgtnaacccanacgggccanaaacaagcttaccagtnagtttggtctcaggancaggngtgtgagcccttggngctggggctattggattggcctacctacatcctcaacagtctccactctgcctgaatgatcctgagggtatcagaaagccctacactgttggtacctgggccccaacatactgtgaacggaaatgggccttctttcttttcctataggttatttgctggcagttctattaaacttgtttctaagagaaaaaaaa

SEQ ID NO: 729 - DNA, 337 bp, Mus musculus
  misc_feature (1)...(337): n = A,T,C or G
gaactgaggtcgtctgtatnacaacataaacttccagcgaaaatcagttgtgggatttgtggagctgactatatttcccacagttgccaacttgaatagaattaagttgaacagtaaacagtgtagaatataccgagtccggatcaatgacttggaggcccgcttttatttataatgatccaaccttagaagtttgtcacagtgaatcgaaacagtaagttgggcacgttaagaagctgctgtttgtacgtcatcctgtcactgttttcagagattgtttttcaacaaactttcccattcttattgtaataaagctatgtgttgtataaaaaaaaaa

SEQ ID NO: 730 - DNA, 362 bp, Mus musculus
  misc_feature (1)...(362): n = A,T,C or G
gaactgaggaccttggcaccatggttaccttgaaccgatatgcaacgtgacctcaaggatccagctactggctgcatcaagcanactgctnnntcctgctncaagatggaatgaancnactnttctgnttatgaanagaancctgnntgaggcnaacgaaccnttganncactgcaccgcctnncagncgctccanaccactcatntcannaagctcagcttttttntactccaggnttaangcagcattancacttcattnntattctgcttttggaaaagcagtgatnagatgaacatttgatcttcnctgtttatcatcanttntttactttggaggananagcttggaagataagcca

SEQ ID NO: 731 - DNA, 434 bp, Mus musculus
gaattgaggctgcttctgggacaacggccacctgtaccgggaggaccagccctcgcccgcgccgggtctccgctgcctcaactggttggccgcgcaaggcagccgcgagtcgctcaccgagcccagcccgggcaaccacaactactgccggaacccggaccaggacccgcgcgggccctggtgctacatcagcagcgagaccggcgtccctgaaaagcggccctgcgaggacgtgagttgcccagagaccacttcccaagcaccaccgccatcctctgccatggagctggaagagaagtctggtgcaccaggtgacaaacaggcacaggtgttccctcctgctaacgccctaccagccaggagtgaggcagccgaggtgcagccagtgatcgggatcagtcagcttgtgaggatgaactccaaggaaaaaaaaa

SEQ ID NO: 732 - DNA, 515 bp, Mus musculus
  misc_feature (1)...(515): n = A,T,C or G
gaactgaggtgcgatgcctctgcctctggcgttgtgaggccacttgcgagtatttgaaatgctggttaattccggactgagtggactcacctgtgacctggaaaggggcctgaatgagccgntgctgaacgcccctgagcagctcttncatctccttctggatgttgcagacgacctcctccatcaaccctacatcagctatccccttccagaacatcaaagtgtcctctggcacaaaccggatgagacagaaggggagcagacacattacccagaaacctcagcacatggctacaacgttcatttccattccaaaggattctgtgttaaaacatgaaagactggctgcttccacaagttggacggttgctgggggtgagaggagatgatcccagttacatcagtgctcatgtttatgaggaaaaccagcagaggtaacaaaggggtctataaccctgagctgtacgggaaagtacctgaaatctcttcagactctttctttataccctcctctg

SEQ ID NO: 733 - DNA, 142 bp, Mus musculus
  misc_feature (1)...(142): n = A,T,C or G
gaactgagagttctgatggtcagtccacagnaagacggccttgcgtctgtgggtgttggagactccattctaaagatataaaaagtgaaagaggngaaggccacctntctacctcaatacgantcattatttgttctaatga

SEQ ID NO: 734 - DNA, 475 bp, Mus musculus
  misc_feature (1)...(475): n = A,T,C or G
gactgagacctcctcaaagaaggcatgctgaccaaacagaacaactcgttccagcgatccaagaggaggtactttaagcttcgagggcgaacactttactatgctaaaactgcaaagtcgattatatttgatgaggtagacctgactgatgccagcgtagcagaatccagcaccaaaaatgtcaacaacagttttacnggtcatcactccatgtaggaagctcatcttgngtgccgacaacaggaaagaaatggaagattggattgcagcactcaagactgtccagaacaaagagcattttgaggttaaaaganangcatatntgtctatgggagcctgctgangnctctcctgccttcattctgatccacccccaccaactccacccttcacccccagagtattcttccccaggggaccccagnatntgtcctngtaaaatngagnncccatggcttctttcttttgataggac

SEQ ID NO: 735 - DNA, 130 bp, Mus musculus
gaactgaggttcgtcttggcaccagaacaaggcactgcaacatcaggcctttgccctaaacagctcacgatgccaggaactggccaattactactttggcttcagtggatggtcgaaaaggatcatcaag

SEQ ID NO: 736 - DNA, 365 bp, Mus musculus
  misc_feature (1)...(365): n = A,T,C or G
gaactgaggtgcaggngtccggggcaggcccgggacaagtcctacttactgccaccacagnccaccaagcagttcctcatctcccctcccgcctcaccccccgtggggtggaagcagagtgaagatgcaatgccagtgatcaactatgacctgctctgcgctgtctccaagctgggcccagnatngnggagatgaaggcacacaatttatnatttgatncnaatgtcangtgtactgnggnntgagancatngcttnnantgaaagaggagcgacaganngcaagaatgtgacccgnaggacactngngattacctntaacaaacgttgatgtttgaaactaataaagaattggtgctttttaag

SEQ ID NO: 737 - DNA, 415 bp, Mus musculus
gaactgaggtaaatttgctatgctagattacactccttcaaatgtcccgctggtgaatactggtctaaagacgtctgttgcaagaactgttctgcaggtacatttgtcaaggcgccctgcgaaatcccccatactcaaggacaatgtgagaagtgtcacccaggaacattcacagagaaagataattacctggatgcttgtatactttgctccacctgtgataaagatcaggaaatggtggccgactgctcagccaccagtgaccggaaatgccagtgccgaacaggtctttactactatgacccaaaatttccagaatcgtgccgcccatgtaccaagtgtccccaaggaatccctgtcctccaggaatgcaactccacagctaacactgtgtgcagttcatctgtttcaaaaa

SEQ ID NO: 738 - DNA, 524 bp, Mus musculus
gaactgaggatctcatctggcggtcgggtactggaagaagcgagggggagagcgcaccgttgcagagagacggcaagagaaaagggagactgagagtcgcgccacgccggcgccccaagacccaggaggagcccgcagcagttaggttgtgctgggaattctcccctcccttttgctccacaatgccttggtgagcggtcacggcgaccgagctactggtggaagaattggggctgagatgtcgcggcgggtggccgcagatggggatgctggagaaggaaggagttcacctcaagttcgcaatctgtgtcggaaagctggtccggcctgggagggcgtttcgagcaccggccgcttccggatctggtggactgatcggctttaaacacttcccctagatacccctccccattccctgttgcgttccctctgtcctgcgccaacccgcttgctaaaacagctgagtggccaagctgacaataaaatctcatgcctttgcggtgaggggcaggggtcatgactca

SEQ ID NO: 739 - DNA, 439 bp, Mus musculus
  misc_feature (1)...(439): n = A,T,C or G
ggctcagtggataaaggcacttgctgccaagtttattgatganaangangangnngnnaccnacgtananacagcnnccagactcacttntgcnnncttantgacctgtacgtgcatgctgggacncactgtacacnctcacacnaatggacnngacccaagatgncacacncanggnggggganannaatacacngattcacngagncataataggtgtcttgcgtactnnntgnncacancatggnaggaangacagccatgctcctggcactgcagaggccagatgttgtggaacgattggttgttgtanacataagcccggtaggaaccacacctggatcacacattggagccttcatagcagccatgaaggctgttgagatcccagagaaggtgccgcactcccaagcccgaaaactggcggataagcagctta

SEQ ID NO: 740 - DNA, 253 bp, Mus musculus
  misc_feature (1)...(253): n = A,T,C or G
aggcacaagggatggaccaccaccngctcagccgaccacgggagagagtgctgctctcctatctctccccatagagaaaacaacaggtntnttcaagccagtgncaccgagctccactgantgtacaggcagcagctacagccaaaagcaggccactcacgggaagtcccttacagaacctgntcaggtcacccaggctaagatactaaatatctttcaaattacacatattcttgaggtaaaaaataaacaa

SEQ ID NO: 741 - DNA, 136 bp, Mus musculus
agaggagagccagcacgcaccatggcaccggccggacacatcctcaccttgctgctctggggtcacctgctggaactctggaccccaggtcactccgcgaacccctcctaccccaggctacgcctgtcacataaag

SEQ ID NO: 742 - DNA, 512 bp, Mus musculus
  misc_feature (1)...(512): n = A,T,C or G
cggacagaactggactttcacgtttgacttctcttttttgagccaagaagaggatctggnatgggcnggagctncggntgcagctgccgggccccatggacatacccactgagggcccactcaccattgacattttccaccaggccaagggggatccagagcagggaccccgctgactgcctggagcgcatttggatggagacgttcaccgtcattncttctcaggtcacgtttgcctcaggcagcacagtcctggaggtgaccaagccactctccaagtggctaaaggaccccagggcactggaaaagcaggtgtccagtcgagcagaaaagttgttggcatcagccctacaccccacctgtacctgtcgccagcaccaatgtgctcatgctctactccaaccggcctcaggagcagaggcagctagggggcgccactttgctttgggaagctgagagctcctggcggccccaggagggacagctgtctgtagagaggggcggatggggca

SEQ ID NO: 743 - DNA, 487 bp, Mus musculus
gaactgagttcctgaagaattcatgtacaaccctttaaccagagtttatggagaacctcacaaaagacctgaagtccagaatgctactattgagtttatggctccttcagaatacatgttacgaccaccacaacctccagtgtacctctttgtatttgatgtatctcacaacgcaatagaaactggatacttgaattcagtttgccagagtttgttagacaatctggatctgcttcctggcaacactaggacaaaaatcggtttcataacatttgatagcactatccatttctacagtcttcaggaaggtctctcccagcctcagatgctaatagtttcagatattgacggtatgtgttatactcaaactcactcaacttcctggagaattgtccctttcagaaatgtttcctgaggtctttgatgtgtcaggaatgtgtgacatgactctagtataataaagagaaatcatttggaaaaaaaaaaa

SEQ ID NO: 744 - DNA, 359 bp, Mus musculus
  misc_feature (1)...(359): n = A,T,C or G
gtgagccaccactctnagntcctgccgccctcattgngaccttgggtcctcagcncatgctcgcacttgtgactacagacttcccagctctagaactacntgctaagnctgagngcccgcagacagatctnaagagggcatcagatcacttggaactggtagttnaagacagctgtgagtcnccatggagatgcngggaatggnaactaggccttctgtgagagcngcagnntggcatagccgcctctccaccncagagttttaaacatanataccctaccgattcncaactgcncctgtcgtgcaagtgtgcagtcccttagntcgctgtttctacttcccttttaaagaaaaaataa

SEQ ID NO: 745 - DNA, 130 bp, Mus musculus
tctcaaagtcataatcctcacactccaacttgtattaacccaggctcccatttgctgaagtaaaagtactgagttttccacttaccagtacttttgagtactgtagcaagtggctgggtcccattaaaaa

SEQ ID NO: 746 - DNA, 349 bp, Mus musculus
gaactgagaaaaatattttgacaaaagaagagactacttaaaattcaaggaaaaatttgaagcaggaggattccagtcttcacagtcgacctgaaaattcctaggctctttctggactttcacacaagtggaatttactcctctggacttgcagaaatgattccaggacaaaagcagcttgagtcacagtgtgtatcagcacttgttctctgtaaacaagagttactaaattgttggaatagcaaaagatctcatcatccaaactatgaaggactgccttaattaaagtgagggatctattctaagaaaaagcataaaatcattatataaaaagtttattataaaaata

SEQ ID NO: 747 - DNA, 215 bp, Mus musculus
gactgagagaagctgatggtccagggcatctaaccaaagtggtagaagaatcaaagctttcaaaagaaaatcaggcaaaagcaaaggaatccgacttatcagacactctgagtccaagcaaggaaaaaagcagcgacgacactacagacgcccagatggatgagcaagacctaaatgaaccccttgctaaggtgtctctattaaaaggtatttta

SEQ ID NO: 748 - DNA, 454 bp, Mus musculus
  misc_feature (1)...(454): n = A,T,C or G
actgagttttgtggctaagcccagactgcttgagtgaaacagccttcagggacgttccattctttcgtttatcccagtgcttacaacccaaaaacaaaattggattgccacaagtcggccactggctgaggaagtgcactcggnccctgacacaaatgcatcagtcaggagaaagagccagcttctcaggtgtcttcagagaaggccagactgacagctgcggtcacccattgcagtgctagcagcnatttcatggactggaagtgcttgttggtacttgatccagaaaactgtttctcaatctcttaagaggtganggcnggatcntganctntaanaaccataaaggcttccgtgcatatangtgacccacaagagctggataaactgcacctgagcagaagtgcaggactcccttggnggctctgntttanataagaaagtgtattttttg

SEQ ID NO: 749 - DNA, 241 bp, Mus musculus
  misc_feature (1)...(241): n = A,T,C or G
aacattgccatcgtgaagccaggcaaaaagggacagccagccgttatcatggcagtggatcctcgataccggggcagagtgagcatctctgagtccagctactccctgcatattagcaatctgacctgggaagactcagggctttacaacgcccaagtcaacttgaagacatctgagtcccacatcacgaagtcttaccatctccgtgtctaccggaggctgtnaaagccccacatcactg

SEQ ID NO: 750 - DNA, 466 bp, Mus musculus
  misc_feature (1)...(466): n = A,T,C or G
gaactgaggttttgtgtggacagtaatgaccgcacgtttccgattgcctgctggcagaacctacaatgtccgagcatcagagttggcccgagacagacagcatacagaggtcgtttgcaacattcttcttctggataacactgtacaggctttcagagttaacaaacatgatcaggggcaagttctgttggatatagtcttcaagcatcttgatttgactgagcgagactattttggtttacagttggctgacgattccacagataacccaaggtggctggatccaaacaaaccaataaggaagcagctaaagagaggatcaccttacaatttgaactttagagtcaaattctttgtaagtgaccccaacaagttacaagaagagtatacaaggttggtttatggaactatttctttngaaaacattatcggagatctaatatgaaaaataaaattaactaggcca

SEQ ID NO: 751 - DNA, 504 bp, Mus musculus
  misc_feature (1)...(504): n = A,T,C or G
gactgaggcattgccttttggcactcggggagnaagatgcagcaggttccgtctcagaagtaatggctggatccctgaaagccaacgggctcggtgtggtccacccgtcagaaggcataggtagatggtgggcatctgcctcagacatagttctcaagttcagccagaagaatgcagcgttgagccctctgcagcccgtgtcgacggaggtgttgtctccaggaagcatcatggaaaggcgtggtggtgcacaagggccttcaggatgcctgcgcctggtgacggntgcgcctcaggttctgggcctgactcggaagggtgcctgtgagcagaagagctggcacgcgcagctcagtggaccatcagtgccaacccgtcctgtaccctggctgaggggtgtgctaannggcanattcctggttggctcctngtaaggcgnggcacaaaatcaccatcgaaagaaagggccttcggnatgccttctgtctggatattgccctgtag

SEQ ID NO: 752 - DNA, 532 bp, Mus musculus
gaactgagaaagtgagtgagaaggtgggaggagcggaaggcaccaagctcgatgatgacttcaaagagatggagaggaaagtggatgtcaccagcagggctgtgatggagataatgacaaaaacgattgaatacctccaacccaatccagcttccagggctaagctcagtatgatcaacaccatgtcgaaaatccgcggccaagagaaggggccaggctaccctcaggcggaagcactgctggcagaggccatgctcaagttcaacctcttggagatggatatagaacaggtgagccagctctccgcacttgttcaggctcagctggagtaccacaagcaggcagtgcagatcctgcagcaggtcactgtcagactggaagaaagaataagacaagcttcatctcagccaagaagggaatatcagcccaaaccacggatgagcctagagtttgccactggagacagtactcagcccaacgggggtctctcccacacaggcacacccaaaccttccaggtgtccaaatggatc

SEQ ID NO: 753 - DNA, 367 bp, Mus musculus
  misc_feature (1)...(367): n = A,T,C or G
gaactgagtagcgctggggacctgtgaccgcngcatccagctaaccatgggacagnttggctctgntcaccagccccttaggagaggactccatcaaggncccactgcnnattccncatcatgnaggccganaggatccttccagccaggggagctgncctttgaaaaccnaggncgngtactgctggtnntccntgccanccaccttcangagaggctcctgttnctgctggcccacggatggggaggngggccctggacgnagnctttgtgtggaaggnagctctgaccctgggnctgaccttctatactactgcttctncataggcancacgttctacnacaaatggntgacaaagagcttcta

SEQ ID NO: 754 - DNA, 389 bp, Mus musculus
  misc_feature (1)...(389): n = A,T,C or G
taagcagtacgctaatggacatagacagcacaatttccagtggacgctcaaccccggccatgatgaatggacaggggagcacgactgcttcaagcaaacacattgcctataactgttgctgggaccagtgccaggcatgcttcaactccagcccagacctggcagaccacattcgctccatacatgtcgatggtcagcgtggaggggtgttcgtttgcttgtggaaaggttgcaaggtgtataacaccccgtcaaccagtcagagttgnctacagcggcacatgctgacacacagtggagacaagcctttcaagtgtgnagttgntggntgcaatgccagctttgcttctcagggagggctagntcnccatgtacccacacacttcagt

SEQ ID NO: 755 - DNA, 797 bp, Mus musculus
  misc_feature (1)...(797): n = A,T,C or G
aagtaaaactgagggncaagtcctccataccaaggaccaggctaaaaccagattacactgcccctccctgggccctattgacttaaagcagccactgtaatcatgggcaatatctttgggaaccttctgaagagcctaatcggcaagaaggagatgcgcatcctgatggtgggcctggatgctgccgggaagaccaccatcctctacaagctgaaactcggggagattgtcaccaccatcccgaccattgggtttaatgtggagacagtggaatataagaatatcagcttcacagtctgggacgtaggtggccaggacaagattcggcccctctggagacactacttccagaacacccaaggcttgatatttgtggttgacagcaatgatcgggagcgagtgaacgaggcccgggaagagctgatgaggatgctggcggaggatgagctccgggatgctgtgctccttgtgtttgcaaacaaacaggatttgccgaatgctatgaatgctgctgagatcacagacaagctggggctgcactccctgcgtcaccgcaattggtacattcaggccacctgtgctaccagcggggacgggctgtatgaaggcttggactggctggccaatcagctcaaaaacaagaagtgagagccaggcagccctctctgactaccccacccactcctgacacacacctgctgnctccttaccccaattttcccttcttcctntttgaaagtggccaaggtcctgggtatcatgtccacatgcccagcaagcgccttgcctttctcctc

SEQ ID NO: 756 - DNA, 452 bp, Mus musculus
  misc_feature (1)...(452): n = A,T,C or G
attgttatctaaagaagctatccctaacagattttactaccaacacagcagtcgaattcagcccattattttggttgctgaggaaggctggacaatcaccctgaacaagtcatcatttaaattaggtgaccatggctatgataattccctgcctagtatgcacccgtttcttgctgcccacggacctgcctttagaaaaggttacaggcagagcaccatcaacactgtggatatttatccaatgatgtgccacatcctgggactaaaaccacatcctaataatggaaccctcagtcataccaagtgcttgttggtggaccagtggtgcattaatctcccanaagccattggaattgttgttanngcactattgntattaaccatgctaacaggcctgatgatattnattgnggagcagancatnccacatcccgnccattctcccgtcttca

SEQ ID NO: 757 - DNA, 247 bp, Mus musculus
agtgggcatatgaacagtgtgcaactgggagaaacttgtctccccggccattaggactagtccttgaaggggttccttcaaacttgagccatttacaggtaccacgcggagcctgagaaaagaaaaaccataaagaccgccacactcaagatggcgttggactcggcaagcgtggcagtgacgtggcgactcggctcacgccgaacaacttcaaaatactatattactccatttttaaaaaaaaaaa

SEQ ID NO: 758 - DNA, 108 bp, Mus musculus
gactgaggctcagacaagagaaaagctctagaagagaccaaagcatatacaactcaatctctagctagtgttgcttatcaaataaatgcattggccaacaaaaaaaaa

SEQ ID NO: 759 - DNA, 468 bp, Mus musculus
tgagcaccatgaaggtgaaggagacaaactcaaagccaaagttggcaagccgtggcacgttccagaggaagggcatcaagattgtggggaaatggaagcaagtgacgatcgacccgaatctgtttgcagatggacagatggatgacctggtgtgctttgaagagctgacagattaccgtttagtcaagaatccctcccgtttgttctcaagtgaggagacaaagaagagaaaggcccaggctgtttcagaagaggaagaagaggaggaaggacagtccagctccccaaagaaaaagatcaagttgaagaagcagagagatgcggcacgagctgccgaaggcgctgctgcccagaatgagtatgaagtcaaagcgtccgagccagaggctcagggagaagtcactgcttgttctgatcaaaaggtcggaggagccaaatcagaaagcctggcccaggctgctccaagaa

SEQ ID NO: 760 - DNA, 153 bp, Mus musculus
  misc_feature (1)...(153): n = A,T,C or G
ctcttatgaactcctagaaggcagngacatctactttccagagattgtgaagcctccccggaatccagagctagttgcccggctagagaagattaagatacagctagccaatgaggagtataagcaggatcactcgcaatgtcacttgtcagg

SEQ ID NO: 761 - DNA, 495 bp, Mus musculus
  misc_feature (1)...(495): n = A,T,C or G
tgaggacttgctgaaaatgaatattgatatgaacaagcatcttgtgactggttctgttcaagccaaggtactgaaatggggtgaagacntagaagacttgatgtcaccagactacatattgatggcagactgtatatactatgaggagtccttggaaccactgctgaaaacattaaaagatctcagtggatctgaaacttgtattatatgttgttacgaacaacgtacaatggggaaaaatccagaaattgagaagaagtattttgagctcctgcagctagactttgactttgaagaaatccctttggacaaacatgatgaagaataccgaagtgaagacattcatattgtgtacatcagaaagaaaaagccaaaaccgccatcatgaagcgcttctccatcttagccacgccccgcaacatccctgggtaaggtcaagatgtgaacggagcatgtgcatacccgcatggaaaactgcctcacagatttctccag

SEQ ID NO: 762 - DNA, 420 bp, Mus musculus
  misc_feature (1)...(420): n = A,T,C or G
agactgagacagctagcagcggtgcctgagttccagggcactagggcctctcttcaagtcctctcctgagccagtggccctcacagagtccgagaccgagtatgtcatccgttgcaccaaacacaccttntctgatcacttggtgttccagnttgactgcacaaacaccctcaatgaccagactntggagaatgtcacagtgcaaanggagcccactgaggcatacgaagtgctctcgtangggcctgcgcggagcttgccctacaaccagnctgggacctgctacacactantggctctgcccactgaagaccccacagntgtggcatgcacgttcagctgtgtgatgaaagttcactgttaagggactggngatcccaacaanaggagaantcgangaaagaaggctatgaggatgag

SEQ ID NO: 763 - DNA, 286 bp, Mus musculus
gaactgagacccattgtgccgaaaatcttctcaaggcggacacttaccggaagtggcgggcagccaaggcaggagagaagaccatctctgtggtcctacagggctggagagatggctcagcggttaagagcactggctgctcttccagaggtcctgagttcaattcccagcaaccacatggtggctcacaaccatctgtaatggaatccgatgtcctcttctcgtgtgtctgaagacagctacagtgtactaatatacataaaataaatgaataaattaaaaaaaa

SEQ ID NO: 764 - DNA, 131 bp, Mus musculus
  misc_feature (1)...(131): n = A,T,C or G
gagatctgacccttgcanaccggcctggnggagggaaatgnatacnnntganaanggnantcacttcttttctgctggccctctaagacatttntgcttcatatttataaacnaaataaacttaaagccgg

SEQ ID NO: 765 - DNA, 408 bp, Mus musculus
  misc_feature (1)...(408): n = A,T,C or G
atataccctagcagacatcctgatgttcgagagtcactaggtcccctgttggcccctacccctctccgtccatggccccagatgacacttcagggcttctctgtgtatcgatggntgtcctanaacttgctctgtagaccaggctggccccaaaatcagagctttgacctgcctctggctcccaagngcagggattaaaggcgtggggcacctctgtcaggctgatacactgtatctcatggatatgtctcttaactttttaatagtccctcagtttcttcattccactttaattactaatccctgtttgtcaagtacagtttgacagagtctgagtttgcttgattagattcaggttaaaatccgnttcttactctattaaacaaagnttaaattacaaaaaaaaaa

SEQ ID NO: 766 - DNA, 191 bp, Mus musculus
gaactgagggttccaagactgccctttggtattcaaacgggagccgctttggactgactggctgccttcctcctcctccccctcctcccccttctgcttcagatttatttattatatgtaagtacactgtagcagttgtaggacacactagacgagggcaccagatctcattgtgggtggttgaaagacac

SEQ ID NO: 767 - DNA, 471 bp, Mus musculus
  misc_feature (1)...(471): n = A,T,C or G
aatacacaaactcgagctacttctaaactacctgttaaatccaaagaagcggatttgcttagacatcttcatccaggagggccagagcctgatgttacaaaagtcaccaaatcgagacgagagaatgggcaggtgaaagcggcagagactgccagcaggaggaacctcagaaacagctacaaaccgtttaataagcaaaaaccggaggaggaactaaaggataaaaatgagctgctggaggctgtcaacaagcagttacaccagaagctgacagagactcagggagagctgaaggacctgacacagaaagtggagctactgggagaagtttcaggataactgcttagcacttttggagagcaaaggtctcaacccaggccaagagaccctggcatcaaagcaggaacccaccacagatcacacggactccatgctgntgctcgaaactttgaaagacgaactgaaggtttt

SEQ ID NO: 768 - DNA, 219 bp, Mus musculus
  misc_feature (1)...(219): n = A,T,C or G
tgacatgtgagcttggggttatactgtgccataattaacattgccttgacnactntggacaccgagactggcctcataagtagaaggctttntttttttttttttaanacagactcnccatnttnntgnatatgcgaaagnantttggtccnncaccccctctntgggacanaaactntntgtgtanccttgggtgnanaccaccgtaggccttgaact

SEQ ID NO: 769 - DNA, 423 bp, Mus musculus
gaactgaggtgactcctgctcaggtcatgccgttatctgctgatccagccggccctgccaggtgaccatgtctgtcctcggcccagttcttctccaggtgttctgggccgggtgtgtcgtcaccctgcggtcccctctgccagctgctttcactgccaatggcacacatctacaacacttggcaagggaccccaccacaggtaccctctatgtaggggccaccaacttcctgttccagttgagccctgggctgcagctggaagccgtggtgtccacgggccctgtgaatgacagccgggattgcctgccacctgtgatacctgatgaatgtccccaagcccagcctactaacaaccctaaccagctgctcctggtgagcccagaggctctggtggtgtgtgggagcatacaccagggcatctg

SEQ ID NO: 770 - DNA, 554 bp, Mus musculus
gaactgagatgatgactttgaccagtttgacaagcctggtgcagaacggtcctggagaagaagagctgctgacgaggactgggacagtgaactggaagatgacttgcttggagaagatttactgtctggcaaaaagaaccagtcggatttgtcagatgaagagctgaatgatgatctgttacagagtgataacgaagaggaagaaaatttcagttctcagggtgtcaccatcagtctgaataccacctctggcatagttacatcatttgagctgtctgacaacaccaatgaccaatccggagaacaggaatctgagtatgaacaaggagatgatgagctagcctaccacaaacctgaggaacaagaactgtatactcaagagtacccagaggaaggacagtatgaaggccatgatgcagagttgacagaagaccaaatagaatatggggatgagccagaggaggagcagctgtacagtgatgaagtgctggacatcgaaatcaatgaacctttagatgaatttacaggaggaatggaaagaggaatgggaaacc

SEQ ID NO: 771 - DNA, 104 bp, Mus musculus
  misc_feature (1)...(104): n = A,T,C or G
gactgaggcatactccagcacccagntggtacagttctgaaacagctacagccaccgccanggngcccaagagagctggaattttataccatggtttactgctg

SEQ ID NO: 772 - DNA, 456 bp, Mus musculus
  misc_feature (1)...(456): n = A,T,C or G
gaactgaggcattttgtgaaagaaggaagatcctgtctgctgcatggataatggactggaacacagaaagaccatgcagggttcggctgtagaaggccggtatctccataggccagannacaccatcangatctcctgggactggagttatanaggttgtgagctgccatgttgaaccaanctggttactgagggacacaggcatgtggatggaaaccctgctgggagaaaaacantanctggtntgtcnctttatagcccncttnatgatgcaagananaatcggctncncacaactngnctctgacctntncatgcttgctgtaccttgtgctcaccctacccacacaccntcgnnatcatnatcaacataacgtttttaaatatannaagtntattcaaatatatgtgtataaatacatntatanataaatatgtgaatacattaaaaaaaaa

SEQ ID NO: 773 - DNA, 163 bp, Mus musculus
  misc_feature (1)...(163): n = A,T,C or G
cacactttcgcgctcagcgtcgacggaggctctgtggctctctacgtaaaactgntgaaagaattccagagggtgccatttgctcgggcctagcagggactggagctagagcgtggcgctggnctctttgnggggtcaggctggaacagggagaccctgactg

SEQ ID NO: 774 - DNA, 400 bp, Mus musculus
  misc_feature (1)...(400): n = A,T,C or G
gactgaggacttaagatgccttctttgntcatacaccgtttaggacatggaagattttcaggtccggtgaagacantgcnacccnagtgagcattncanatcatgtntaaagagaggtaagagatatgctgcanagtccaaagaacaagatgggtgtggctcagatggctttacatatttcaaganccaagaccccctgtctgctacaactctgctgtgcangccctgagcacanctgcagctttctctgacacaccatcccacagggactttctcacctatacagtgttctcatgtcaaataagatcgttgtacaaggtctttgttacattgaaccctgaatttattcagttatgttaagngcaataaaatacctgggttttgtacttgcagaaaaaaa

SEQ ID NO: 775 - DNA, 181 bp, Mus musculus
  misc_feature (1)...(181): n = A,T,C or G
gactgagggtttctctgngtagccctggctgtcctggactcactctgtncaccaggacccnnacccaagagatctgcttgcttttgcacctcangactaaatctaangaaagatggatacctacaatggtgaatttgnggactctgtttgagcagcttgtgcagccggatggagattatca

SEQ ID NO: 776 - DNA, 172 bp, Mus musculus
  misc_feature (1)...(172): n = A,T,C or G
gatatccctgagatggacacagtccacctgngtggcctaaggatgcagggcagaggaactgcccatgggcagtggttcatcggccaagctgagagtgtgtattttggatttccggtttgcggagatgctctcttccgggtgttctgcccaaaataaaggacctggacaagaa

SEQ ID NO: 777 - DNA, 318 bp, Mus musculus
gaactgaggtacaccaaattgaagaccgccccaacatctacatcttcaatctggctttggctgatgcgctggccaccagcacgctgcccttccagagcgccaagtacttgatggaaacgtggccgtttggcgagctgctgtgcaaggctgtgctctccattgactactacaacatgttcactagcatcttcaccctcaccatgatgagcgtggaccgctacattgctgtctgccatcctgtcaaagccctggacttccggacaccagccaaggccaagctgatcaatatatgcatctgggtcttggcaaaaaaaaaaa

SEQ ID NO: 778 - DNA, 453 bp, Mus musculus
  misc_feature (1)...(453): n = A,T,C or G
gaaagcagttatgatgccgncaaagccctgcancgcctggtgaagaagcctgtgcccaagctgatcgagaagtgctggacagaggatgaagtgaaacgcttcgttaaggggctccgacaatactggcaagaacttcttcagaatcagaaaggagctgcttcccagtaaggaaaccggggaactgattaccttctattactactggaagaaaacaccagaggnnttcagctnccgtgcccaccgncgccaccgcaggcaggcagtgttcacgaagaattaagactcgcacagcgtccacccctgtnaacacgccctncagaccaccttccagtgaattcttggacctgagttcaaatcccagcaaccacatggtggctnacaaccatcctgtaanagagatctgactccctcttnctggagtgtctgaagacagctacaagcgtacttacatat

SEQ ID NO: 779 - DNA, 571 bp, Mus musculus
ggtttcactatgtagaccctggtcggcctggaactctatagaccagactggcctcgagctcagatccgtccccctctgctgtcccagcacggggattaagaacgcgccaccactacagctgaccggagcttcactttggttagacacaaacacaacacagcttgtcgtcaaacaccgtgacatgaatgagaaggaattagaagcccaggaggcacgaaaggcccagttggaaaaccacgaaccagaggaagaggaggaggaagagatggaggctgaagagaaagaagctgggggctcagatgaggaacaggagaagggtagcagcagcgagaaagaaggcagcgaggatgaacactctggaagtgagagtgaccgagaagagggtgacagagatgaggcaagtgacaagagtggcagcggtgaggatgaaagcagcgaggatgaagcacgtgctgcccgggacaaagaggagatttttggtagtgatgctgattcaaaagatgatgctgactcttgatgatgaggacagagggcaggcccacaggggcagtgacaatgactcggacagcgg

SEQ ID NO: 780 - DNA, 145 bp, Mus musculus
  misc_feature (1)...(145): n = A,T,C or G
cagcgccggtggctggcgacttcacggctgnngacgtcatgacgagcgaggngattgangatgacananagttttattnganggccatgacctactgtaagcagatcccacccacggtggacggcatgctcggggggaatgtcca

SEQ ID NO: 781 - DNA, 302 bp, Mus musculus
  misc_feature (1)...(302): n = A,T,C or G
cagcacgtcttaaaacatttactgctaaccagattgatgaantaaaagacccttcaggactcttttatatccttccttttcaaaagggatacttggngaatngggatgnccagcgacangnttgggattatctttttggaaaagaaatgtatcaggnngagtctttanatactaatattantatnacagaaccatattttaactncactncnatccaagantcantgaangaantcctattcgaagaanancaggtccaagcaggattaagagtaaatgccggggctctcagcgcacacagg

SEQ ID NO: 782 - DNA, 182 bp, Mus musculus
  misc_feature (1)...(182): n = A,T,C or G
tcattgaacacatccaggtgaacaaggcacctaagatgcnccgacgaacctacngagctcatggncggattaacccatacatgagctccccctgccacattngagatgatcctcantgagaaggaacagattcgttccaaanccacnaataggaggttngcacagaacaaaaagatatccca

SEQ ID NO: 783 - DNA, 453 bp, Mus musculus
gaactgaggcctctaattttaagaagacaaccatggcatccagtgcccagagaaaaaggatgagtcctaagcctgaacttactgaagaccagaaacaggaaatccgggaagcttttgatctctttgatgctgatggaactggaactatagacatcaaagaactgaaggtggcaatgagggccctgggctttgagcccaagaaagaagaaattaagaaaatgataagtgaaattgataaagaaggaacaggaaaaatgaacttcagtgactttttgacagtgatgactcagaaaatgtctgagaaagacactaaagaagaaatcctgaaagctttcaagttgttcgatgatgatgagactgggaaaatatcattcaagaatcttaagcgcgtggccaaggagctgggtgagaacctgacagatgaggaactgcaggaaatgattgatgaagctg

SEQ ID NO: 784 - DNA, 260 bp, Mus musculus
gaactgaggattcccgaggaccggtgcagcagcctttggaggatcggctcttcccacctacggtctctgcagtctatagcacggtccctgtagtcctggctatcctggagcttgctgtataaaccactgtggccacaagctcccagagatgttcctgcctctccctcccatactggaactaaagctgttcatcatcacattcctttcaataagcgtttctttaataatataacttggaagccatatgaaataataaaaaa

SEQ ID NO: 785 - DNA, 115 bp, Mus musculus
gaagaagatggactcacatgagcacaagttcagaaatgaatagtttgaaagtaggttcttggaaaataaataaataaataaataaataaagacaatggaatggaaagaaaaaaaa

SEQ ID NO: 786 - DNA, 400 bp, Mus musculus
  misc_feature (1)...(400): n = A,T,C or G
actaactgatgatgatgttgaacagagcccaggcaatgccttatgtggatcggcagaaccgaatctgtgggnttctggatattgaagacaatgagaacagtggcaaattcctccggagatactttatcctggatacccannncaactgcctcctctggtacatggacaatccccagaacctggccgttggggcaggagctgtnggatctctgcagctgacctacntcncnaaggttntnatagntnccccaaagcagaaacctaaaacaccattntgcttcnntatcaatgccctgtctnagagatnttttnttcaagccantgaccanaaagatctganggactgggnncnaagacnttgnaccaagcccanccaagattacctgtacccaaagctgcg

SEQ ID NO: 787 - DNA, 542 bp, Mus musculus
gaactgaggcacgggcagtggttctgtatcccacgcaatcatccgcagcgttgcccccactggccacctacacacagtagagttccaccagcaacgggcaggacaaggcccgggaggaattccaggagcatcggctgagccagtgggtgactgtgcacacccaggatgtgtgctgcagtggcttcggtgtggtccacgtggctgacgctgtcttcttggatatcccatcaccctgggaagctgtgggccatgcctgggatgccctcaaggttgaaggtgggcgtttctgctccttctctccgtgcattgagcaggtgcagcggacatgccaggcgctggcagcccatggcttcacagagcttagcaccctggaggtactgccacaggtctacaatgtgcgcactgtcagtctgcccttgcccgacctgggggccaacaacttagagaccaacatgggctctgatgccagccccttccgtagtggcacacccatgaaggagactgtgggccacactggctacctgacttttgccactaaaacc

SEQ ID NO: 788 - DNA, 300 bp, Mus musculus
  misc_feature (1)...(300): n = A,T,C or G
aagaatcatggtgagcacaangaagaaataantgaagcggngccagggggtggttcctcatgagatgnaaggccaccanaggaaagcacccannaaatgatggaggaggaggaagaaataccgnnacctanttcagcggtggcaccaccaggtgctccnaagaaggaacatgtnaatgtngtgttcatcgtacatgttgatgcnggcaagtcaaccattgtaggacaaataatgtatttnactggnntggtngacaaaaggacacttgnnaagtatgatcganaagctaataaaaaaaaa

SEQ ID NO: 789 - DNA, 359 bp, Mus musculus
gaactgagatccctgctgtctgaatggaggaccacagtaggccacgggaggaggagaatcaagatgctgcagttgacagtcaggtgaccctctgaagctcatctgcctcttcaaccatcaggtctaggtggctgtgctgtagactctgttaccctgtgacgtgctggtgcggggagttccatagggaggacgctccatgtcctggaagccagaaatggggctggtgtcatttgaggatatagctgtggaattcacctggcaggaatggcaggacctgaatgaaggtcagagaactctctacagggatgtgatgctggagaactacagcaacttgctgttcttggggcacaaaaaaaaaa

SEQ ID NO: 790 - DNA, 245 bp, Mus musculus
  misc_feature (1)...(245): n = A,T,C or G
aatctcaggatgcacataagntgctttgnnngacaccntggaggaaganaacgagaaggatctggtagagactagctaaacgcnttaanagagaaaaagagantggtggcatcattaattntgnggaanaggangcgaacanaangaagcaganagnnttngggagcacactgaatggcaaggatggaactgggtcccatctagngacagngcctnctggacctagcttggctgatgctctcatc

SEQ ID NO: 791 - DNA, 533 bp, Mus musculus
  misc_feature (1)...(533): n = A,T,C or G
atggagctaatgcacccatccgaagaacaaagaagagcccaaagcccggcccttgagagtgggtgacacagaaaagccagagcctgaacggtcccctcccaaccgcaagcgtcctgctaatgaaaaggccacagatgactaccactatgagaagttcaagaagatgaacaggcgctactngaggcagcggtaggggctggataacgatgtgatgcacaggggtgcgtttgtgtgcatgtgcaagcgtgggggcacacacacatgtcctcttgcataaaacctgcttattagctcttgagttccccatcagatagtgcttctgctcagtggctggatttctgttctggttttggttggagactgtagctctctgtgtatcctaggccatgaactccctcctcctccctactactgggattttagaccccccaccccctgcattttcaagcactaactttgtaactattnggaagaaaacaatggtgtctatgtctatctatattgtttccctaccctagggcatgacactgata

SEQ ID NO: 792 - DNA, 498 bp, Mus musculus
  misc_feature (1)...(498): n = A,T,C or G
gactgagacagctcagtgtanaanagagangacggagaagganatggaganggagaanganaaggagnngangaagannaagaagacgatcancatgtttgatggcagacgtcttcttncnncganctccctgtccttaggaaaagagggaaacngatggaacanggntataggcctaagacnggntttctgtgtgtacccctgnctgtcttggaactcactctgtagacgaggctgnccttgnattcagaaatcnncctgcctctgcttcctgantgctgngattanaggcgtgcaccaccatgcccngctggaattcaacacttntatcttttgcttggtgacttattatagtttgctgagaaaaaaagttcaatttcttttgtttaaaaattgtgaatgcacatttgcatatttgagacagcatcttgctatgtatcccagtctacccttgctgttttgttaaaatacaagtaacaaacttgtcaaaaaaaaaaa

SEQ ID NO: 793 - DNA, 427 bp, Mus musculus
  misc_feature (1)...(427): n = A,T,C or G
aactgagagacggaagcagctacttgaggcgagccacagctcagatcatggaaatcagtatgcaagcccaggagaactgcaggagttggcatcatattcatgtannatcagtgntaactctgggctacngagacgaatgttggactactatgtgtctcactgacaagacactgnnggtcancgatgggagacagaactcaccatttaaaaagatnaagaangaacaccacccnactaagatatgaaaaaggaagcacganggnaangctgagagaaacagatgctacccacagggcagcggagccatttggttgttttggtctttgtaccancaactcccactcctgtgaacggngtccagttccagctgnactacacggtttttccttgcagtcaaaacgcgaacaatggaccccactcctccagc

SEQ ID NO: 794 - DNA, 446 bp, Mus musculus
  misc_feature (1)...(446): n = A,T,C or G
gaactgagactgaaggncttttaccagagtctctgctgttctcctcggattcttcctcctcctcatcgtcatcggagtacttcttctttggggnctttctccgcaaacgcctgctttgcctcattggccgagaaggatgccgcctcagtcttcggctgcagaagtcagtgtcgctgncctcattggatggtggctcttcttcactttcatctggattttcatcagacacaacaaactcatcctgagatctgtccaggaaaagccagatacatcacgatggtacacttagagtttggtntgntttggnacattnagaagngataaccagtttttcaaagntccaaacacattgtaaagaacccaggaaaacattaagtaaatntaaagatatatttggaaaattatgtcaactttgccaataaaatgtatgttctaccatctaaaaa

SEQ ID NO: 795 - DNA, 219 bp, Mus musculus
  misc_feature (1)...(219): n = A,T,C or G
cttcacatcatttggnctgnaccatangcntgnnngnntggtnntctncagngcctggacccattgaccagccccctgagggggaggatgagcacatggagtctctggacttagaggctgacaggatccggcaggagctgatactcactcatttcagttagaatgagggtagagggaggaggaaggctttccagaataaaccgacaatttcccccctta

SEQ ID NO: 796 - DNA, 462 bp, Mus musculus
  misc_feature (1)...(462): n = A,T,C or G
gaactgaggtctactgcgcgccgcgcgagcatacgccgccgtccagaaaccagtccaacccaagcaggacgatgaaccaccctcctctgcgttcatcaaagaatacaaggacatcatccccaacatagagaaggttgntgatnntgtgnntggaangttgnctttgganatggccgnccngactganntnnnanaggtcccgnagngaacaatncgatgaacnanatggtggntnaccctgangngtncngagcntngnnggcttattcnngagccttgaatgacaggntccgcgttnttgtatacctcatgcagatacatnnaaaggacaaagcccacaaacgccatctgntgatgagcatcgaccggaggaaaaagttgntcaaaatcctccgtcagaccaactatgatgtcttcgaaaagacatgcanggagctaggggtggnatataccttaccccct

SEQ ID NO: 797 - DNA, 500 bp, Mus musculus
  misc_feature (1)...(500): n = A,T,C or G
actgagacctataggatnagnnataatgggagcatttttagacaagccnaagcatgnagaancntaatgcccangggcaggngantgggttaccatacngcctacgcngcatgcaaggttgncgagttgaaatggaggacagcacacacggctgttgatctngtttgccaagtggacttgagacatggncattctttgctgtatatgatgggcatgctggttctcaggttgccaaatactgctgtgagcacttgttagatcacatcaccaataaccaggatttcagaggatctgcaggagcaccttctgtggagaacgtaaagaatggaatcagaacagggtttctggagattgatgaacacatgagagttatgtcagagaagaaacatggtgcagatagaagcgggtcaacagctgtgggcgtcttaatctctncccaacatacttatttnattaactgtggagactcgagaggtttacttttgtaggaataagaaaag

SEQ ID NO: 798 - DNA, 524 bp, Mus musculus
  misc_feature (1)...(524): n = A,T,C or G
tgagttgctctaggtcgtaaccagcctttgaaaaaggagaaaccaaaatggaaaagtgactatcccatgacagatggacaactgcgcagcaagagggatgaattctgggacactgcaccagcttttgaaggccggaaagagatctgggatgccttgaaggccgctgcacatgcttttgaaagcaatgatcatgaactggcacaggcaatcattgacggtgcaaacataacactaccacacggtgcactgacagagtgctacgatgagctggggaaccggtaccagctgccagtctattgcctggcaccaccaatcaacatgatagaggaaaagagtgacataaagactcttgatattcctgagccacctcccaattctggacacnaatctcagctccgcctgcggctctccacaggcaaagacctcaggctcgtggtccgcagcacagacacagtgtttcacatgaagaggcgcttacacgccacaaagggcgtggagccaggcagccagcgcttggttcttct

SEQ ID NO: 799 - DNA, 407 bp, Mus musculus
  misc_feature (1)...(407): n = A,T,C or G
aactgaggacttttcccgcaggctgcgaggggacccacagttcgtggccacttgcctcctggggagggcgactctcctcccatccactcaagatgctcaggggtccgggacccgggcggctgctagctgctggcagtcctgtgcctggggacctcggtgcgctgcaccgaagccgggaagagcaagaggcaggctcagcaaatcgtgcagcctcaatccccggtggctgtcagtcanagcaagcctggctgttttgacaatgggaagcactatcagataaatcagcagtgggaacggacctacctaggcaacgccctggtttgtacctgctatggaggaagccggggttttaactgcganagcaagccctgngcngtaagtggaaggcanntctggggccctgggca

SEQ ID NO: 800 - DNA, 513 bp, Mus musculus
  misc_feature (1)...(513): n = A,T,C or G
gattggcataagatatttcttcaagattggacagctgctggcctgctacttctgttagctgcaagagataacaccatcatggatctgcagacacagctgaaggaagtattaagagaaaacgatcttttgaggaaagatgtggaagtaaaggagagcaaattaagttcatcaatgaatagtatcaagaccttctggagcccagagttaaagaaagagcgggccttganaaaagatgaagcttccaaaatcaccatttggaaggagcagtaccgagttgtacaggaggaaaaccaggttagtttcatttatacatttatctttatgggctgagtccatgtatataaacaaaaagaggcattccttttccatcccgtcttagatttcccccatctttggtcttttattttttttatttatcctttttactttagaactctgtttctantatcttagcctttttaagacttgttcctgtacatggtgcaaaacactcacacatataaaattaaaaaa

SEQ ID NO: 801 - DNA, 490 bp, Mus musculus
  misc_feature (1)...(490): n = A,T,C or G
gaactgagggtctgcactgaacctgagctcaccagttcagctagactgcctggccagtgaactccaaagactgacctgtctctcccttcccgttgctggaattacagaaagtatttgctggcctcctgtcctggaaaaatagctggactttccaggatacagtgagtgagtacaagcaagttgctgtagagtgacaagaacctcacaagctaaaagaagagaaagcattttggacattgcgtaacagaagagaatatctactaaggtgcttgtcagctgtgaaaatgtctgacaagaaccaaatcattgccagggcttccttgattgagcaactggtgtctaaaaggttttttgaagaaatcancttgnagctgtcaagtctganttatattttggtatattntntccatatggcattccagggtggntcaccatggngcaatgtgntagtcctttacngagngacttacagagtcatcaacaactcaag

SEQ ID NO: 802 - DNA, 414 bp, Mus musculus
  misc_feature (1)...(414): n = A,T,C or G
caatgcttacctgctnttgnccgaagatctgaanagcgtgnggtatgggagggaccaggcagcaattacctgacaacccagagcgattngtcagtcctgcaanctgtgttgggagcccanattttcacctgcggnagacactactgggaagtgcaagnngngaaagaagacggagtggcaagtggngatctgcanggactttgngaacagaaaggggantttcncccaaanctgnccggggacctcttcncactcataggcttaaaaatcgncgaccnattacaagtctcttggnttncatctcccctgaaagggcaacatgtcncggagnancccgtgncntacaggctggngnagttntttggactatgactctgnancccataanccttatacaattgcgaacaggatgga

SEQ ID NO: 803 - DNA, 318 bp, Mus musculus
  misc_feature (1)...(318): n = A,T,C or G
cacaaacgtgtatcttggtatgcaggggnntaccagtaggacaacttatagaattggctctctccttnnntgngggtcccanagacagnactccggccatcagacttggaagccagcacctctaccagctgaatcatctttgcctgccttaaagtgaatttccttaagagggagaatgttctaggngtggcttcacagagacagtggttaccggccngttttntnagcgnagcttgaagtgggaggctgggtggcacacatgggagaccaaagagatgtgacttntttccctagcctcttttggtgtgtgtgtgtgtg

SEQ ID NO: 804 - DNA, 167 bp, Mus musculus
  misc_feature (1)...(167): n = A,T,C or G
aactgagggaactggaagctgcaagcatcctcagctgacaactgcacctccgcgaatggctgggtctctgtagtaaaactgcggtggnaggccattgtgattgtnatagacccngagaaacctgnactgacgcccttttaaactgcagtcaggaggaggctgactaa

SEQ ID NO: 805 - DNA, 494 bp, Mus musculus
aactgagattgccttgggtccccggtattcacgcccatcaaggcagacataagcggtaacatcaccaaaatcaaagccgtatatgacaccgacccagccaagttcaagaccctgcagaacatcctggaggtggagaaggggatgtatggggcggagtggcctaaagtgggggccactctggcactgctgtggctgaaaaggggcctgcgcttcatccaggtgttcctgcagagcatctgcgatggggaacgcgacgagaaccaccccaacctcatccgagtcaacgccaacaaggcctatgagatggccctgaagaagtaccatggctggctggtgcagaagatcttcaaggccgccctgtacgcagcaccttacaagtccgacttcctcaaggcactctccaaggggcagaacgtgacagaggaggagtgcctggagaagatccgcctcttcctggtcaactacacggccaccattgacgccatctacgacat

SEQ ID NO: 806 - DNA, 265 bp, Mus musculus
  misc_feature (1)...(265): n = A,T,C or G
aactgaggaaggggaagaagaaggagatgaggaaaacgatccagactatgacccaaagaaggatcagaacccagccgagtgcaagcagcagtgagcagtgactggccttgaggacggccgccctggcctaccttctgctttcctggaaaagatgaatatacatcatatgaacctattttgagtttttctgttgtttgtttgcctaattttgtttttatagcctaaaataaaaatatgacacagntttagctctcaaaaaaaaaaa

SEQ ID NO: 807 - DNA, 115 bp, Mus musculus
  misc_feature (1)...(115): n = A,T,C or G
gtagtcccgcttccaccaaaactctgtgtttcctttctctcaaaataaaactcaacaagccataaagcttncgacgccggtggaganggcgcccncgnatttatggcccctgaat

SEQ ID NO: 808 - DNA, 544 bp, Mus musculus
  misc_feature (1)...(544): n = A,T,C or G
gactgagagctgctctcaganctgctgctgctgctgctgctggcacganctntngtccgagtccnntgaggaggaggacgaagaggangaataggaccangnacaggccgaggaagaggaggaggaggagganctagagaggctttcatctgagtcactgtcttctgaagaggatgaggcggatgtctcactctctgatgaagactcactggccgaactgtcactgctagagatcnnctctnngtagananngttcttctttgtcttctttgcgacaaggtctcacgtancctaggctangcctgaagatgctgctgaacgcttgacccctccgcttcccttacacagcgtcacggatggatctcagaaccttgtgcatgctaggcaagcnctctgcaaactgagctacatctcancccgacttctaccctgtgcttggacctcaaaggtttcccaaatgantctctgctccctgatgaggagagtagctcgatcctgccncanacganccagcncgggcttcactctaaccatctccaaaaaa

SEQ ID NO: 809 - DNA, 338 bp, Mus musculus
  misc_feature (1)...(338): n = A,T,C or G
tcccacctgtacctgtgtctgtgtccagccaggaagacnngttgcctnanctacctcccctgcctaanttaccanacaccggntgagaactacgtgactcccattgnagactccccagcagccgaatacgtgaatcaggatgngcctctctccaatcaggcggtccntntgaagnccaagaagccngccnggntccnagcanaacnccnatagncatnagttgtgcctangccagatctcttagccatnnccngngttgaggaccaggangctaggtggaagntcntnccaggcttgctnccttgggacaagacttgnggntatcaccgctgagctgg

SEQ ID NO: 810 - DNA, 240 bp, Mus musculus
  misc_feature (1)...(240): n = A,T,C or G
gactgagaatagtatacaagcttcaggagggactccggaagccaagggcattgagaaacgcaagctttctcagaaacgaaggacactgtagctgccagcagcagtccatgagcatctccaagcccaagtacctggagcaactagaaaactacctgcgcaaggaactcctcctgctggacctgagcacggattctgcacaggaactgaggctgcaggtnaaagcccacagacccaacatgt

SEQ ID NO: 811 - DNA, 133 bp, Mus musculus
  misc_feature (1)...(133): n = A,T,C or G
gagatcttcgaagaactgttgctgtaactcttggtatatttggaacttatggcntacacaatccacangcatacagatactgtgaagcagatttttgctttagaataaaaaagctgtgatacaagaaaaaaaa

SEQ ID NO: 812 - DNA, 512 bp, Mus musculus
actgagtgtatcctgagggtaccggcgggtgtttgaggagtacatgcgggttatcagccagcggtatccagacatccgcattgaaggcgagaattatctccctcaaccaatttatagacacatagcatctttcctgtcagtcttcaaactagtattaataggcttaataattgttggcaaagatccttttgcttttttcggcatgcaagctcctagcatctggcagtggggccaagaaaataaggtttatgcatgtatgatggttttcttcctgagcaacatgattgagaaccagtgtatgtcaacaggtgcatttgagataactttaaatgatgtgccagtgtggtctaagctggaatctggacatcttccatccatgcaacaacttgttcaaattcttgacaatgaaatgaaactcaatgtgcatatggattcaatcccacatcatcgatcatagccctacctatcagcaccgaaaactcttttacactaagggattttgcaagagcagg

SEQ ID NO: 813 - DNA, 410 bp, Mus musculus
gaactgagagtcagtcgctttgaatccaagatccatgacttgcgggaacaaatgatgaacagcagcatgagttctgggtctgggtctctccgaaccagtgagaagaggtccttgtatgtcagggccctgtttgattatgatcggactcgtgacagctgtctaccaagccagggactcagttgaaggcaaagagggataaagtgacagatgattgtgactctcgcgagggggggtgagccgggaagtgttgattctctgatggtaactgacagcagtggacagtgctcaatcctccctcccagggaagaaaagcaaagattcgaagtaagcatgataatagttggtttaccagtgttccttccaaggagacatatattttttaataaatgatagttgcaatgaaaaaaaaa

SEQ ID NO: 814 - DNA, 100 bp, Mus musculus
gaactgagctctaagaacaggtggaattagctatggataattcatacaatttaaatgaacaaagctcatggaatggaatctcatctgagaagaaaaaaaa

SEQ ID NO: 815 - DNA, 152 bp, Mus musculus
  misc_feature (1)...(152): n = A,T,C or G
ttgattcaagatgtcacagacagtncttcccnnntggggagtctgacatttgaagaggaagaactgactcgagctgatcctgagcaatgccatgtgcccctgtgagtagaacgaatgatgatgcagtcattcctgaaaaatgtgggacaaga

SEQ ID NO: 816 - DNA, 365 bp, Mus musculus
gaactgaggtttagagtcaggtcctcccggccaatgctactggggaaatagcccaggcgggctgccaggtctccatagtaacctccctgaacctggagaagaagttaaaagtccatctggggaaacggcacttatgcttgctgaacgcctgtgtacaatcttccctcttacttgaatctcagcttaaatgccatttgctcatggatagaatgttgccactacaactaatcctcacaatgtcatgtttgctgccacttttgatggcttgctggttgcctgcctctacccctttgttcactgttgtatcccgggtatgaaaaagagctttggcgctaagcttctctgtaatttctaaagaaaaaaaa

SEQ ID NO: 817 - DNA, 546 bp, Mus musculus
  misc_feature (1)...(546): n = A,T,C or G
gaactgagattggcttgggcaaaagaatccagagaccctaggatttcagaaggccagcagtctccactggagaagaagatcctgagttttggtggtgtgcatacaacagcagcaagacacttgataactcagaaatgccatgaggagagcaaagcactctgcaaggagcaagctaattctctggactactggcttgcaaaagcagagtcgtattataataagagaatggtggagagaatgaaaaaagacacaggtgatgaaacaaagacaaaagtgcaagaggaaccaccatcaccatcaccaccaccaccaccaccaccacgcagagagagacagaaacagcattactgggtgcctgagagagagaagaagcaaatagaaaggcacattcatagaacaagccatgccagagaatttacagaccaaccctggagacagccacgactcttcagtgaaacaacactacccaaaattgtgctagaggaggaaagcatcccacaagcacagaaaagaaggcaggcncacgagagagaactgctccaaatt

SEQ ID NO: 818 - DNA, 239 bp, Mus musculus
aactgagatggatgtccatggcagaataagagaaattcttgcagagactatacgagaagaattagcaccggatcaacaacatttatcaacagaagatctgatcaaagctctaagacgtcgaggaatcattgatgatgtaatgaaagaacttaattttgtgactgatagtgttgatcaagaactcccttcttctccaaagcaaaccgttggttttgataagcagtcaacattaaaaaaaa

SEQ ID NO: 819 - DNA, 457 bp, Mus musculus
gaggctcgacttcggatggtagacattgtggagaaggaagatgtaaatgaagccataaggttgatggagatgtcgaaggactcccttctaggtgaaaagggacagacagctaggacccagaggccagcagatgtgatatttgccaccatccgtgaattggtgtcacagggggcggagtgtccacttttctgaggctgaacagcgctgcatatctcgtggcttcacaccagcccagttccaggcagctctggatgaatatgaggagctaaatgtctggcaggttaatacttctcggacacggatcacctttgtctgatagccagtttttacaccctcctgcctacaccagagggcatgctgaatgccccctcaagaaaaaatctcattctcccatgctgcacttttttattctgccaataaagtgttttgattgcctcttgcttgttaaaaaaaaaaa

SEQ ID NO: 820 - DNA, 125 bp, Mus musculus
  misc_feature (1)...(125): n = A,T,C or G
tgggattacagatgtttgctgagggaaaatggccagaaaatcctcattcctacgtttgttccttanaaaggctgttggggcaggtaatccagctcctgcacagttctgctgagagccacataaaa

SEQ ID NO: 821 - DNA, 253 bp, Mus musculus
  misc_feature (1)...(253): n = A,T,C or G
gactgaggctatatcggcgtggttaaccgaagccagaaagacatcgagggcaaaaaggacatccgggctgctctggcagccgagaggaaattcttcctctcccacccagcctaccggcacatggctgaccgcatgggcaccccacacttgcagaaaaccctgaaccagnaantgaccaaccacatccggagagtcactgtcgacccttcgcannaagntgnagagccaactgctgtccctggagaaggaagtg

SEQ ID NO: 822 - DNA, 495 bp, Mus musculus
  misc_feature (1)...(495): n = A,T,C or G
gaactgaggagtgccagtgcacaaccaaggnaagtccatcagctttattaacccatcaaaatcaagtgcagggaaaggatatgctgcctcaaacccaaacaaattggccgtatcaagaactatgggttttttaactgacatgaagccggcagaaaaactaaactgttcctagcgctaaaacagtacaggtgcttgattcaaaaggagttgatgtcacaccccgacctctctaccatccagatccacatgctgcgtcagcaaagccaaacaaactcttgacatcacaagaaggatcgcttggatcagattatatttcttcctatagcctctatcanaatacactaaaccctagcatgttagggcagtacacaaggtcagtcctggggagcagntcagtgtcgaagtcaagcatctcaacaactgagtcgatgtcagaggaccttgaagattcctcttacaagaggggatagattggccagttttacagatgtacga

SEQ ID NO: 823 - DNA, 182 bp, Mus musculus
  misc_feature (1)...(182): n = A,T,C or G
tacagcaagtccaaaggantgccnactctccanaggatttaactggnagnggnatgagacttctgatttcacagctcgntggctacctgaaagngaaaacaaaaattcannncatnttttatctatggcatgncngcaaancatcaaaanaaagcgattcttaaacccgtgaaaaaaaaaaa

SEQ ID NO: 824 - DNA, 313 bp, Mus musculus
  misc_feature (1)...(313): n = A,T,C or G
ccacaggtgtcggccagcagtatcctgacccagcaacggcatggtgnnatgtggggctatatgccaagagganagccgtctatnngganaactctctctatcagcctctttcttgcctcggganatncgngctcttctcaataagcctaaggctttactgntaagaatcccaggaggtttctgggataagacaccattgcttaaactcggggnatgcaggaaaggaaagggcagacactgngaagggctcatagaaaancactttcaacaattttgggaataccaaggagaagaatttcctagagaaaaagaa

SEQ ID NO: 825 - DNA, 336 bp, Mus musculus
  misc_feature (1)...(336): n = A,T,C or G
ctgagactgagtaagagagagagagaagagagaagnagaaggagaagatgaagaagaagaagaagaagaagaagaagaagaactgctttccctcgtntccttgctggtctgtcctccccnangaccctgacttgnaacaccnaanccntncacaaccagcancccanccangcancaaatgacacacaccaaggctcatctggctcctgaggttcaaagtgagttttnnttgtgttttattctaacatgttttttcagagttttctgctttgaaaatgaagttcttttttagctggngaagaataaataatatttccaagagccgccctcccagga

SEQ ID NO: 826 - DNA, 520 bp, Mus musculus
  misc_feature (1)...(520): n = A,T,C or G
tcatcagaaaagctaagtttgctatacagtaaggatcaggagatctgaccctggttgcagaaccattcctggttgcagaatacagggttgtattcccacttcatctttccagaccatcctaggaaatactaggaatccttggaagggaaagaaggaacattgttggttggaataaaaacagggttgaatgagttccagaaagcaggcttctcaaactcgtggacaacaatctgctgaagaagacaactttaagaaaccaactcgaagcaatatgcagagaagtaagatgagaggagctgcctcgggaaagaagtcagctggttcgcagccaaagaatctcgatccagccctgcccggaagatggggaggnccgctctgctgagaaccccccttccggttctgtgcggaagaccaggaagaaccaagcagaaggctcctggcnaacggagacggaggcagtacccaggggaagtcccccagcccccctcggaagaaaaagggcacggggctgacccccact

SEQ ID NO: 827 - DNA, 360 bp, Mus musculus
  misc_feature (1)...(360): n = A,T,C or G
actgagactgaggtatcagtccctcacctgaccctgttcctgaacacagtgcacccatctatgagttccggtgacagaggttcagaacagtggccaaataaaactgaacttggcaacaaananctttganaggaaattatgtacctgccatgaaccaggagaggtgtgttcctgtttgttcacgagcagacttcatcagaaagcatgaatgttaacctacaggatccaaggagcatggccaacctgcaggcaggcagagggagctgttcacctcccaccctccttcctctcagtggcattgtggtcaccatctgtttctgaacaaccttaaatgcactttccttcctgcacagctaactt

SEQ ID NO: 828 - DNA, 421 bp, Mus musculus
  misc_feature (1)...(421): n = A,T,C or G
acactcacgtccttgtgcagccttcgccaccatcctccttcagaactcattcatctctcacagttgaacctttgcncacatgaaacagtnagtctctctttttaaagataanttnctattttgattatacatgtggatgcagatgcccttggagaccagacatgtnggagttgnggtgaagatggttataaaccactngangtggngttgtnctggctgccctataactcactntggggnccaggctagcctgaactgcgatgtcttttacctaggttttttcctcccagttcactgatcctgtnntgcatgncnnannnnacctcccttaaanacatatannccaccannntntgcancaatttcagaaggctcaaggngggggacgccctggccctatnttgaccaggtgaaactgcag

SEQ ID NO: 829 - DNA, 536 bp, Mus musculus
  misc_feature (1)...(536): n = A,T,C or G
gaactgagagtcctttggacagaactacccagtaggtaagctgatgggactctggactgcatcagcatggccctgacctgcacctttaacagaagaccctnaataaannnctgntggccntccaccgnatgcagtcncaanctncttgatggaggaaggggctngtgtcagagaanaanaggcacncttcannacanggnncccatncaccaaagangaaacntanacccaccantatanggntncaaggagagccgaatgatgaantccatccactactgngngtaaagggggatggcaaatcgaaanaagantcaagcangccggcctaaaggatnaaaagcaggaggagccatctcagaactgctgngaanaccgtggaacccttggttcttctcactctgtcatcaggtancctttggacagngtggccaatcatctgagattggctttagttgaaaacaaaggcctntgcctcccacccaggaggtgaangagnggattttnngtttgaatgacgaaggcgaattatgccg

SEQ ID NO: 830 - DNA, 333 bp, Mus musculus
  misc_feature (1)...(333): n = A,T,C or G
gaactgaggaagaaatggaagccagaatnaagcaggtactccgctccctgcgtctggagctgggtgccgaggtactggtggaaggactggttcttcagtacctttaccaggaaggaattntgacagaaaaccacattcaagaaatcaaagctcaaaccacaggcctccggaagacaatgctgttgctggacatcctgccttccaggggccccaaagcttttgacaccttcctcgattccctccaggaatttccctgggtaagagagaagctggagaaggcgagagaggaagtcacagccgagctgcctacaggagaaaaaatccctggaagca

SEQ ID NO: 831 - DNA, 226 bp, Mus musculus
  misc_feature (1)...(226): n = A,T,C or G
gatgcgtttggcagagggagcagtggagattctangtcngggcttaaggcctggggcttccaatgatnctctggtnatggntggangcctagatgcctcaccgcanggngcaggacgngngggtcctcncttaantgcacacnggcagcaanangcacaggcggagggaggaaatatagnccnggctgtcactcaccntntcggntgtgtctcccctggttgacca

SEQ ID NO: 832 - DNA, 111 bp, Mus musculus
ctagactgagtccaggctgatactgatttgggcgccttggctgcagaaatagaaggagctggtgctgccaaggagcaggagcctcagaagtcaaagggaaagaagaaaaaa

SEQ ID NO: 833 - DNA, 385 bp, Mus musculus
  misc_feature (1)...(385): n = A,T,C or G
agcacacagctggggcttccctaggcgaggagagatggatctgatccgtatgggtttgaaaggcccgaagactttgactatgcggcttatgaagaattnttttccacgtacctggtgatactcaccaagagggcgatcaaatggtccaaacttctaaaaggaaacggtggtgtcccggaaaagcgtgacagngaaacgctatgtccngaagggaatccccgntggaacatcgtgnccnnncgtgtggatggctcgngagtgganctcaggccccggatggaccanagccntgnatacttaccatcgactgctnccanggcnagatcagctccaggtctggacnaggtcncattaggacannacttgtaccggacattccctgact

SEQ ID NO: 834 - DNA, 303 bp, Mus musculus
  misc_feature (1)...(303): n = A,T,C or G
gaactgagtgtcttctaattgttgccatcatgacggagtttgtatgccttgatacctttatgctgtttaagaatcatgtctttgggggactggagaaatgactcaacagttaaaaaagcactgctgctcttccagaggacccaagtctgattcccagcacccacatgatgttcacaactatccttaactccagttccaggggatntaatgctctcttctgatcttntggagtaccaagcacacacatgatgtacagacatatatacaggcaaagtacacatacaataaaatgaataaatctaa

SEQ ID NO: 835 - DNA, 316 bp, Mus musculus
  misc_feature (1)...(316): n = A,T,C or G
gaactgagagcatacctgccctgctggagttcatacccattcctgagcaacactggtttttgccatggaatccgtanatccagcttgtctgtgcagtaggccactgttcaaaatgaaganaaggaaaaaagagagtggcaacacggaagccagccacacacctgggattgcnggctctattcgtggatcttaatgagtaacaaggaaaagcaaaatgctatttatctttttggcaaaaattaattgntagtatttcatgattctagagatgtatttctccaactanaataaaatttttggatcgtaaaaaaaaaaa

SEQ ID NO: 836 - DNA, 286 bp, Mus musculus
gaactgagggcacagagaccgtgaagtctgcaacacggctgctcatcagcttccagcattcccagccatcgaagtccacagtggagtggctgctccctgggcacctgtgaagattcaggaggaacccacggtcacgctttatgcctttcaagtcattccatctggaaagatgaacagaatgagaaatgtgtgtgtgtgtgccatagaagttgttagttcatacgttcaaatgccttggcatttattagttttgagtaaaaacaacgttacctttcaaaaaaaaaaa

SEQ ID NO: 837 - DNA, 432 bp, Mus musculus
  misc_feature (1)...(432): n = A,T,C or G
gactgagaccctgtcccatcatttgaggtggcgcggactctagacgacagactgcagcgtcccagctttgaccccagtgggcagcgcctacaagacgtgcacatcgcgtggaagaggtgcccanagccacccantgccagagagcanaangaggggacagtgtcccacggggcacacgccatncaanggtaaccacntgccanagtcccgaggacaccctcgtgacagacaccttgctgcagaagaatgaggccgagagacagangggtctccaagaagtccacatcaccttggcagccaggctgggggaggcagaggagaaaatcaaggtgttacattcagcgctaaaggccacacagacagagctgctggagctgaggaggaaatacgatgaggaggctgcttccaaggccgatgaggtaaaaaaaaaaa

SEQ ID NO: 838 - DNA, 188 bp, Mus musculus
  misc_feature (1)...(188): n = A,T,C or G
agactgagtacaagacagcaaaatacaactgccaattatacagctgcggaaagtatgggctgaagcagtgcactacgtatctgggctaaaggaagactacagcaggctcttccagggacaaagagcagcaatgttnnnagattgtgttagaagcgtagnagccatgtgtatcatgtactcaatgaatt

SEQ ID NO: 839 - DNA, 457 bp, Mus musculus
  misc_feature (1)...(457): n = A,T,C or G
atatatctatctggcctttncaaattggnccctgcnnantggcttncacnaagaagggttntngtangaaganggnccatcatcgaggtggtgatccangantacaccatcntcattnacnagcncattcntagngtgganttcattangccgtgctccttggacactcnnaganatccanangttngctatgaagganntgnacgattccngatgtncatatngacaccnggctcattaaagncatgtgggtcaagggaataaggaatgctccattgcatctgagtatgttcgtccagaanacatagtacggaggaggagtcaccagacaagctttacacattgataacttactgnctgtnaccacattcaaactctacagaangtcaatgtggatgaganctaacctgctgantgtcaaataangttgcagaactgccaaacaggtaaaaaaaaa

SEQ ID NO: 840 - DNA, 325 bp, Mus musculus
  misc_feature (1)...(325): n = A,T,C or G
gaactgaggtacctttcagatgaaggcgttgaagcctgcacaagctctcctgggaaaggcagcataaatgacatcatcctgattgctctcaatgnagannaaggcacctatgatcacaccattgnntnccgcaaaggaggtgatggacagncacaaaggnntactnggccancgcagnaantntaccaanctnctcgagctnggaactaangngaangggcttttgagaaggaagatccactgactganccatnctggaagcccactgatngattttccacttnttcagagagagactcaatatgctggatatgatttttggaga

SEQ ID NO: 841 - DNA, 470 bp, Mus musculus
  misc_feature (1)...(470): n = A,T,C or G
tctgacctggcgtttgatgananagattggaagctccaagtcagatcntgaaganctttnangctcctccacaaatctcnncgaccacnncccttcatcctggcgagaccacgatgacgcaacctcaacgcactccgcaggcaccccaggaccctccagtgggggccatgcttcccagagtggagacaacagcagtgagcaaggcgatgggttagacaacagcgtagcttcacctggcacaggtgatgacgacgatccagacaaggacaaaaaacgccagaagaaaagaggcatattccccaaagtcgcgacaaatatcatgagagcgtggctcttccagcatctcacacacccgtacccttcagaagaacagaagaaacagttagcgcaagacacgggactgacaattctgcaagtgaacaactggtttatcaatgccagaagaagaatagtgcagcccatgattgacc

SEQ ID NO: 842 - DNA, 260 bp, Mus musculus
  misc_feature (1)...(260): n = A,T,C or G
ctgagactgagcatgcatgctttatacantccataacacatacgatgacattgaaancactnncggttgcagatctaggatgnggctgtggngtacttatnanntngaacaggcaatgctaggagcagggttgngtgttggatttgacatanatgaanatgcactagnnaatatttaatatngaatngcggaanagtgngagctaacantggttgntatgatncntagtgatgtgtactcattatctaacagaatgtcca

SEQ ID NO: 843 - DNA, 358 bp, Mus musculus
gaactgagatggccgccaagagcgagttatgtttgacaaaattacatcacgaatccagaaactctgttatggactcaacatggactttgttgatcctgctcagatcaccatgaaagtaatccaaggcctatatagtggggtcaccacagtggaactggacaccctggctgctgagacagccgcgaccttgaccacgaagcaccctgactatgccatcctggcagcaaggatagccgtctctaacttgcacaaagaaacaaagaaagtgttcagtggtacgtctggtataaaaacatgagaaagcgtttaatagtgtcataaaattataggaataataagtgaattttaaaaaaaaaaa

SEQ ID NO: 844 - DNA, 543 bp, Mus musculus
  misc_feature (1)...(543): n = A,T,C or G
tgaggtgccctatgagacactgaacaaacgcttccgagctgctcagaaaaacatcgatcgagagactagccacgtcaccatggtggtagctgagcttgagaagaccttgagtagttgcccagctgtggactctgtggtcagcctattggatggtgtggtggagaagctgagtgtcctcaagaggaaggcagtagagtccatccaggccgaggatgagagcgccaagctctgcaaacgtaggatcgagcacctcaaggagcacagcagtgaccagccagcagcagccagcatgtggaagcggaagcgcatggaccggatgatggtggagcatctgctacgctgtggctactacaacacagctgtgaagctggctcgccagagtggcatcgaggaccttgtgaatatcgagatgttccctgacagccaaagaagtnggaggagtccttggagaggcgtgagacagccacctgccttgcctggtgccatgataacaagtcccgactccggaagatgaagagctgcctagagtttcagcctcaggtc

SEQ ID NO: 845 - DNA, 275 bp, Mus musculus
gaactgagggccggcacatgggtgagggggtaccccctggggcgtgagctgccccgcacaggatgccccgtgccccccactccatgcccctgctgctcctgctgctgttgctgtcatcgctcccccaagcccaggctgcctttccccaggaccccacccctctgttgacctctgaccttcaaggtgcctctccatcatcctggttccggggcctggaggacgatgctgtggctgcggaacttgggctggaaaaaaaaaatttttaaaaaaaaaaa

SEQ ID NO: 846 - DNA, 537 bp, Mus musculus
  misc_feature (1)...(537): n = A,T,C or G
tgggaaaccggcaccagtcgactctacagtgctctcgccaagaccgtcaacagcagtgcagctgctcagcacccggagtacttggtgtcagctgaccccgaacatctggagcccatcgatcctaaggaacttctggaggaatgcagggctgttctgcacactcggccaccccgctaccagcggggattttgtggacctgaggacagattgctccagcagccactctcccattcgcgtcatgcagtggaacatcctcgcccaagctctcggagaaggcaaagacaactttgtgcagtgccccgtggaagcgctcaaatgggaagagaggaagtgcctgatcctggaggagatcctggcttaccagccagacatactgtgcctccaggaaagtggaccnctacttttgacacctttccagccacctcctcagtagactggggctaccaangcaccgttttttccccaagccctggtcaccatngnctagantgtggaancacaacaacggtccaagatggctggtgccttatttttttc

SEQ ID NO: 847 - DNA, 158 bp, Mus musculus
  misc_feature (1)...(158): n = A,T,C or G
gaactgaggttgctgttctggctgaggccatctatggccctggnagtgttttcattcaagacaaagtccattccatctttatttattagctgagcaaattcagctaataattttcaagacccagattcaagcaataaaacatttctctgcaataccaa

SEQ ID NO: 848 - DNA, 123 bp, Mus musculus
  misc_feature (1)...(123): n = A,T,C or G
gactgaggtgctgctgacttctgataaagctgcggccaatttctcagatcgttctttgctgaagaacttgggacattggttaggaatgatcacattagctaanaataaacccatcttacacac

SEQ ID NO: 849 - DNA, 543 bp, Mus musculus
  misc_feature (1)...(543): n = A,T,C or G
aactgaggtgaagagactgttttctgaggctgcgttggaggctgtgaccgagcagagagcccatgtgaactgatggggaggctttctgaataacatggccttttgccattagccttgccatgaccacatttttcaccagcagtccctccctggattcaagatgcaaagcaggaggaggaagtgggctaggaaactagttcccaggcctcggggccgggaggcagagagtcaagtgaagtgccaatgtgaaatctcggggacaccattttcaaatggggagaagctaaggcctcacagccttccccatccggagcagagaccatatagctgtcctcagctgcactgcggcaaggcttttgcctccaagtacaagctgtataggtaggtgacactcccattcctctttcagtttccaggttcctggagctcacaaccagagactccattgagagtgaacggtaaagntgtcctgtgagtatactatgaactctaaccatttgaagtcactcactaaatagtaataatggtttagctggatatgga

SEQ ID NO: 850 - DNA, 307 bp, Mus musculus
  misc_feature (1)...(307): n = A,T,C or G
gtgttccaatttgcaaggcaagaaggagttcatggtttttatgacttagcagtggaagccaaactatcactttcaccaaattcaacgtattaacagcaagtcaccaaccattcagggctggagaggtggttnagnagntagggctgctcttccagaggatcccagttcaaatcccagcatccacatggnagctcacagcantgtgtaactncagttctggggattccagcncccccatacagactgtacacgcngccaaaacaactaagacataaaatantgataaatacattctttaaaaaaaaaa

SEQ ID NO: 851 - DNA, 546 bp, Mus musculus
  misc_feature (1)...(546): n = A,T,C or G
gaactgaggagtaatggagtccaagcatcaaggcgtgaaaaatctcaacatggagaatgaccatcagaaaaaggaggagaaggaagaaaagccacaagataccatcagaagggagccagctgtggccctgatctccgaggctggcaaaaactgtgcgcctagaggaggtcgcaggcggttccgggttcggcagcccatcgctcactatagatgggacctgatgcagagggttggggagccccagggaaggatgagagaggagaacgtacagaggtttgggggtgatgtgagacagctcatggagaagctgagggaaaggcagctgagccacagcctgcgggcggttagcactgacccgcctcatcatgaccaccatgatgagttttgcctcatgccctgagtcctgagcttttctctgaagatgacatggggacccctgccttgtgatgtacaaattttgaggttactcctttttatgtagatctctccctgttaccagcttctaattgaaacttgngntttttactcggcctataattaagcaaa

SEQ ID NO: 852 - DNA, 352 bp, Mus musculus
  misc_feature (1)...(352): n = A,T,C or G
gaactgaggtgacaggacaccctcaagggctttgatgagaaggcgtacctgtcagccaagcagctgaagcctggagaggacccatacagacaacatgccttcaaccaactggagagtgacaaactgagctcagaccgacccatcagggacacccgccattacaggtatggcatctgctgagtggacaggaagggaaaactaagaaaagaccagtgttggncagcttggtttntttcactcatatgtgtttattgatttattacatttatttaccttattcatgtgtctgtgtgtgtgtgatcgtgcacacatgcaaactgtatatattttacaaataaatatgatgtcctca

SEQ ID NO: 853 - DNA, 286 bp, Mus musculus
  misc_feature (1)...(286): n = A,T,C or G
cgttcctggaagaggaggaggaggaggaggaggagagaccaggattcgaactcaggcccagcttcgatacgctatggaatcactgncagggccagcttcccctgtggctgagttcctgtctttgatagatttctgtgaaatcctcacagccttctcaattagcagctggcatctttattgattgatcaaaaaccaattggggacaaggaccttcagcatttggacaagcagattcccgattaaatcaaagcattagaaccaattctcaacagtgtaaaaaaaaaaa

SEQ ID NO: 854 - DNA, 479 bp, Mus musculus
  misc_feature (1)...(479): n = A,T,C or G
cgttctgacgccagcactgagattctcttacctgcctttgtagttattcacatcaaagatcttctttgcacggtantacacgagcttccagggccngancagatgcccttccctgcagagaggtggaanacagtcagagaggcggcntgnggccatcgtgagatggtcacaaacaccaaaggaacctactggagccctgcgtgacatggttaagttcatgtgcagcagtgtgcagaggcatgctggacatanaggcatgctggggctncatgcncctgcnctgtcagaacaggtacagttaacactgccttgaggagcaagngcacgctgggtttgcagatccactcaccggattgccacttnaacatcatctgagaggcaccaagctcattatcncccagncaangcctccctacctnccctctctnnanaagagccttctcctgcccnctgccctncctgtgtaaactgaaaacact

SEQ ID NO: 855 - DNA, 194 bp, Mus musculus
  misc_feature (1)...(194): n = A,T,C or G
atcagtcaagaagaattanaagaataaggttgggatccactgntnccacanatgaagacntgacncccagcccagaccnnganccagaccctaccgaganntntgncggaaataatggagacctgtcaccctgcaaggtggaagaaaacncngcccagaagaatacagaaacagcctctggcatcggggatgaa

SEQ ID NO: 856 - DNA, 439 bp, Mus musculus
  misc_feature (1)...(439): n = A,T,C or G
aactgagatggccgccccattggggtggagggcatccaggtgctgggcacaggaaaccttatcatctcagatgtgactgtccagcattcaggtgtctatgtctgtgcagccaacaggcctggcacccgggtgagaaggacggcccagggtcggctggtggtacaagctccagcagagtttgttcancatccccagtccatctccaggcctgctgggaccacagccatgttcacctgccaagngcagggggagccaccgcctaatgtcacatgnttgaagaatggacaagngctgggggcaggaggccatgtnaggctcaagaacaataacagcacacttgagcatttctggagtcggacctgannatgaggccatctaccaatgtgtggctganaacattgctggctcgncncaagccagcgccaggctgaccgtgctg

SEQ ID NO: 857 - DNA, 104 bp, Mus musculus
  misc_feature (1)...(104): n = A,T,C or G
aactgaggtgtttggccttggttccgtggctcacatggttttaggagaaaactctggcagctatctcggcgtcnacttgggttttggattcggagtgaccatgg

SEQ ID NO: 858 - DNA, 301 bp, Mus musculus
  misc_feature (1)...(301): n = A,T,C or G
gactgagggtaaacttgatgattaccaggaacgaatgaataaaggggaaaggctcaatcaagaccagctggatgcngtatctaagtaccaggaagtcacaaataatttggagtttgcaaaggaattacagaggagtttcatggcattaagtcaagatgtaagtaaaaaatactgtgtaaaatataaaaattaagttagtggaaactatttggagttagtctgtacattactgttgatgagttctctgccaggtgtggaaattggattaaatcatttcttattaatttgccaaaaaaaaaaa

SEQ ID NO: 859 - DNA, 321 bp, Mus musculus
  misc_feature (1)...(321): n = A,T,C or G
actgagggctggagggacatctgaagaggtctcatcatggcggacagcnctctggggacagctttngngatgcctanacngagggntggntactgnaccatgaacacantggaggatncctcctttgcttctttncgcttttctcctgntttaaaaccccatagtaaagtactttctttntctccttctttcttttattaacattcagaaatacacctgtnntgngctgagttattntggaggataaatnatagtttttgctttngtgaagnnggcattnncacctttgccctaanaaaatngagtgtagaaataaaaaaa

SEQ ID NO: 860 - DNA, 497 bp, Mus musculus
  misc_feature (1)...(497): n = A,T,C or G
aactgagcattggaaaaaggtcagcaaggatgtctcagtgggtaaaggcaagcctgatgttctatcaggtacctgcctgtaaggaaagaatggacttctgaaaaatgaaacaggagagtcagagcctctgagcaagacctgggaggtgctctggagtgctctgatgaaaccttacagaaaaaagtgaagtcggactcacctagttcccaaagagtgggaagacctcaatgacctcttactgcccagggaaaaaggattggaactcattaactctttgtgaaccagagagaaaagaaaggaagaaaattggaggctggagagatggctcagctgttaagaacactgactgctcttccaaaggtctangagttcaattcccagcaaccacacagtggttcactatcatctttaatgggatccgatgcccttttctggtgtgtctgaagacagtgtactcatatgcataaaataaataaatatttctacaaacaaaaaaa

SEQ ID NO: 861 - DNA, 411 bp, Mus musculus
  misc_feature (1)...(411): n = A,T,C or G
aggaggaaagcctggaangctcccaccaagcggcggcggtncacggntntgatggagggaaagnacatgntccacatgcccaaggggaccacngccccgcatccgtgangatnctggagcaccancangcagcctaaggtctccatanaacatcncgtagtttnatcgncttggngnttgattncanagcagacaccnagcacagggactangcctttttgagttttcagccgcttagggnctaccccanagatggatgagtgatctgnccttgggacagtnacaatgacggnnngcantagcttctgtnctagcantatnctagnnntcctganngaaactaggaccangccccaaccaaggccangtgcccaaccagnnctgactgccaaagccaaaagccaccaagct

SEQ ID NO: 862 - DNA, 436 bp, Mus musculus
  misc_feature (1)...(436): n = A,T,C or G
gacgtgaacctggaactnaccaactctgctagactgngnagcnntgncaatngctctagcacagatnaactnngagggcntnanncccactgaaataagccagngacaaaaagaccgnatgttgtgatcacacttaaataaggnangtanactgtctggtcaagcangtaggggcanacgagcagaacggctagcccgctangggatgtggggagggcagaatgggcatttgttgnttaatggatgtaaaggttcagttntgcaaaaggaaaaggtgaggcttgatgacattgctctctagcagaacgctcanctagcatttctgagacctganntcatgtncaacagctcatgcacacacacatgcacaggagaaagaatggaangaanctaccccttttacttaaaaaaattgnattnattttttattttatgg

SEQ ID NO: 863 - DNA, 524 bp, Mus musculus
  misc_feature (1)...(524): n = A,T,C or G
gaactgaggtgtgatccgctcactgcatgagcatgggccgattggagcataccttctgcacagagacccggccctacaaccagggagcccggctgacggccttcgagctggtgtacgagcagatccctgcaactctcatcactgacagcatggcggcggctgccatggcacaccggggtgtgtcagccgtggtggtgggagctgaccgtgttgttgccaatggagacacagccaacaaaataggcacctatcagctggccattgtcgccaaacaccacggtgtccccttctatgtggctgcccccagttcatcctgtgacctccatctggagactggaaaggagattgtcatagaggaacgccccagccaggagctaactgacctgaatggagtcaggattgctgcacaggaaatccggntttgggaatcctgcctttgatgtcaccccccacgaactcatnacaggnggnatnatcactgaactgggtgnntttgcccctgangagctccgaggagccctaag

SEQ ID NO: 864 - DNA, 107 bp, Mus musculus
tgagactgaggggaggcacacggtccaagtccttttactttctgaggatgggcagatcctggcagaagcagatggactgagcacaaaccactgggtaaaaaaaaaaa

SEQ ID NO: 865 - DNA, 454 bp, Mus musculus
  misc_feature (1)...(454): n = A,T,C or G
ggtgtcaacatctccagggggcccgaancgccatctcccgcgccgccagtgtcttcgttttgtatgccacatcctgtgccaataacttcgcantgaaaggaaagcgcaagactctcaatgccagtgacgtgctgtcagccatggaagagatggaattccagcgattcataacaccattgaaagaagctctagaagcgtacaggcgggagcagaaaggcaagaaggaggcttcggagcaaaagaagaaggacaaagacaaaaaggattccgaagagcaagacaaaagcagagaggaggaggaggaggatgaagagagactggacgaggacgaccagaacgaagaggaggaaatagacaactgaatggagagagtaggcanactgtggcgtggtggaggggaccctgagatcctgagacatgtctgtgtgaaggctttccctgcaaggcagagcca

SEQ ID NO: 866 - DNA, 295 bp, Mus musculus
  misc_feature (1)...(295): n = A,T,C or G
ccctgttccccaggcagagtacanaactnagcnttgctggtgaaggtccnactggaggacacccaagttcancatgatcttgacagacctgtgactgacctgaccctccctncagtcctgtgccactgacacatatgcttgatgccttctgctttgtcctagtacccaagacggtgagtgaagcaggggtcaaaagttcagttctcagctgcatagtgagtttgaggccagcctgggttaaatgatacactgctttaacacaatgaaataaaggacaggagagattggtttggaa

SEQ ID NO: 867 - DNA, 361 bp, Mus musculus
  misc_feature (1)...(361): n = A,T,C or G
gactgaggtttatcatggcttccagcccaccgtgttgatgcggctggtagcttcagcatattctatcgctcaaatggtactatnnttaagngaagaantgaaagagggttagttccaantgactgtccaggtcattctgtgcacgtcancattnctgccgataaaccaganttcttcagggcacagagncttaagttactccagantttcagtngtnatgntttttcacagtantttttaaaatntntgatnccaaataagaangcatatagntgatattattnatgtcngatttatgggggaaagttnctctaaaancatagtcctatctttncatattatatatcagatttttttcaaa

SEQ ID NO: 868 - DNA, 504 bp, Mus musculus
gccgaggaccagcagccaagcactaggactggacatcccagcaatgaccagctctcctgtctccagagttgtctacaacggcaagaggaatagcagtccccgctctcccaccaacagcagtgagattttcacaccggcacacgaagagaatgtgcgcttcatttatgaagcctggcagggtgttgagcgagacctgcgcagccagctgtccagtggtgagcggtgcctagtggaggagtatgtggagaaagtccccaaccccagcctgaagaccttcaagcccatcgacctgagtgacctgaagccgccggaacacgcaggatgccaagaagtcctagagctcctggcgccctcccctggcctccagaagctcaggtgcacttgatggtggtttcctgtgttcctgtggcgctgctcctggggggaagagtgtgctctagcaggccgggatcccagagccagcaggattgatgaccagggcccttcctttcctctccagggtct

SEQ ID NO: 869 - DNA, 405 bp, Mus musculus
  misc_feature (1)...(405): n = A,T,C or G
actgaggtggggtctttcacgatgacccgagagtgacgacgacgacgaggaggaggacatggtctcggaggaggacgaggaggaggaggaggatggctgacgccgaggagacccaggattccgaggacgaggaggaagatgacatggaagaagacgacgatgactccgattatccggaggagatggaagacgacgacgacgacgctagttactgcacggaaagcagcttcaggagccatagtacatacagcagcactccaggtaaccacccaggccgccgggagactccttccccacctcctccctccccctggcttgctccctctccagtgcagtgcatcctgcttcctanctccccccccccttcccggcctgtcccccacgcggaggccacggcgcgtttag

SEQ ID NO: 870 - DNA, 556 bp, Mus musculus
  misc_feature (1)...(556): n = A,T,C or G
gactgaggtgaccataggacngnggcttaccggcacntaccacctgatatcanaggtntgccctctgatgatttcccnaagccacctgaggnntgngaancataaatcctantgncacngaaggaccattatggaccataggaccctgaataggtgtttgnctatncagagaacnctannctcctattaacnggtatattntnttcgancngnagaccntntncntgtcggggtgccccgtnganagcagagtaaacggntggataatagagtacgatcgacanctgaagaagacntgagnagagtttagggtccctgagcttgccccgaatanngancnaagagngggaggacataagngctcgagaactaagaggantttcagttcgccttggnttagagaatcttccagccgagggctcctcagatctcagatgcccactggctaatgtcgccagtaaacccattgagccagagaagttgcatggcaaaggtgacgcatcgagtcagtccttgggatgcttgggncctgggctgacttctctgaaggcgaacg

SEQ ID NO: 871 - DNA, 461 bp, Mus musculus
  misc_feature (1)...(461): n = A,T,C or G
ggttgcagacaatgatggtgagttcctgttaatagaagcggccgactttctccctaagtggttggaccctgataacagngccaacagggtgttttttcatcatggggagctgtgcattattcctgtgccgaggaaatctgaaaggataccatggttacccatgacaccaccaacaatccagcaagctttgagtataatctcggcacacccagaagctgttttggcttnnnaatctatacaagctgctgtggacaggcgcgtcagtgggtacccagaaagggtcgaagcatccctccatcgagcacactgcttccttccagccggcattgtggcggtgctcaagcancagcccaggctgctgtcggcagccgtccanggccttctacctacgggaccccatttgacctaagagcctgtcgtgtnttcaagacatttttgcctgaaacacgaaataatggcct

SEQ ID NO: 872 - DNA, 289 bp, Mus musculus
  misc_feature (1)...(289): n = A,T,C or G
tgaggtctgtttctgcaggcatctncgtggctccatgantcttccctttgatgacagagacctggnctgtgagtgggagagngaagacctgcccctgaccattcagcttccattccgncttatcctgcntgtngggnacctgccctttgatgaatactggatccccctgtgcangaactcttggctaatgacatgcatggattgatttaccttacatagcgctattgagctcaaattgtgataatgctgccattgagagaaactcacccacgaactgcttataaaaaaa

SEQ ID NO: 873 - DNA, 465 bp, Mus musculus
gactgaggaatttaactaagatgatggcaacttgtactccaagaaagaggaagcgatatacactgaatgctgataatgatgatagcctattaacagatatttcatcaagcaaattaagatgtgctgaaaatctatttccatcacctaataagaaacataattatcaaagcagtgttcaaaaggaagacaagtcttgctcccaccaactccatttcccttcaagtccactcaaaacaactgaaaatagtaggttttcatttgcaaaccacagctcattatttaaaccagctatgtctactgtatctttctacagtaaagagaaatactacctcaatccactcgagaggaagttgataagagagtgtagatctatttgtctagcaactgaaagtggagataaacctattcccagtgtgacagaaaatattcagagaaaaccagtctgtaccaagaagaataaaaaaa

SEQ ID NO: 874 - DNA, 386 bp, Mus musculus
  misc_feature (1)...(386): n = A,T,C or G
gactgagtttttagttgcatttttaaagaaggaaggctcatggtgcaaattgttatttccagtacgggggctgaaggcctggcagaatgggtgctgatgganctacagggngagatcgaggctcgctacagcaccgnattagctggaaacctcctgggagacctacattacaccactgagggangagatgtnatcnacnntacccnagcctnaatcctgcttnctcnnatngnagnnttnngctcattcgntntnccacgcccactgntggaacntgaggagccacacacngncacctttctactggaccagagattattnggcngggaantnggctcccctccnncttgcttcatnactggtgtcgcaacaataaaattagaacc

SEQ ID NO: 875 - DNA, 533 bp, Mus musculus
  misc_feature (1)...(533): n = A,T,C or G
tctcctggtctgtgttctgattcttctttggaggagagtttaaggatcacagttggcaatgaccacttctgtgttagcacaccagagcggcggcggntcagtgaccggctgggatctccagtggatggtctgcaggacatggacagggatgacctgactgatgattctgtcttcactcgaagctcccagtgttctcgggggtcttgaacgatacatttctcgggaggagggaccacttagtccctttcttgggacaacttggatgaggactaccgaacaaggagaaacttttctgcatcgacctggagtttagtccccagagcagttgtcatgatgaattngttgagggaaacagagcggtatnagaaacaaacttaaaagctcctcctattccatacgatctgaggaaagaggccgggaggccaaaaaggcccccgtttttgatgacncagagaagggtacnccagctcaaggaggggggatcacttcaangttttnacatcnngggactcgaaaacctatcggggaggcgg

SEQ ID NO: 876 - DNA, 306 bp, Mus musculus
  misc_feature (1)...(306): n = A,T,C or G
gaactgagatggcctcacggtacatgatccagctcacttctgtgttcaaggatgtctgtctaacgccagaagagatgaatctcgtggtggccaaagtgttgacgatgttctcaaagttgaatcttcaagaaattccacctttggtctatcagcttctggttctctcttccaagggacgcanaaggagtgttcttggacgnatncatagccttcttcagggagctggataagcagcacagatacattttttccacataagaatgcactttggcttatacaataaaactttaaacattaaaaaaaaaa

SEQ ID NO: 877 - DNA, 541 bp, Mus musculus
  misc_feature (1)...(541): n = A,T,C or G
cggagcaccataacatggngtgggaagtgaagacaaatcannatgcctaatgcagtncagaaactcctgctggtgatggacaagagagcaccagggatgagngactcgctggagttgctgcagtgtaatgagaatttgccatcctctccaggatacaactcctgtgatgagcacatggagcttgatgaccttcctgaacttcaggctgttcaaagngaccctacccaatctgncatataccagctaagttcagatgtatctcatcaagaatatccaagatcatcttggagccagaatacctnagatataccggaaaatactcaccgtgaanatgaggnggactggctaacagagttangcaaatattgccactagtncacaaagtcccctgatgcantgctcattttataacaggtaggagcatggattatgttttgcnttntcanacttgcatttattaggggccagcaatnatactantgtaagctncanntgggttaangaanccttgcccaccaagcnctgaagggcgcaaaccttg

SEQ ID NO: 878 - DNA, 340 bp, Mus musculus
cacaacacagtcggattcttctcaacagcctttattgcaggatcgccttgatgcttctacggggaccccaggcgcccagggagccctgcttatatgtaccccagcacaggagaaggctccgtggccccctggggttggtcagtctgccagcacccaatttgcatgcacccgcttgggaggggttggccccaaattcgaattgacgcctgcgcagtggtgttgtttatgacaacggcaaccggaaaccggcgccatcttggagttggcttcccacagagaggagagggagaggagagggagaggagaggaagaggaagaggaagaggaagaggaagaggat

SEQ ID NO: 879 - DNA, 537 bp, Mus musculus
  misc_feature (1)...(537): n = A,T,C or G
gaactgagagatgaagcagatgaatcttgtcactgggaaacagngcctaataaagagggctcctttctccatcagtgccttcagttacatctgtgagaatgaggccatccccatgccaacccactggganaacgtgaaccctgacgttccctaccanctggtttccctgcagaaccagacgcacgagtataatgaagtggccagtctttttgggaaaacaatggatcggaaccganttaaaaggattcaaaggatccaaaacttanacttatgggagttcttttgcaggaaaaaggctcagctcaagaaaaagagaggtgtcccccagatcaatgagcagatgctatttcatggcaccagcagtgaatttgtggaagcaatttgcattcacaactttgactggagaatcaatggtgtgcacggngctgtctttggaaaaggaacctatttcgctagagatgctgcgtactccagtcgtttctgcaaagatgacatcaagcacgggaacacattccagattcatggggtcagcttgaa

SEQ ID NO: 880 - DNA, 481 bp, Mus musculus
  misc_feature (1)...(481): n = A,T,C or G
ctgaggactggattcagcatcaaaatacctcccccacattgagagctgtcggcagttaaggcaacaataccctgattggaatcctgagatcctcccatccagaagaaatgagagcaataggaaagaaaatgaaactccaagaagacgttctcattcccccagccccagacattctaggagatcaagctcaggtcacagaatccgtcgctctcgaagtccagtccgttatatctatagaccaaggagtcgaagtccaagaatttgccaccgtttcatttctaaatatagatccagatccaggtccagatccaggtcccgttctccatatcgtagtcggaatctccttagaagaagtccgaagtcctatcggtcagctagcccagagaggacatcaagaaaatcagtgagatcaggtacaccgatnntcctaatgccatcattttttttgagttttggaaatgagatattggattttaattaa

SEQ ID NO: 881 - DNA, 429 bp, Mus musculus
  misc_feature (1)...(429): n = A,T,C or G
gaactgaggaagcatcatgacgaagaggcattggaaacgctgacagtatcccccatctgggttccgcacccatggggcagacacatgctcatcaccagcaagatcagcttcttcattttgacaacattgcactagctgctccggaacccacactggctctgcgattctgtggaaacatgcaaacagaccctctcgcccagaccaacacgggatctggaccattccatgatcctgtaagcacatccttccatttagcataccccagattactggggctgttattagcatgtcgttcagcagcagagcgnccttccttttccaattgcaaaaaatttaaggtaaaaagagctaaggagagtcttatctttggggtatggccatagcctattccccctttttgttttattaataattcttttaaggttctaa

SEQ ID NO: 882 - DNA, 537 bp, Mus musculus
  misc_feature (1)...(537): n = A,T,C or G
gaactgagcaggctcctctncncatgctcctccaaggaatggtaaggtggcccttgacccacacacatatctatcaagaactcaagagcacagacntgaagtaccnggaaccgtgtgcgcagccgcatcagcnacctganggaccccangancccatgcctgaggcggantgtgctcagtggggccatttccccatagctcattgccangatgacagcggangaaatggncagtgatgagctgngggagctgaggaacgccatgactcaggaggccatccgtnagcaccantatggcgaagacaggcggcaccaccactgacctcttgcggtgtatcaagtgcaagaagaagaattgtacctataaccaggtgcanacacgcagtgccgatgagcccatgaccacttttgtcttatgcaatgaatgngggaatcgctggaagttctgctgacggaaccacccaccaggatggcagaggataaggtgaagaagacccagcaggaagcacagaccacgtgacccagagcaaaaaaaaaa

SEQ ID NO: 883 - DNA, 220 bp, Mus musculus
  misc_feature (1)...(220): n = A,T,C or G
ggacccaccaggtttcagctgctcccaccagctctgcaggggatggattcaaggcaaacctcgtctttaaggagattgagaagaagcttgaagaggaaggngaacagnncgtgaagaaaatcacggcggcatttttgccttgaaagtgaaagatggccctggaggcaaagaagctacctggntggtngatgtaaacaatggcaaaggatctgtgcttccc

SEQ ID NO: 884 - DNA, 264 bp, Mus musculus
  misc_feature (1)...(264): n = A,T,C or G
gaactgagggcttcttggtaatgcccaggctaaggcactaggaccagcctcagagcagtcagagaatgagaagaagcccaagagngnagngaagaagaaaaggtggaatcntcccaaaatgtctcttgcccanaacntttnatcgggttgcctcaanagaaggcaagcttcctcagnagctcacgganaggtcctgctngaaacctnnccgcngntttctatgnannaatttntttcatacnttttttttgggggggggggccc

SEQ ID NO: 885 - DNA, 123 bp, Mus musculus
  misc_feature (1)...(123): n = A,T,C or G
cagaaagaaagcttttagtaaaacancctttttaatagcctggccacgcaagaaggtgaatgtttnggctcttaagaggaanaagttcaaaaaataaattgtcttgtaatgtaaaaaaaaaaa

SEQ ID NO: 886 - DNA, 440 bp, Mus musculus
  misc_feature (1)...(440): n = A,T,C or G
gaactgaggaacttaagaagtatggagttaccacantagtacgagtatgtgaagcaacttacgacactactcttgtggagaaagaaggcattcatgttcttgactggccttttgatgatggtgcaccaccatccaaccagattgtcgatgactggctaagtcttgtgaagattaagtttcgtgaagaacctggttgctgtattgctgtccattgtgtcgcaggccttggcagagctccggtgcttgttgccctagcattaattgaaggtggaatgaaatatgaagatgcagtacaattcataagacaaaagcggcgtggagcttttaacagcaagcaacttttgtatctggagaagtaccgtccgaaaatgcggctccgcttcaaggattccaatggtcatagaaacaactgttgtattcaataaaactggggtgcctga

SEQ ID NO: 887 - DNA, 389 bp, Mus musculus
  misc_feature (1)...(389): n = A,T,C or G
catggaaaagtgccggcttccagccctttgcggcttttgcagtggatgaccgtgtggagatgctaagcggtggacagcggcagaacactaggaaacagaaggcttgggacaaagttaatagcgcctgttcacaaagttcacaaaggacccgttttcactgtgtcctacaattgggtcatgtagggctggccaggtggctcagcgtttaagaacacagttgctcttccagaaggcctgcttctattcttcctagcacccacatgggtgctcacaaccactagggtcactctgcttccaagggtttgatgttctcttntggggctntgaggactgagcaaagaggnggtgcctggttatacatacaggcaaaacattcataaaataatcag

SEQ ID NO: 888 - DNA, 361 bp, Mus musculus
  misc_feature (1)...(361): n = A,T,C or G
aagaaaacttcaaactccacagtcggaggagagtgacttgcattatgcggacattcatgtgctcaggcaaattcggccacactctctccacacagtgaaatgcctgcattcagaatctgctacagagtacncnncncttcnnttcccccaggccacacctcantatgacagcaacaacggaaccctggtgtgagccctcgggaagacgttccgatttctacgattttaccatcctgtgggaataacctactgttctcgagaacaaggacaatggccatgttttaatcttaaaggacaccagagcatctggaattatgggtctccctactctttccactatgaatcttttggtttgccactg

SEQ ID NO: 889 - DNA, 137 bp, Mus musculus
  misc_feature (1)...(137): n = A,T,C or G
aatacatttcaactgtatgaaattggtttattgttcatcanaaatgttatttgccttcacaaagtctggtttcaatgaaccagctatgtaatgggatattaaataaacatggaattttacatgttcaaaaaaaaaaa

SEQ ID NO: 890 - DNA, 174 bp, Mus musculus
gaactgaggtatagctccatgaataacttaaacgaccccccaaattggaatatcaggcccaatgccagggctgatgggggtgatggaagcaagtggaattatgccctattggtgccaatgctcggattggcagcatttcgttggatttggtctagggaatcccaaaaaaaaaaa

SEQ ID NO: 891 - DNA, 188 bp, Mus musculus
tccttctgtgtgctgcagtcttgtggcgttctgcaggccccctgccagtgaccccaggctttttatggctgtgagactcattaaaagctcaggggtgagacgtgacctttggaggtgactataactgaagattgaattacagaagccaaaaaattaaataaataaaaggtaaaattataaaaaaaaaa